(19) United States
(12) Patent Application Publication
(10) Pub. No.: US 2014/0093901 A1
(43) Pub. Date: Apr. 3, 2014

(54) GENOME-SCALE METABOLIC NETWORK MODEL RECONSTRUCTION OF KLUYVEROMYCES MARXIANUS AND STRATEGIES FOR ENGINEERING NON-NATIVE PATHWAYS FOR 3-HYDROXYPROPIONATE PRODUCTION IN KLUYVEROMYCES MARXIANUS

(71) Applicants: Korea Advanced Institute Of Science and Technology, Daejeon (KR); Samsung Electronics Co., Ltd., Suwon-si (KR)

(72) Inventors: Kyu-sang LEE, Ulsan (KR); Tae-yong KIM, Daejeon (KR); Seung-bum SOHN, Daejeon (KR); Hyun-min KOO, Seoul (KR); Jae-chan PARK, Yongin-si (KR); Sang-yup LEE, Daejeon (KR)

(21) Appl. No.: 13/952,439

(22) Filed: Jul. 26, 2013

(30) Foreign Application Priority Data
Jul. 27, 2012 (KR) 10-2012-0082816

(51) Int. Cl. G06F 19/12; C12P 7/42

(52) U.S. Cl. CPC G06F 19/12; C12P 7/42
USPC 435/26; 435/34; 435/25; 435/254.2; 435/146; 702/19

(57) ABSTRACT

Use of a metabolic network model for analyzing metabolic characteristics of microorganisms for producing a metabolic product, such as 3HP enabling estimation of productivity and cell growth speed of microorganisms, optimizing new metabolic pathway, and providing transformed microorganisms that may produce a specific metabolic product with high efficiency.

### CROSS-REFERENCE TO RELATED APPLICATIONS

This application claims the benefit of Korean Patent Application No. 10-2012-0082816, filed on Jul. 27, 2012, in the Korean Intellectual Property Office, the entire disclosure of which is incorporated herein by reference.

### BACKGROUND

1. Field

The present disclosure relates to a metabolic network model for analyzing metabolical characteristics of a microorganism Kluyveromyces marxianus for producing 3-hydroxypropionate (3HP) and a method of estimating a new metabolic pathway that enhances productivity of 3HP using a simulation based on the model.

2. Description of the Related Art

In some instances, a desired substance may be produced more efficiently or inexpensively using a biological process (i.e., using microorganisms) instead of using a traditional chemical process.

However, if a specific metabolic product is excessively produced, the growth of microorganisms may be inhibited, the microorganisms may no longer produce the desired metabolic products, or the microorganisms may produce undesired byproducts.

Recently, there have been attempts to transform microorganisms using genetic engineering technologies for metabolic products that are originally not produced by the microorganisms, and to obtain the desired metabolic product from the transformed microorganisms. In one example, a nucleic acid that codes pyruvate carboxylase was introduced into E. coli to produce succinate in a high quantity. In another case, the activity of lactate dehydrogenase and phosphotransacetylase enzyme (PTA enzyme) was removed to produce succinate in a high quantity. However, it is difficult to estimate how metabolic pathways in microorganisms will change after the insertion or deletion of specific genes, and a lot of time may be needed to verify any changes. Hence, a lot of effort is spent in making microorganisms with desired characteristics.

Gene interactions may affect, e.g., protein expression, signaling pathways, and metabolism. The interaction of various elements of organisms is called a biological network, which may involve gene networks, protein-protein interactions, and signaling/metabolic pathways.

Proteins are products of gene expression. Multiple proteins may coordinate in a complex way to express functions, such as a form where two or more polypeptides are combined via an attraction among amino acids to form a protein complex, or proteins may function in a singular form. The connectivity among proteins is called protein-protein interaction, and this protein-protein interaction is a unit of expressing various functions for the survival of organisms, and is essential information to grasp or analyze the functions of genes.

A signaling/metabolic pathway is an aggregate of genes and proteins that performs a specific function, such as metabolism, movement, proliferation, survival, differentiation of cells, or movement of optic neurons, and includes the gene network, protein-protein interaction, or gene-protein interaction. A metabolic network model is a model of genes and proteins combined, including transcription factor proteins that control expression of genes in such a signaling/metabolic pathway.

Therefore, to produce desired substances using microorganisms and to estimate metabolic pathways more effectively, a method of using protein-protein interaction and signaling/metabolic networks is desired.

### SUMMARY

Provided are methods to estimate an optimized metabolic pathway that may produce a metabolic product in quantity by modifying a reaction network within a microorganism.

Provided are microorganisms having an estimated metabolic pathway through the optimized metabolic pathway.

Additional aspects will be set forth in part in the description which follows and, in part, will be apparent from the description, or may be learned by practice of the presented embodiments.

Unless otherwise defined, all technical and scientific terms used herein have the same meaning as commonly understood by one of ordinary skill. In general, the terms used in the present specification are well known in the art and are commonly used.

A method of identifying an optimized metabolic pathway for the production of a metabolic product by modifying a reaction network within a microorganism is provided.

The method includes: (a) acquiring a metabolic pathway in a microorganisms and a biomass synthesis equation (cell growth rate equation) using at least one information of a culture condition of microorganisms, metabolic products produced by microorganisms, and cell composition of microorganisms, based on a database including information on biochemical reactions in which enzymes are involved in a metabolic network within microorganisms; (b) providing a primary modified metabolic pathway by introducing a biochemical reaction pathway that does not exist in the acquired metabolic pathway; (c) providing a secondary modified metabolic pathway by modifying at least one enzyme reaction in the primary modified metabolic pathway; (d) acquiring information about a metabolic product and/or biomass produced by the secondary modified metabolic pathway; (e) acquiring a metabolic product-biomass correlation equation based on the acquired metabolic product information and/or biomass information; (f) repeating steps (c)-(e) for different enzyme reactions; and (g) identifying the secondary modified metabolic pathway that provides the optimum metabolic pathway for production of the metabolic product based on the product-biomass correlation equation.

The metabolic pathway estimating method is as follows:

First, an operation of acquiring a metabolic pathway in microorganisms and a biomass synthesis equation using at least one information of a culture condition of microorganisms, metabolic products produced by microorganisms, and cell composition of microorganisms, based on a database including information on biochemical reactions in which enzymes are involved in a metabolic network within microorganisms is provided.

The term “metabolic network” means a set of metabolic processes or physical steps that determine physiological and biochemical characteristics within cells. Also, such a network may include chemical reactions of metabolites and relationships between chemical reactions. For example, the network may include protein-protein interactions or enzyme operating mechanisms. The metabolic network may be a network of compounds and a network of enzymes at the same time.

The term, “information on biochemical reactions” means all information on reaction processes catalyzed by specific enzymes. This information may be collected from enzyme commission numbers, and in case of a newly discovered enzyme, this information may be acquired from experiments.

The term “culture condition” means a condition for culturing microorganisms. Culture conditions include, for example, a carbon source, a nitrogen source, or an oxygen condition used by the microorganisms. Carbon sources may include monosaccharides, disaccharides, or polysaccharides. Specifically, glucose, fructose, mannose, galactose, etc. may be used. Nitrogen sources may include organic nitrogen compounds, inorganic nitrogen compounds, and the like. Specifically, the nitrogen source may be amino acids, amides, amines, nitrates, and ammonium salts. Oxygen conditions may include an aerobic condition with normal oxygen partial pressure, a low-oxygen condition which includes about 0.1-about 10% oxygen in the atmosphere, or an anaerobic condition that does not include oxygen. The metabolic pathway may be modified according to the carbon sources and nitrogen sources that are actually usable by the microorganisms.

The term “metabolic product” means any substance produced by a metabolic reaction in a microorganism. The metabolic product may be an intermediate product of a metabolic reaction of a microorganism, or may be a final product of a metabolic reaction of a microorganism. Examples of the metabolic product may include succinate, lactate, lysine, threonine, arginine, 1,4-butanediol, 3-hydroxypropionate (3HP), and the like, but are not limited thereto.

The metabolic network model for analyzing metabolic characteristics of a microorganism is constructed as follows: First, genomic information and information on each gene annotation are collected, enzyme reaction equations that exist in the microorganism, for example, Kluyveromyces marxianus (K. marxianus) are organized based on the genomic sequence information, and relations of enzymes that perform the enzyme reaction equation and genes that code the enzyme, that is, a GPR relationship, is organized.

The term “GPR relationship” means a Gene-Protein-Reaction Relationship, which is a relationship among genes and products thereof, and the enzyme reaction equations that are performed by the enzymes coded by the genes to produce the products.

At this time, the collected gene annotation information may be directly analyzed. Also, the collected and analyzed information may be used as information of a metabolism-related database, the collected information may be used as a standard used during an adjustment process.

Based on the organized GPR relationship, a draft model is constructed. Generally, DNA replication, transcription, a translation and synthesis equation of various elements that constitute cells (such as phospholipids constituting a cell membrane, a cell wall, an amino acid content of all proteins in a cell, and an overall macromolecular composition of cells), and a biomass synthesizing equation of all the cells are not organized in the metabolism related database. However, these equations are essential for constructing models, and a composition ratio of each component that constitutes a cell's synthesis equation must be known to make such equations. This may be obtained through reference texts or may be identified by analyzing samples that are obtained through actual fermentation.

At this time, the fermentation condition may be equivalent to the fermentation condition that is used in the actual adjustment process. The constructed draft model is examined and adjustment work is performed. The metabolism-related database is constructed by analyzing gene annotation information with computer programs through a bioinformatics method. Hence, there is a high possibility for such database to have incomplete or wrong metabolism information, which is based on the following reasons:

First, the gene annotation information itself may be incomplete. This is because most of the gene annotation work is performed based on homology by comparing the gene sequences and amino acid sequences of enzymes with previously identified functions with the sequences to be analyzed using statistical methods. If there is an unidentified gene in a strain, it will be difficult to identify the functions of such a gene solely using bioinformatics methods.

Second, there is a possibility of error occurring in the process of automatically analyzing the gene annotation information and turning it into a metabolic pathway database. For example, when homology of an amino acid sequence among the genes of a strain is analyzed through BLAST analysis, it may show high homology with proteins of other strains, even though the function of this gene does not actually exist in the database.

Therefore, an adjustment is performed based on information acquired from published strain related articles, biochemistry related textbooks and texts, and traits discovered through actual fermentation, instead of information acquired from bioinformatics methods.

The following points mainly focus on adjustments.

1) Errors of a metabolic product coefficient in enzyme reactions

2) Cases where a metabolic pathway that produces elements of cells is lost

3) Maintenance energy used for maintaining life activity and growth of cells

4) Other typographical errors

Here, the maintenance energy of 3) is obtained through continuous chemostat culture. Continuous chemostat culture may maintain cell concentration at regular levels, and may also artificially control growth of cells by adjusting a dilution rate. In case such continuous culture is impossible, information about other strains may be used. However, the amount of maintenance energy may differ for strains, so information about strains with a close relationship should be used. Otherwise there is a high possibility of inaccurate results for the actual result and the metabolic flux analysis result.

To mathematically express the constructed metabolic network, a metabolic flux vector (metabolic flux of vj^{th}, j^{th} metabolic reaction) may be calculated using every metabolic product that forms the constructed metabolic network model, the metabolic products' metabolic pathway and stoichiometric matrix S in the metabolic pathway (Sij, time-dependent stoichiometric coefficient of i^{th} metabolic product in j^{th} reaction).

Herein, the time-dependent change of metabolic product concentration X may be expressed as a sum of flux of every metabolic reaction. If it is assumed that there is no time-dependent concentration change of X, that is, under the assumption of semi-normal status, the time-dependent change of metabolic product concentration may be defined as the following mathematics equation 1.

$$\frac{d X}{d t} = S \cdot v ( X ; k ) \qquad 〈 Mathematics equation 1 〉$$

(where Sv: Time-dependent change of X, X: Concentration of metabolic product, t: time, and k: constant)

From the formed stoichiometric matrix, a reaction of optimization, that is, to maximize or minimize, is set to be an object function and metabolic flux in cells is estimated using linear programming (Kim et al., Mol Biosyst. 4(2):113, 2008). Linear programming is a technique for the optimization of a linear objective function, subject to linear equality and linear inequality constraints. In an embodiment, an enzyme reaction related to producing compositions of cells was set to be an object function in a matrix S to optimize cell growth rate.

The linear programming for analyzing metabolic flux must be done under the assumption that only the nutrients used for the actual fermentation of the strain is supplied. It is very difficult to quantitatively identify the compositions of the components of the generally used complex medium, so the previously optimized synthetic medium may be used.

The term “biomass synthesis equation” means a general metabolic reaction of microorganisms. Specifically, it shows the relationships of proteins, nucleic acids, lipids, which are living body constituents, and cell biomass. The biomass synthesis equation is an unique value for microorganisms that vary depending on the applied microorganisms. In the case the microorganism is K. marxianus, the reaction may be the following reaction I.

0.56 PROTEIN+0.107 RNA+0.007 DNA+0.052 PHOSPHOLIPID+0.03 COF+0.110 CW+0.265 CARBOHYDRATE+70.37 ATP→BIOMASS+70.37 ADP+70.37 Pi [Reaction I]

A method to construct a trade-off curve that shows the relationship between metabolic substances and biomass is obtained by modifying algorithms suggested by the related art (Burgard et al., Biotechnol Bioeng 84, 647-57, 2003). Preferentially, a range of an accepted useful product forming rate is found by finding the values when the useful product forming rate is maximized and minimized. Next, a method of constructing a trade-off curve between two object functions by maximizing the biomass equation within the accepted range of the product forming rate is used. To identify the yield of useful products by considering the growth rate, a trade-off curve for a product formation rate and a specific growth rate was found under the consideration of two object functions needed for applying metabolic flux controlling technology and the result is shown in FIG. 1.

Also, the microorganisms may be wild type organisms existing in nature, transformed microorganisms, etc. Also, the microorganism may be K. marxianus.

Also, the method may include acquiring a primary modified metabolic pathway by introducing a biochemical reaction pathway that does not exist in the microorganism.

The term, “biochemical reaction pathway” means a biochemical reaction catalyzed by a specific enzyme. The biochemical reaction pathways may be catalyzed by one or more enzymes, and holoenzymes, coenzymes, and cofactors.

The term “primary modified metabolic pathway” means a metabolic pathway that is modified via introduction of new biochemical reaction pathways to metabolic pathways in microorganisms. Specifically, one or more enzymes that do not exist in microorganisms are introduced, and the enzymes may use intermediate products of metabolic reactions or final products of metabolic reactions of the microorganisms.

The biochemical reaction pathway that does not exist in the microorganisms may be at least one pathway selected from the group consisting of a malonyl-CoA pathway, a β-alanine pathway, and a glycerol pathway.

The introducing of the malonyl-CoA pathway is to introduce a metabolic pathway that produces malonyl-CoA (Malonyl-CoA pathway) from glucose. Malonyl-CoA is known to have a neutral oxidation/reduction relationship, but may not acquire ATP and may not use NADP (uses NADPH only). To introduce the pathway, one or more enzymes selected from the group consisting of 3-Hydroxyisobutyl-CoA hydrolase (EC 3.1.2.4), 3-Hydroxyisobutyrate dehydrogenase (EC 1.1.1.31), 3-Hydroxypropionyl-CoA hydrolase (EC 3.1.2.-), 3-Hydroxypropionyl-CoA dehydratase (EC 4.2.1.-), Acetyl-CoA carboxylase (EC 6.4.1.2), Aspartate decarboxylase (EC 4.1.1.11), CoA transferase (EC 2.8.3.1), Malonyl-CoA reductase (EC 1.1.1.-, 1.2.1.-), PEP carboxylase (EC 4.1.1.31), 3-oxopropanoate:NADP+oxidoreductase (EC 1.2.1.18) and 3-hydroxypropionate dehydrogenase (EC 1.1.1.59) may be introduced, and 3-oxopropanoate:NADP+oxidoreductase (EC 1.2.1.18) and 3-hydroxypropionate dehydrogenase (EC 1.1.1.59) may be introduced, and preferably, 3-oxopropanoate:NADP+oxidoreductase (EC 1.2.1.18) and 3-hydroxypropionate dehydrogenase (EC 1.1.1.59) may be introduced.

In order to introduce the above enzymes into a microorganisms, the genes coding the above enzymes may be introduced into the microorganisms.

The introducing of the β-alanine pathway is to introduce a metabolic pathway that produces β-alanine (β-alanine pathway) from glucose. The β-alanine pathway is known to have a neutral oxidation/reduction relationship, but may not acquire ATP. To introduce β-alanine pathway, one or more enzymes selected from the group consisting of 3-Hydroxyisobutyrate dehydrogenase (EC 1.1.1.31), 4-Aminobutyrate aminotransferase (EC 2.6.1.19), acetyl-CoA carboxylase (EC 6.4.1.2), aspartate aminotransferase (EC 2.6.1.1), aspartate decarboxylase (EC 4.1.1.11), glutamate dehydrogenase (EC 1.4.1.2), OS17 enzyme (EC 6.2.1.17), pyruvate carboxylase (EC 6.4.1.1), β-Alanyl-CoA ammonia lyase (EC 4.3.1.6), and 3-hydroxypropionate dehydrogenase (EC 1.1.1.59), and preferably 3-hydroxypropionate dehydrogenase (EC 1.1.1.59) may be introduced.

In order to introduce the above enzymes into a microorganisms, one or more genes coding the above enzymes may be introduced into the a microorganisms.

The glycerol pathway is a case of using glucose as a carbon source, running through 3-hydroxypropionaldehyde, and it is known to be a direct linear pathway from substrate to product. To introduce the glycerol pathway, glycerol dehydratase (EC 4.2.1.30) and aldehyde dehydrogenase (EC 1.2.1.3) may be introduced.

In order to introduce the above enzymes into microorganisms, one or more genes coding the above enzymes may be introduced into the microorganisms.

Also, the method may include acquiring a secondary modified metabolic pathway by modifying at least one enzyme reaction involved in the primary modified metabolic pathway.

The term “secondary modified metabolic pathway” means modifying at least one enzyme reaction related with the primary modified metabolic pathway. The terms of “transformation” or “modifying” of enzyme reaction may be reinforcing a reaction of enzymes by introducing enzymes, or removing one or more enzymes involved in the metabolic pathway.

One or more enzyme reactions may be added or removed for analyzing metabolic characteristics. The enzymes that may be used for transformation are one or more enzymes selected from the group consisting of: polyphosphate polyphosphohydrolase, diphosphate phosphohydrolase, urea-1-carboxylate amidohydrolase, acetolactate synthase, catalase, trehalase, pyruvate dehydrogenase, cytochrome c peroxidase, cellobiose glucohydrolase, porphobilinogen synthase, riboflavin synthase, ferrocytochrome-c:oxygen oxidoreductase, ferrocytochrome c2:oxygen oxidoreductase, benzenediol:oxygen oxidoreductase (laccase), hydroxymethylbilane synthase, ATP diphosphohydrolase, ATP synthase, mitochondrial, ATP synthase, vacuole, adenylate cyclase, ferric-chelate reductase (NADH), glutamate synthase (NADH), glutathione:NAD+oxidoreductase, cytochrome-b5 reductase, NAD+phosphohydrolase, NAD kinase, NADPH:ferricytochrome oxidoreductase, glutathione:NADP+oxidoreductase, ADP phosphohydrolase, adenosine tetraphosphate phosphodiesterase, ATP adenylyltransferase, adenylate kinase, dephospho-CoA kinase, carbonate dehydratase, ATP:nicotinamide-nucleotide adenylyltransferase, UDP phosphohydrolase, UMP kinase, UTP phosphohydrolase, FAD nucleotidohydrolase, FAD synthetase, pyridoxal kinase, L-methionine S-adenosyltransferase, S-adenosylmethionine decarboxylase, AMP aminohydrolase, Adenosine 5′-monophosphate phosphohydrolase, adenosine kinase, P1,P3-bis(5′-adenosyl)-triphosphate adenylohydrolase, adenosine 3′,5′-bisphosphate 3′-phosphohydrolase, NAD synthetase, AMP:diphosphate phospho-D-ribosyltransferase, adenosine 3′,5′-phosphate 5′-nucleotidohydrolase, S-Adenosyl-L-homocysteine hydrolase, S-lactate dehydrogenase (cytochrome), D-lactate dehydrogenase (cytochrome), pyruvate kinase, malic enzyme (NAD), malic enzyme (NADP), oxaloacetate carboxy-lyase, L-serine deaminase, pyruvate decarboxylase, S-acetolactate synthase, acetyl-CoA hydrolase, acetyl-CoA synthetase, acetyl-CoA acetyltransferase, glutamate 5-kinase, glutamate dehydrogenase (NAD+), L-Glutamate 5-semialdehyde:NAD+oxidoreductase, glutamate dehydrogenase (NADP+), 5-oxoprolinase (ATP-hydrolysing), N-carbamyl-L-glutamate amidohydrolase, L-Glutamine amidohydrolase, NAD synthetase (glutamine-hydrolysing), alanine transaminase, N-acteylglutamate synthase, mitochondrial (predicted), L-glutamate 1-carboxy-lyase, 2,5-dioxopentanoate:NADP+5-oxidoreductase, Isocitrate:NADP+oxidoreductase (decarboxylating), oxalosuccinate carboxy-lyase (2-oxoglutarate-forming), homocitrate synthase, glutathione:hydrogen-peroxide oxidoreductase, superoxide dismutase, Pyridoxamine-5′-phosphate:oxygen oxidoreductase (deaminating), pyridoxine 5-phosphate:oxygen oxidoreductase, UDP-glucose glucophosphohydrolase, UDP glucose pyrophosphorylase, UDP-glucose 4-epimerase, protoheme ferro-lyase (protoporphyrin-forming), ATP:acetate adenylyltransferase, guanosine-diphosphatase, guanylate kinase, GTP phosphohydrolase, phosphoenolpyruvate carboxykinase (ATP), malate dehydrogenase, pyruvate carboxylase, citrate synthase, L-aspartate transaminase, glycine:oxygen oxidoreductase (deaminating), alanine-glyoxylate aminotransferase, Glycine:2-oxoglutarate aminotransferase, succinate dehydrogenase, methylisocitrate lyase, UDP-N-acetylglucosamine diphosphorylase, UDP-N-acetyl-D-glucosamine 4-epimerase, GTP 7,8-8,9-dihydrolase, GTP cyclohydrolase II, GTP 8,9-hydrolase, succinate:CoA ligase (GDP-forming), GTP diphosphate-lyase, ureidoglycolate hydrolase, malate synthase, Isocitrate lyase, aspartate kinase (predicted), irreversible, L-aspartate:ammonia ligase, L-asparaginase, L-aspartate 1-carboxy-lyase (beta-alanine-forming), glutathione gamma-glutamylaminopeptidase, glutathione synthase, galactose 1-phosphate uridyltransferase, 3′-phosphoadenylyl-sulfate sulfohydrolase, 3′-Phospho-5′-adenylyl sulfate 3′-phosphohydrolase, adenylyl-sulfate kinase, cytidine-5′-monophosphate phosphohydrolase, cytidine 5′-phosphotransferase:ATP, CDP phosphohydrolase, cytidine 5′-phosphotransferase:UTP, cytidine 5′-phosphotransferase:GTP, S-Formylglutathione hydrolase, sulfate adenylyltransferase, pantothenate 4′-phosphotransferase, riboflavin-5-phosphate phosphohydrolase (acid optimum), riboflavin kinase, arginase, CTP phosphohydrolase, CTP synthase, UTP:L-glutamine amido-ligase, carbamoyl-phosphate synthase (glutamine-hydrolysing), asparagine synthase (glutamine-hydrolysing), O-phospho-L-serine phosphohydrolase, serine-pyruvate transaminase, L-Serine hydro-lyase, S-Adenosyl-L-methionine:tRNA guanine N2-methyltransferase, methanol:hydrogen-peroxide oxidoreductase, ATP:thiamine-diphosphate phosphotransferase, ATP:thiamine diphosphotransferase, 2-oxoglutarate dehydrogenase, L-galactonolactone oxidase, homocysteine S-methyltransferase, enolase, UTP:pyruvate 2-O-phosphotransferase, Ornithine transaminase, Ornithine Decarboxylase, L-arogenate hydro-lyase, L-Phenylalanine:2-oxoglutarate aminotransferase, Farnesyl-diphosphate:farnesyl-diphosphate farnesyltransferase, 1-pyrroline-5-carboxylate dehydrogenase, 1-pyrroline-5-carboxylate:NADP+oxidoreductase, isocitrate dehydrogenase (NAD+), acetaldehyde:NAD+oxidoreductase, acetaldehyde:NADP+oxidoreductase, succinate-semialdehyde:NAD+oxidoreductase, succinate-semialdehyde:NADP+oxidoreductase, saccharopine dehydrogenase (NAD+, L-lysine-forming), saccharopine dehydrogenase (NAD+, L-lysine-forming), mitochondria, ITP phosphohydrolase, ITP:pyruvate 2-O-phosphotransferase, succinate:CoA ligase (IDP-forming), L-tyrosine:2-oxoglutarate aminotransferase, acetyl-CoA:carbon-dioxide ligase, threonine aldolase, ethanol:NAD+oxidoreductase, D-fructose 6-phosphotransferase, D-fructose-6-phosphate amidotransferase, urea carboxylase, L-Cysteine hydrogen-sulfide-lyase, beta-fructofuranosidase-like protein, Succinyl-CoA:acetyl-CoA C-acyltransferase, 5-aminolevulinate synthase, D-glucose-6-phosphate:NADP+1-oxidoreductase, alpha,alpha-trehalose-phosphate synthase (UDP-forming), sn-glycerol-3-phosphate phosphohydrolase, glycerol-3-phosphate dehydrogenase, glycerol kinase, glycerol phosphate dehydrogenase (FAD), glycerol-3-phosphate O-acyltransferase, sulfite reductase (NADPH), hydrogen-sulfide:ferredoxin oxidoreductase, GDP-mannose mannophosphohydrolase, mannose-1-phosphate guanylyltransferase, glutamate-cysteine ligase, L-Cysteine:2-oxoglutarate aminotransferase, cysteine synthase, L-cysteine,glutathione:NADP+oxidoreductase (disulfide-forming), 3-aminopropanal:NAD+oxidoreductase, beta-alanine-pyruvate aminotransferase, beta-alanine:2-oxoglutarate aminotransferase, propanoate:CoA ligase (AMP-forming), propionyladenylate:CoA propionyltransferase, acetyl-CoA:propanoyl-CoA 2-C-acetyltransferase, 2-methylcitrate synthase, 5,6,7,8-tetrahydrofolate:NAD+oxidoreductase, 5,6,7,8-tetrahydrofolate:NADP+oxidoreductase, tetrahydrofolate:L-glutamate gamma-ligase, tetrahydrofolic formylase, 5,10-Methylenetetrahydrofolate:glycine hydroxymethyltransferase, ADP-glucose Glucose-1-phosphohydrolase, alpha-D-Glucose 1-phosphate 1,6-phosphomutase, IDP phosphohydrolase, cytidine 5′-phosphotransferase:ITP, Uridine 5′-monophosphate phosphohydrolase, uridine kinase (ATP:Uridine), orotidine-5′-phosphate carboxy-lyase (UMP-forming), uridine 5′-phosphotransferase:UTP, uridine 5′-phosphotransferase:GTP, uridine 5′-phosphotransferase:ITP, cytosine deaminase, anthranilate synthase (chorismate pyruvate-lyase), (2R,3S)-3-methylmalate:NAD+oxidoreductase, L-threonine ammonia-lyase, O-Succinyl-L-homoserine succinate-lyase (deaminating, L-cystathionine cysteine-lyase, dolichyldiphosphatase, dolichyl-phosphate beta-glucosyltransferase, UDP-N-acetyl-D-glucosamine:dolichyl-phosphate N-acetyl-D-glucosamine phosphotransferase, GDPMANNose:dolichyl-phosphate O-beta-D-mannosyltransferase, dolichyl-phosphate-mannose-protein mannosyltransferase, endoplasmic reticular, glycerone phosphate phosphohydrolase, glycerone kinase, glycerone-phosphate O-acyltransferase, triose-phosphate isomerase, dolichol kinase, choline kinase, sn-Glycero-3-phosphocholine glycerophosphohydrolase, glycerol:NAD+oxidoreductase, glycerol dehydrogenase (NADP+), ribose-phosphate diphosphokinase, ribokinase, ADP-ribose ribophosphohydrolase, pseudouridylate synthase, ribose-5-phosphate isomerase, D-Ribose 1,5-phosphomutase, D-glyceraldehyde-3-phosphate:NAD+oxidoreductase (phosphorylating), beta-D-fructose-1,6-bisphosphate D-glyceraldehyde-3-phosphate-lyase (glycerone-phosphate-forming), ATP phosphoribosyltransferase, am idophosphoribosyltransferase, anthranilate phosphoribosyltransferase, biotin:CoA ligase, biotin synthase, fumarate hydratase, adenylosuccinate lyase, argininosuccinate lyase, L-Leucine:2-oxoglutarate aminotransferase, galactokinase, galactan galactohydrolase, diphosphomevalonate decarboxylase, isopentenylpyrophosphate isomerase, inosine 5′-monophosphate phosphohydrolase, IMP cyclohydrolase, IMP dehydrogenase, IMP:diphosphate phospho-D-ribosyltransferase, adenylosuccinate synthase, dATP:pyruvate 2-O-phosphotransferase, agmatinase, L-Histidinol:NAD+oxidoreductase, butanoyl-CoA:oxygen 2-oxidoreductase, butanoyl-CoA:acetyl-CoA C-butanoyltransferase, ferredoxin-NADP+reductase, chitinase, isopropylmalate synthase, L-Valine:2-oxoglutarate aminotransferase, 2-dehydropantoate formaldehyde-lyase (3-methyl-2-oxobutanoate-forming), methylenetetrahydrofolate dehydrogenase (NAD+), methylenetetrahydrofolate dehydrogenase, glycine synthase, 5-methyltetrahydrofolate:NADP+oxidoreductase, 5,10-Methylenetetrahydrofolate:3-methyl-2-oxobutanoate hydroxymethyltransferase, guanosine 5′-monophosphate phosphohydrolase, GMP:diphosphate 5-phospho-alpha-D-ribosyltransferase, xanthosine-5′-phosphate:ammonia ligase, xanthosine-5′-phosphate:L-glutamine amido-ligase, GDPglucose sugarphosphohydrolase, guanosine 3′,5′-cyclic phosphate 5′-nucleotidohydrolase, arylsulfatase, adenosine ribohydrolase, pyrroline-5-carboxylate reductase, pyrroline-5-carboxylate reductase (NADPH), glutathione:L-amino-acid 5-glutamyltransferase, N-ribosylnicotinamide ribohydrolase, palmitoyl-CoA hydrolase, palmitoyl-CoA:oxygen 2-oxidoreductase, palmitate:CoA ligase, serine palmitoyltransferase, cystathionine L-homocysteine-lyase, L-cystathionine L-homocysteine-lyase, O-succinyl-L-homoserine succinate-lyase (adding hydrogen sulfide), chorismate pyruvate-lyase, phosphatidylcholine acylhydrolase, phospholipase D, phosphatidylcholine 2-acylhydrolase, S-Adenosyl-L-methionine:phosphatidyl-N-dimethylethanolamine N-methyltransferase, citrate hydroxymutase, ATP:D-mannose 6-phosphotransferase, phosphoglycolate phosphatase, L-Ornithine:2-oxo-acid aminotransferase, ATP:propanoate adenyltransferase, prephenate dehydratase, carbamoyl-phosphate:L-aspartate carbamoyltransferase, ornithine carbamoyltransferase, irreversible, 5′-methylthioadenosine nucleosidase, 5-Methylcytosine aminohydrolase, D-xylose reductase, cholesterol acyltransferase, O-phospho-L-homoserine phosphate-lyase, sn-Glycero-3-phosphoethanolamine glycerophosphohydrolase, sphingosine N-acyltransferase, UDP-glucose:N-acylsphingosine D-glucosyltransferase, phosphoglycerate kinase, 3-Phospho-D-glycerate:NAD+2-oxidoreductase, phosphoglycerate mutase, D-ribulokinase, phosphogluconate dehydrogenase, ribulose 5-phosphate 3-epimerase, arabinose-5-phosphate isomerase, ATP:dAMP phosphotransferase, cytidine 5′-phosphotransferase:dATP, uridine 5′-phosphotransferase:dATP, adenosine aminohydrolase, adenosine:phosphate alpha-D-ribosyltransferase, thymidylate 5′-phosphohydrolase, D-arabinose 1-dehydrogenase [NAD(P)+], beta-D-glucose 6-phosphotransferase, aldose 1-epimeras, lysine N-acetyltransferase, acetyl-CoA:[acyl-carrier-protein] S-acetyltransferase, Malonyl-CoA:[acyl-carrier-protein] S-malonyltransferase, xylulokinase, sedoheptulose-7-phosphate:D-glyceraldehyde-3-phosphate glycolaldehyde transferase, 4-aminobutanoate:2-oxoglutarate aminotransferase, spontaneous, 10-Formyltetrahydrofolate:L-glutamate ligase, methenyltetrahydrofolate cyclohydrolase, geranyl pyrophosphate synthase, 3-phospho-D-glycerate 1,2-phosphomutase, dCMP aminohydrolase, 2′-Deoxycytidine 5′-monophosphate phosphohydrolase, guanine aminohydrolase, guanosine ribohydrolase, lactose galactohydrolase, 3-Sulfo-L-alanine carboxy-lyase (taurine-forming), dihydrolipoamide:NAD+oxidoreductase, pyridoxine 4-dehydrogenase, pyridoxamine:oxygen oxidoreductase (deaminating), pyridoxine:oxygen oxidoreductase (deaminating), chorismate synthase, chorismate mutase, chorismate:L-glutamine aminotransferase, nicotinate D-ribonucleotide:diphosphate phosphoribosyltransferase, prephenate dehydrogenase, (R)—S-Lactoylglutathione hydrolase, gluconokinase, D-Glyceraldehyde:NAD+oxidoreductase, inosine ribohydrolase, homoserine kinase, L-Homoserine:NAD+oxidoreductase, L-Homoserine:NADP+oxidoreductase, homoserine acetyltransferase, glucokinase, dextrin 6-alpha-D-glucanohydrolase, 5,6,7,8-tetrahydrobiopterin:NAD+oxidoreductase, 5,6,7,8-tetrahydrobiopterin:NADP+oxidoreductase, CTP:phosphatidate cytidyltransferase, phosphatidylserine synthase, glycerophosphate phosphatidyltransferase, phosphatidylinositol synthase, D-mannose 1,6-phosphomutase, 3-deoxy-D-arabino-heptulosonate 7-phosphate synthetase, sedoheptulose-7-phosphate:D-glyceraldehyde-3-phosphate glyceronetransferase, beta-D-Fructose 6-phosphate:D-glyceraldehyde-3-phosphate glycolaldehyde transferase transketolase, sedoheptulose 7-phosphate 1-phosphotransferase:ATP, ATP:dGDP phosphotransferase, dGTP:pyruvate 2-O-phosphotransferase, purine-nucleoside phosphorylase, dihydroorotate dehydrogenase (Fumarate dependent), orotate phosphoribosyltransferase, cytidine aminohydrolase, uridine 5′-phosphotransferase:dGTP, choline-phosphate cytidylyltransferase, D-xylulose reductase, isocitrate:NADP+oxidoreductase, pyridoxine 5′-phosphotransferase, spermidine synthase, sphingosine kinase, thiosulfate sulfurtransferase, 2-oxoadipate dehydrogenase complex, homoisocitrate dehydrogenase, (1R,2S)-1-hydroxybutane-1,2,4-tricarboxylate:NAD+oxidoreductase (decarboxylating), L-2-aminoadipate:2-oxoglutarate aminotransferase, argininosuccinate synthase, L-Kynurenine:2-oxoglutarate aminotransferase, ATP:D-glucosamine 6-phosphotransferase, 2′-Deoxyguanosine 5′-monophosphate phosphohydrolase, deoxyguanosine:orthophosphate ribosyltransferase, (S)-3-Hydroxybutanoyl-CoA:NAD+oxidoreductase, hydroxymethylglutaryl-CoA synthase, 4-Aminobutyraldehyde:NADP+oxidoreductase, dihydroorotase, farnesyl pyrophosphate synthetase, NADPH:oxidized-thioredoxin oxidoreductase, 2′-Deoxyadenosine 5′-diphosphate:oxidized-thioredoxin 2′-oxidoreductase, 2′-Deoxyuridine 5′-diphosphate:oxidized-thioredoxin 2′-oxidoreductase, 2′-Deoxyguanosine 5′-diphosphate:oxidized-thioredoxin 2′-oxidoreductase, 2′-Deoxycytidine diphosphate:oxidized-thioredoxin 2′-oxidoreductase, phosphoadenylyl-sulfate reductase (thioredoxin, phosphogluconolactonase, CTP:ethanolamine-phosphate cytidylyltransferase, phosphatidylethanolamine phosphatidohydrolase, phosphatidylethanolamine 2-acylhydrolase, phosphatidylserine decarboxylase, phosphatidylethanolamine methyltransferase, glucosamine-phosphate N-acetyltransferase, geranylgeranyl pyrophosphate synthase, (R)-Mevalonate:NADP+oxidoreductase (CoA acylating), 2′-Deoxyadenosine 5′-monophosphate phosphohydrolase, cytidine 5′-phosphotransferase:dGTP, dTDP phosphohydrolase, ATP:dTDP phosphotransferase, thymidylate kinase, dTTP nucleotidohydrolase, cytidine 5′-phosphotransferase:dTTP, uridine 5′-phosphotransferase:dTTP, uridylate kinase (dUMP), dUTP diphosphatase, thymidylate synthase, 2′-Deoxyuridine 5′-monophosphate phosphohydrolase, urate oxidase, UDP-glucose-sterol glucosyltransferase, retinol:NAD+oxidoreductase, XMP:pyrophosphate phosphoribosyltransferase, guanosine:phosphate alpha-D-ribosyltransferase, cytochrome 2 reductase, NADH dehydrogenase, succinate dehydrogenase (ubiquinone), S-adenosyl-L-methionine:2-hexaprenyl-3-methyl-5-hydroxy-6-methoxy-1,4-benzoquinone 3-O-methyltransferase, L-Isoleucine:2-oxoglutarate aminotransferase, stearoyl-CoA 9-desaturase, dihydrofolate:NAD+oxidoreductase, dihydrofolate:NADP+oxidoreductase, 7,8-dihydropteroate:L-glutamate ligase, 1,2-Diacyl-sn-glycerol 3-phosphate phosphohydrolase, acyl-CoA:1-acyl-sn-glycerol-3-phosphate 2-O-acyltransferase, mevalonate kinase, triacylglycerol acylhydrolase, 1,2-diacylglycerol acyltransferase, 5,6-Dihydrouracil amidohydrolase, N2-Acetyl-L-ornithine:L-glutamate N-acetyltransferase, acteylornithine transaminase, irreversible, mitochondrial, aspartate-semialdehyde dehydrogenase, irreversible, N-Ribosylnicotinamide:orthophosphate ribosyltransferase, nicotinate D-ribonucleoside:orthophosphate ribosyltransferase, Xanthosine:orthophosphate ribosyltransferase, S-Aminomethyldihydrolipoylprotein:(6S)-tetrahydrofolate aminomethyltransferase (ammonia-forming), formyltetrahydrofolic cyclodehydrase, saccharopine dehydrogenase (NADP+, L-glutamate-forming), ATP:dCDP phosphotransferase, uridine 5′-phosphotransferase:dCTP, uridine 5′-phosphotransferase:dUTP, chitin amidohydrolase, chitin synthase, (1S,2R)-1-C-(indol-3-yl)glycerol 3-phosphate D-glyceraldehyde-3-phosphate-lyase, N-D-ribosylpurine ribohydrolase, xanthosine ribohydrolase, NADPH:quinone reductase, cytidine 5′-phosphotransferase:dCTP, cytidine 5′-phosphotransferase:dUTP, Tyramine:o2 oxidoreductase(deaminating)(flavin-containing), carnitine O-acetyltransferase, butyrobetaine hydroxylase, Itaconate:CoA ligase (ADP-forming), Itaconate:CoA ligase (GDP-forming), Itaconate:CoA ligase (IDP-forming), L-Cystine L-Cysteine-lyase, raffinose fructohydrolase, shikimate kinase, shikimate dehydrogenase, ubiquitin thiolesterase, allantoate amidinohydrolase, allantoin amidohydrolase, allantoinase, L-cysteate:2-oxoglutarate aminotransferase, L-ribulokinase, L-arabinitol 2-dehydrogenase, D-Ornithine:oxygen oxidoreductase (deaminating), sphinganine-1-phosphate pamlmitaldehyde-lyase, 3-Sulfino-L-alanine carboxy-lyase, 2-dehydropantoate 2-reductase, pantothenate synthetase, holocytochrome-c synthase, deoxyuridine:orthophosphate 2-deoxy-D-ribosyltransferas, deoxycytidine aminohydrolase, pyridoxal 5′-phosphotransferase, aminoacetone:oxygen oxidoreductase(deaminating), lactoylglutathione lyase, phenylacetaldehyde:NAD+oxidoreductase, phenylacetaldehyde:NADP+oxidoreductase, 2-phenylacetamide amidohydrolase, acyl-CoA:sphingosine N-acyltransferase, 4-aminobutyraldehyde:NAD+oxidoreductase, deoxyadenosine aminohydrolase, deoxyadenosine:orthophosphate ribosyltransferase, D-Fructose 1-phosphate D-glyceraldehyde-3-phosphate-lyase, acetyl-CoA:dihydrolipoamide S-acetyltransferase, dihydrolipoamide succinyltransferase, Glutaryl-CoA:dihydrolipoamide S-succinyltransferase, 2-Deoxy-D-glucose 6-phosphate phosphohydrolase, phenethylamine:oxygen oxidoreductase (deaminating), 2,3-Dehydroacyl-CoA:sn-glycerol-3-phosphate O-acyltransferase, 3-sulfino-L-alanine:2-oxoglutarate aminotransferase, acetylglutamate kinase, acetylglutamate kinase, mitochondrial, ADPmannose sugarphosphohydrolase, Indole-3-acetaldehyde:NAD+oxidoreductase, (3S)-3-Hydroxyacyl-CoA hydro-lyase, 1,2-diacyl-sn-glycerol acylhydrolase, 4-Hydroxyphenylacetaldehyde:NAD+oxidoreductase, 4-Hydroxyphenylacetaldehyde:NADP+oxidoreductase, D-arabinono-1,4-lactone oxidase, Xanthosine 5′-phosphate phosphohydrolase, tryptophan synthase (indoleglycerol phosphate), fructose-2,6-bisphosphatase, phosphofructokinase 2, beta-D-Glucose-6-phosphate:NADP+1-oxoreductase, glucose-6-phosphate 1-epimerase, alpha-D-Glucose 6-phosphate ketol-isomerase, 1-Acyl-sn-glycero-3-phosphocholine acylhydrolase, 2-Acyl-sn-glycero-3-phosphocholine acylhydrolase, deoxyinosine:orthophosphate ribosyltransferase, acylglycerone-phosphate reductase, trehalose-phosphatase, D-O-Phosphoserine phosphohydrolase, sirohydrochlorin ferrochelatase, D-hexose 6-phosphotransferase, S-adenosylmethioninamine:spermidine 3-aminopropyltransferase, presqualene diphosphate:farnesyl-diphosphate farnesyltransferase, squalene monooxygenase, 1,4-beta-D-Glucan glucohydrolase, D-Proline:oxygen oxidoreductase, creatinine iminohydrolase, D-Arginine:oxygen oxidoreductase (deaminating), 2-Propyn-1-al:NAD+oxidoreductase, (R,R)-butanediol dehydrogenase, D-Glucuronolactone:NAD+oxidoreductase, NAD(P)H dehydrogenase (quinone), ATP:sphinganine 1-phosphotransferase, 3-dehydrosphinganine reductase, dTDP glucose 4-epimerase, Deamino-NAD+nucleotidohydrolase, histidinol-phosphatase, pantothenate kinase, 4-Nitrophenyl phosphate phosphohydrolase, (S)-3-Hydroxybutanoyl-CoA hydro-lyase, pantetheine-phosphate adenylyltransferas, Dephospho-CoA nucleotidohydrolase, 3-Hydroxypropionyl-CoA hydro-lyase, 2-Acetolactate pyruvate-lyase, acetohydroxy acid isomeroreductase, mitochondrial, 5,6-Dihydrothymine amidohydrolase, leukotriene-A4 hydrolase, 4-aminobenzoate synthase, 2-amino-4-hydroxy-6-hydroxymethyl-7,8-dihydropteridine:4-aminobenzoate 2-amino-4-hydroxydihydropteridine-6-methenyltransferase, 2-amino-4-hydroxy-6-hydroxymethyl-7,8-dihydropteridine-diphosphate:4-aminobenzoate 2-amino-4-hydroxydihydropteridine-6-methenyltransferase, 3-dehydroquinate synthase, 3-dehydroquinate dehydratase, indole-3-acetamide amidohydrolase, L-2-aminoadipate 6-O-adenylyltransferase, L-2-Aminoadipate-6-semialdehyde:NAD+6-oxidoreductase, L-2-Aminoadipate-6-semialdehyde:NADP+6-oxidoreductase, O3-Acetyl-L-serine acetate-lyase (adding hydrogen sulfide), primary-amine oxidase, 3-hydroxyisobutyryl-CoA hydrolase, uroporphyrinogen-III synthase, 4-Guanidinobutanamide amidohydrolase, dethiobiotin synthetase, S-adenosyl-L-methionine:uroporphyrin-III C-methyltransferase, uroporphyrinogen decarboxylase, lanosterol synthase, O-Acetyl-L-homoserine succinate-lyase (adding cysteine), coproporphyrinogen III oxidase, protoporphyrinogen oxidase, thiamine-phosphate diphosphorylase, (3R)-3-Hydroxyacyl-CoA hydro-lyase, 7,8-diaminonanoate transaminase, D-tagatose-6-phosphate 1-phosphotransferase:ATP, 5-Amino-2-oxopentanoate:2-oxoglutarate aminotransferase, phosphomevalonate kinase, O-Succinyl-L-homoserine succinate-lyase (adding cysteine), phosphopantothenoylcysteine decarboxylase, 4-Trimethylammoniobutanal:NAD+oxidoreductase, L-hydroxyproline reductase (NAD), L-hydroxyproline reductase (NADP), 3,4-Dihydroxyphenylacetaldehyde:NAD+oxidoreductase, 3,4-Dihydroxyphenylacetaldehyde:NADP+oxidoreductase, lathosterol oxidase, glutamate-5-semialdehyde dehydrogenase, ATP:pseudouridine 5′-phosphotransferase, beta-D-Glucose 6-phosphate ketol-isomerase, nicotinate D-ribonucleotide phosphohydrolase, 3-Hydroxy-2-methylpropanoyl-CoA hydrolase, beta-D-Galactosyl-1,4-beta-D-glucosylceramide galactohydrolase, 1-Palmitoylglycerol-3-phosphate:NADP+oxidoreductase, 1-phosphatidylinositol 4-kinase, phosphatidylinositol 3-kinase, D-myo-Inositol-1,4,5-trisphosphate 5-phosphohydrolase, guanosine 3′-diphosphate 5′-triphosphate 5′-phosphohydrolase, S-Adenosyl-L-methionine:unsaturated-phospholipid methyltransferase (cyclizing), 1-Acyl-sn-glycero-3-phosphoethanolamine aldehydrohydrolase, L-2-Lysophosphatidylethanolamine aldehydrohydrolase, S-Adenosyl-L-methionine:phosphatidyl-N-methylethanolamine N-methyltransferase, glycine decarboxylase, D-myo-Inositol 1,3,4,5-tetrakisphosphate 5-phosphohydrolase, phosphoinositide phospholipase C, N-acetyl-g-glutamyl-phosphate reductase, irreversible, mitochondrial, imidazoleglycerol-phosphate dehydratase, 5-amino-6-(5-phosphoribitylamino)uracil:NADP+1′-oxidoreductase, diaminohydroxyphosphoribosylaminopyrimidine deaminase, 3-phosphoshikimate 1-carboxyvinyltransferase, irreversible, 1-phosphatidylinositol-4-phosphate 5-kinase, 4-Carboxymuconolactone carboxy-lyase, hydroxymethylpyrimidine kinase, N-acetylglucosaminylphosphatidylinositol deacetylase, 2-amino-4-hydroxy-6-hydroxymethyldihydropteridine diphosphokinase, dihydroneopterin aldolase, indole-3-glycerol-phosphate synthase, phosphoribosylanthranilate isomerase (irreversible), RX:glutathione R-transferase, D-Glucoside glucohydrolase, Hydrogen selenide:NADP+oxidoreductase, selenocysteine lyase, stachyose fructohydrolase, alanine tRNA synthetase, arginine-tRNA synthetase, asparagine-tRNA synthetase, cysteine-tRNA synthetase, glutamine-tRNA synthetase, glycine-tRNA synthetase, histidine-tRNA synthetase, isoleucine-tRNA synthetase, leucine-tRNA synthetase, lysine-tRNA synthetase, methionine-tRNA synthetase, phenylalanine-tRNA synthetase, proline-tRNA synthetase, serine-tRNA synthetase, threonine-tRNA synthetase, tryptophan-tRNA synthetase, L-tyrosine:tRNATyr ligase, valine-tRNA synthetase, aspartate-tRNA synthetase, glutamate-tRNA synthetase, methionyl-tRNA synthetase, glycogenin glucosyltransferase, beta-Lactamhydrolase, octanoyl-CoA:oxygen 2-oxidoreductase, octanoyl-CoA:acetyl-CoA C-acyltransferase, dihydrolipoylprotein:NAD+oxidoreductase, Lauroyl-CoA:(acceptor) 2,3-oxidoreductase, Lauroyl-CoA:acetyl-CoA C-acyltransferase, (S)-Methylmalonate semialdehyde:NAD+oxidoreductase, [cytochrome c]-lysine N-methyltransferase, 4-Carboxymethylenebut-2-en-4-olide lactonohydrolase, (5-Glutamyl)-peptide:amino-acid 5-glutamyltransferase, sucrose 6-phosphate fructohydrolase, methionyl-tRNA formyltransferase, precorrin-2 dehydrogenase, 2-Isopropylmalate hydro-lyase, gamma-Glutamyl-beta-aminopropiononitrile amidohydrolase, estrone 3-sulfate sulfohydrolase, tetradecanoyl-CoA:(acceptor) 2,3-oxidoreductase, myristoyl-CoA:acetylCoA C-myristoyltransferase, glycylpeptide N-tetradecanoyltransferase, 3-Isopropylmalate hydro-lyase, phosphoribosyl-ATP diphosphatase, 1-(5-phospho-D-ribosyl)-AMP 1,6-hydrolase, glutaminyl-peptide cyclotransferase, imidazole acetaldehyde:NAD+oxidoreductase, S-(2-Hydroxyacyl)glutathione hydrolase, peptide-L-methionine:thioredoxin-disulfide S-oxidoreductase [L-methionine (S)—S-oxide-forming], 3-Hydroxyisopentyl-CoA hydro-lyase, phosphoribosylglycinamide synthetase, glutamyl transpeptidase, 1-Alkyl-2-acyl-sn-glycero-3-phosphate phosphohydrolase, (S)-3-Hydroxydodecanoyl-CoA hydro-lyase, 3-Hydroxy-L-kynurenine:2-oxoglutarate aminotransferase, phosphoserine transaminase, L-Alanine:3-oxopropanoate aminotransferase, (2S,3S)-3-hydroxy-2-methylbutanoyl-CoA:NAD+oxidoreductase, (2S,3S)-3-Hydroxy-2-methylbutanoyl-CoA hydro-liase, phosphoribosylformylglycinamidine cyclo-ligase, phosphoribosylaminoimidazole carboxylase, dolichyl-diphosphooligosaccharide-protein glycosyltransferase 37 kDa, beta, gamma, alpha, and epsilon subunit, cis-4-Hydroxy-D-proline:oxygen oxidoreductase (deaminating), (S)-3-Hydroxyisobutyryl-CoA hydro-lyase, (R)-4′-Phosphopantothenate:L-cysteine ligase, tetrahydrofolyl-[Glu](n):L-glutamate gamma-ligase, 4-(2-Aminoethyl)-1,2-benzenediol:o2 oxidoreductase(deaminating)(flavin-containing), 2,4-dienoyl-CoA reductase (NADPH), phosphoribosylglycinamide formyltransferase, homoaconitate hydratase, 1-(5′-Phosphoribosyl)-5-amino-4-imidazolecarboxamide:pyrophosphate phosphoribosyltransferase, L-2-Aminoadipate-6-semialdehyde:NAD(P)+6-oxidoreductase, 5-Methyltetrahydropteroyltri-L-glutamate:L-homocysteine S-methyltransferase, methionine synthase, methylthioribose 1-phosphate isomerase, 2-methylcitrate dehydratase, (2S,3R)-3-Hydroxybutane-1,2,3-tricarboxylate hydro-lyase, 3-Isopropylmalate:NAD+oxidoreductase, S-Adenosyl-L-methionine:zymosterol C-methyltransferase, (R)-2,3-Dihydroxy-3-methylbutanoate:NADP+oxidoreductase (isomerizing), (R)-2,3-Dihydroxy-3-methylbutanoate hydro-lyase, L-1-pyrroline-3-hydroxy-5-carboxylate dehydrogenase, L-1-pyrroline-3-hydroxy-5-carboxylate dehydrogenase (NADPH), hydroxyethylthiazole kinase, 1-Alkyl-2-acetyl-sn-glycero-3-phosphocholine acetohydrolase, phosphoribosylformylglycinamidine synthase, 4,4-dimethyl-5alpha-cholest-7-en-3beta-ol, NADH:oxygen oxidoreductase (hydroxylating), GDPMANNose:chitobiosyldiphosphodolichol beta-D-mannosyltransferase, 3alpha,7alpha-Dihydroxy-5beta-cholestan-26-al:NAD+oxidoreductase, ATP:4-amino-2-methyl-5-phosphomethylpyrimidine phosphotransferase, Imidazole-glycerol-3-phosphate synthase, 1-(5′-Phosphoribosyl)-5-amino-4-(N-succinocarboxamide)-imidazole AMP-lyase, AICAR transformylase, biotin-[acetyl-CoA-carboxylase] ligase, biotin-[methylmalonyl-CoA-carboxytransferase] ligase, biotin:apo-[propionyl-CoA:carbon-dioxide ligase, SAICAR synthetase, biotin-[methylcrotonoyl-CoA-carboxylase] ligase, 2-Amino-4-hydroxy-6-(erythro-1,2,3-trihydroxypropyl)dihydropteridine triphosphate phosphohydrolase (alkaline optimum), D-Galactosyl-N-acetyl-D-galactosaminyl-(N-acetylneuraminyl)-D-galactosyl-D-glucosylceramide galactohydrolase, 2-Amino-4-hydroxy-6-(erythro-1,2,3-trihydroxypropyl)dihydropteridine triphosphate hydrolase, phosphoribosylformiminoaminophosphoribosylimidazolecarboxamide isomerase, (S)-2-Acetolactate pyruvate-lyase, (S)-2-Aceto-2-hydroxybutanoate pyruvate-lyase, 5-adenosyl-L-methionine:3-hexaprenyl-4,5-dihydroxylate O-methyltransferase, 4a-hydroxytetrahydrobiopterin hydro-lyase, (S)-3-Hydroxyhexadecanoyl-CoA:NAD+oxidoreductase, (S)-3-Hydroxyhexadecanoyl-CoA hydro-lyase, (S)-3-Hydroxytetradecanoyl-CoA:NAD+oxidoreductase, (S)-3-Hydroxytetradecanoyl-CoA hydro-lyase, (S)-3-hydroxydodecanoyl-CoA:NAD+oxidoreductase, (S)-hydroxydecanoyl-CoA:NAD+oxidoreductase, (S)-hydroxyoctanoyl-CoA:NAD+oxidoreductase, (S)-Hydroxyoctanoyl-CoA hydro-lyase, (S)-hydroxyhexanoyl-CoA:NAD+oxidoreductase, (S)-Hydroxyhexanoyl-CoA hydro-lyase, Hexanoyl-CoA:(acceptor) 2,3-oxidoreductase, Decanoyl-CoA:(acceptor) 2,3-oxidoreductase, selenomethionine methanethiol-lyase (deaminating), L-selenomethione S-adenosyltransferase, phosphofructokinase, fructose-bisphosphatase, 3-Ketolactose galactohydrolase, alcohol dehydrogenase, 3alpha,7alpha,24-trihydroxy-5beta-cholestanoyl-CoA:NAD+oxidoreductase, 3alpha,7alpha,12alpha,24-tetrahydroxy-5beta-cholestanoyl-CoA:NAD+oxidoreductase, sulfatide sulfohydrolase, O3-acetyl-L-serine:thiosulfate 2-amino-2-carboxyethyltransferase (reducing, L-cysteine-forming), 3,4-dihydroxyphenylethyleneglycol:NAD+oxidoreductase, 3,4-Dihydroxymandelaldehyde:NAD+oxidoreductase, 3,4-Dihydroxymandelaldehyde:NADP+oxidoreductase, 3-Methoxy-4-hydroxyphenylacetaldehyde:NAD+oxidoreductase, 3-Methoxy-4-hydroxyphenylacetaldehyde:NADP+oxidoreductase, 3-Methoxy-4-hydroxyphenylglycolaldehyde:NAD+oxidoreductase, 3-Methoxy-4-hydroxyphenylglycolaldehyde:NADP+oxidoreductase, 5-Hydroxyindoleacetaldehyde:NAD+oxidoreductase, ATP:adenylylsulfate 3′-phosphotransferase, selenate adenylyltransferase, selenocystathionine Lysteine-lyase (deaminating), (5-L-glutamyl)-peptide:Se-Methylselenocysteine 5-glutamyltransferase, Se-Adenosylselenohomocysteine hydrolase, selenocystathionine L-homocysteine-lyase, O-phosphorylhomoserine phosphate-lyase (adding selenocysteine), O-succinyl-L-homoserine succinate-lyase (adding selenocysteine), Cyanoglycoside glucohydrolase, Uroporphyrinogen I carboxy-lyase, 3-Methylimidazole acetaldehyde:NAD+oxidoreductase, beta-D-Glucosyl-2-coumarinate glucohydrolase, polyisopentenylpyrolinate:4-hydroxybenzoate nonaprenyltransferase, GDPMANNose:glycolipid 1,3-alpha-D-mannosyltransferase, formamidopyrimidine nucleoside triphosphate amidohydrolase, 2,5-Diaminopyrimidine nucleoside triphosphate mutase, N4-Acetylaminobutanal:NAD+oxidoreductase, L-erythro-4-Hydroxyglutamate:NAD+oxidoreductase, L-erythro-4-Hydroxyglutamate:2-oxoglutarate aminotransferase (predicted), (S)-3-Hydroxyisobutyryl-CoA hydrolase, 3-Hydroxy-2-methylpropanoate:NAD+oxidoreductase, ketol-acid reductoisomerase (2-Aceto-2-hydroxybutanoate), mitochondrial, (S)-2-Aceto-2-hydroxybutanoate:NADP+oxidoreductase (isomerizing), (R)-2,3-Dihydroxy-3-methylpentanoate hydro-lyase, (S)-2-Acetolactate methylmutase, O-Phospho-4-hydroxy-L-threonine:2-oxoglutarate aminotransferase, O-Phospho-4-hydroxy-L-threonine phospho-lyase, NADPH:cytochrome-P-450 oxidoreductase, Gal-beta1->3GalNAc-beta1->4Gal-beta1->4Glc-beta1->1′Cer galactohydrolase, Biotinyl-5′-AMP:apo-[carboxylase] ligase, trans-3-Chloro-2-propene-1-ol:NAD+oxidoreductase, cis-3-chloro-2-propene-1-ol:NAD+oxidoreductase, aldehyde dehydrogenase, Chloroacetaldehyde:NAD+oxidoreductase, Spermidine:e1F5A-lysine 4-aminobutyltransferase (propane-1,3-diamine-forming), phospholipid:diacylglycerol acyltransferase, protoanemonin lactonohydrolase, amidase, aminodeoxychorismate lyase, (3S)-3-Hydroxyacyl-CoA:NAD+oxidoreductase, acylamide aminohydrolase, flavin-containing monooxygenase, 4,4-dimethyl-5a-cholesta-8,24-dien-3b-ol:NADP+D14-oxidoreductase, ergosterol:NADP+D24(241)-oxidoreductase, nitric oxide dioxygenase, 1-phosphatidylinositol-3-phosphate 5-kinase, Non-enzymatic, D-alanine:oxygen oxidoreductase (deaminating), GDP-Man:Man1GlcNAc2-PP-Dol α-1,3-mannosyltransferase, ALG2 (gene name, ambiguous), mannosyl-oligosaccharide glucosidase, mannosyl-oligosaccharide 1,2-alpha-mannosidase, methylamine:oxygen oxidoreductase (deaminating) (copper-containing), L-Allothreonine acetaldehyde-lyase, squalene synthase, penicillin hydrolase, Aldehyde:NAD+oxidoreductase, cis-2-Methyl-5-isopropylhexa-2,5-dienoyl-CoA hydro-lyase, sphingosine-1-phosphate palmitaldehyde-lyase, 3-sn-phosphatidate phosphohydrolase, sedoheptulose-7-phosphate:D-glyceraldehyde-3-phosphate glycolaldehydetransferase, alpha 1,2-mannosyloligosaccharide alpha-D-mannohydrolase, D-xylulose-5-phosphate:thiamin diphosphate glycolaldehydetransferase, D-Sedoheptulose-7-phosphate:thiamin diphosphate glycolaldehydetransferase, 1-hydroxymethylnaphthalene:NAD+oxidoreductase, (2-naphthyl)methanol:NAD+oxidoreductase, (S)-3-hydroxyacyl-CoA:NAD+oxidoreductase, S-(hydroxymethyl)glutathione dehydrogenase, (1R)-hydroxy-(2R)-glutathionyl-1,2-dihydronaphthalene glutathione-lyase (epoxide-forming), (1R)-glutathionyl-(2R)-hydroxy-1,2-dihydronaphthalene glutathione-lyase (epoxide-forming), (1S)-hydroxy-(2S)-glutathionyl-1,2-dihydronaphthalene glutathione-lyase (epoxide-forming), Spontaneous-1-Naphthol, (1R,2S)-naphthalene 1,2-oxide hydrolase, (1S,2R)-naphthalene 1,2-oxide hydrolase, 1-nitro-7-hydroxy-8-glutathionyl-7,8-dihydronaphthalene glutathione-lyase (epoxide-forming), 1-nitro-7-glutathionyl-8-hydroxy-7,8-dihydronaphthalene glutathione-lyase (epoxide-forming), 1-nitro-5-hydroxy-6-glutathionyl-5,6-dihydronaphthalene glutathione-lyase (epoxide-forming), 1-nitro-5-glutathionyl-6-hydroxy-5,6-dihydronaphthalene glutathione-lyase (epoxide-forming), 1-nitronaphthalene-5,6-oxide hydrolase, Glutathione:5-HPETE oxidoreductase, Glutathione:15-HPETE oxidoreductase, 3,4-dihydro-3-hydroxy-4-S-glutathionyl bromobenzene glutathione-lyase (epoxide-forming), 2,3-dihydro-2-S-glutathionyl-3-hydroxy bromobenzene glutathione-lyase (epoxide-forming), bromobenzene-3,4-oxide hydrolase, bromobenzene-2,3-oxide hydrolase, benzo[a]pyrene-7,8-oxide hydrolase, 4,5-dihydro-4-hydroxy-5-S-glutathionyl-benzo[a]pyrene glutathione-lyase (epoxide-forming), 7,8-dihydro-7-hydroxy-8-S-glutathionyl-benzo[a]pyrene hydrolase, S-(2,2-dichloro-1-hydroxy)ethyl-glutathione 2,2-dichloroacetaldehyde-lyase (glutathione-forming), 1,1-dichloroethylene-epoxide:glutathione S-(chloroepoxyethyl)transferase [2-(S-glutathionyl)acetyl-chloride-forming], chloroacetyl-chloride:glutathione S-chloroacetyltransferase, 2-(S-glutathionyl)acetyl-chloride:glutathione 2-(S-glutathionyl)acetyltransferase, trichloroethene:glutathione S-(1,2-dichlorovinyl)transferase, Chloral:NAD(P)+oxidoreductase, trichloroethanol:NAD+oxidoreductase, 1,2-dibromoethane:glutathione ethylenetransferase (episulfonium-forming), 2-bromoacetaldehyde:glutathione S-(formylmethyl)transferase, D-arabinitol 2-dehydrogenase, methylglyoxal reductase (NADPH-dependent), S-(hydroxymethyl)glutathione:NADP+oxidoreductase, 5-methyltetrahydrofolate:NAD+oxidoreductase, thiol-containing-reductant:hydroperoxide oxidoreductase, S-adenosyl-L-methionine:uroporphyrinogen-III C-methyltransferase, S-adenosyl-L-methionine:precorrin-1 C-methyltransferase, solanesyl-diphosphate:4-hydroxybenzoate nonaprenyltransferase, 2-lysophosphatidylcholine acylhydrolase, sn-glycerol-1-phosphate phosphohydrolase, inositol-3-phosphate synthase, 5,6,7,8-tetrahydropteridine:NAD(P)H+oxidoreductase, acireductone synthase, 2-hydroxy-5-(methylthio)-3-oxopent-1-enyl phosphate phosphohydrolase, 5-(methylthio)-2,3-dioxopentyl-phosphate phosphohydrolase (isomerizing), cephalosporin C:oxygen oxidoreductase (deaminating), cysteine desulfurase, spermidine:NAD+oxidoreductase, [eIF5A-precursor]-deoxyhypusine:NAD+oxidoreductase, dehydrospermidine:[enzyme]-lysine N-4-aminobutylidenetransferase, N-(4-aminobutylidene)-[enzyme]-lysine:[eIF5A-precursor]-lysine N-4-aminobutylidenetransferase, 4,4-dimethyl-9beta,19-cyclopropylsterol-4-alpha-methyl oxidase, Delta14-sterol reductase, L-methionine:thioredoxin-disulfide S-oxidoreductase, cis-stilbene-oxide hydrolase, carbamoylphosphate synthetase II, lipoyl synthase protein N6-(octanoyl)lysine:sulfur sulfurtransferase, lipoyl synthase octanoyl-[acp]:sulfur sulfurtransferase, ATP:lipoate adenylyltransferase, lipoate protein ligase, 5-fluoromuconolactone lactonohydrolase, 4-fluoromuconolactone lactonohydrolase, phosphoacetylglucosamine mutase, phosphoglycerate dehydrogenase, 5′-deoxy-5-fluorocytidine aminohydrolase, 5,6-dihydro-5-fluorouracil am idohydrolase, 5-fluorouridine monophosphate:diphosphate phospho-alpha-D-ribosyl-transferase, ATP:5-fluorouridine 5′-phosphotransferase, 6-thioinosine 5′-monophosphate:NAD+oxidoreductase, 6-thioxanthine 5′-monophosphate:L-glutamine amido-ligase, tamoxifen,NADPH:oxygen oxidoreductase (N-oxide-forming), 4-glutathionyl cyclophosphamide hydrolase, alcophosphamide:NAD+oxidoreductase, carboxyphosphamide:NAD+oxidoreductase, carboxyphosphamide:NADP+oxidoreductase, 2-phenyl-1,3-propanediol monocarbamate:NAD+oxidoreductase, 3-carbamoyl-2-phenylpropionaldehyde:NAD+oxidoreductase, 4-hydroxy-5-phenyltetrahydro-1,3-oxazin-2-one:NAD+oxidoreductase, S-adenosylmethioninamine:cadaverine 3-aminopropyltransferase, tryparedoxin:hydroperoxide oxidoreductase, trypanothione:hydroperoxide oxidoreductase, 2′-deoxyribonucleoside-diphosphate:tryparedoxin-disulfide 2′-oxidoreductase, 2′-deoxyribonucleoside-diphosphate:trypanothione-disulfide 2′-oxidoreductase, purine-nucleoside:phosphate ribosyltransferase, R-nitrile:glutathione R-transferase, R-sulfate-ester:glutathione R-transferase, NADPH:ferricytochrome-b5 oxidoreductase, 2-Oxoglutarate dehydrogenase complex, cytochrome P-450 reductase, choline dehydrogenase, 2-Aminobut-2-enoate aminohydrolase (spontaneous), 2-aceto-2-hydroxybutanoate synthase, sn-glycerol 3-phosphate:ubiquinone oxidoreductase, glycerol-1-phosphate phosphohydrolase, 2-Aminoacrylate aminohydrolase, S-adenosyl-L-methionine:3-Polyprenyl-4,5-dihydroxylate 5-O-methyltransferase, 5,10-methylenetetrahydromethanopterin:glycine hydroxymethyltransferase, 2,3,5-trichlorodienelactone lactonohydrolase, dienelactone hydrolase, (2E,6E)-farnesyl-diphosphate:isopentenyl-diphosphate farnesyltranstransferase, geranylgeranyl-diphosphate:isopentenyl-diphosphate transferase, 5-methyltetrahydropteroyltri-L-glutamate:L-selenohomocysteine Se-methyltransferase, 2,5-diamino-6-(5-phospho-D-ribitylamino)pyrimidin-4(3H)-one:NAD+1′-oxidoreductase, 2,5-diamino-6-(5-phospho-D-ribitylamino)pyrimidin-4(3H)-one:NADP+1′-oxidoreductase, epoxide hydrolase, microsomal epoxide hydrolase, xylitol:NAD+oxidoreductase, hydantoin racemase, fatty-acid-CoA ligase (octanoate), fatty-acid-CoA ligase (decanoate), fatty-acid-CoA ligase (dodecanoate), fatty-acid-CoA ligase (tetradecanoate), fatty-acid-CoA ligase (tetradecenoate), fatty-acid-CoA ligase (hexadecanoate), fatty-acid-CoA ligase (hexadecenoate), fatty-acid-CoA ligase (octadecanoate), fatty-acid-CoA ligase (octadecenoate), fatty-acid-CoA ligase (octadecynoate), fatty-acid-CoA ligase (n-C24:0), fatty-acid-CoA ligase (n-C26:0), fatty-acyl-ACP hydrolase, 6-phospho-D-glucono-1,5-lactone endoplasmic reticular transport via diffusion, ceramide-1 (C24) endoplasmic reticular transport, ceramide-1 (C26) endoplasmic reticular transport, ceramide-2 (C24) endoplasmic reticular transport, ceramide-2 (C26) endoplasmic reticular transport, dolichol phosphate endoplasmic reticular transport via proton symport, ergosterol endoplasmic reticular transport, Ergosta-5,6,22,24,(28)-tetraen-3beta-ol endoplamic reticular transport, glucose 6-phosphate endoplasmic reticular transport via diffusion, H2O endoplasmic reticulum transport, O2 transport, endoplasmic reticulum, sphinganine 1-phosphate endoplasmic reticular transport, squalene-2,3-epoxide endoplamic reticular transport, squalene endoplamic reticular transport, diphosphate endoplasmic reticulum transport, FAD endoplasmic reticulum transport, proton endoplasmic reticulum transport, formate endoplasmic reticulum transport, FADH2 endoplasmic reticulum transport, NADH endoplasmic reticulum transport, NAD endoplasmic reticulum transport, CO2 endoplasmic reticulum transport, SAH endoplasmic reticulum transport, 2-oxoglutarate reversible transport via symport, 3mop reversible trasport, acetaldehyde reversible transport, acetate reversible transport via proton symport, acetate transporter, adenine transport in via proton symport, adenosine transport in via proton symport, allantoate irreversible uniport, allantoin irreversible uniport, alpha-ketoglutarate/malate transporter, ammonia reversible transport, ATPase, cytosolic, Biotin uptake, C080decanoate (n-C18:0) transport in via uniport, C080decenoate (n-C18:1) transport in via uniport, C080decynoate (n-C18:2) transport in via uniport, choline transport via proton symport, citrate reversible transport via symport, CO2 transporter via diffusion, cytidine transport in via proton symport, cytosine transport in via proton symport, D-arabinose reversible transport, deoxyadenosine transport in via proton symport, deoxycytidine transport in via proton symport, deoxyguanosine transport in via proton symport, deoxyinosine transport in via proton symport, deoxyURIdine transport in via proton symport, D-fructose transport in via proton symport, D-galactose transport in via proton symport, D-lactate transport via proton symport, D-mannose transport in via proton symport, D-sorbitol transport via passive diffusion, dTTP reversible uniport, D-xylose reversible transport, ethanol reversible transport, fatty acid transport, formate transport via diffusion, glucose transport (uniport), glutathione transport, glycerol transport via channel, glycerol transport via symport, glycine reversible transport via proton symport, Glycoaldehydye reversible transport, glyoxylate transport, guanine reversible transport via proton symport, guanosine transport in via proton symport, H2O transport via diffusion, hexadecanoate (n-C16:0) transport in via uniport, hexadecenoate (n-C16:1) transport in via uniport, hypoxanthine reversible transport via proton symport, iron (II) transport, L-alanine reversible transport via proton symport, L-arabinoase extracellular transport, L-arganine reversible transport via proton symport, L-asparagine reversible transport via proton symport, L-aspartate reversible transport via proton symport, L-cysteine reversible transport via proton symport, L-glutamate transport via proton symport, reversible, L-glutamine reversible transport via proton symport, L-histidine reversible transport via proton symport, L-isoleucine reversible transport via proton symport, L-lactate reversible transport via proton symport, L-leucine reversible transport via proton symport, L-lysine reversible transport via proton symport, L-malate reversible transport via proton symport, L-methionine reversible transport via proton symport, L-phenylalanine reversible transport via proton symport, L-proline reversible transport via proton symport, L-serine reversible transport via proton symport, L-sorbitol transport via passive diffusion, L-sorbose reversible transport, L-threonine reversible transport via proton symport, L-tryptophan reversible transport via proton symport, L-tyrosine reversible transport via proton symport, L-valine reversible transport via proton symport, maltose transport in via proton symport, N,N-bisformyl-dityrosine transport (extracellular), NADP transporter, Nicotinic acid transport, nmntp, o2 transport (diffusion), orntithine reversible transport in via proton symport, oxaloacetate transport, oxidized glutathione irreversible uniport, pantothenate reversible transport via proton symport, PAP reversible uniport, peptide transport in via proton symport, phenethyl acetate transport (extracellular), phenylacetaldehyde transport (extracellular), phosphate reversible transport via symport, potassium reversible transport via proton symport, pyruvate exchange, diffusion, pyruvate transport in via proton symport, riboflavin transport in via proton symport, ribose transport in via proton symporter, sodium proton antiporter (H:NA is 1:1), spermidine excretion (cytosol to extracellular), spermidine transport in via proton antiport, spermine transport via proton antiport irreversible, succinate transport via proton symport, sucrose transport in via proton symport, sulfate irreversible uniport, sulfite transport (efflux, cytosol to extracellular), taURIne transport, thiamine transport in via proton symport, thymidine transport in via proton symport, thymine reversible transport via proton antiport, trehalose transport in via proton symporter, tryptophol transport (extracellular, uracil transport in via proton symport, urea reversible transport via proton symport (2H+), URIdine transport in via proton symport, xanthine reversible transport, xanthosine transport in via proton symport, xylitol transport via passive diffusion, zymosterol reversible transport, 2-methylbutyl transport (extracellular), 2-Methylbutanal transport (extracellular), 2-methyl-1-butanol transport (extracellular), 2-methylpropanal transport (extracellular), 2-phenylethanol reversible transport, 2-Isopropylmalate transport, diffusion, 3-methylbutanal transport (extracellular), 4-Aminobenzoate mitochondrial transport via diffusion, 5-Aminolevulinate transport in via proton symport, 8-Amino-7-oxononanoate reversible transport via proton symport, L-arabinitol transport via passive diffusion, 4-aminobutyrate reversible transport in via proton symport, acetic ester transport (extracellular), S-adenosyl-L-methionine transport in via proton symport, (R,R)-butanediol transport, 7,8-Diaminononanoate reversible transport via proton symport, ePisterol reversible transport, ergosterol reversible transport, ethanolamine transport via diffusion (extracellular), fecosterol reversible transport, fumarate reversible transport via symport, glycero-3-phosphocholine transport (extracellular to cytosol), glycero-3-phospho-1-inositol transport (extracellular to cytosol), D-glucosamine 6-phosphate reversible uniport, isoamyl acetate transport (extracellular), isoamyl alcohol transport (extracellular), isobutyl acetate transport (extracellular), isobutyl alcohol transport (extracellular), indoleacetaldehyde transport (extracellular), inosine transport in via proton symport, inositol transport in via proton symport, lanosterol reversible transport, melibiose transport in via symport, S-methylmethionine permease, ammonia transport (efflux, cytosol to extracellular), putrescine transport in via proton antiport, irreversible, putrescine excretion (cytosol to extracellular), CO2 Golgi transport, GDP-mannose antiport, GDP Golgi transport via proton anitport, phosphatidylethanolamine Golgi transport, phosphatidylserine Golgi transport, UDPgalactose transport (Golgi apparatus), diphosphate Mitochondrial transport, 2-Dehydro-3-deoxy-D-arabino-heptonate7-phosphate mitochondrial transport via diffusion, 2-Dehydropantoate mitochondrial transport, OBUT transporter (mitochondrial), 2-oxoadipate transport out of mitochondria via diffusion, chorismate mitochondrial transport, 3-(4-hydroxyphenyl)pyruvate mitochondrial transport via proton symport, 2-Isopropylmalate transport, diffusion, mitochondrial, 3-Carboxy-4-methyl-2-oxopentanoate transport, diffusion, mitochondrial, 3-Hexaprenyl-4,5-dihydroxybenzoate transport, mitochondrial, 3-methyl-2-oxobutanoate transport, diffusion, mitochondrial, 3-Methyl-2-oxopentanoate transport, diffusion, mitochondrial, 3-C080prenyl-4-hydroxybenzoate mitochondrial transport, 4-aminobutanal mitochondrial transport via diffusion, 4-aminobutanoate mitochondrial transport via diffusion, 4-Hydroxybenzoate mitochondrial transport, trans-4-hydroxy-L-proline mitochondrial transport via diffusion, 5-Aminolevulinate mitochondrial transport, acetaldehyde mitochondrial diffusion, acetate transport, mitochondrial, adenine reversible transport, mitochondria, S-adenosyl-L-homocysteine reversible transport, mitochondrial, alanine transport from mitochondria to cytoplasm, S-Adenosyl-L-methionine reversible transport, mitochondrial, arginine mitochondrial transport via proton symport, asparagine mitochondrial transport via proton transport, aspartate-glutamate transporter, aspartate mitochondrial transport via proton symport, ADP/ATP transporter, mitochondrial, citrate transport, mitochondrial, CO2 transport (diffusion), mitochondrial, CoA transporter (mitochondrial), irreversible, CTP/CMP antiport, D-lactate/pyruvate antiport, mitochondrial, D-lactate transport, mitochondrial, dihydroxyacetone phosphate transport, mitochondrial, dihydrofolate reversible mitochondrial transport, dhnpt mitochondrial transport, dihydropteroate mitochondrial transport via diffusion, L-erythro-4-hydroxyglutamate mitochondrial transport via diffusion, D-erythrose 4-phosphate mitochondria transport via diffusion, ethanol transport to mitochondria (diffusion), fatty-acyl-ACP mitochondrial transport, FAD/FMN antiport, iron (II) uptake (mitochondrial), formate mitochondrial transport, fumarate reductase, cytosolic/mitochondrial, farnesyl diphosphate transport (mitochondrial), glycolaldehyde mitochondrial transport, L-glutamate transport into mitochondria via hydroxide ion antiport, glutamate transport (uniporter), mitochondrial, glycerol-3-phosphate shuttle, glycine mitochondrial transport via proton symport, guanosine mitochondrial transport via proton symport, GTP/GDP translocase, mitochondrial (electroneutral), guanine mitochondrial transport via diffusion, H2O transport, mitochondrial, all-trans-hexaprenyl diphosphate transport, mitochondrial, histidine mitochondrial transport via proton symport, hydroxymethylglutaryl-CoA reversible mitochondrial transport, isoamyl alcohol transport (mitochondrial), isobutyl alcohol transport (mitochondrial), indole-3-acetaldehyde mitochondrial transport via diffusion, Isoleucine transport from mitochondria to cytosol, indole-3-acetate mitochondrial transport via diffusion, tryptophol transport (mitochondrial), Isopentenyl diphosphate transport, mitochondrial, L-lactate transport, mitochondrial, Lysine mitochondrial transport via proton symport, malate transport, mitochondrial, methionine mitochondrial transport via proton symport, NH3 mitochondrial transport, NMN mitochondrial transport via proton symport, oxaloacetate transport, mitochondrial, ornithine mitochondrial transport via proton antiport, 2-oxodicarboylate transporter, mitochondrial, phenylacetaldehyde transport (mitochondrial), panthetheine 4′-phosphate reversible mitochondrial transport, pantothenate mitochondrial transport, adenosine 3′,5′-bisphosphate mitochondrial transport, phosphatidate reversible transport, mitochondrial, all-trans-Pentaphenyl diphosphate transport, mitochondrial, phosphatidylethanolamine mitochondrial transport, phenylalanine mitochondrial transport via proton symport, phosphate transporter, mitochondrial, phosphate transport via hydroxide ion symport, mitochondrial, protoporphyrinogen IX mitochondrial transport, L-proline transport, mitochondrial, PRPP reversible transport, mitochondrial, phosphatidylserine mitochondrial transport, pyruvate mitochondrial transport via proton symport, quinolinate reversible mitochondrial transport, riboflavin reversible mitochondrial transport, serine mitochondrial transport via proton symport, succinate transport, mitochondrial, succinate-fumarate transport, mitochondrial, thiamine diphosphate transport, mitochondria, threonine mitochondrial transport via proton symport, tryptophan mitochondrial transport via proton symport, tyrosine mitochondrial transport via proton symport, UTP/UMP antiport, valine reversible mitochondrial transport via proton symport, acetyl-CoA transport, nuclear, 2-oxoglutarate nuclear transport via proton symport, AMP transport via diffusion (cytosol to nucleus), aspartate nuclear transport via proton symport, L-aspartate nuclear transport via diffusion, N-carbomoyl-L-aspartate transport, diffusion, carbamoyl phosphate nuclear transport via diffusion, CDP nuclear transport, CO2 nuclear transport via diffusion, coenzyme A transport, nuclear, DADP nuclear transport, dCDP nuclear transport, dGDP nuclear transport, dUMP nuclear transport, GDP nuclear transport, glutamine nuclear transport via proton symport, glutamate nuclear transport via proton symport, hydrogen peroxide nuclear transport, H2O transport, nuclear, bicarbonate nuclear transport via diffusion, 1D-myo-Inositol 1,4,5-trisphosphate nuclear transport via diffusion, inositol hexakisphosphate nuclear transport (diffusion), NAD transport, nuclear through pores, ammonia nuclear transport, phosphate nuclear transport via proton symport, phosphatidyl-1D-myo-insoitol nuclear transport, phosphatidyl-1D-myo-4-inositol nuclear transport, UMP nuclear transport, 3-(4-hydroxyphenyl)pyruvate peroxisomal transport via proton symport, 4-hydroxy-2-oxoglutarate peroxisomal transport via diffusion, acetate transport, peroxisomal, AKG transporter, peroxisome, aspartate-glutamate peroxisomal shuttle, AMP/ATP transporter, peroxisomal, ADP/ATP transporter, peroxisomal, citrate/malate antiport into peroxisome, citrate/isocitrate antiport into peroxisome, CO₂ peroxisomal transport, cystathione peroxisomal transport, L-erythro-4-hydroxyglutamate peroxisomal transport via diffusion, fatty acid peroxisomal transport, glyoxylate transport, peroxisomal, H₂O transport, peroxisomal, homocysteine peroxisomal transport via proton symport, malate/oxaloacetate shuttle, ammonia peroxisomal transport, NMN peroxisomal transport via proton symport, phosphate peroxisomal transport via proton symport, pyruvate peroxisomal transport via proton symport, oxidized thioredoxin peroxisomal transport via diffusion, reduced thioredoxin peroxisomal transport via diffusion, tyrosine peroxisomal transport via proton symport, L-arginine transport in via proton antiport (vacuolar), L-asparagine transport in via proton antiport (vacuolar), L-asparagine transport out via proton symport, vacuolar, L-aspartate transport out via proton symport, vacuolar, CO₂ vacuolar transport, glycogen vacuolar ‘transport’ via autophagy, glucose transport, vacuolar, L-glutamine transport in via proton antiport (vacuolar), L-glutamine transport out via proton symport, vacuolar, L-glutamate transport out via proton symport, vacuolar, reduced glutathione via ABC system (vacuolar), H₂O transport, vacuolar, L-histidine transport in via proton antiport (vacuolar), L-isoleucine transport in via proton antiport (vacuolar), L-isoleucine transport out via proton symport, vacuolar, L-cystine transport via proton symport (vacuolar), L-leucine transport in via proton antiport (vacuolar), L-leucine transport out via proton symport, vacuolar, L-lysine transport in via proton antiport (vacuolar), phosphatidylethanolamine vacuolar transport, phosphate vacuolar transport via proton symport, phosphatidylserine vacuolar transport, taurocholate via ABC system (vacuolar), trehalose vacuolar transport via proton symport, L-tyrosine transport in via proton antiport (vacuolar), L-tyrosine transport out via proton symport and vacuolar.

In an embodiment, the gene deletion simulation in the constructed metabolic network model sets up a cell growth rate as an object function under the condition of fixing the corresponding metabolic flux of the enzyme reaction equation to be inhibited in the metabolic flux vector (v) as 0 (=vj) and runs the linear program to maximize the cell growth rate.

Also, the method may include acquiring metabolic products and/or cell composition information produced based on the secondary modified metabolic pathway.

At this time, the metabolic product may be a metabolism intermediate product or a metabolism final product. Specifically, the metabolic product may be succinate, lactate or 3HP, and specifically may be 3HP.

Also, the method may include acquiring a metabolic product-biomass correlation equation based on the acquired metabolic products and cell composition information.

The term “metabolic product-biomass correlation equation” is an equation that shows the relationship between the metabolic products and biomass produced by microorganisms. Also, a graph showing this relationship is called a trade-off curve.

Through the metabolic product-biomass correlation equation and trade-off curve, the relationship between increase and decrease of biomass and produced quantity of metabolic products may be found.

Also, the method may include acquiring an optimized metabolic product-biomass correlation equation by repeating from the acquiring of the secondary modified metabolic pathway to the acquiring of the metabolic product-biomass correlation equation.

The secondary modified metabolic pathway may be acquired by modifying activities of various enzymes involved in the metabolic pathways in microorganisms, an metabolic product-biomass correlation equation according to the pathway may be acquired, and a optimized metabolic product-biomass correlation equation may be acquired by using the metabolic product-biomass correlation equation. The term “optimized” means the time of producing the largest quantity of metabolic product while biomass is maintained at a certain level or increased.

Also, the method may include acquiring a secondary modified metabolic pathway that is a foundation of the optimized metabolic product-biomass correlation equation.

Also, the secondary modified metabolic pathway that may acquire the optimized metabolic product-biomass correlation equation may be acquired to acquire the most efficient reaction to produce metabolic products.

As such, the metabolic pathways to produce the optimized metabolic products may be estimated, and transformation of microorganisms may be planned using these pathways.

A transformed microorganism including a secondary modified metabolic pathway that may efficiently produce metabolic products is provided.

The microorganisms may be E. coli and yeast, and preferably may be K. marxianus.

The microorganisms may produce the desired metabolic products. For example, the microorganisms may produce succinate, lactate, and 3HP, and in particular 3HP.

A method of producing specific metabolic products using the transformed microorganisms is provided. The method may include: including culturing the transformed microorganism; and recollecting desired metabolic products from the culture. Also, the desired metabolic product may be 3HP.

The carbon sources able to be used by the microorganisms may be monosaccharides, disaccharides, or polysaccharides. Specifically glucose, fructose, mannose, galactose, etc. may be used. Also, the nitrogen source able to be used by the microorganisms may be organic nitrogen compounds, inorganic nitrogen compounds, etc. Specifically, the nitrogen source may be amino acids, amides, amines, nitrate salts, ammonium salts, etc. The oxygen condition for culturing the microorganisms may be an aerobic condition of normal oxygen partial pressure, a low-oxygen condition that includes about 0.1-about 10% oxygen in the atmosphere, or an anaerobic condition with no oxygen.

Provided is a method of identifying an optimized metabolic pathway for the production of a metabolic product, the method comprising (a) providing a metabolic network model of a microorganism; (b) identifying a metabolic pathway in the network model; (c) providing a primary modified metabolic pathway in the network model by introducing a simulated biochemical reaction pathway that did not previously exist in the network model; (d) modifying at least one enzyme reaction in the primary modified metabolic pathway to provide a secondary modified metabolic pathway; (e) determining the effect of the secondary modified metabolic pathway on the production of a metabolic product, growth of the microorganism, or both; (f) calculating a metabolic product-biomass correlation equation based on the information; (g) repeating steps (d)-(f) for different enzyme reactions in the primary modified metabolic pathway; and (h) identifying the secondary modified metabolic pathway that provides the optimized metabolic pathway for production of the metabolic product based on the product-biomass correlation equation.

The metabolic network model may be provided by constructing a metabolic network model of a microorganism based on the cell composition of the microorganism and GPR relationships for enzyme reaction equations of the microorganism. Constructing the metabolic network model may involve determining the cell composition of the microorganism and determining the biomass synthesis equation.

### BRIEF DESCRIPTION OF THE DRAWINGS

These and/or other aspects will become apparent and more readily appreciated from the following description of the embodiments, taken in conjunction with the accompanying drawings in which:

FIG. 1 is a graph of a trade-off curve when three external enzyme reactions are introduced independently to K. marxianus, illustrating the 3HP productivity of each external enzyme reaction in K. marxianus;

FIG. 2 is a graph of a trade-off curve when the three external enzyme reactions introduced independently to Saccharomyces cerevisiae (S. cerevisiae), illustrating the 3HP productivity of each external enzyme reaction in S. cerevisiae;

FIG. 3 is a graph of a trade-off curves for 3HP productivity when of Malonyl-CoA and glycerol enzyme reaction equations are introduced to K. marxianus and S. cerevisiae;

FIG. 4 is a schematic that illustrates various 3HP producing pathways;

FIGS. 5A-5BX are tables that illustrate enzymes and reaction equations; and

FIGS. 6A-6CH are tables that illustrate explanations on abbreviations used for the enzyme reaction equations.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

### Example 1

### Cell Composition Analysis of K. Marxianus

A biomass synthesis equation of cells that is essential for a metabolic network was constructed using the information of various texts, and information acquired from reference to strains in close relation or direct analysis of samples acquired from actual fermentation for sections without information from texts.

First, each of macromolecular compositions that form cells was collected. It was assumed that cells for the embodiment consist of protein, RNA, DNA, phospholipids, cell wall (polysaccharides), and other compositions of small quantities.

The amino acid composition analysis of proteins was acquired by requesting analysis of the sample acquired from fermentation of K. marxianus to the Proteomics Team of the Korea Basic Science Institute (KBSI). Also, the composition analysis of nucleotides that form DNA was acquired by analyzing the composition of a nucleic base sequence, since the nucleic base sequence was already fully completed. The RNA composition analysis was also acquired by analyzing the composition of the nucleic base sequence with a known ratio of mRNA, tRNA, and rRNA. Next, the compositions of fatty acids and polar groups of phospholipids were measured through GC (gas chromatography). Finally, other compositions were used based on the data found in texts (Composition of the cell walls of several yeast species, Nguyen T H et al., Appl Microbiol Biotechnol (1998); Appropriate sampling for intracellular amino acid analysis in 5 phylogenetically different yeast, Bolten C J and Wittmann C, Biotechnol Lett (2008); Ethanol tolerance and membrane fatty acid adaptation in adh multiple and null mutants of Kluyveromyces lactis, Heipieper H J et al., Res. Microbiol. (2000); Simple control of specific growth rate in biotechnological fed-batch processes based on enhanced online measurements of biomass, Dabros M et al., Can. J. Microbiol. (2010); The heterogeneity of Glucan Preparations from the Wall of Various Yeasts, Manners D J, Masson A J and Patterson J C, Journal of General Microbiology (1974); The structure of a beta-1-3-D-Glucan from Yeast Cell Walls, Manners D J, Masson A J and Patterson J C, Biochem. J. (1973); A systematic study of the cell wall composition of K lactis, Backhaus K et al., Yeast (2010)).

From the information obtained, the cell compositions of Table 2 to Table 9 were found. The following biomass synthesis equation was constructed based on the obtained cell compositions.

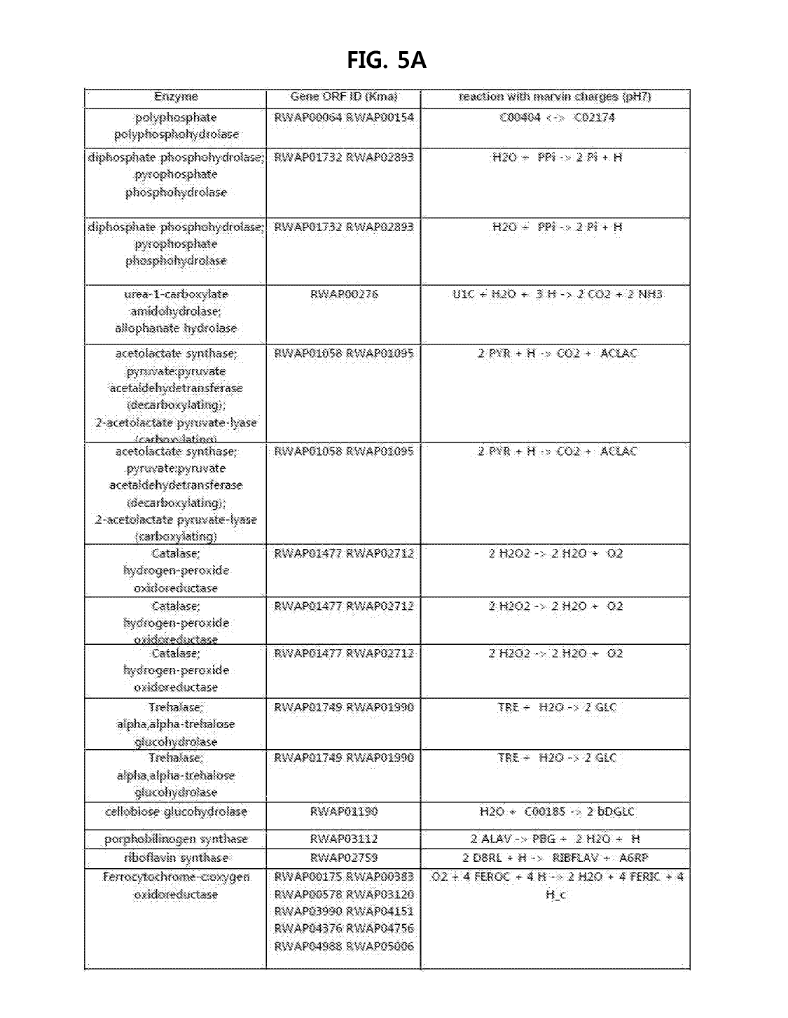

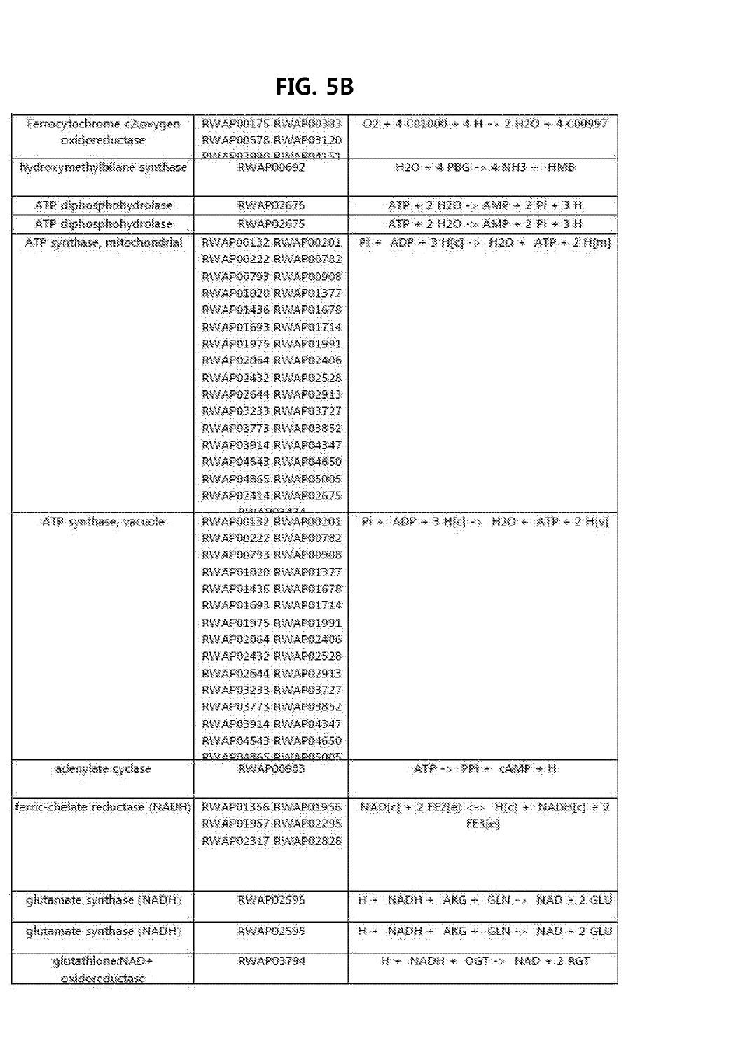

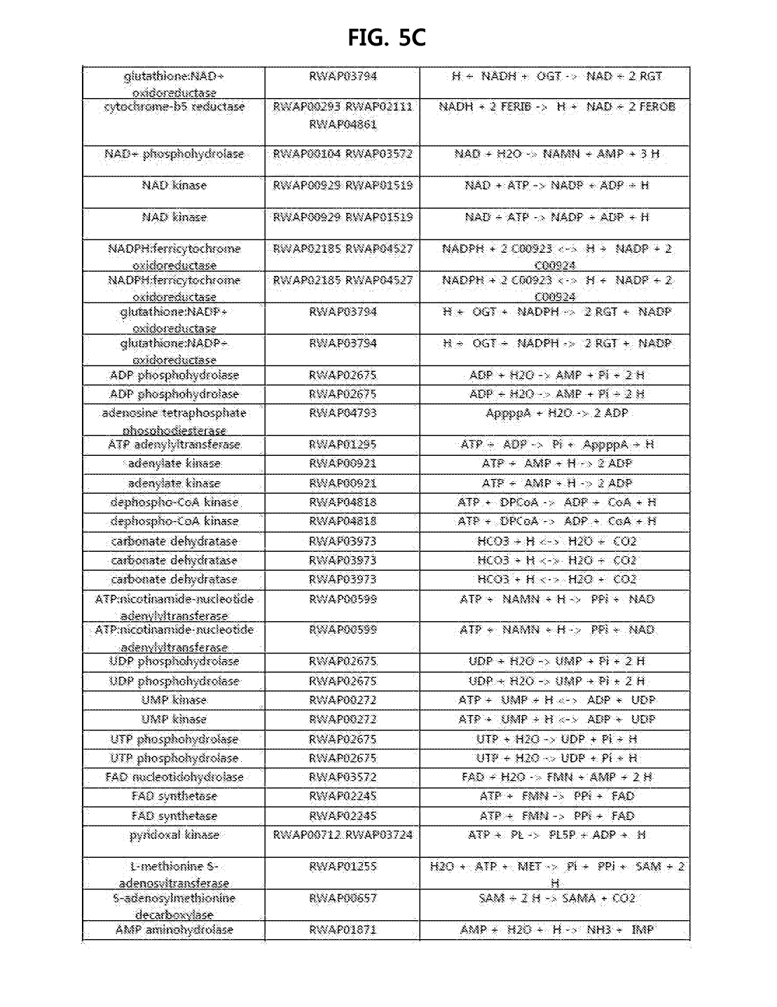

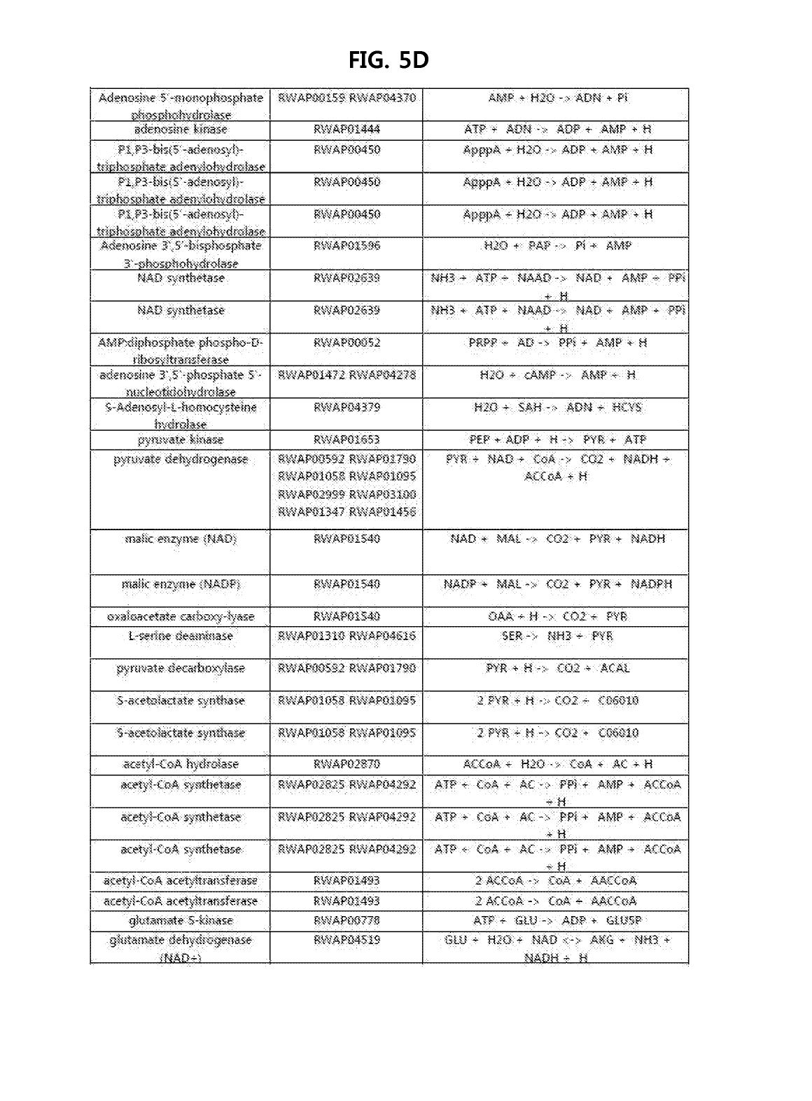

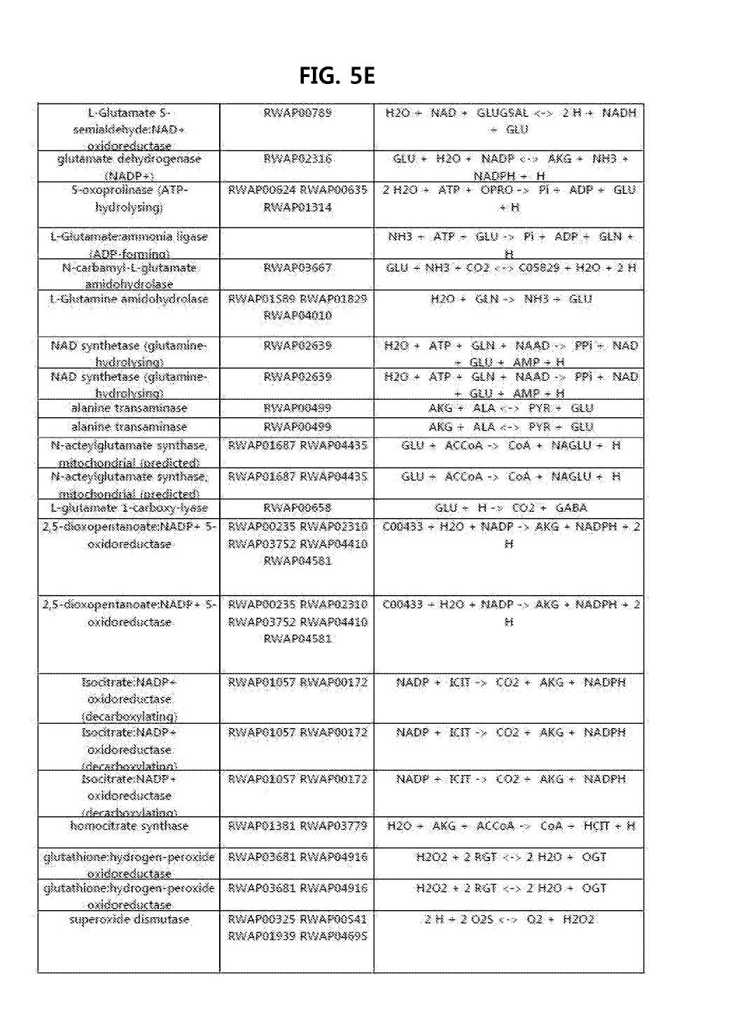

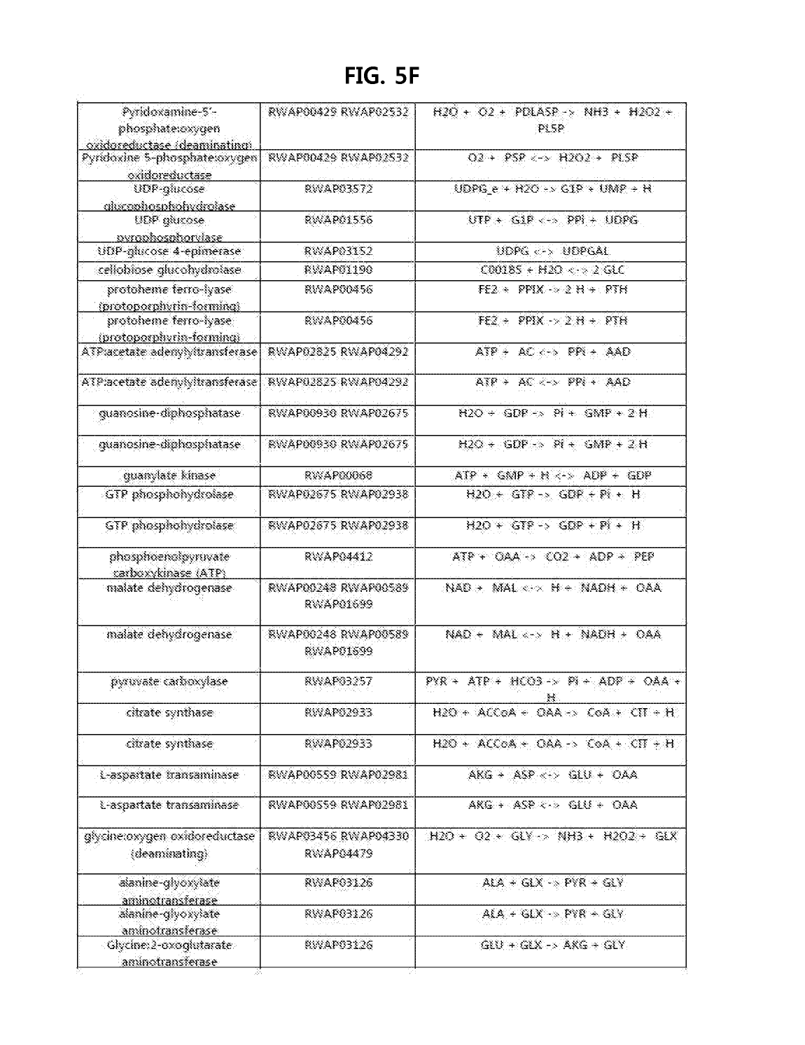

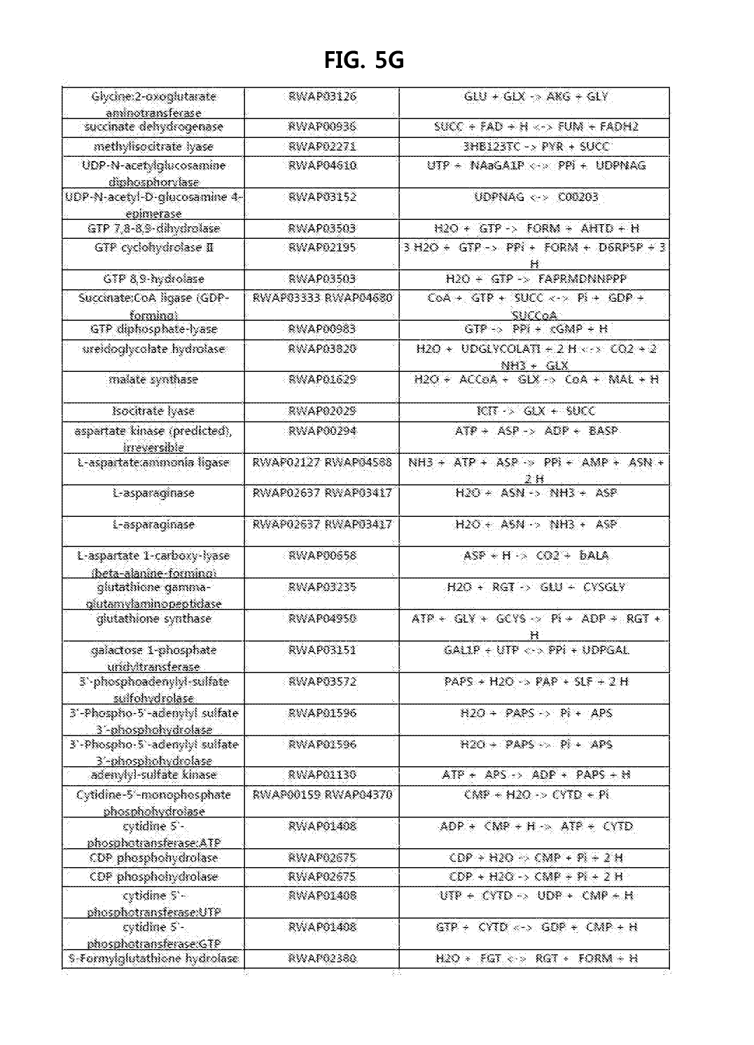

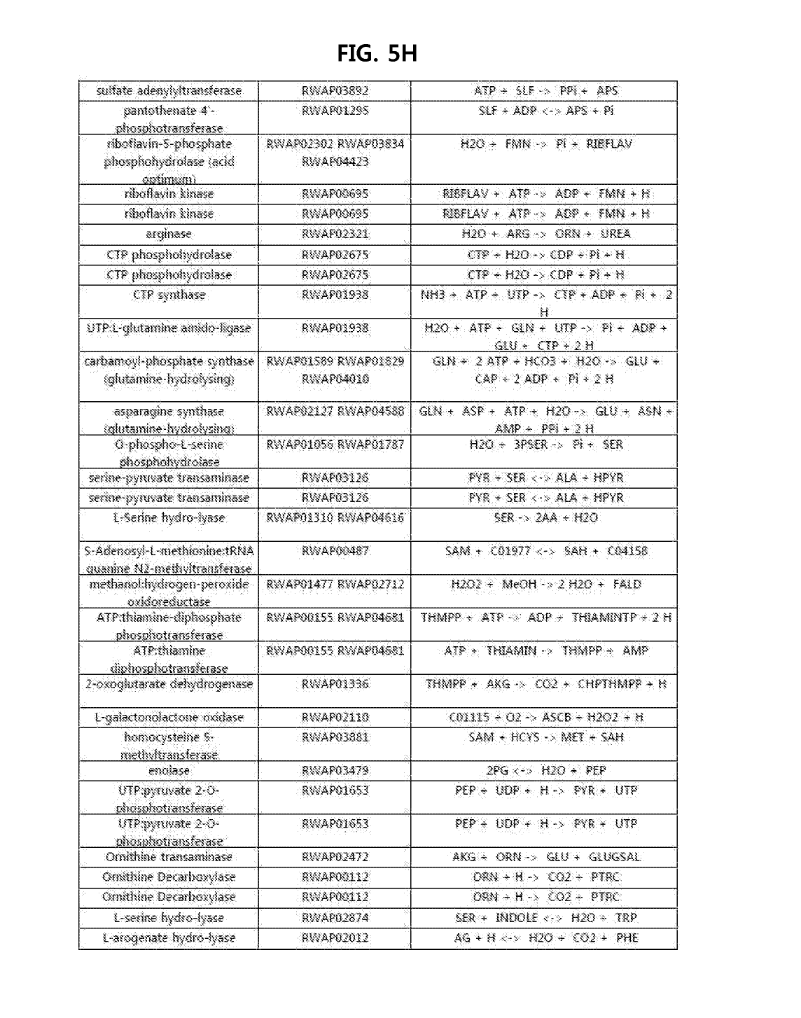

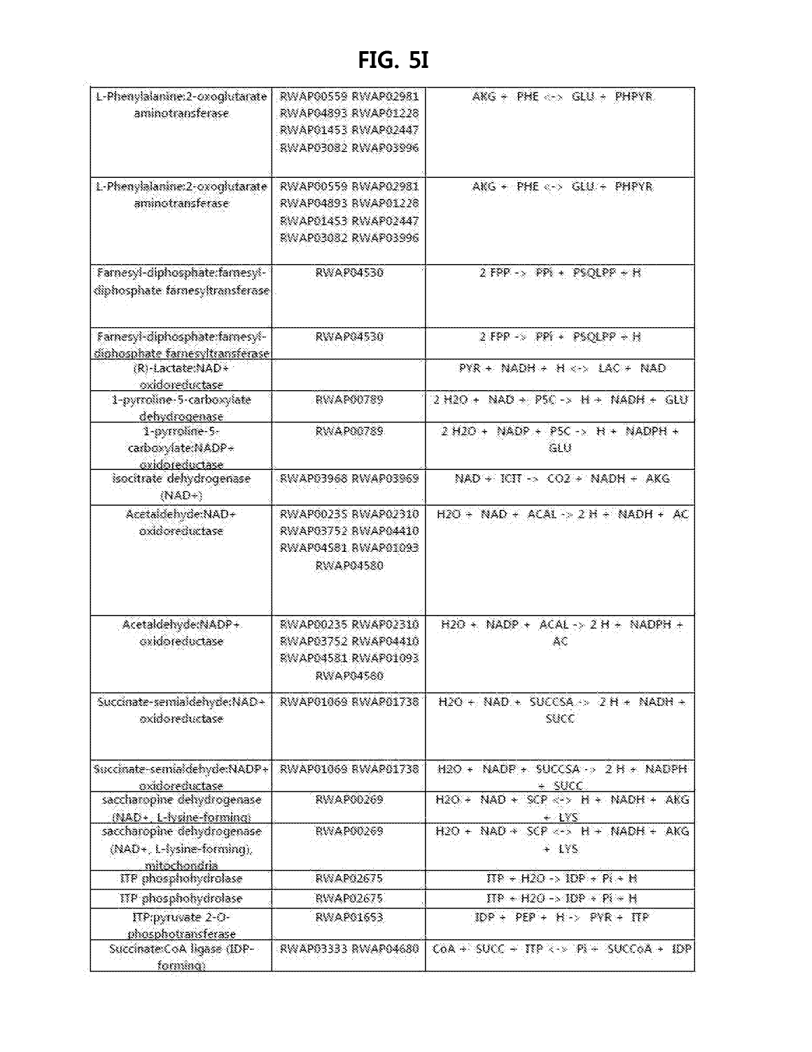

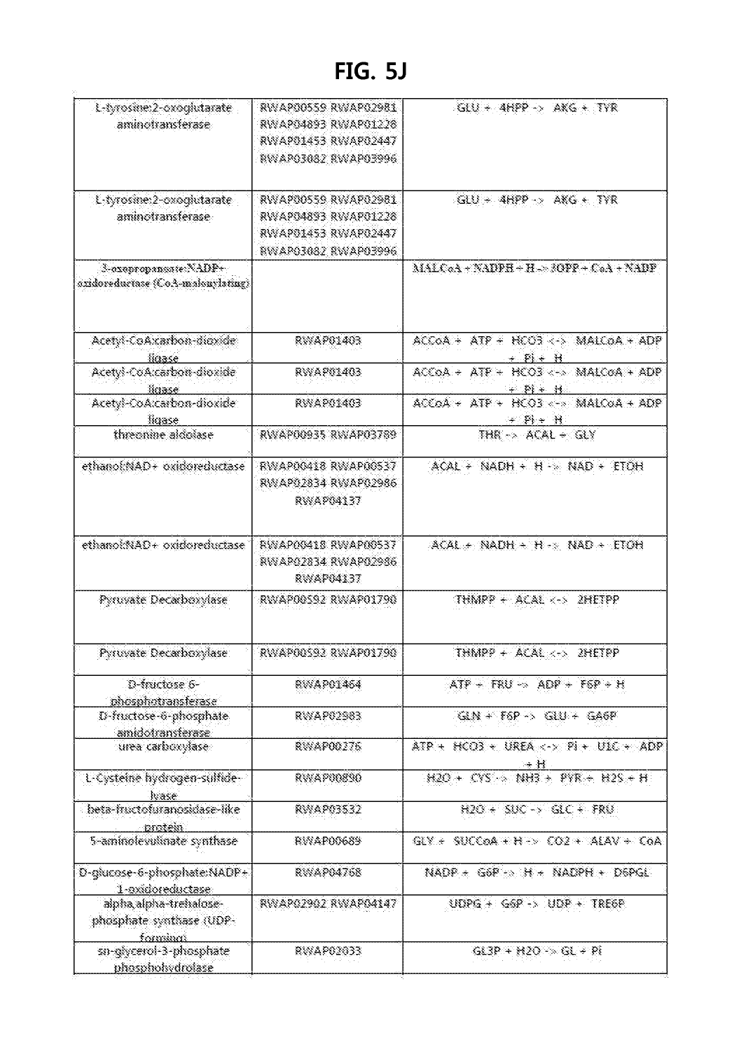

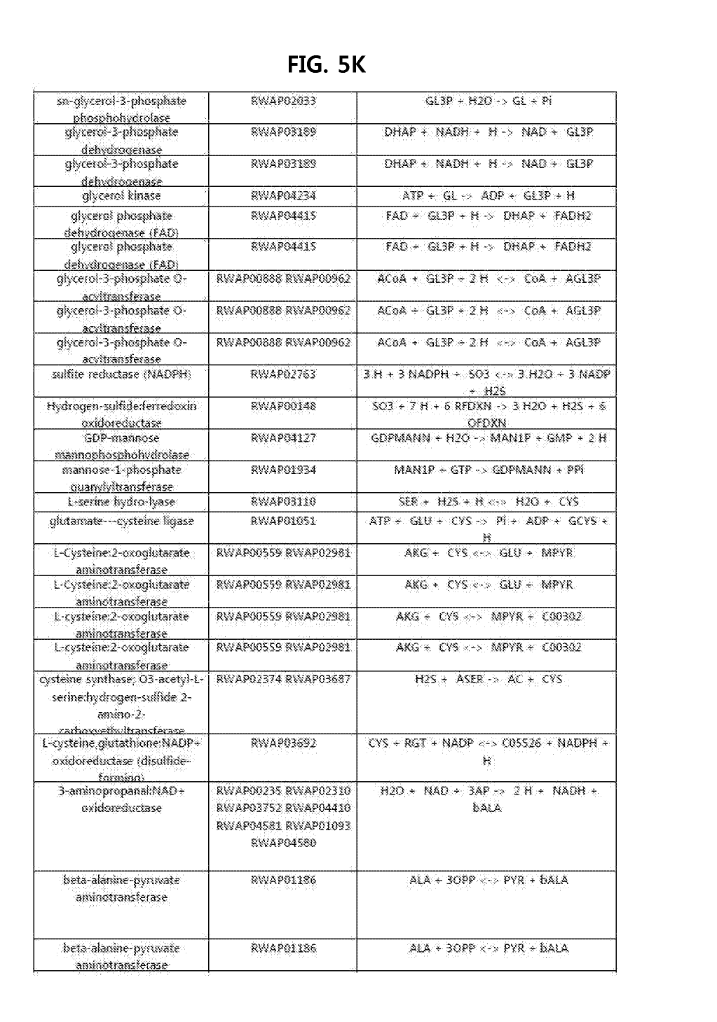

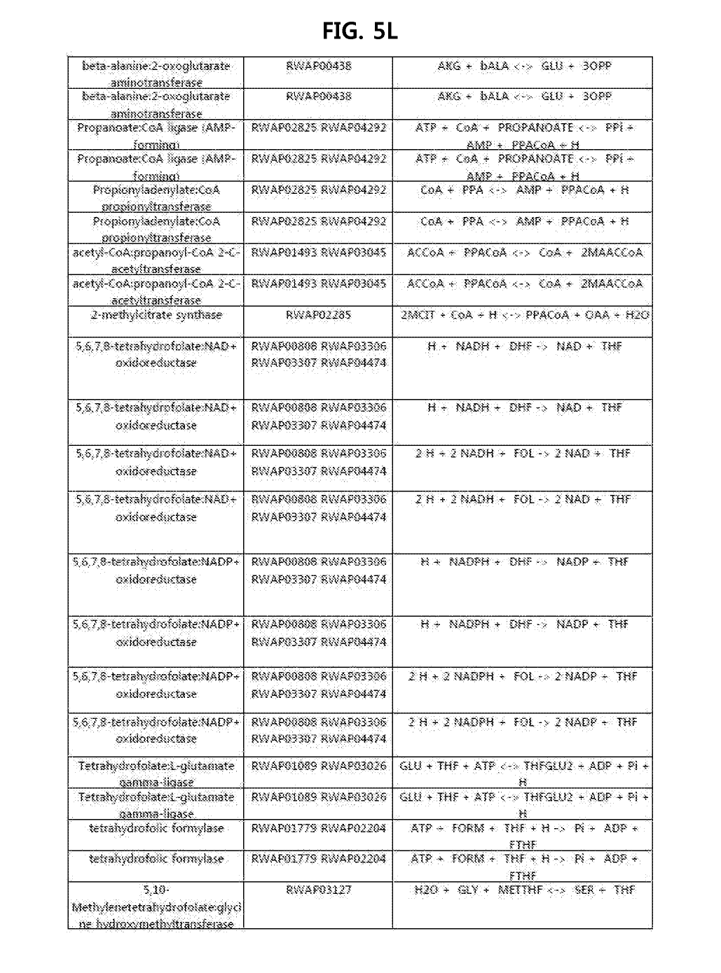

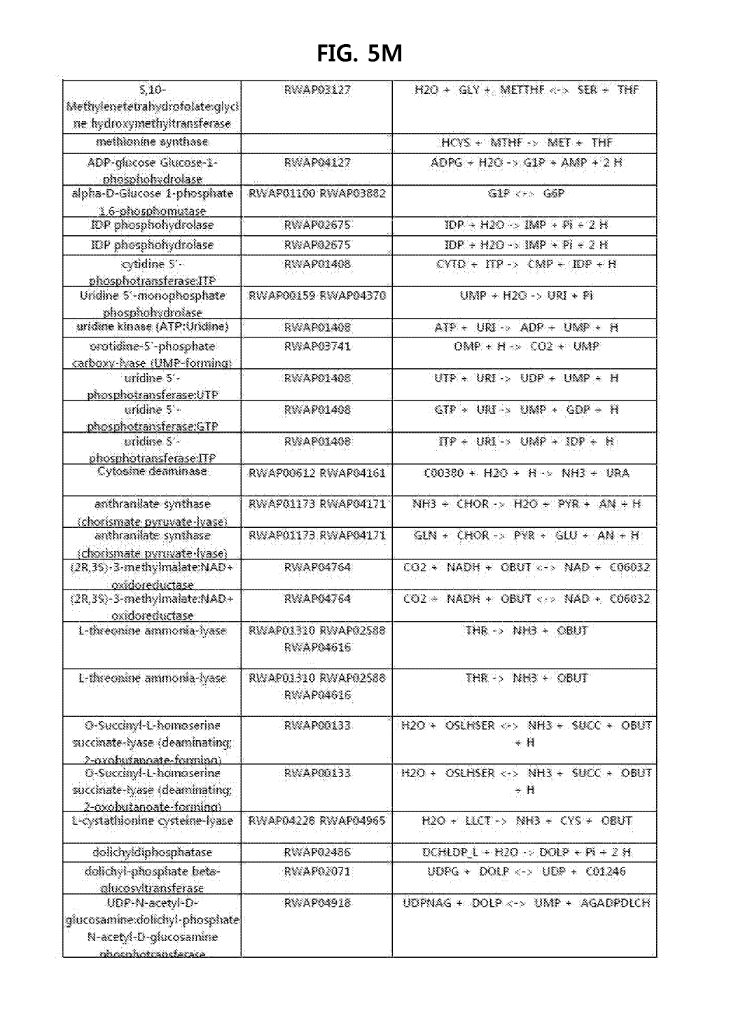

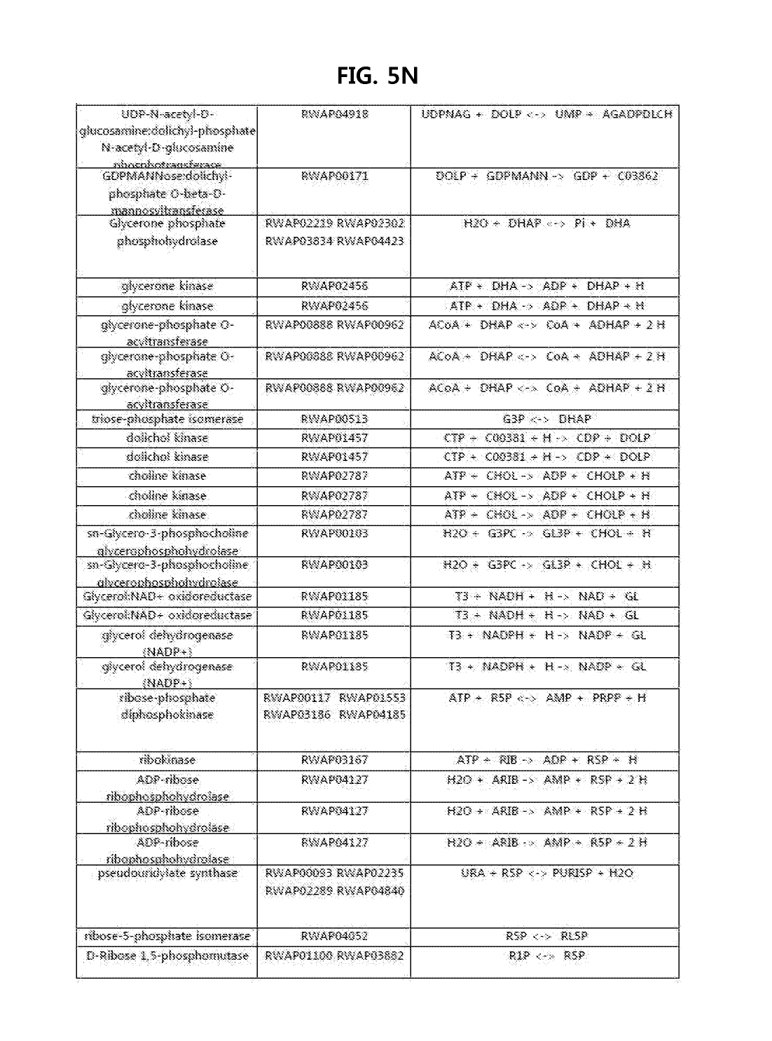

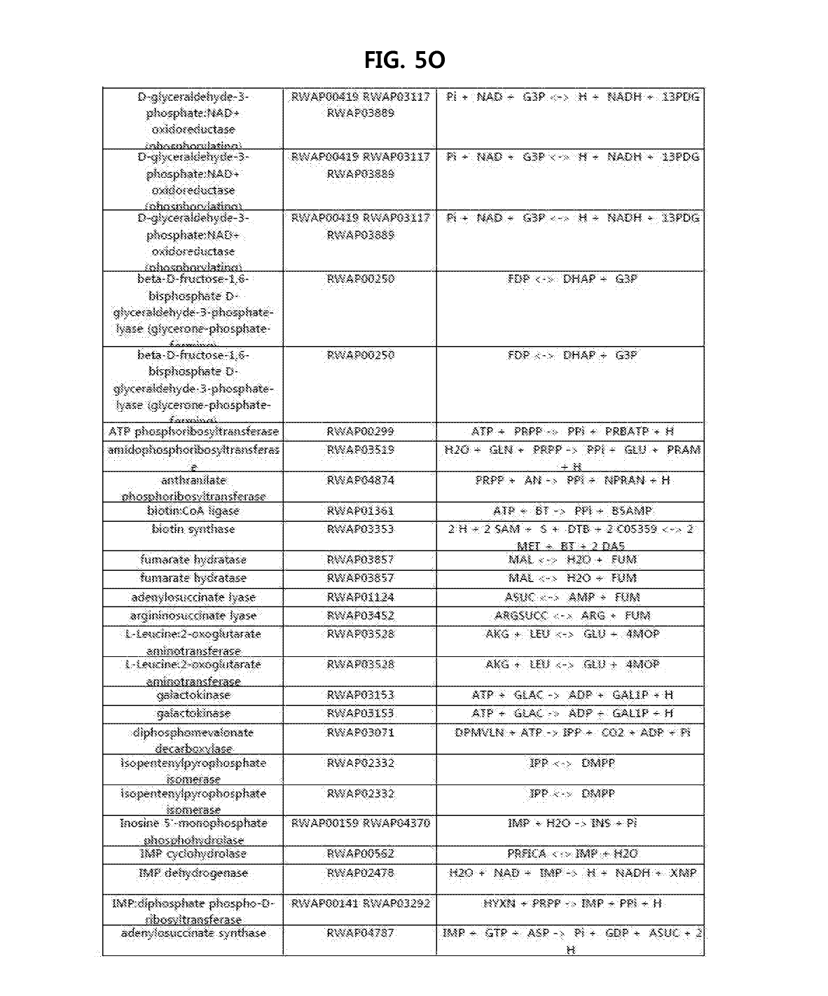

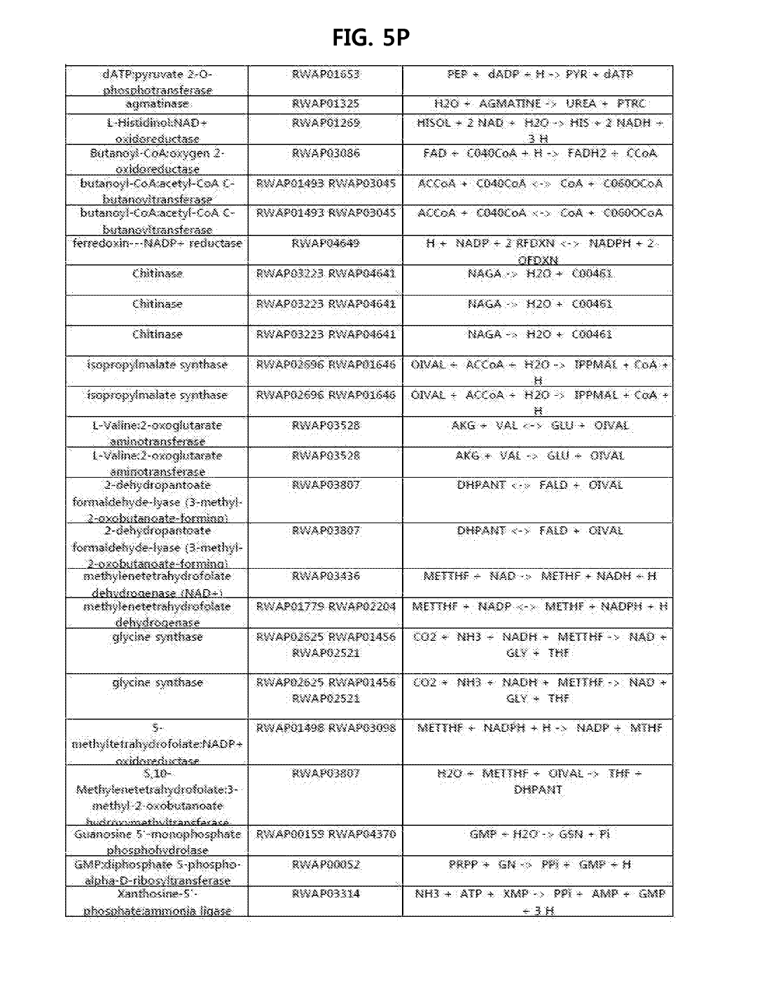

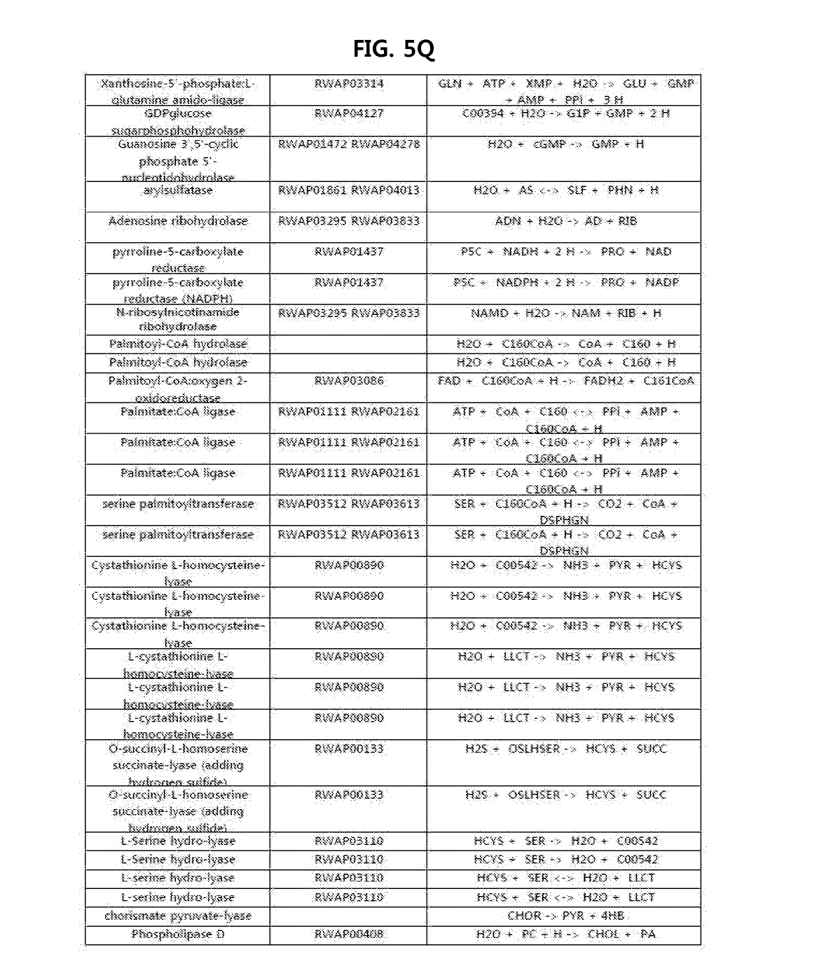

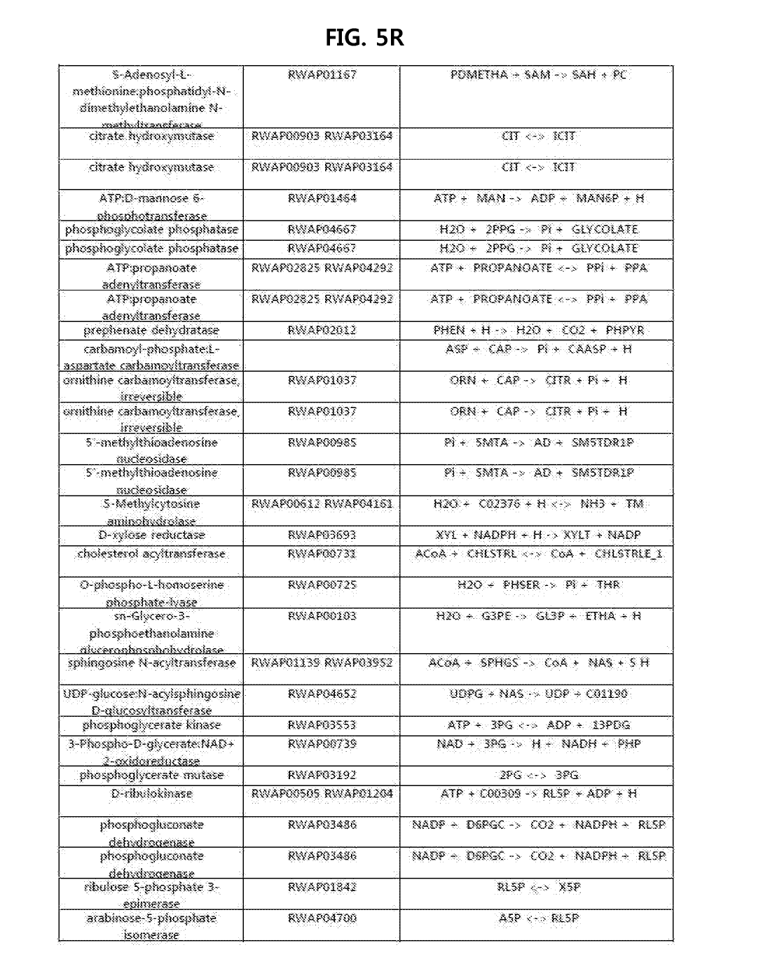

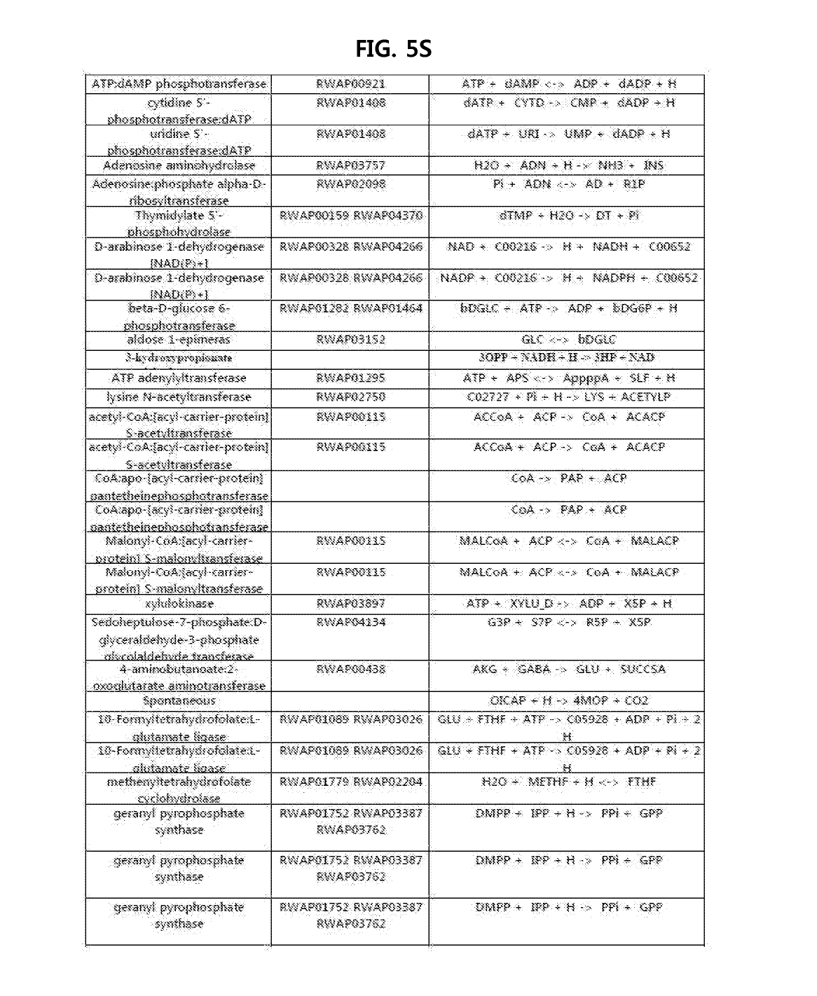

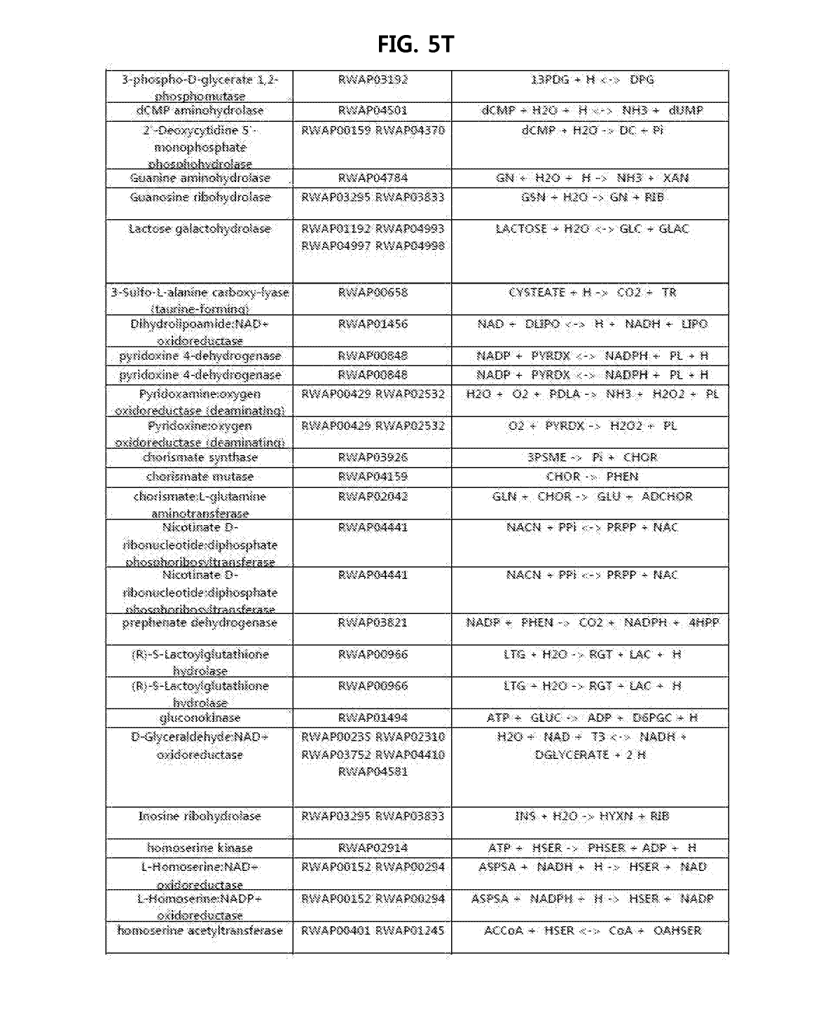

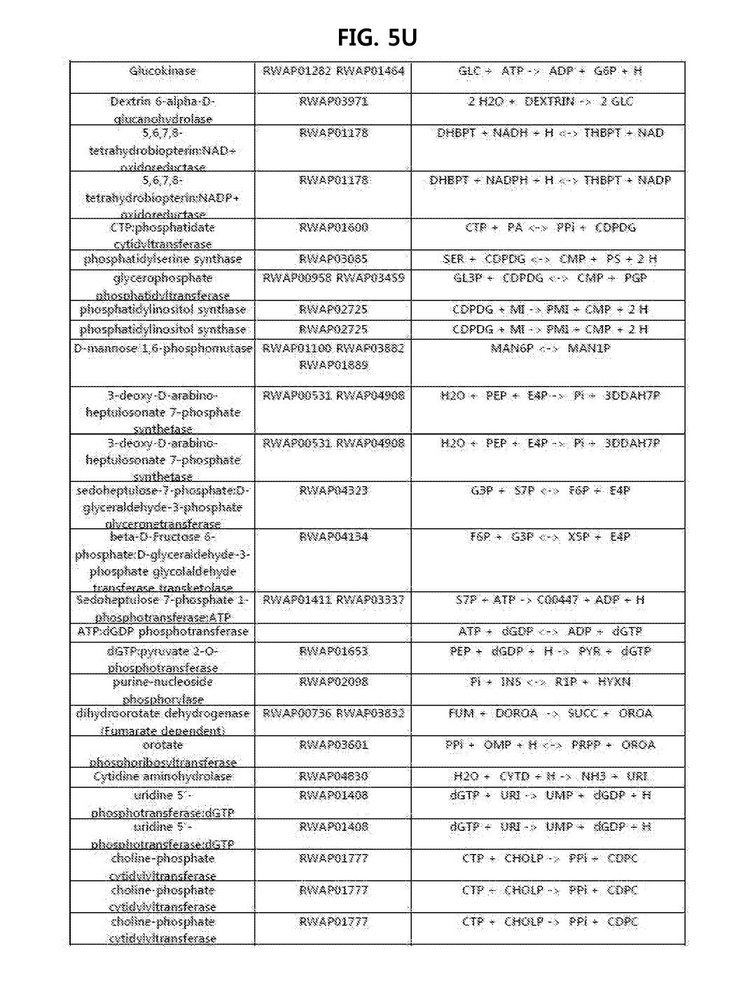

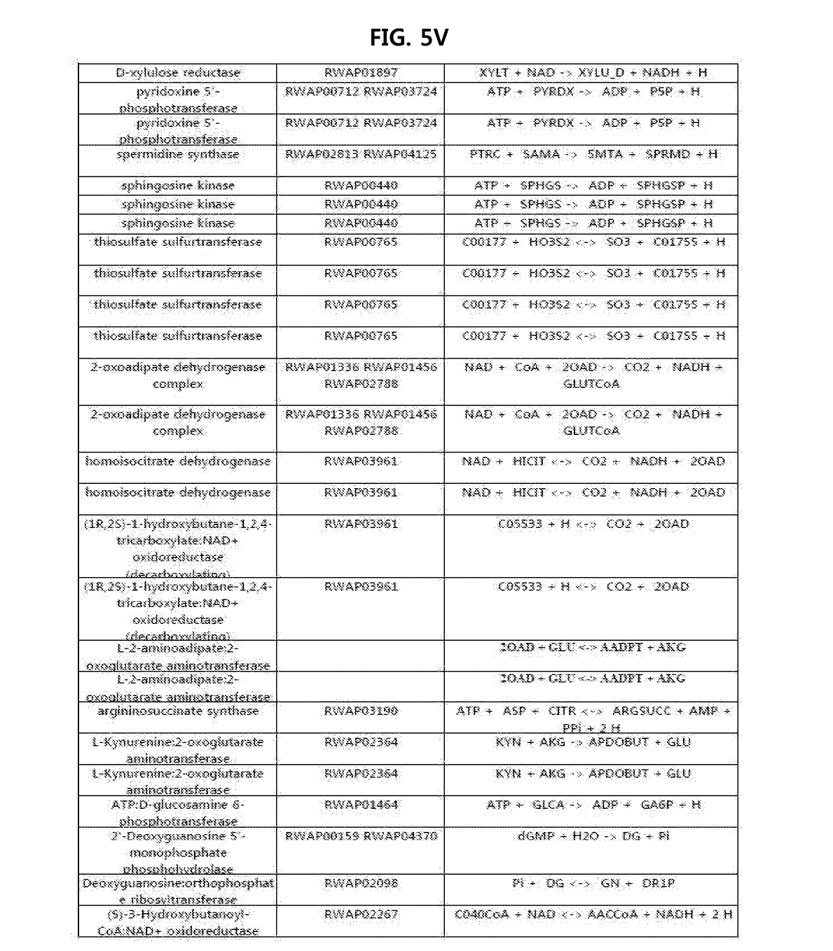

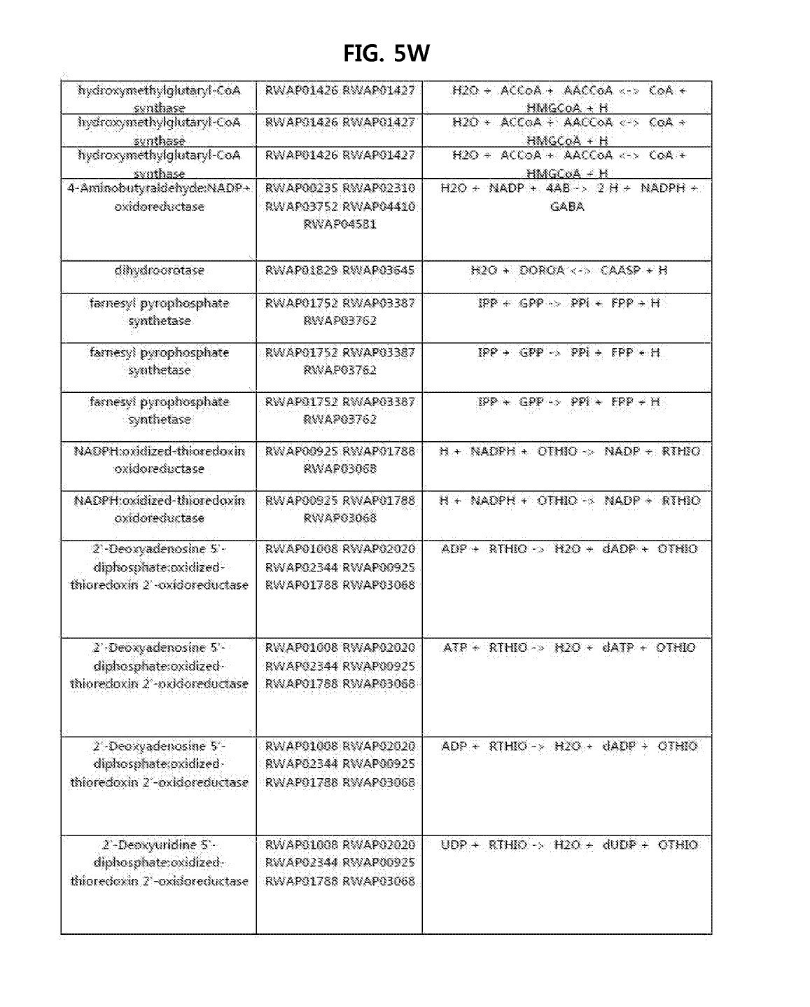

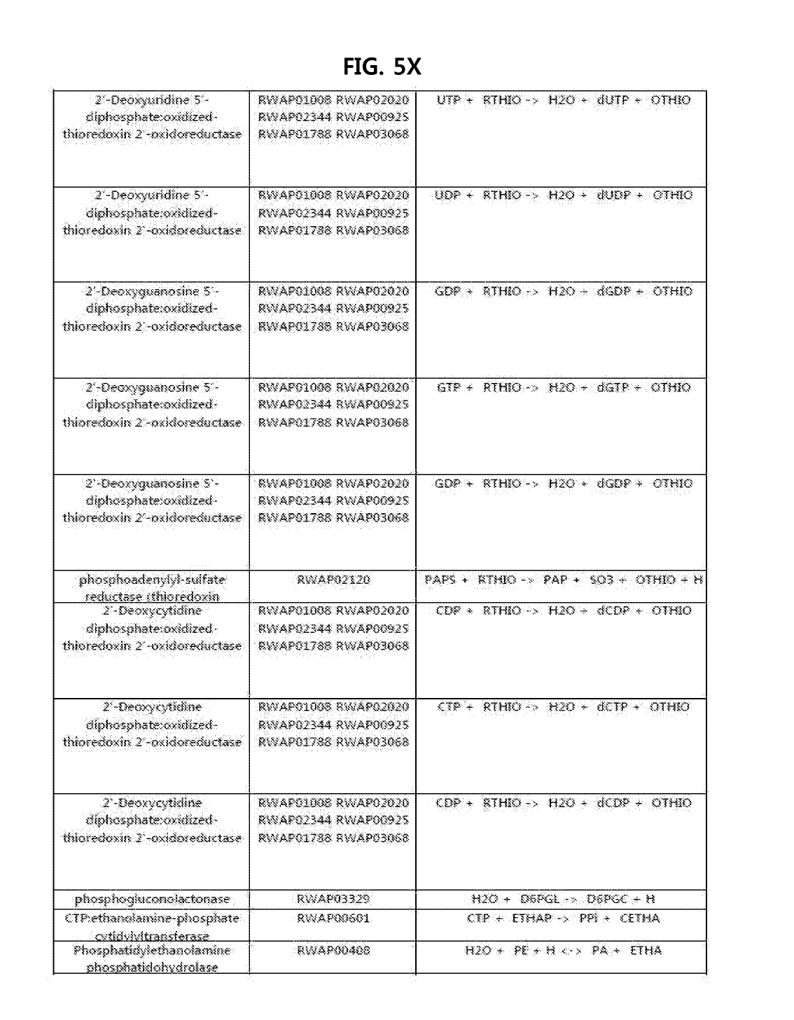

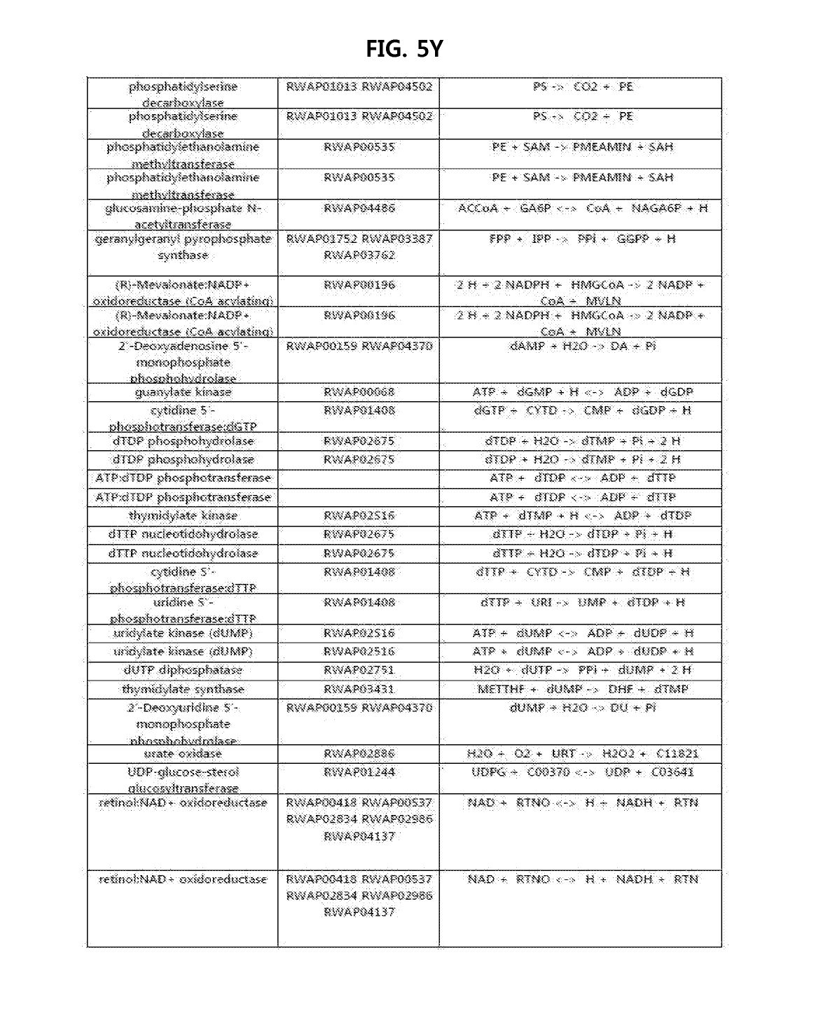

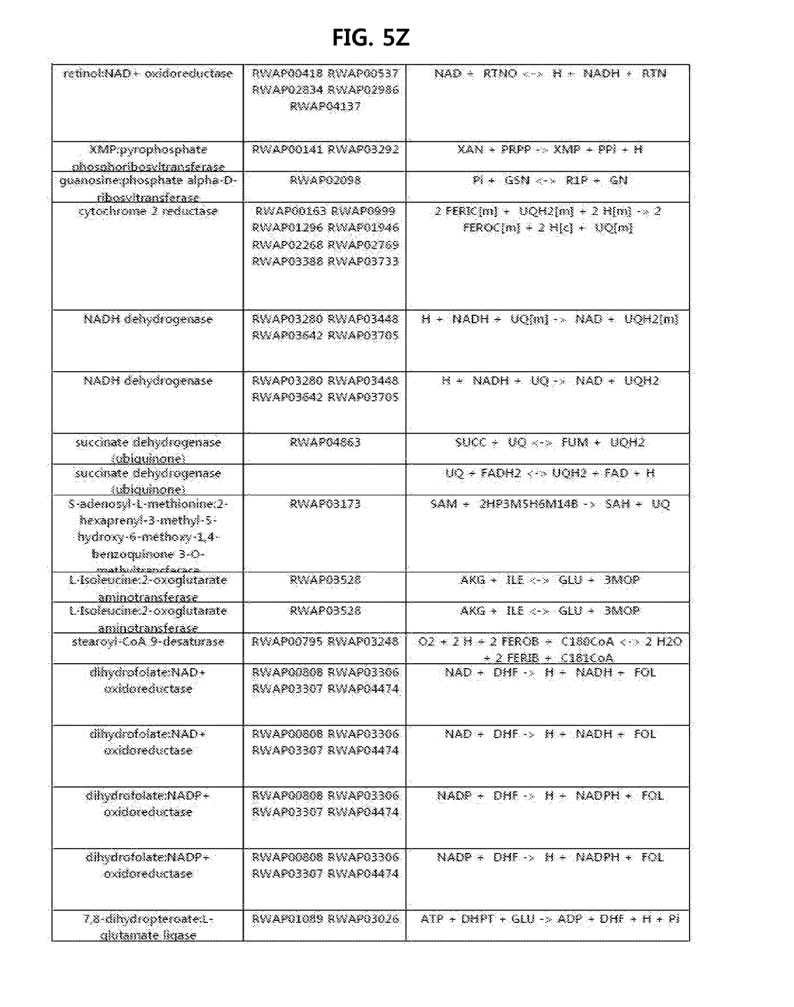

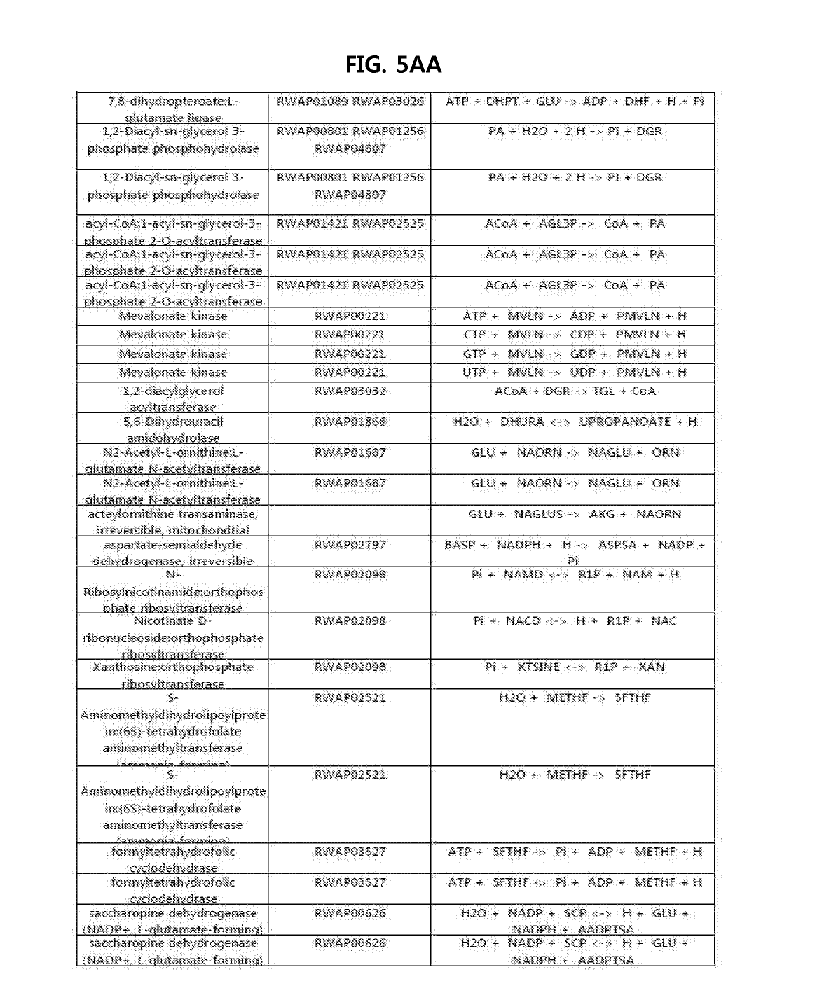

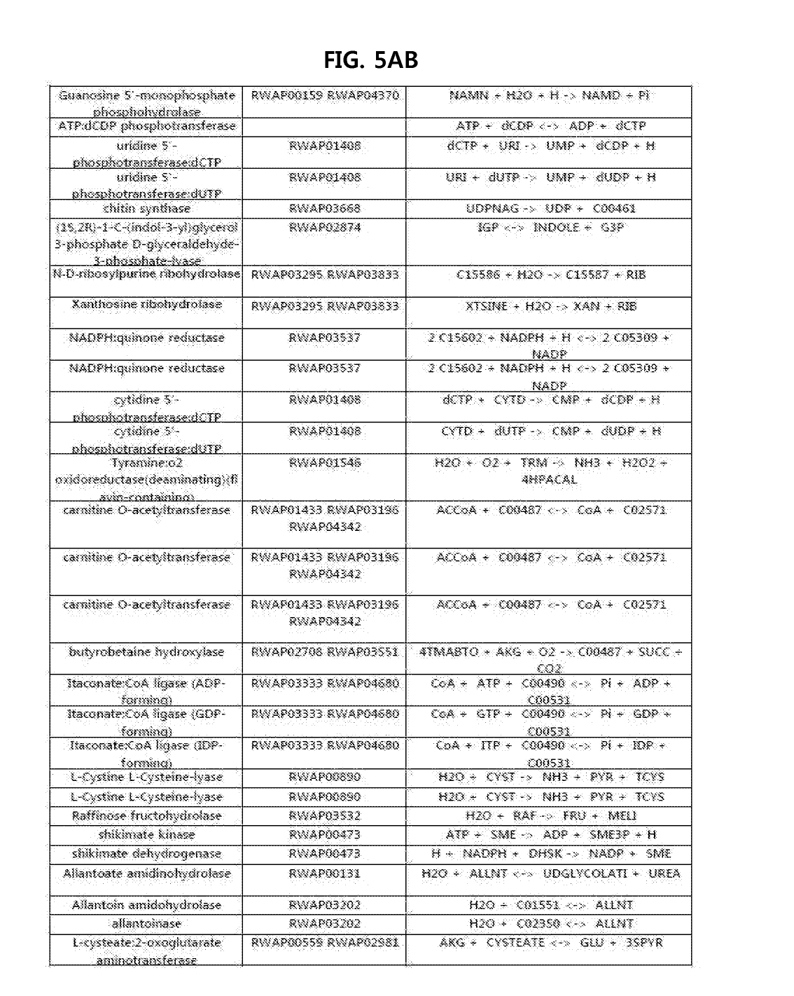

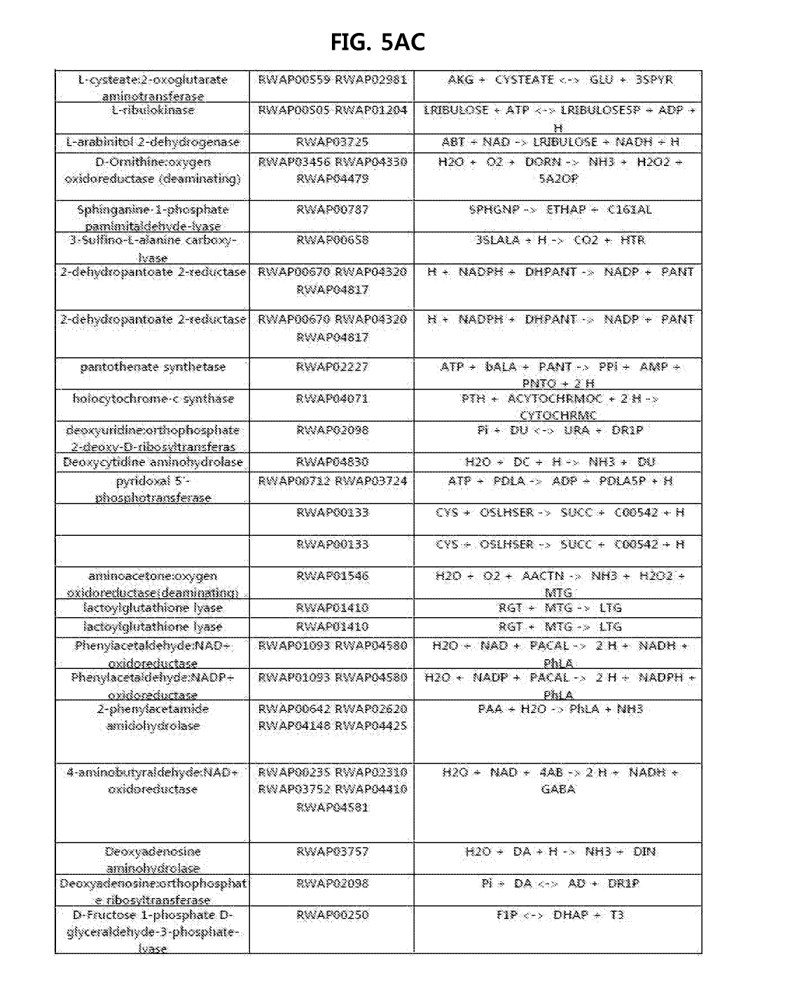

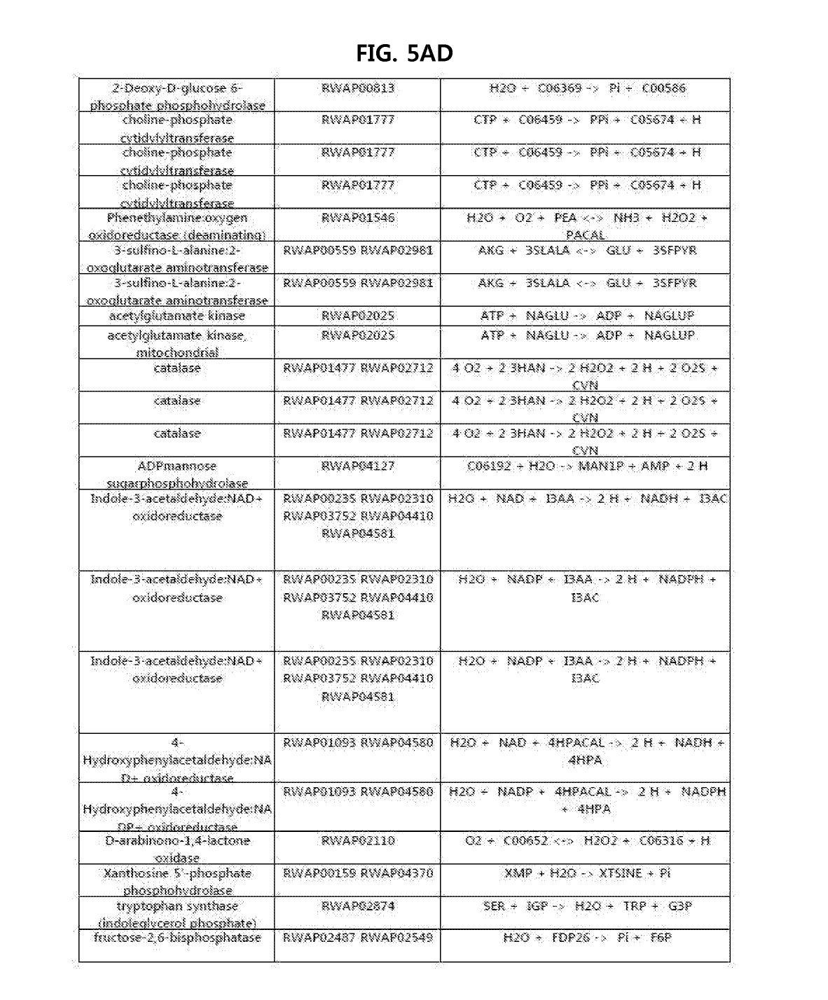

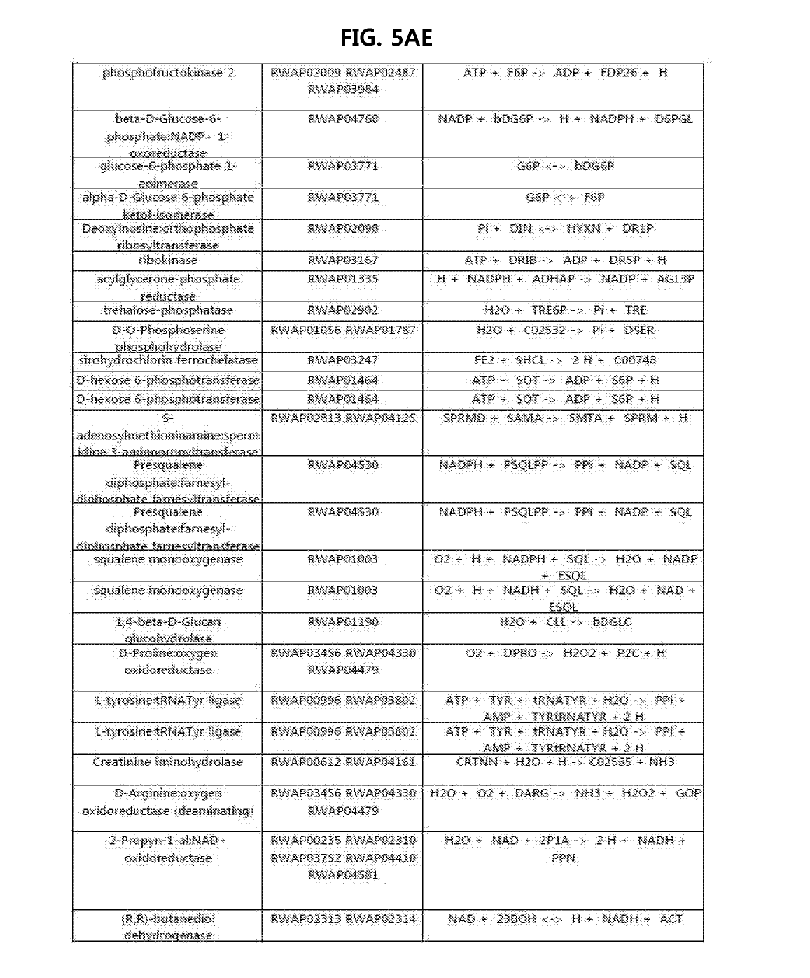

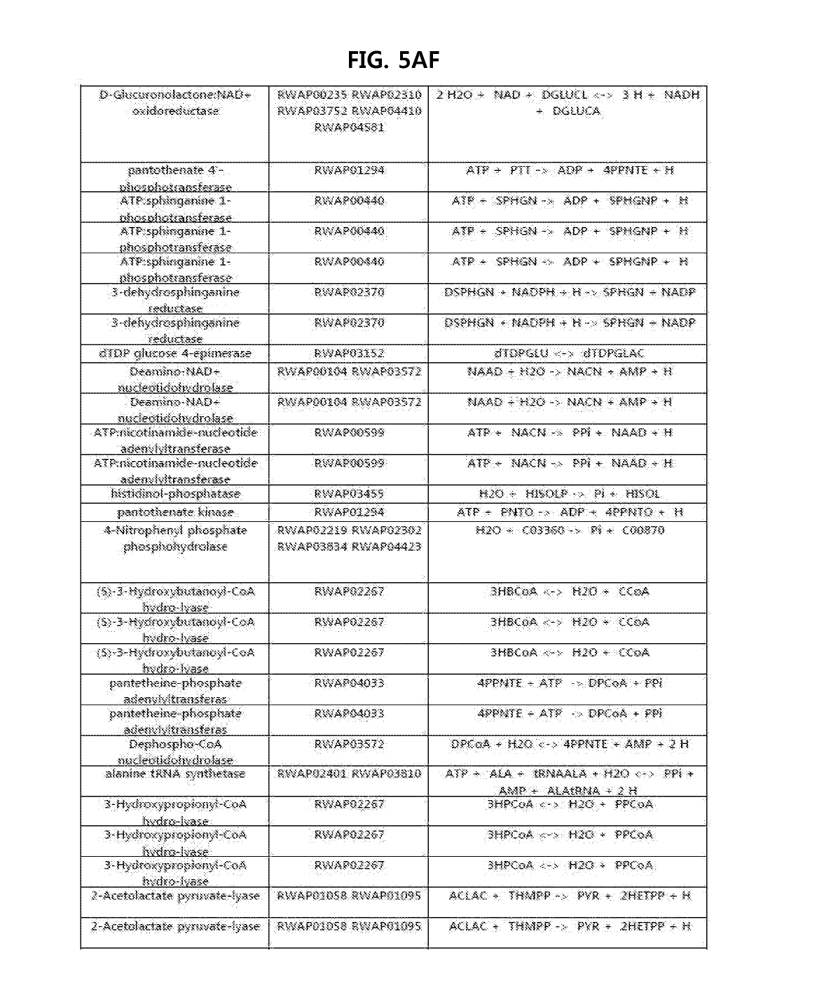

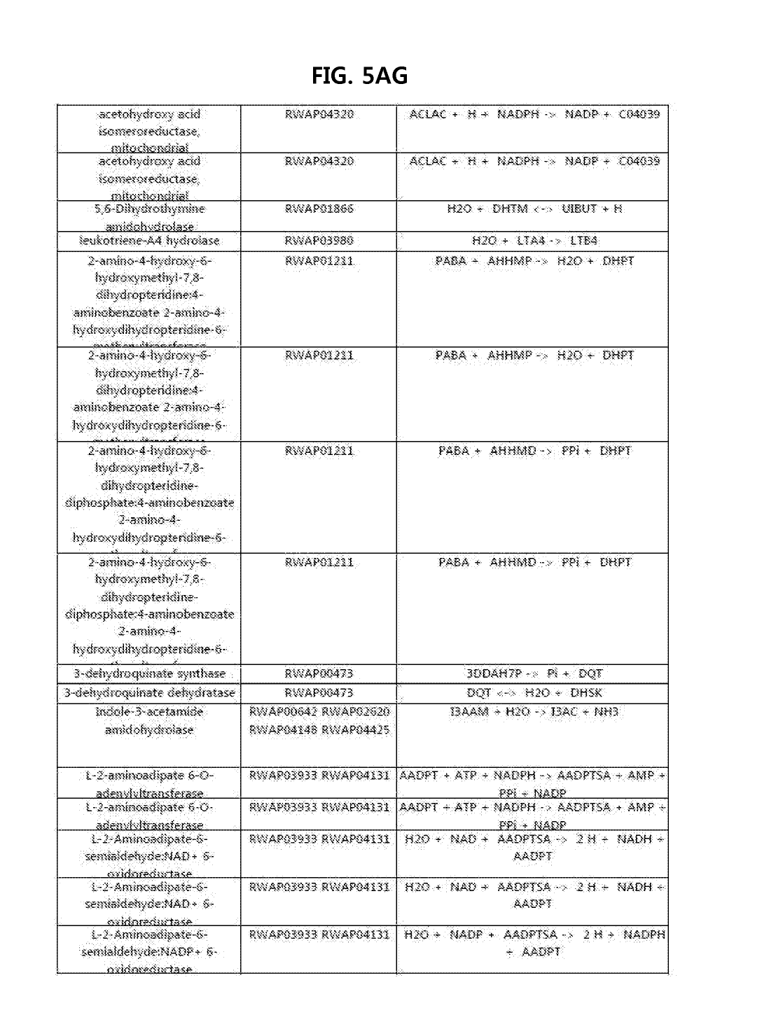

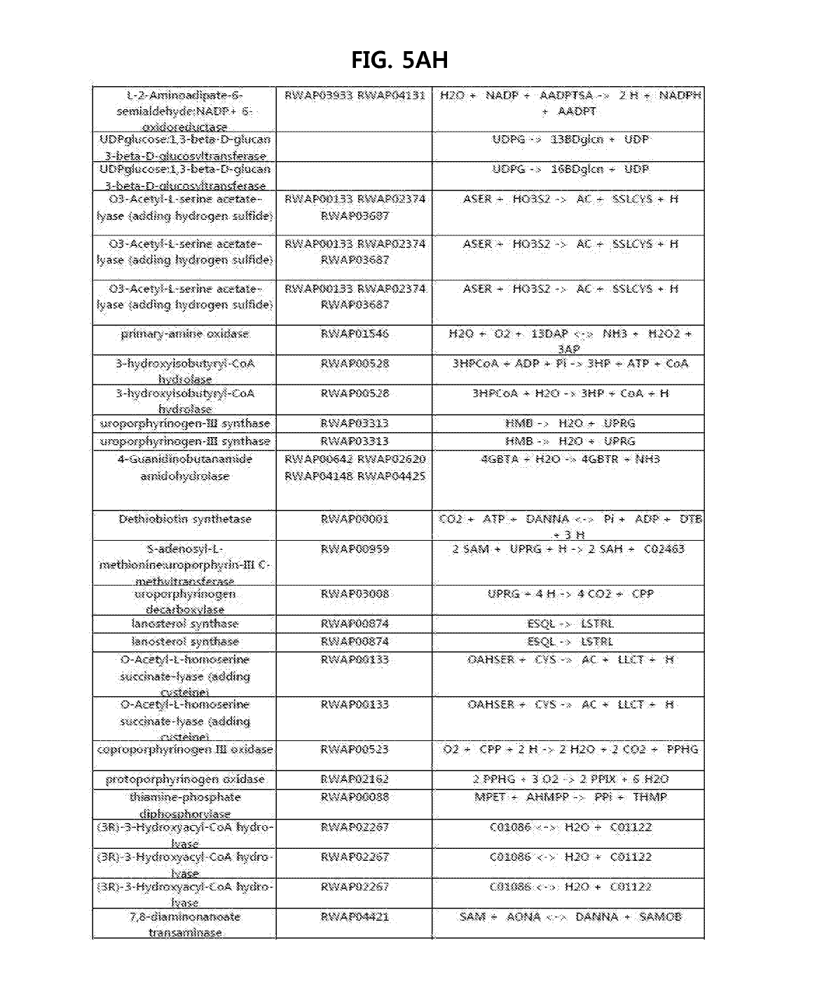

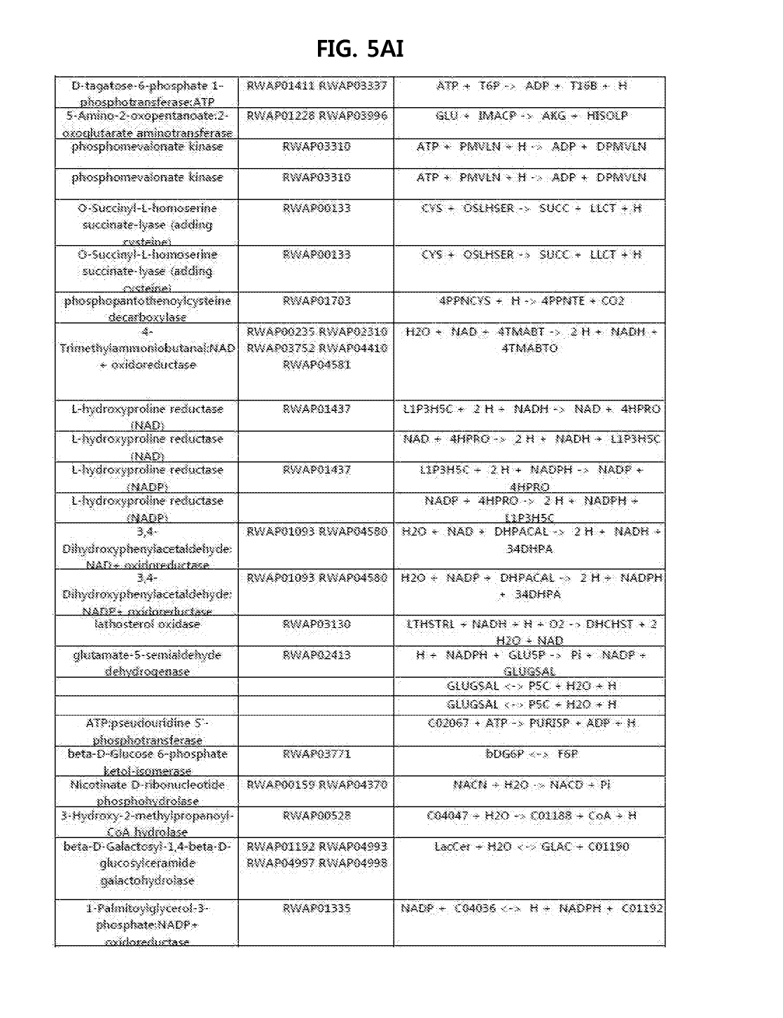

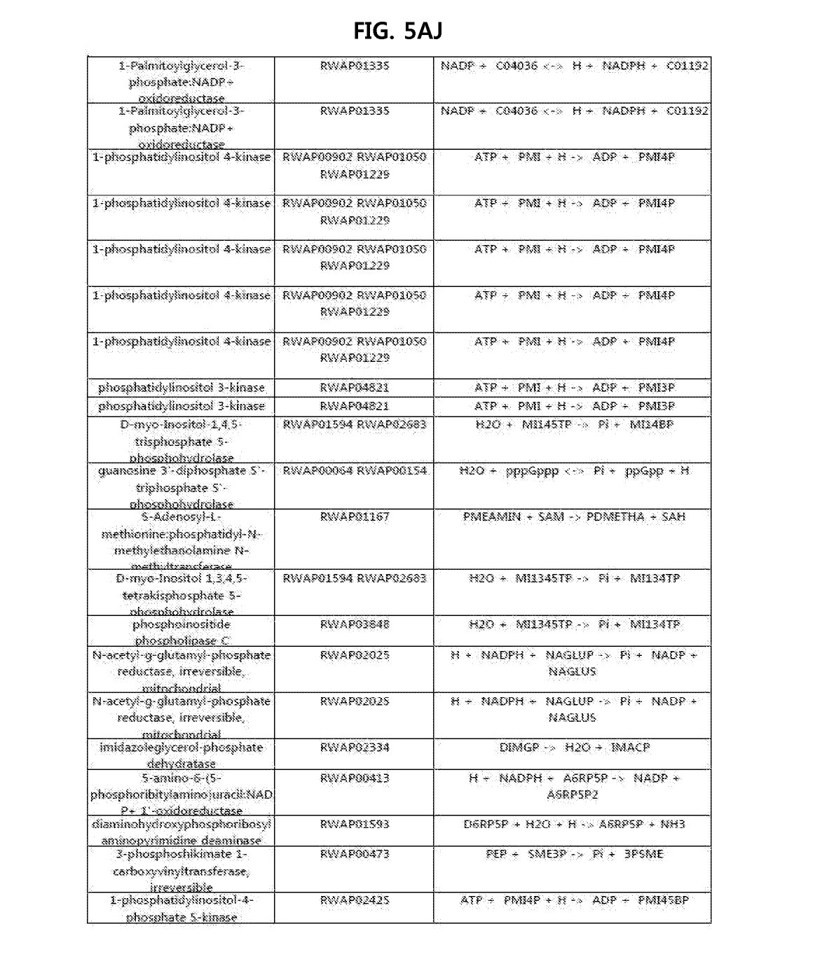

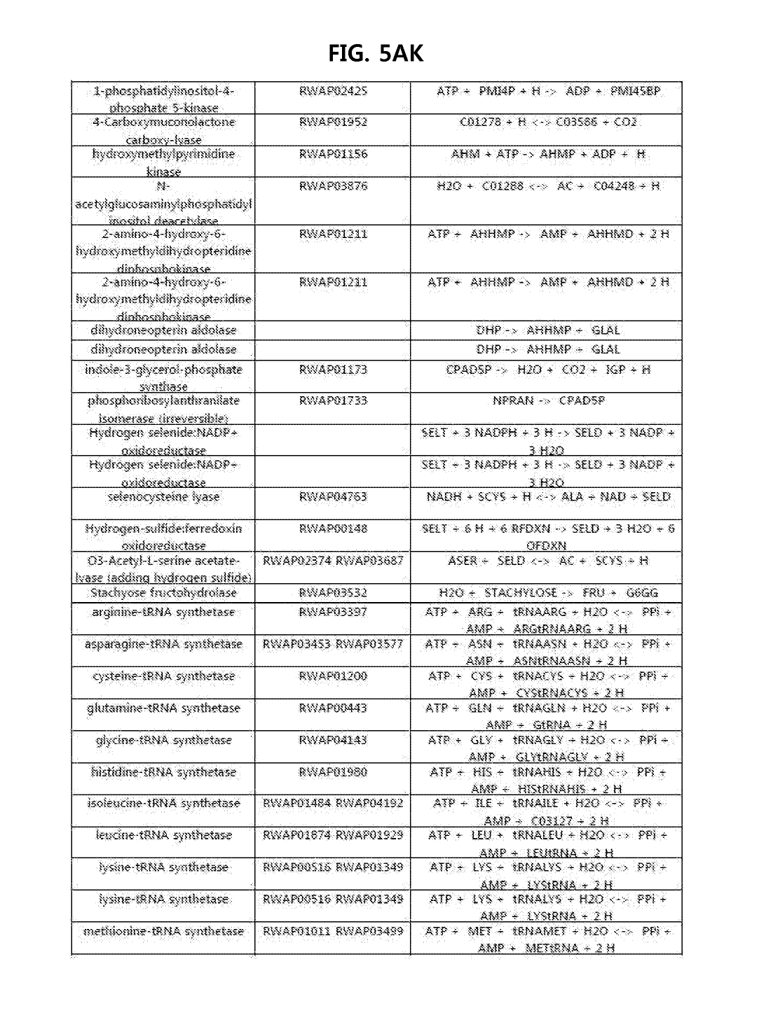

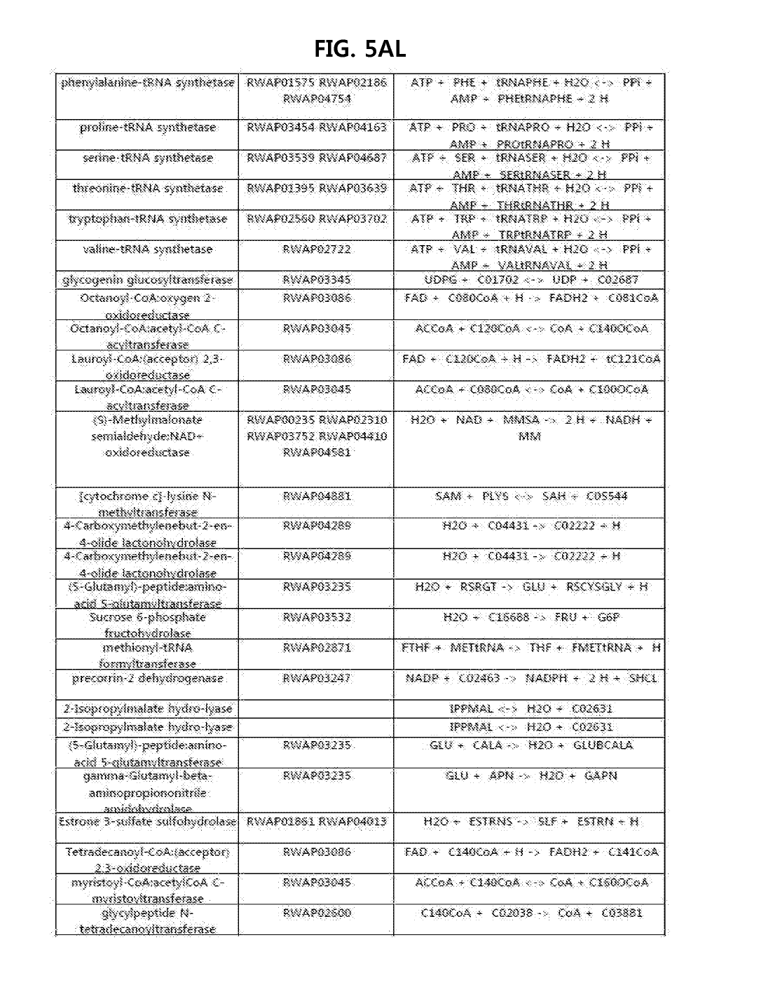

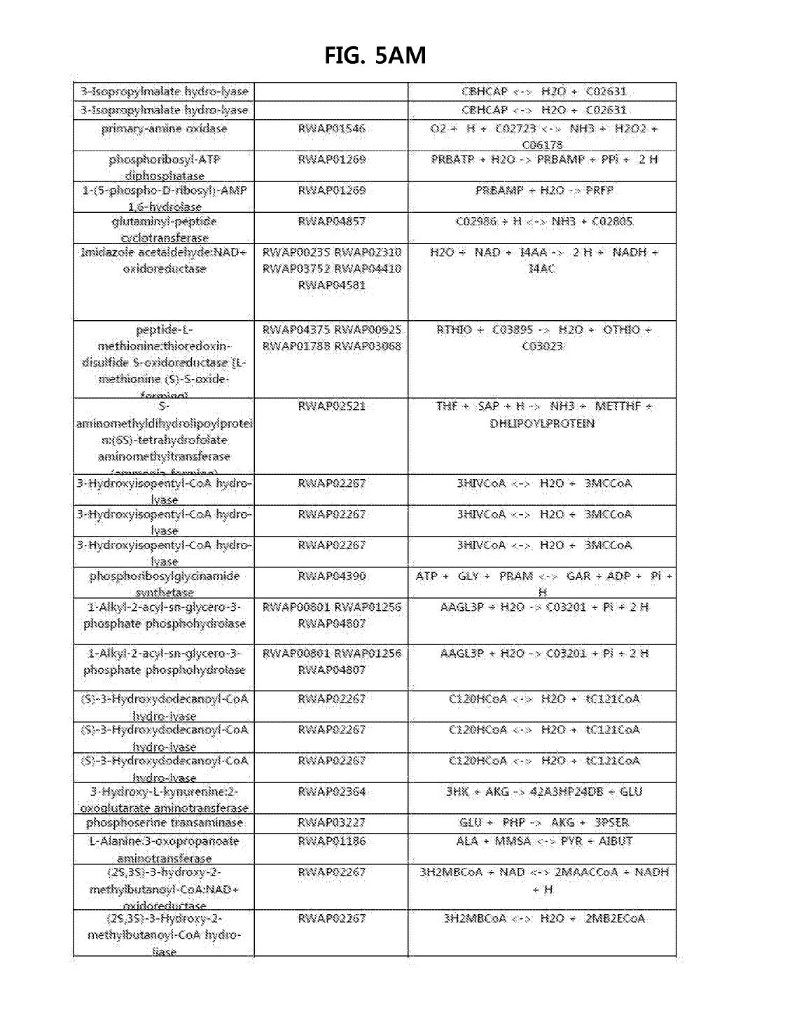

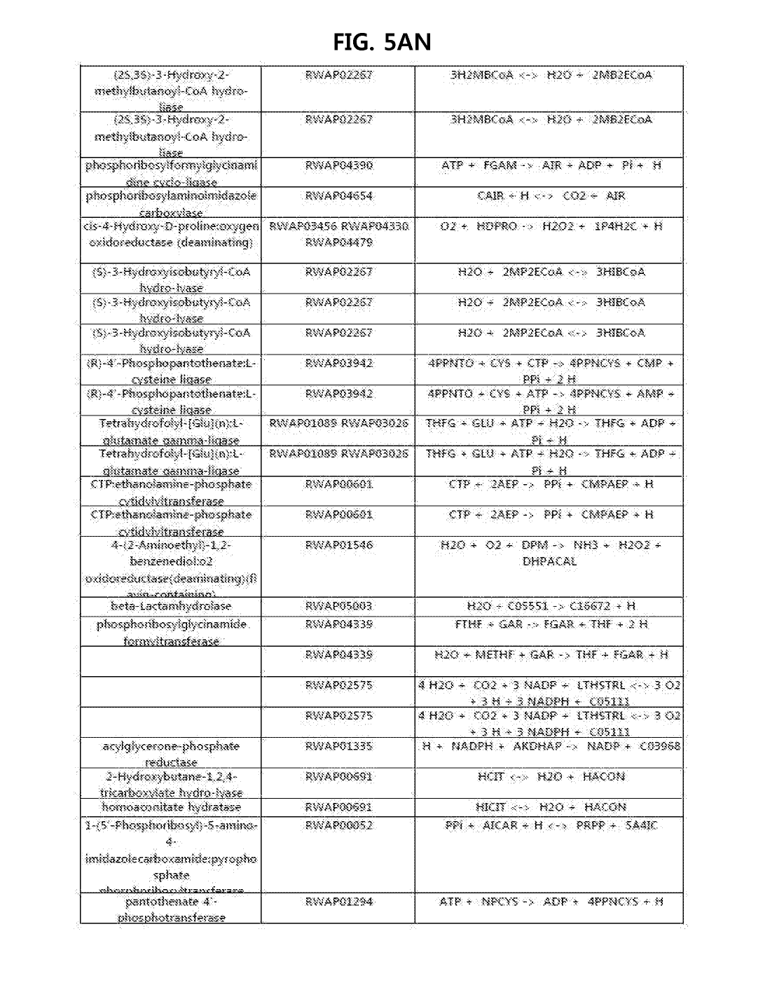

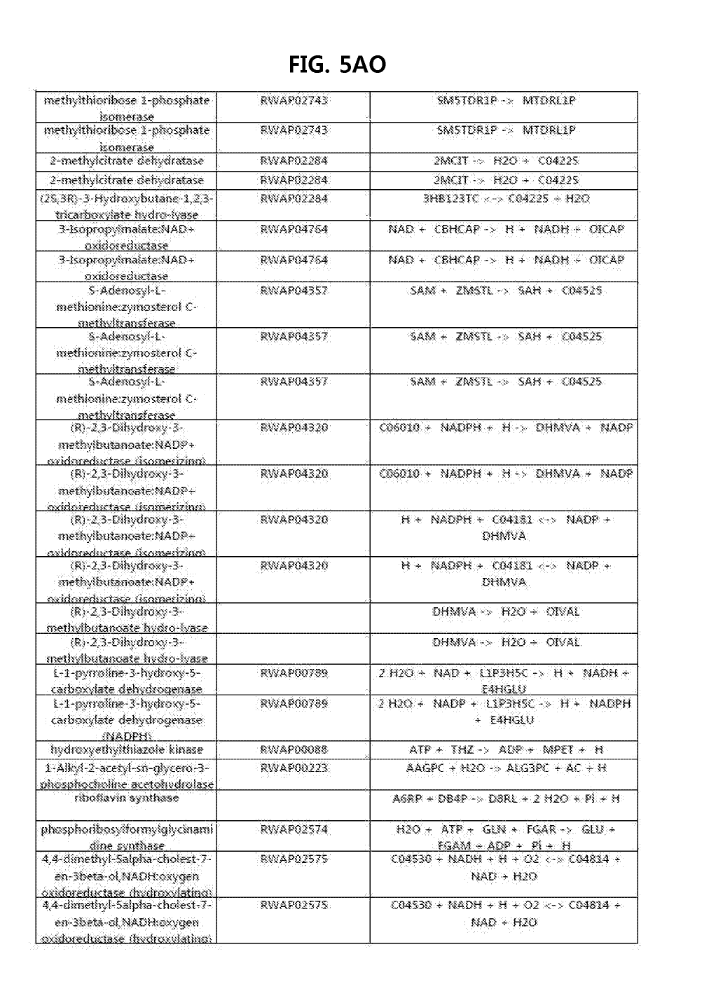

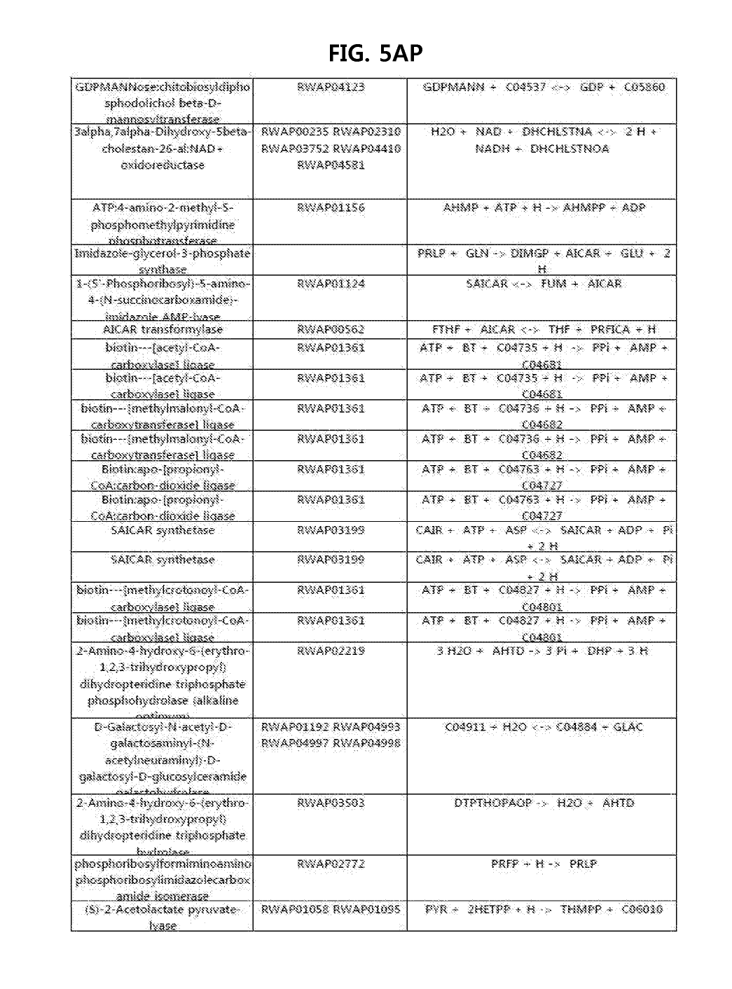

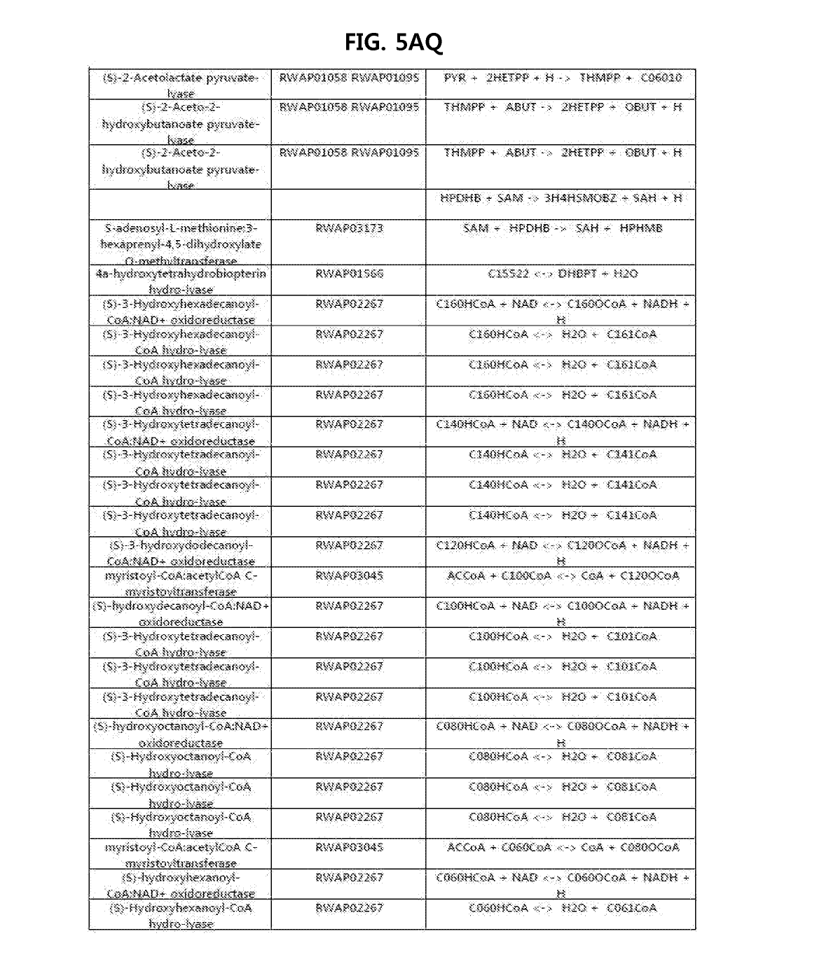

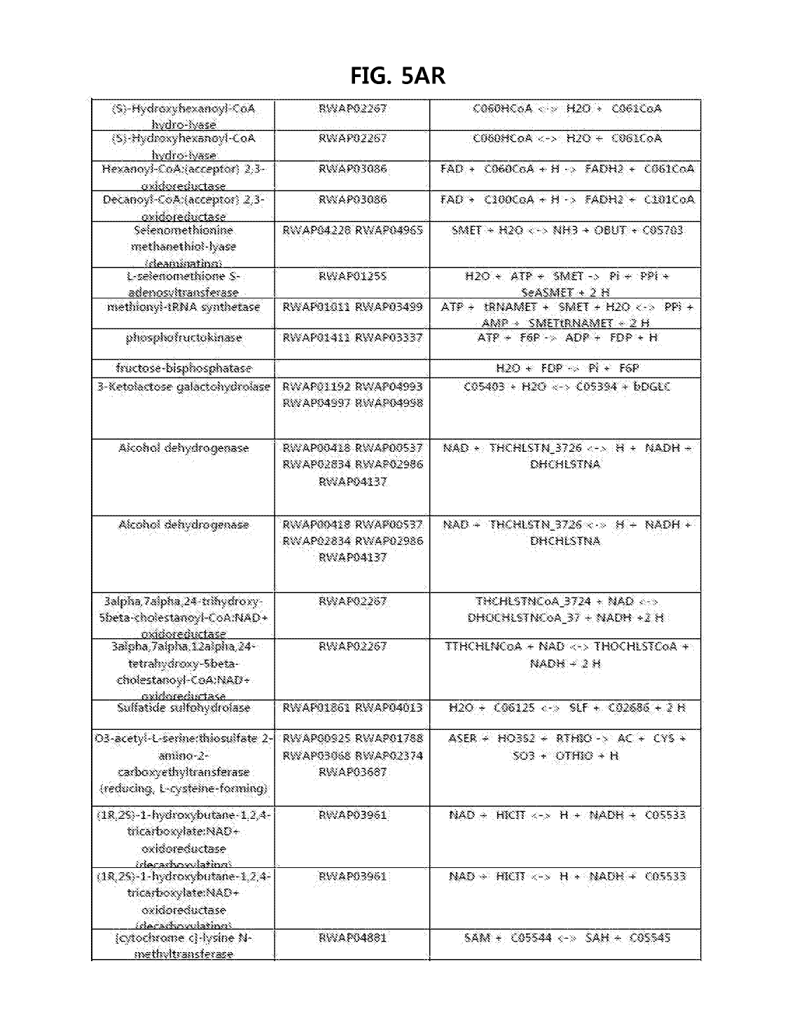

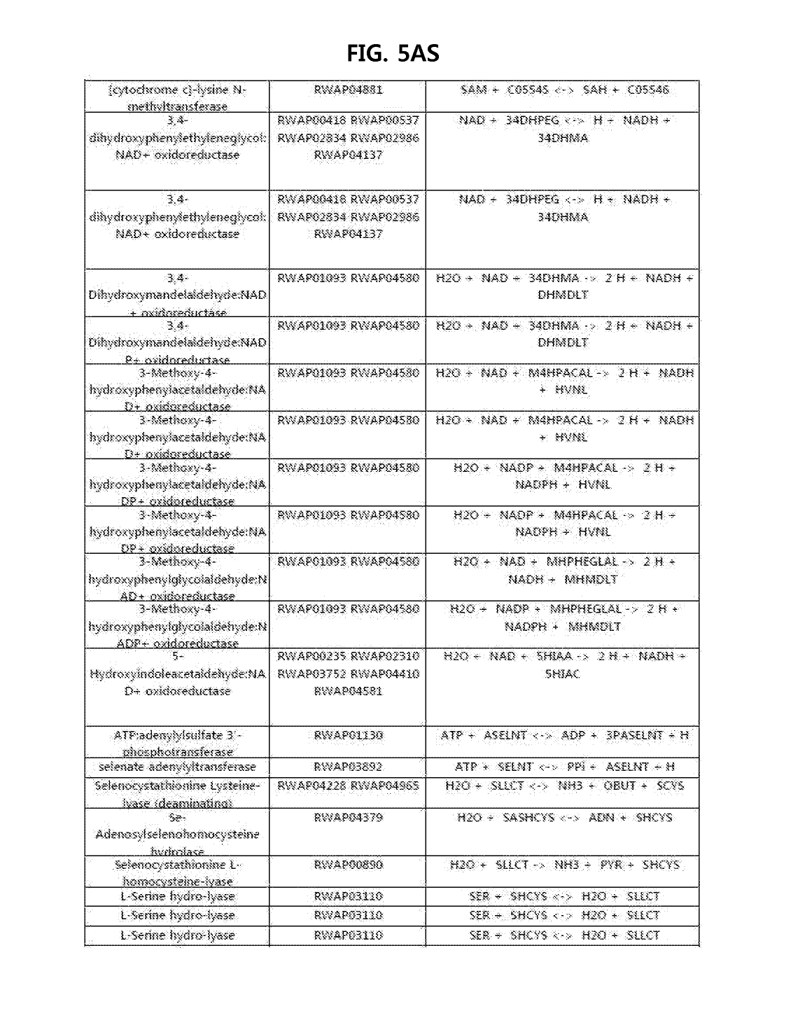

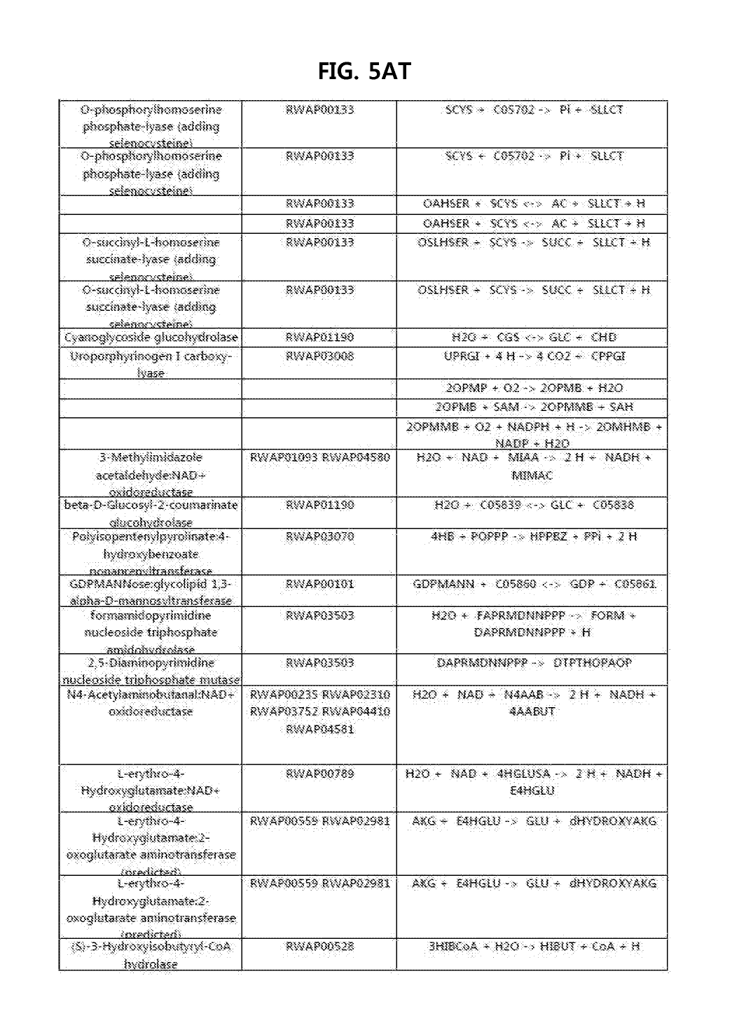

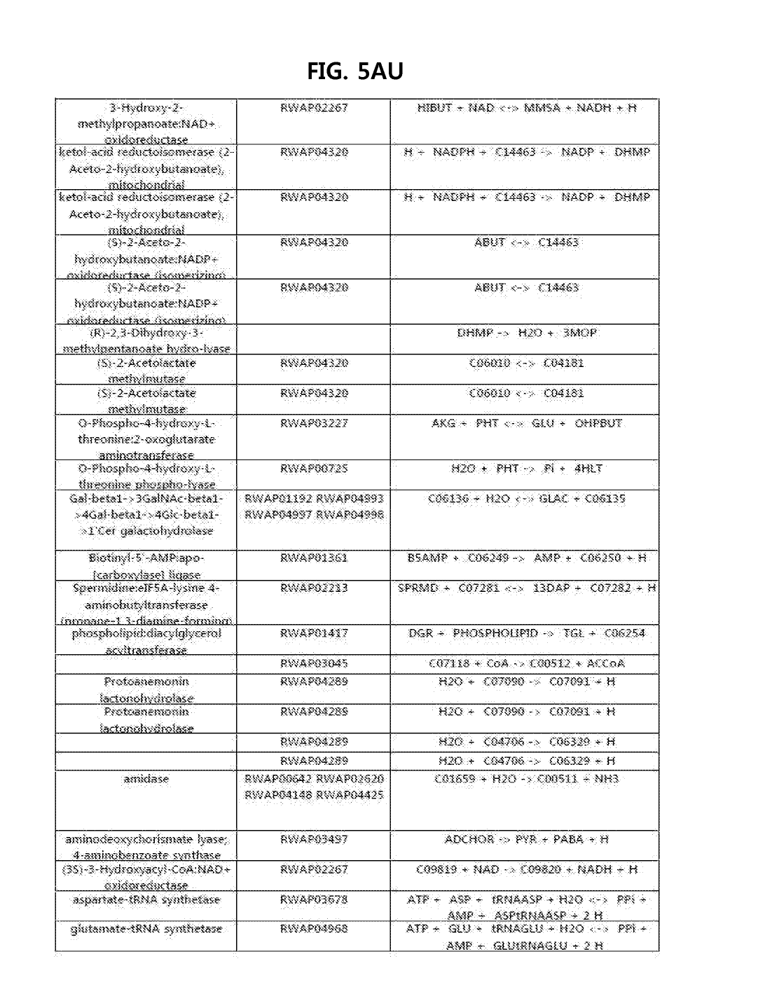

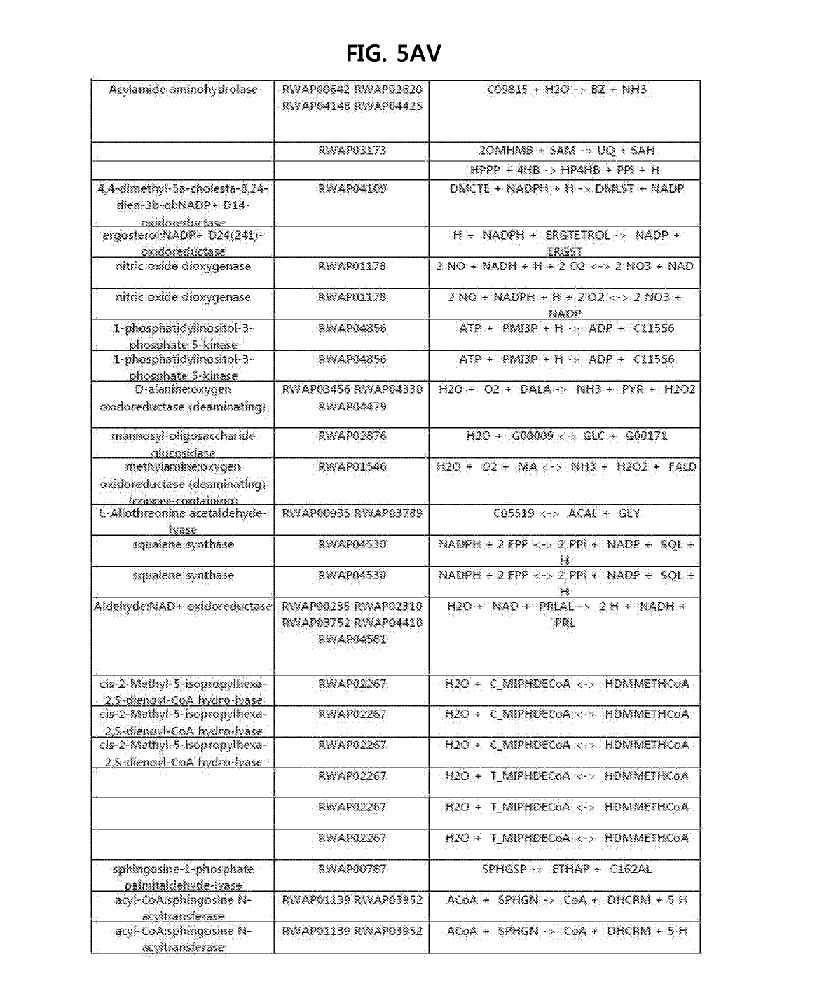

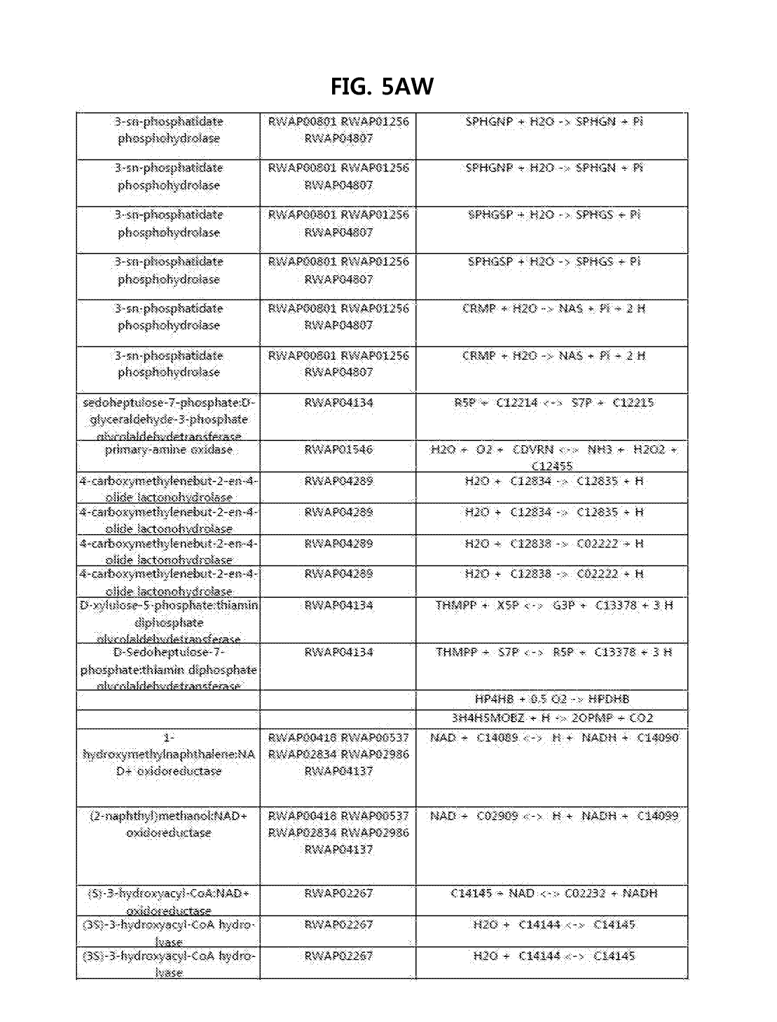

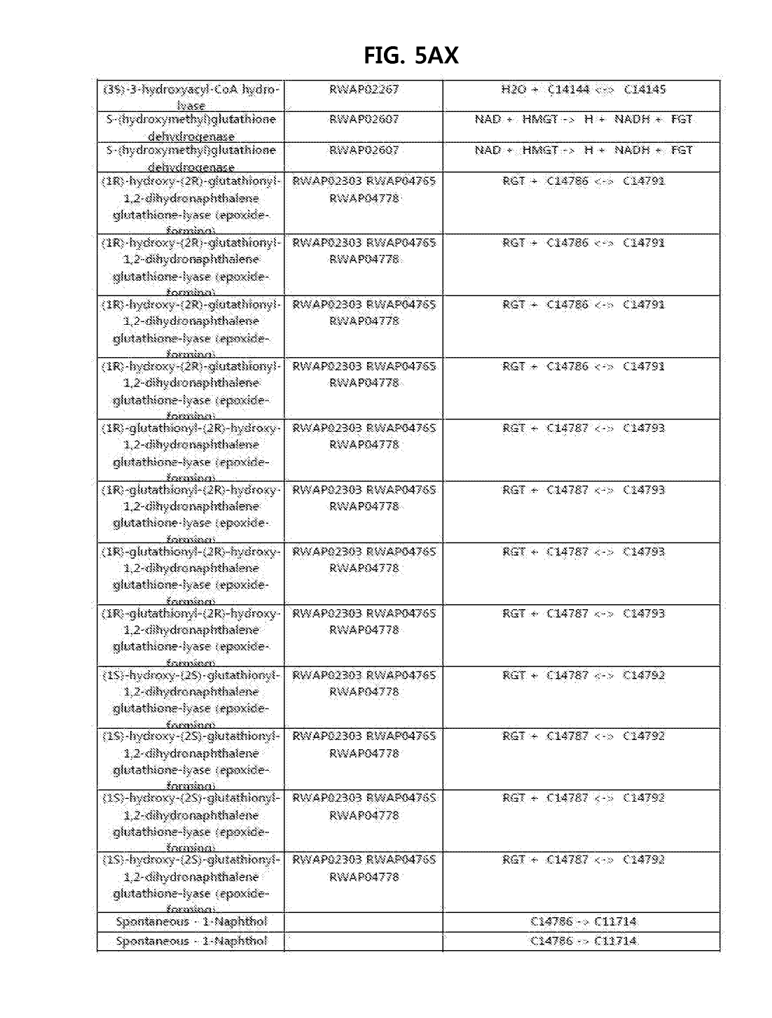

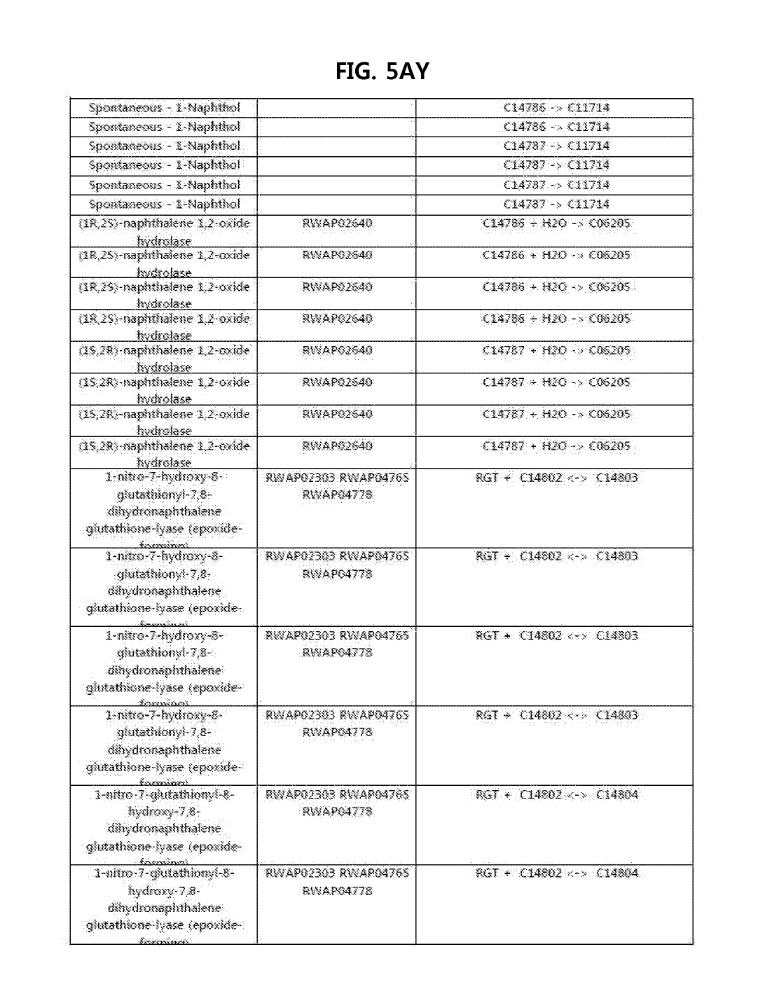

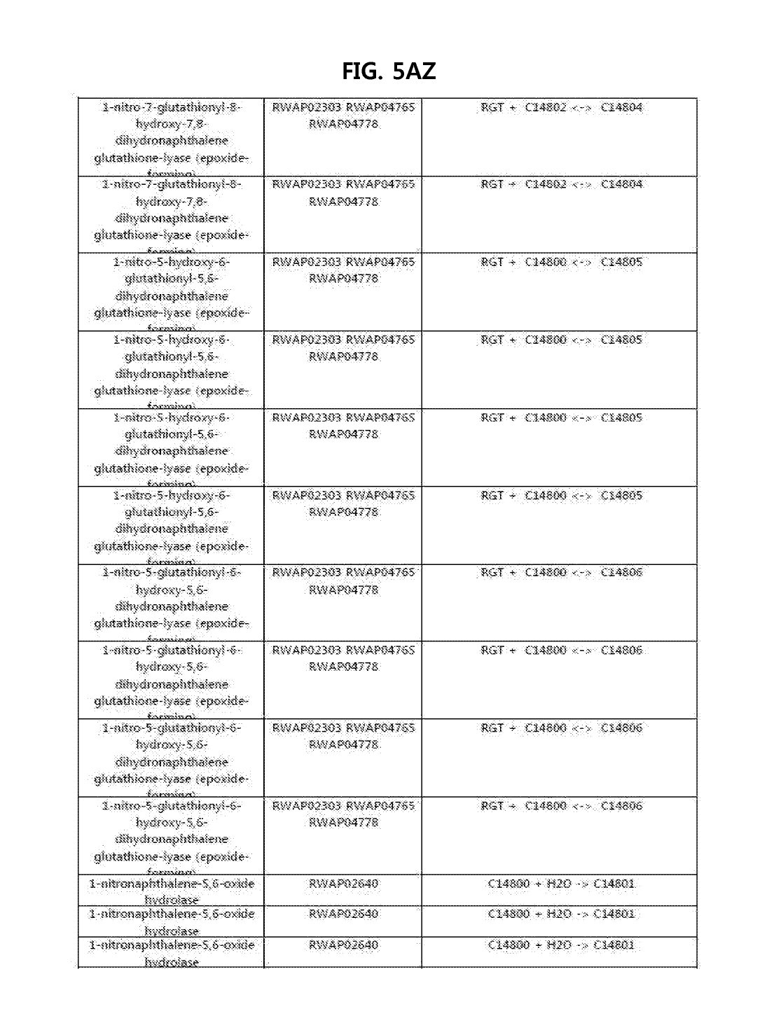

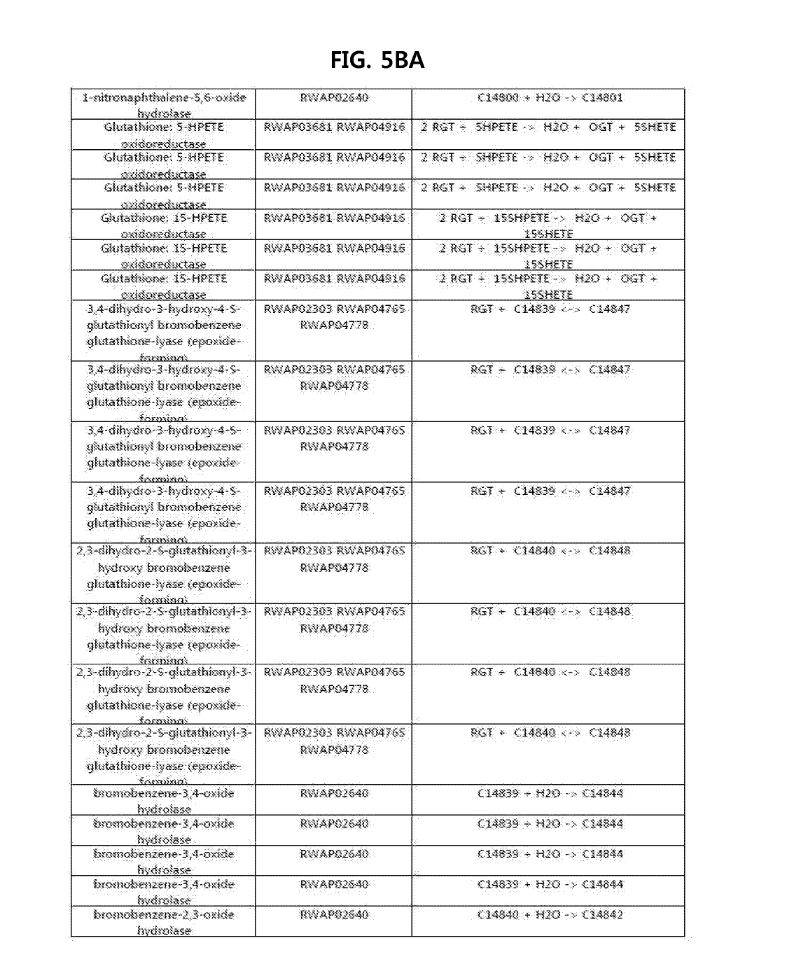

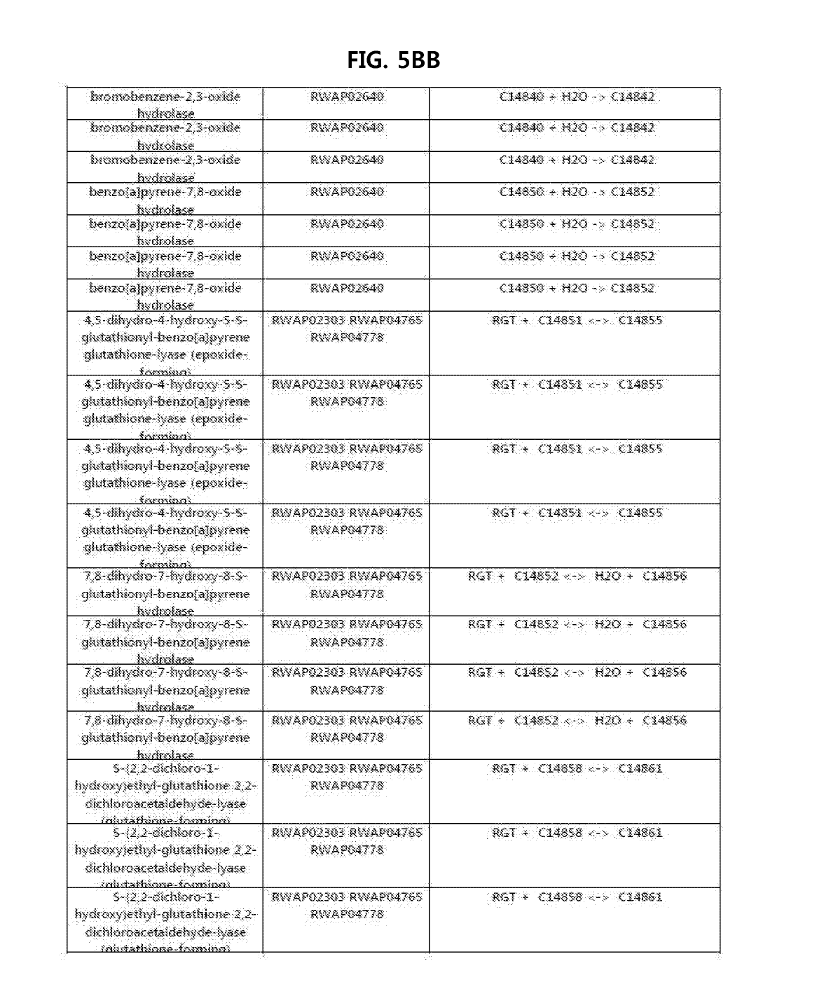

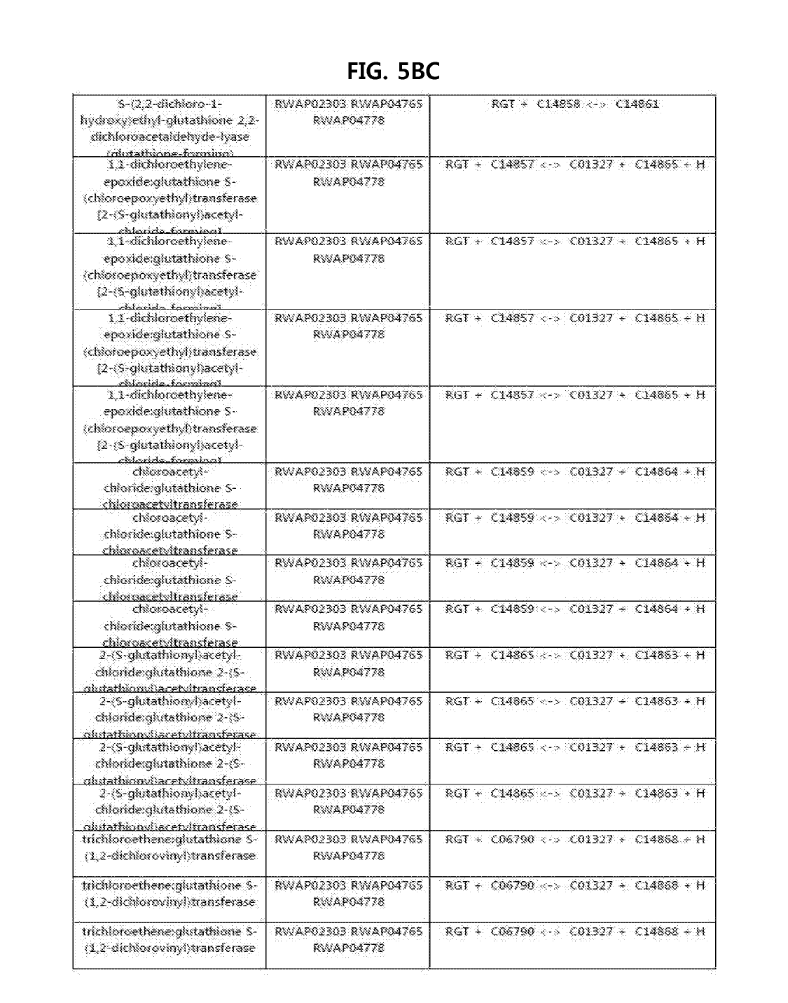

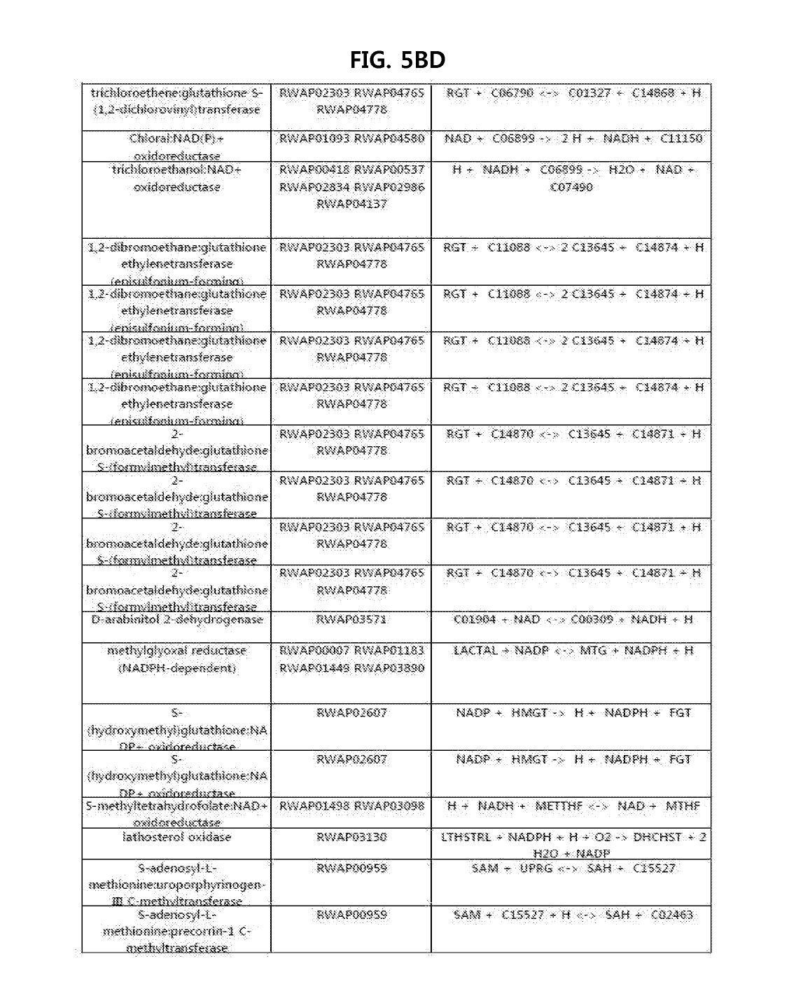

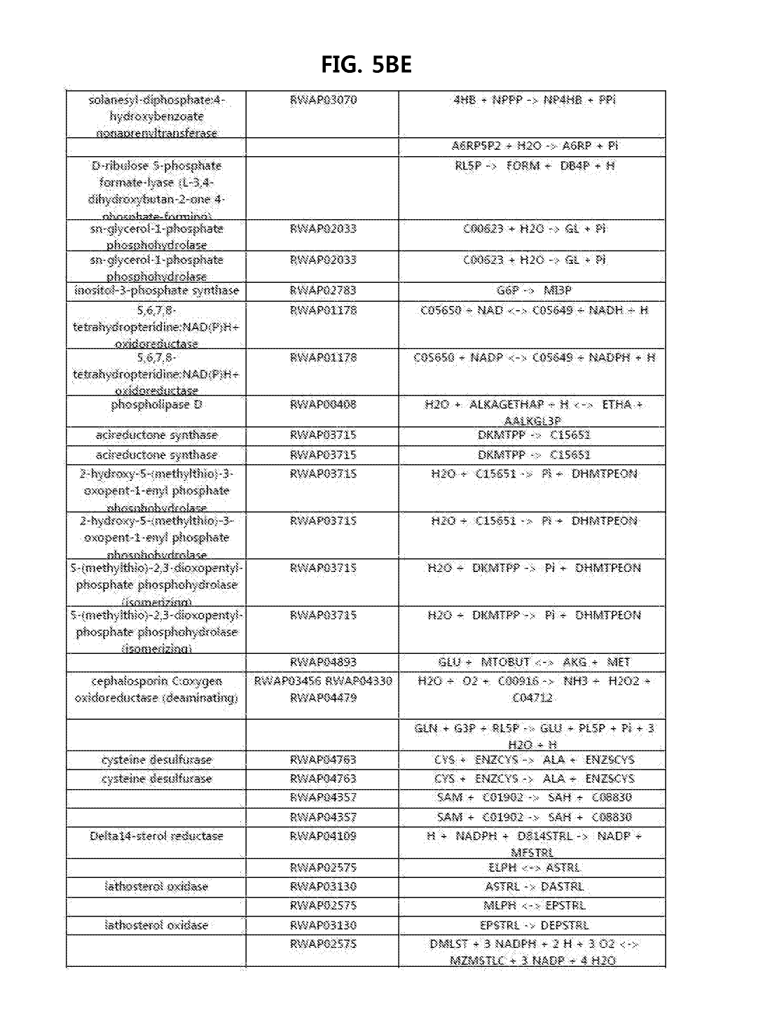

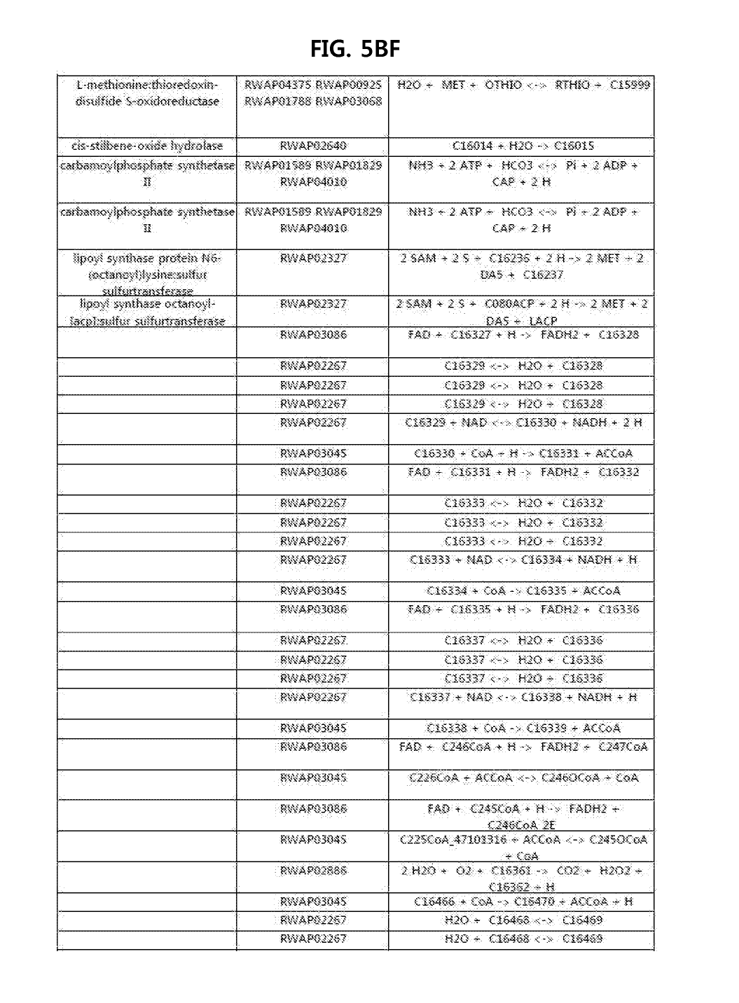

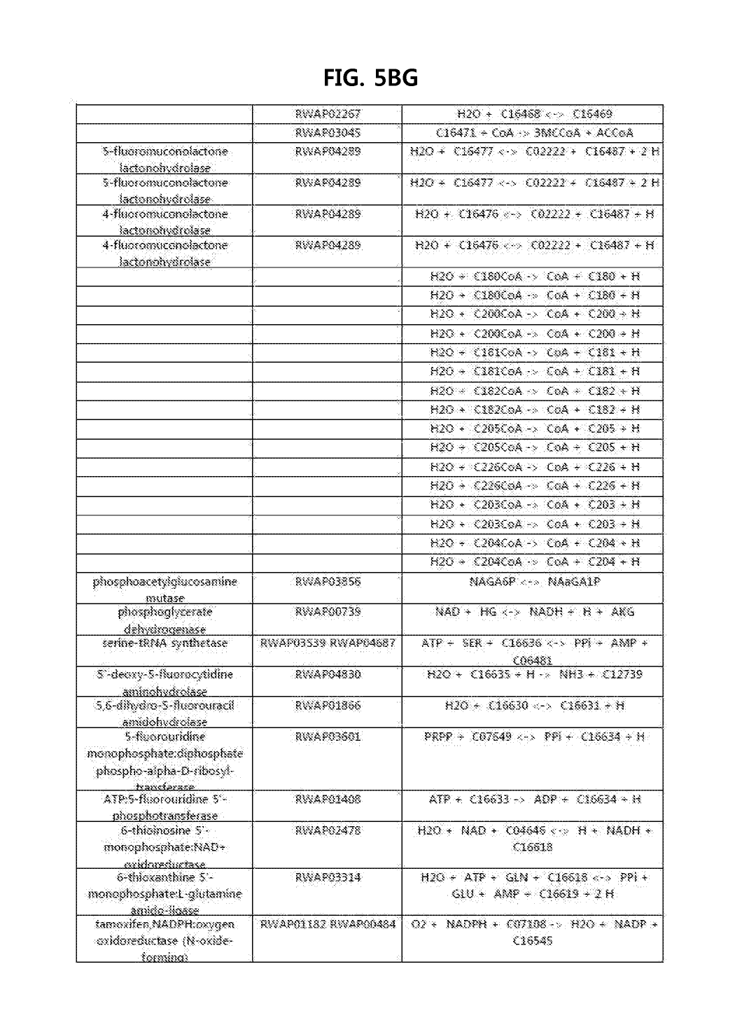

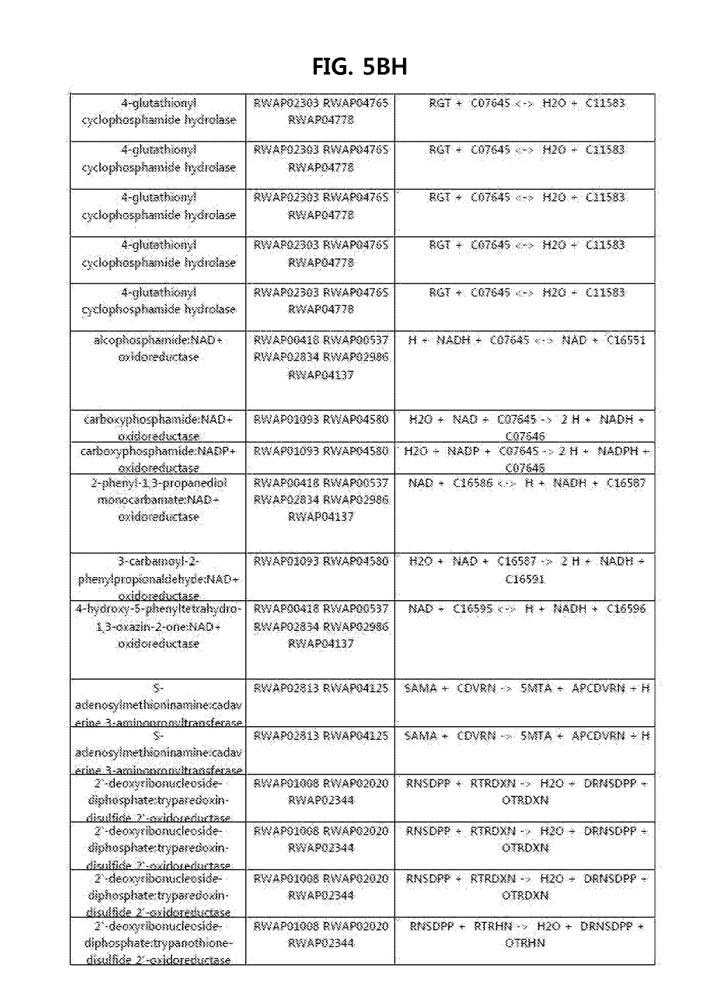

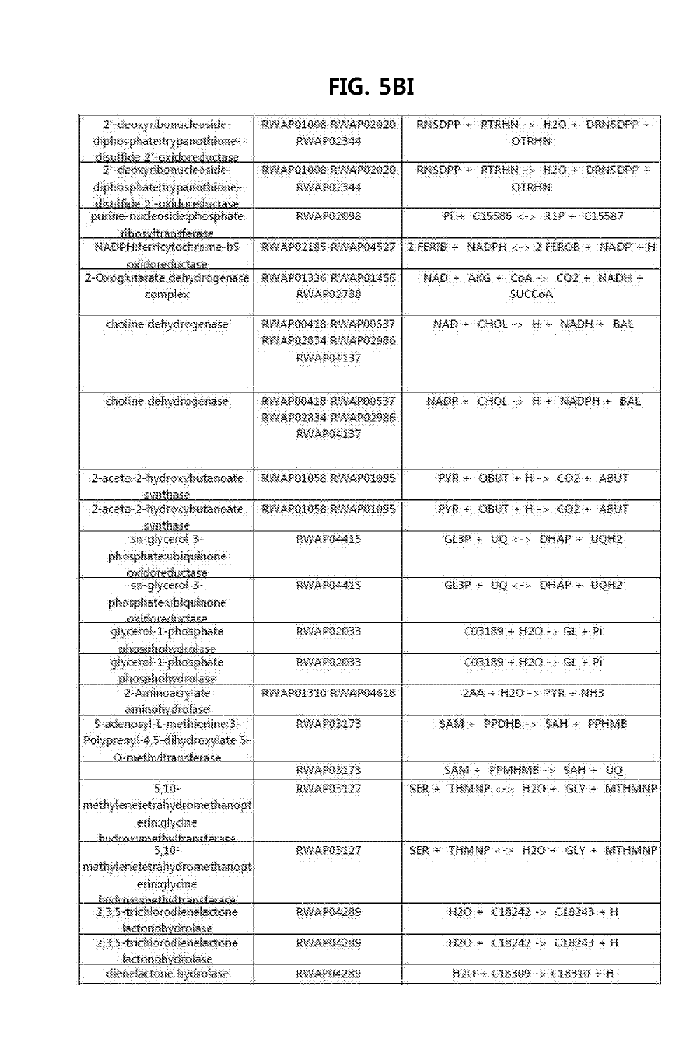

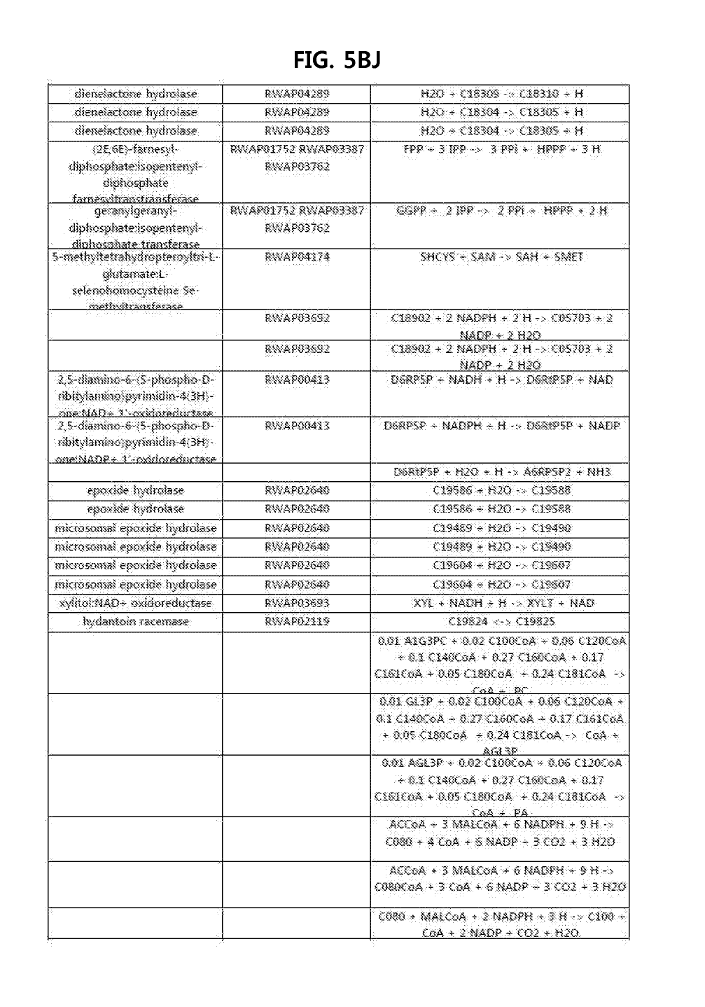

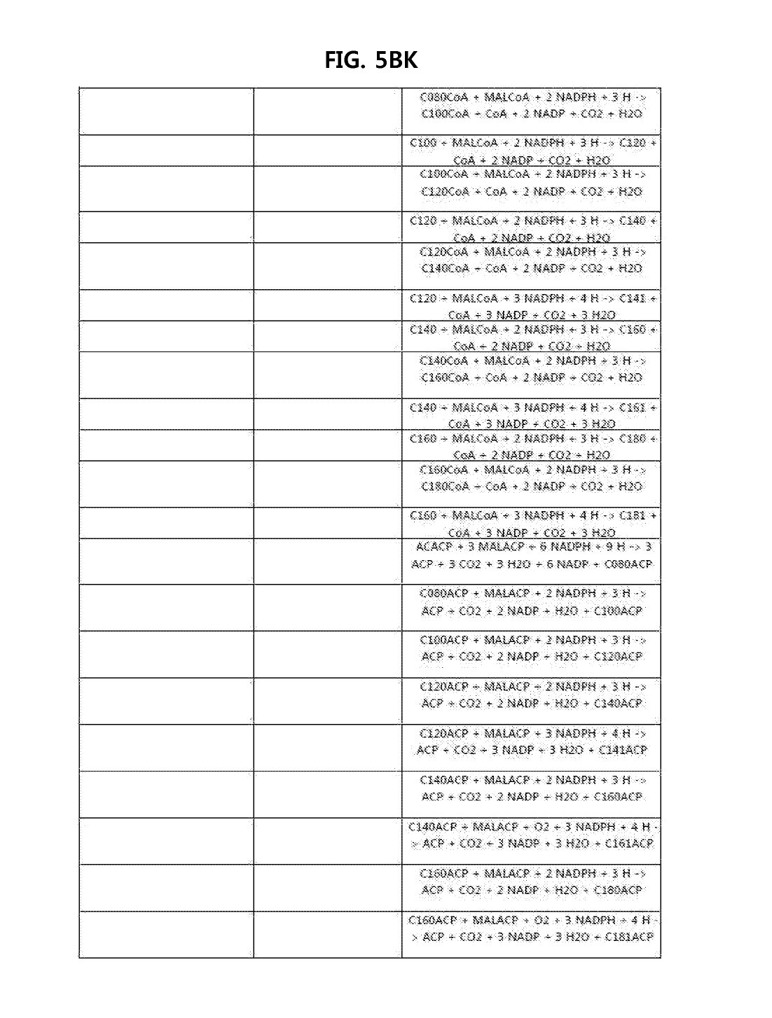

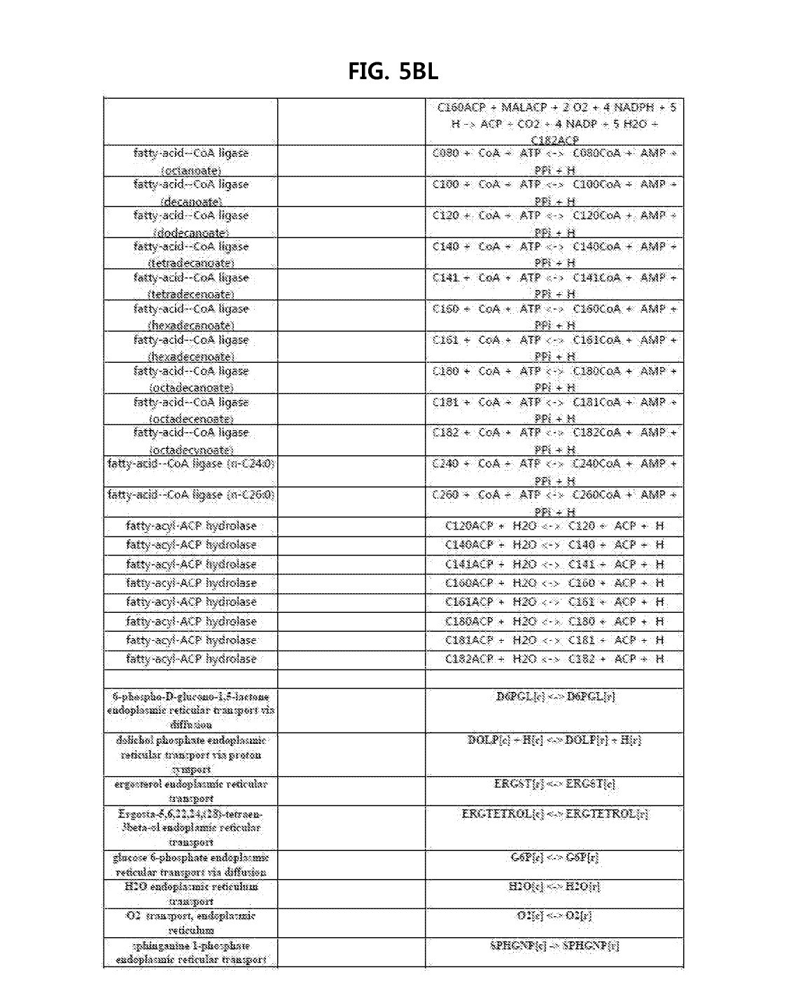

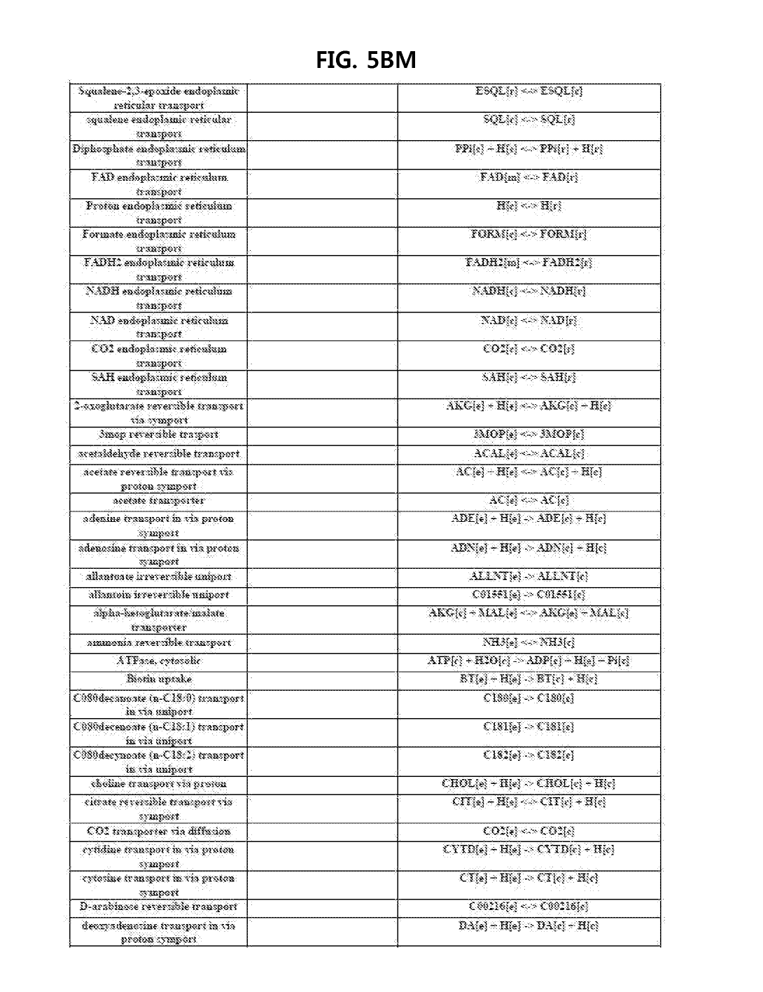

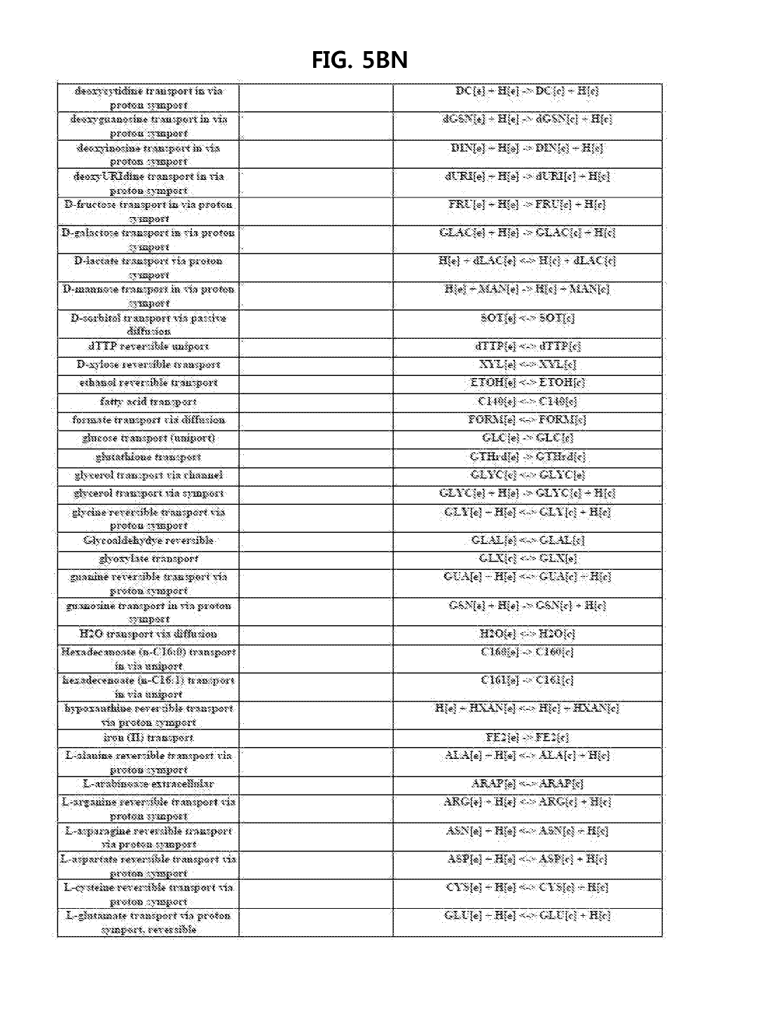

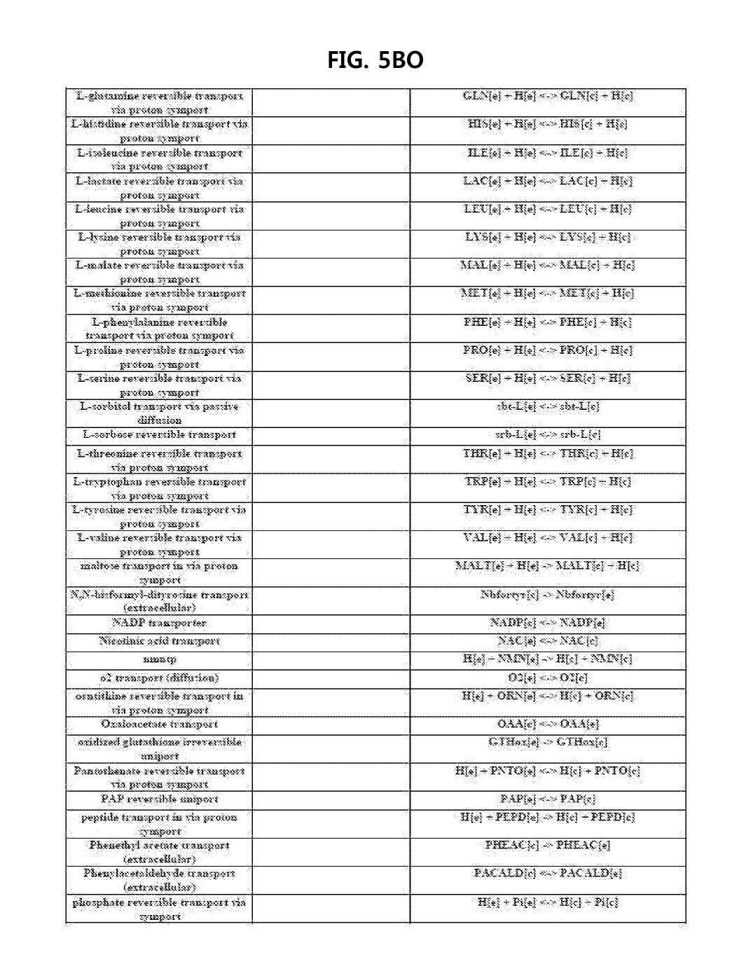

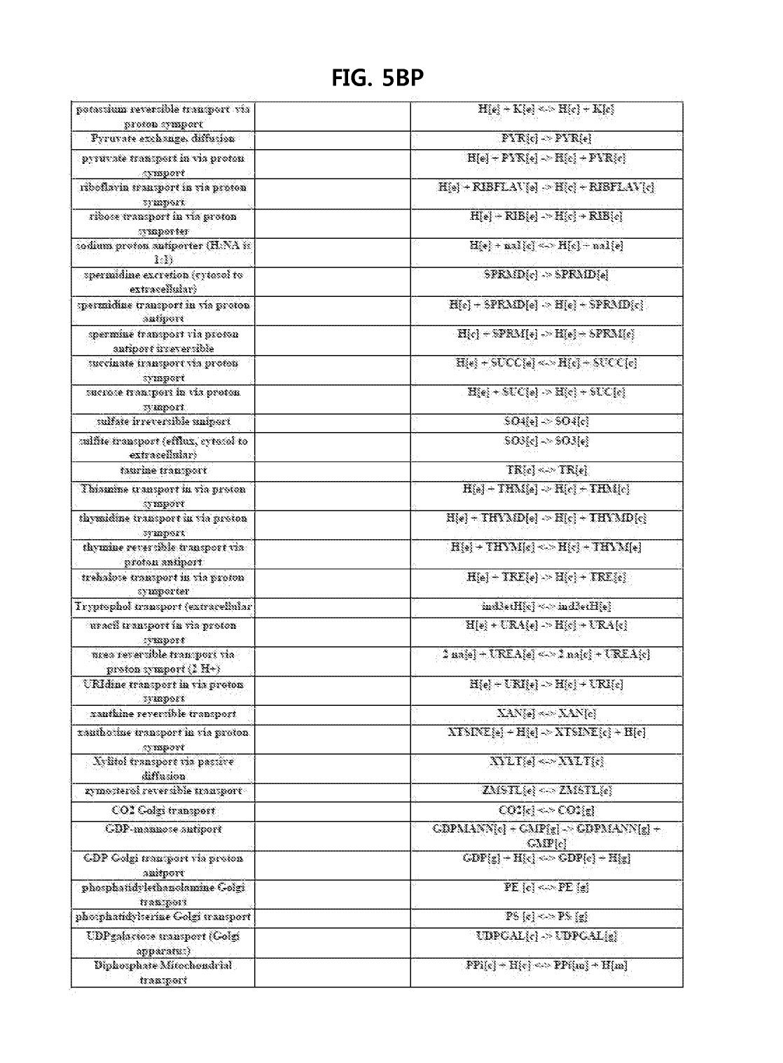

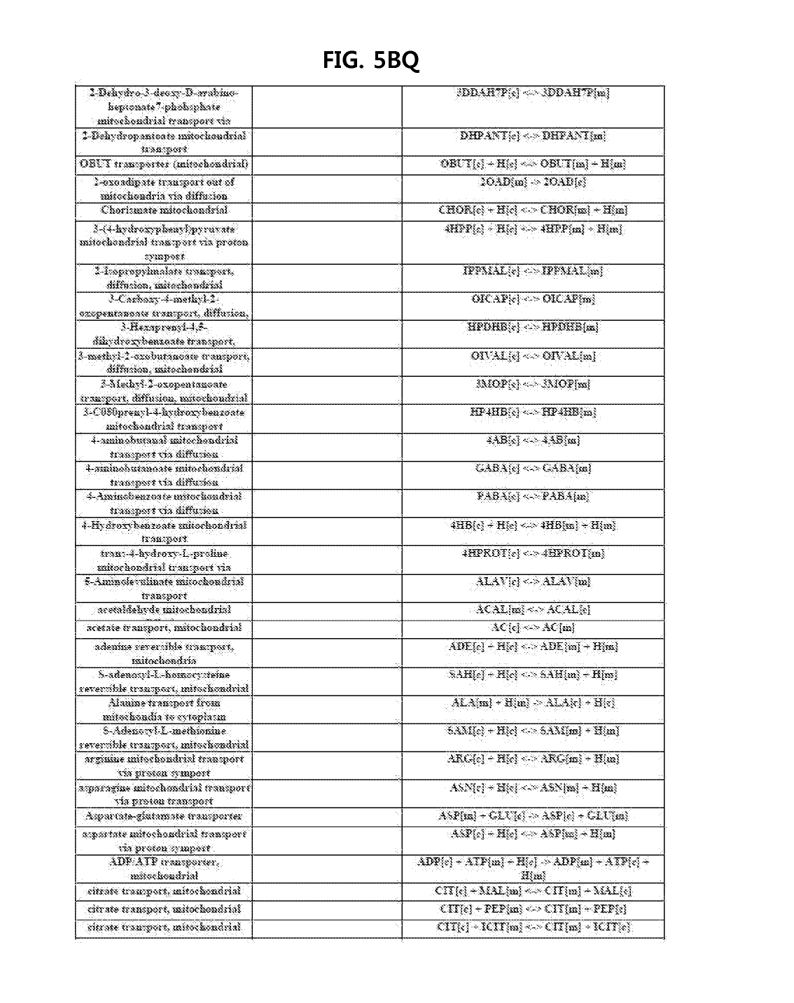

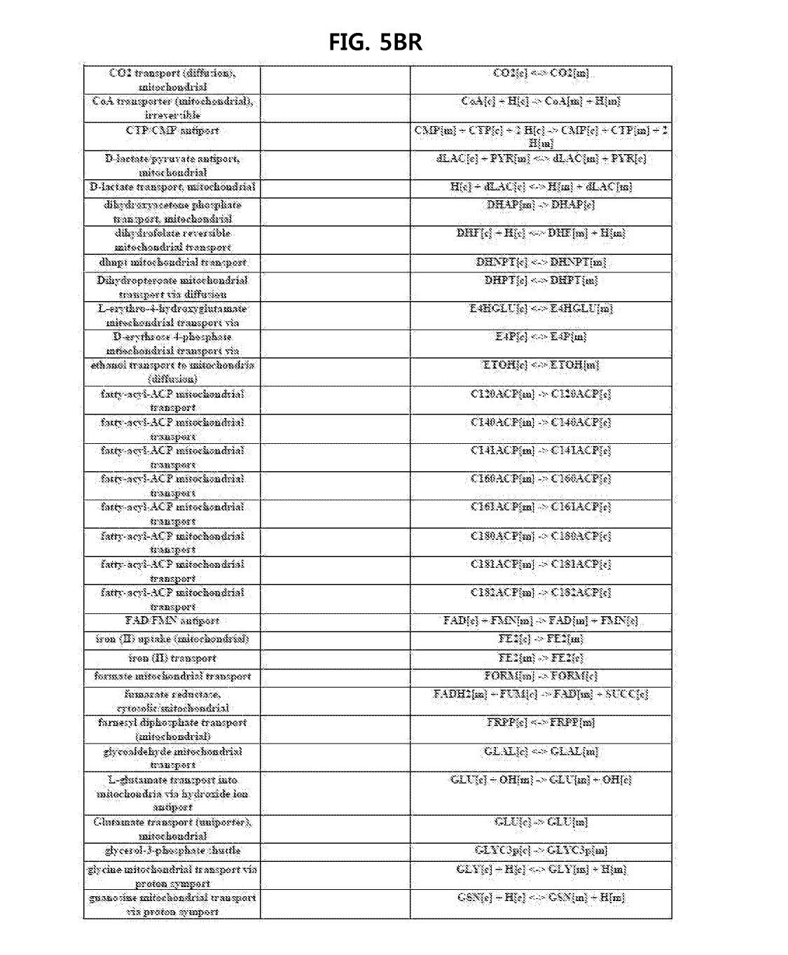

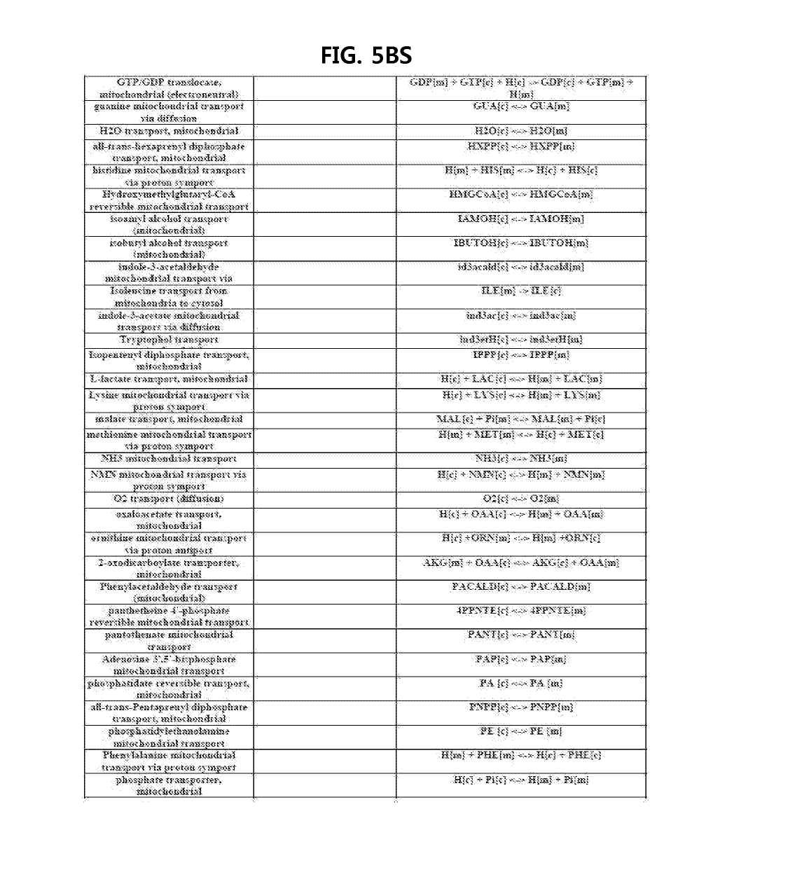

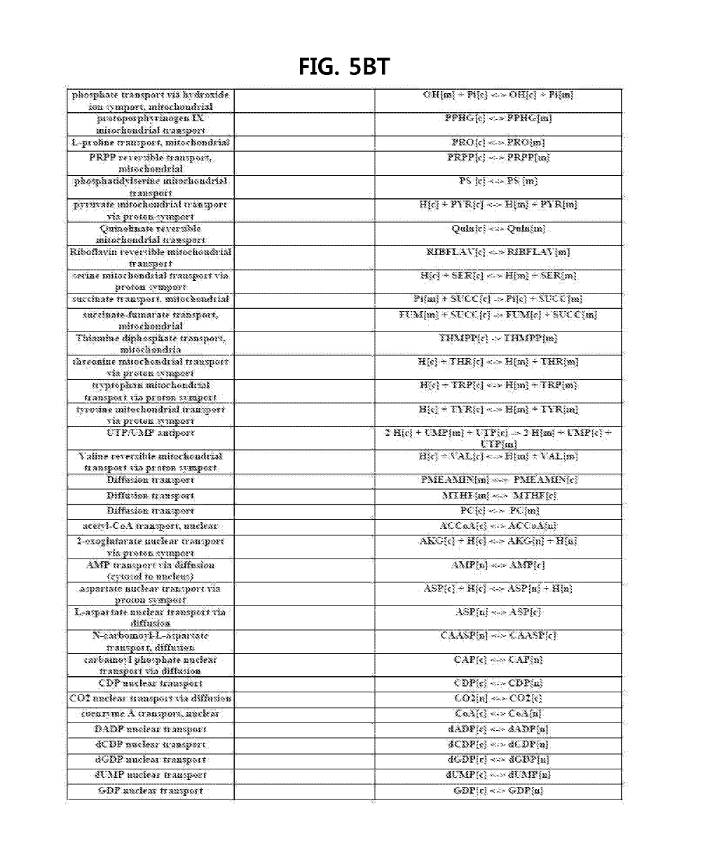

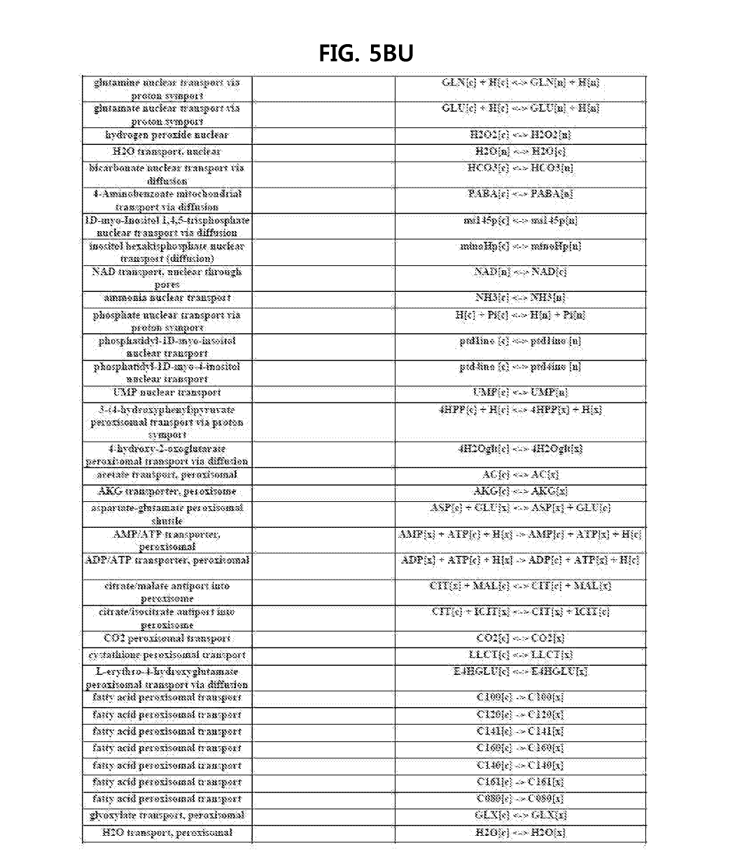

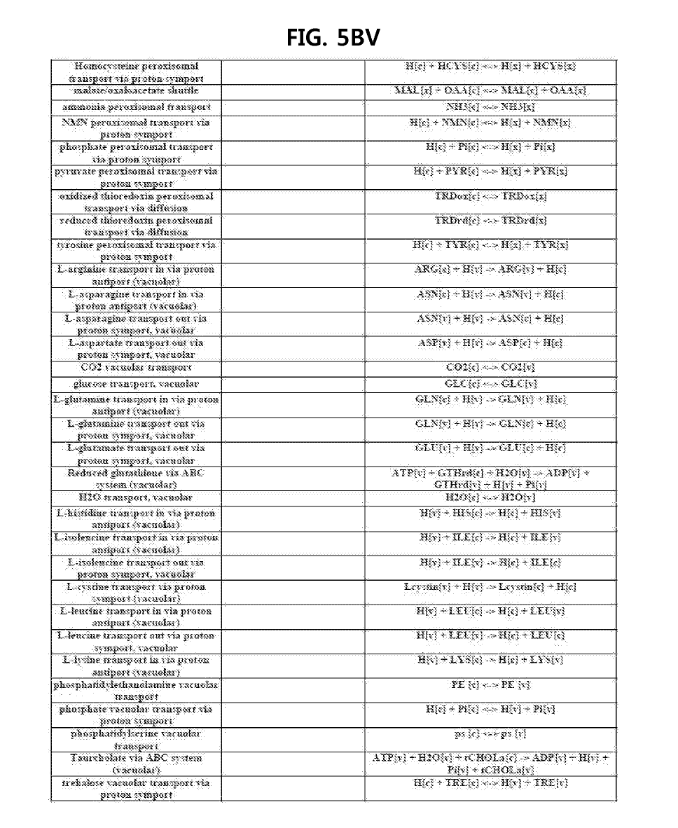

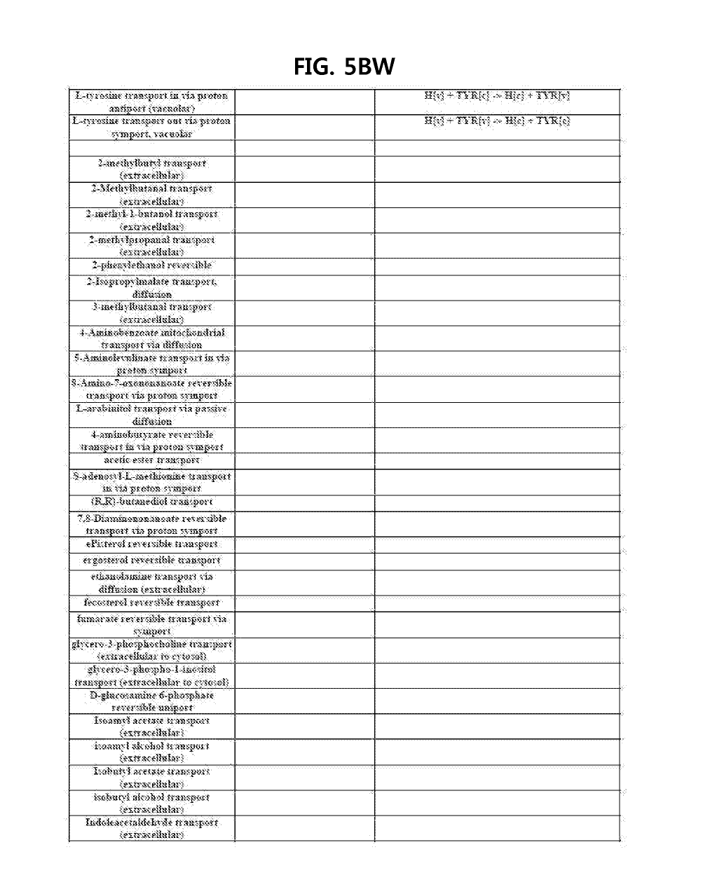

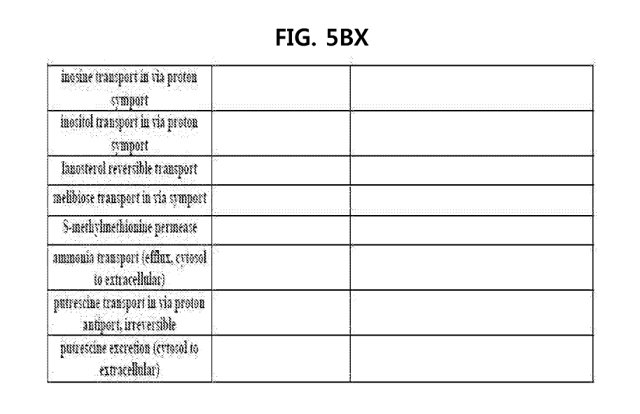

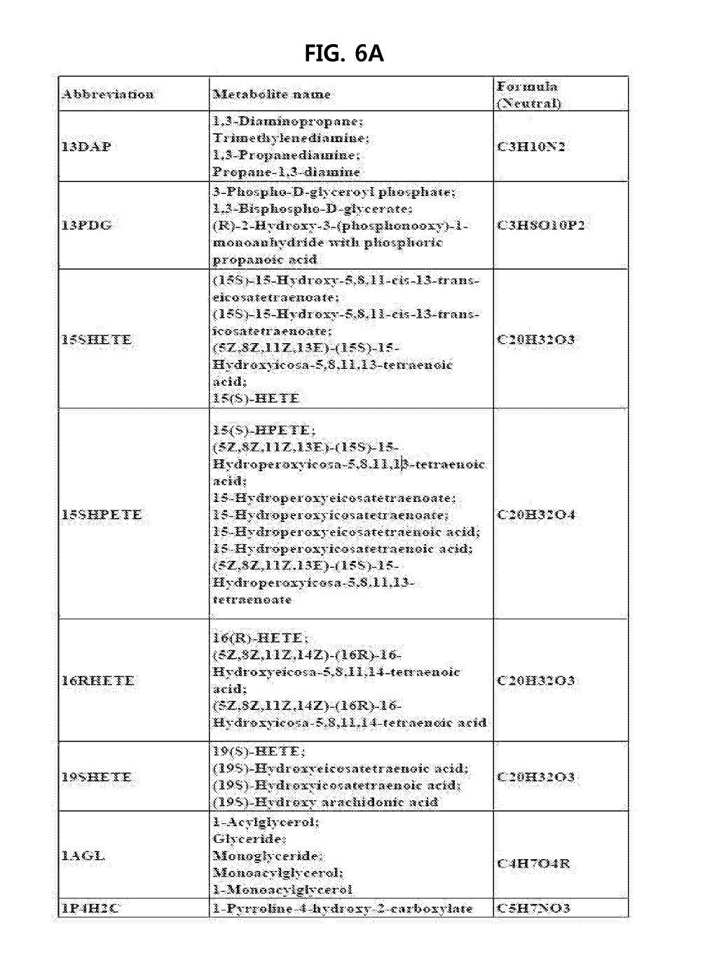

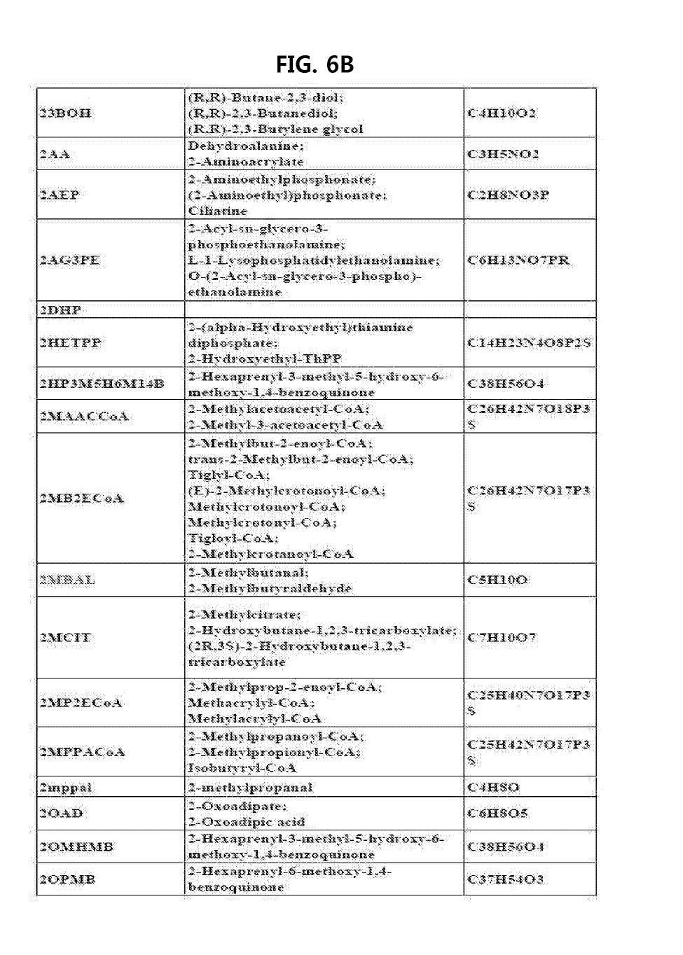

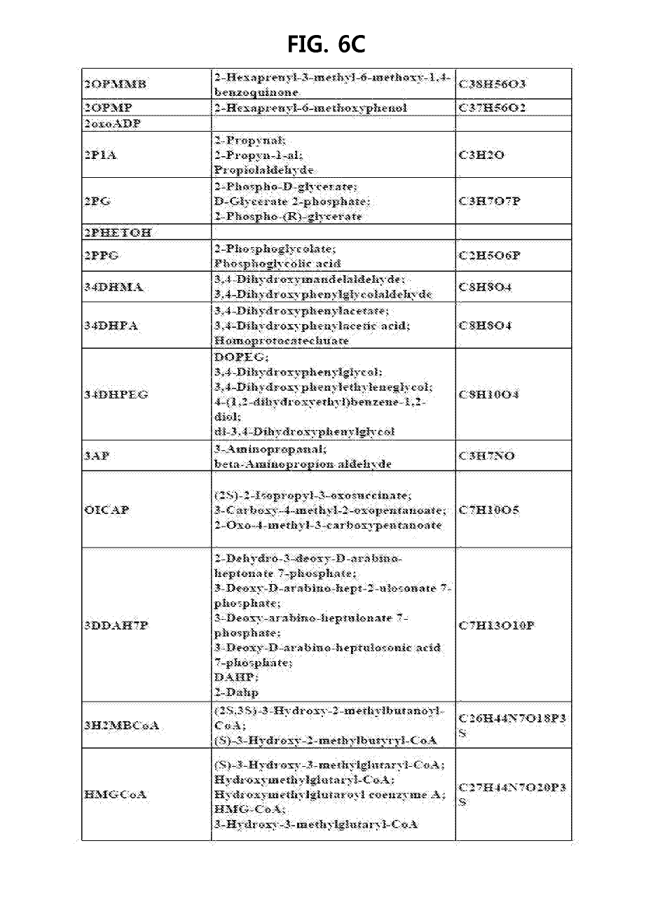

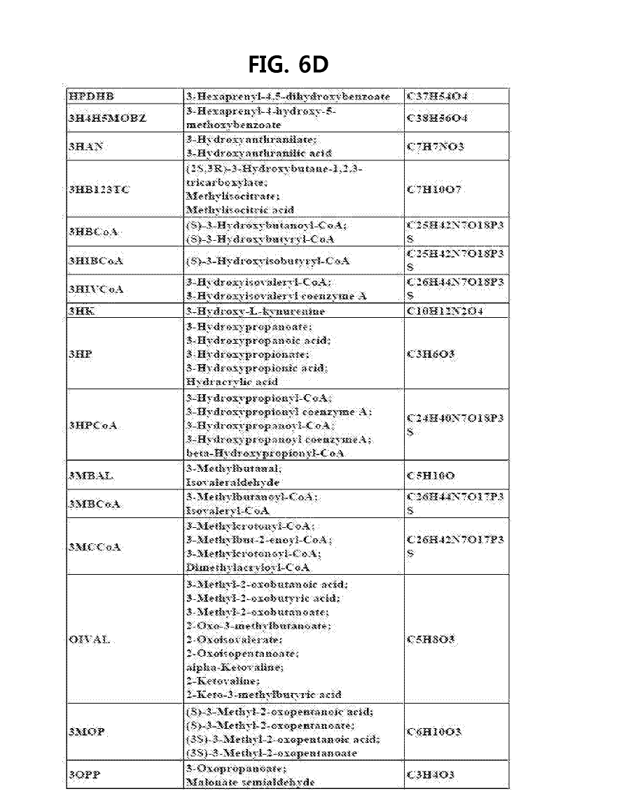

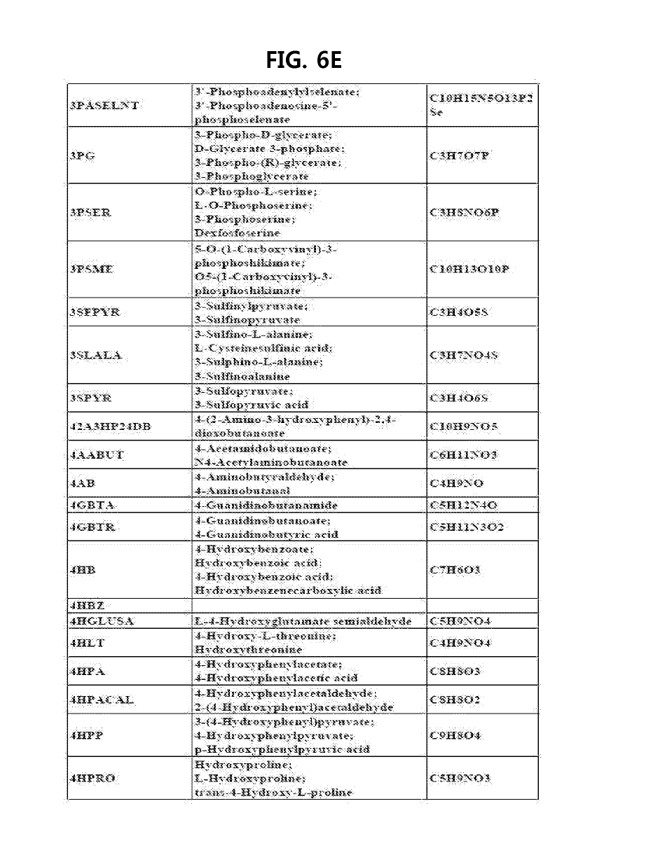

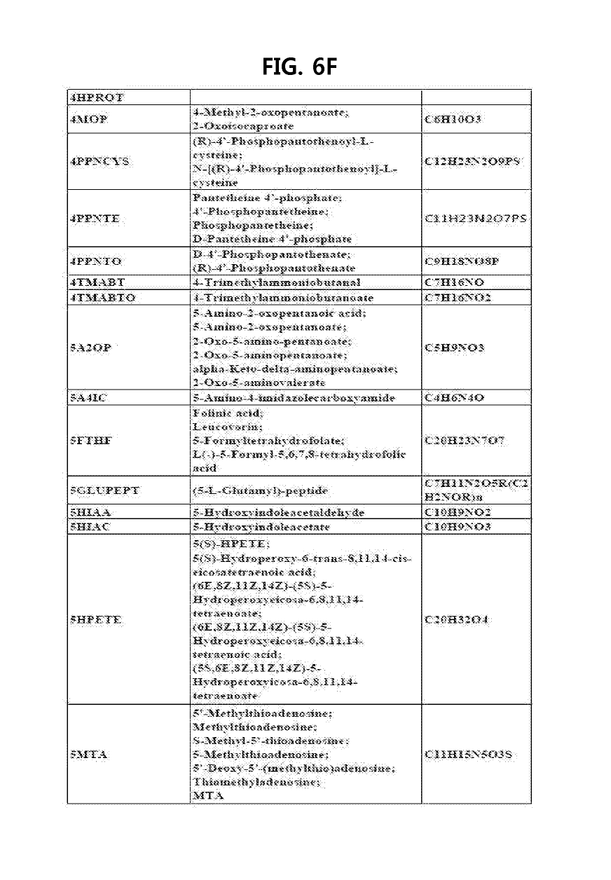

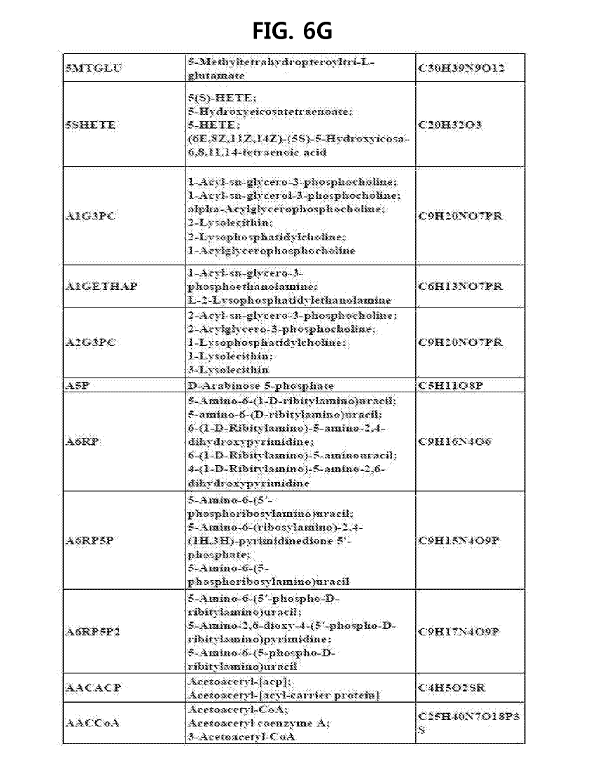

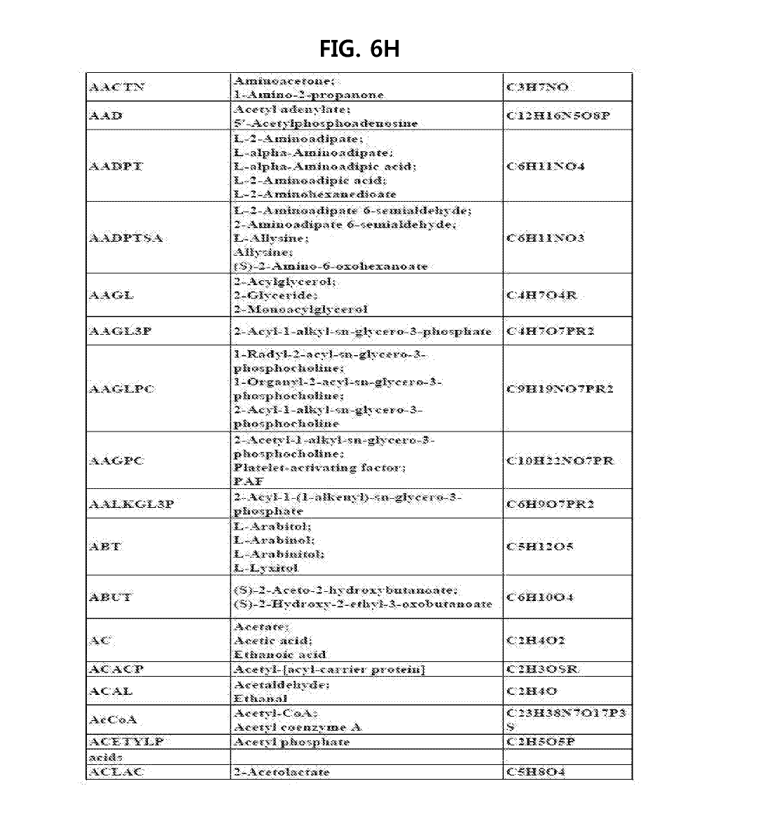

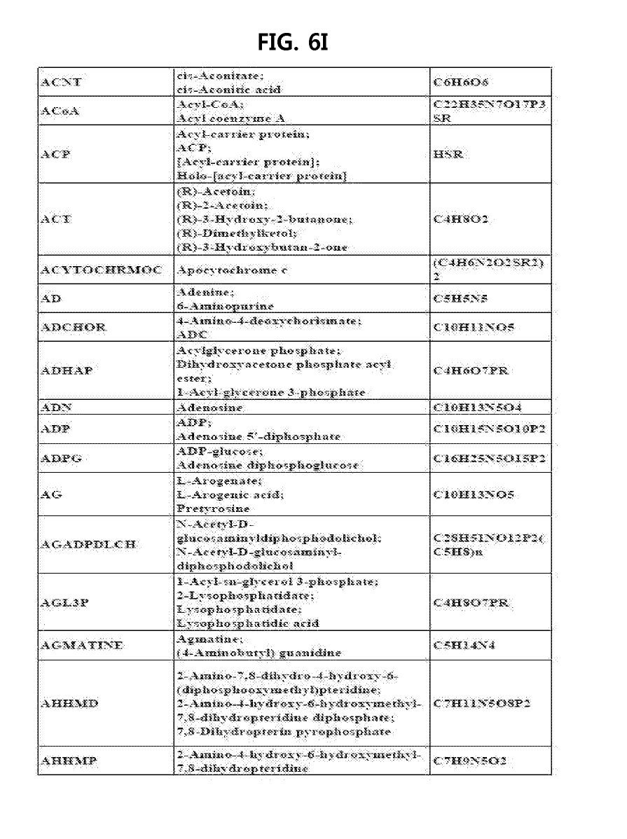

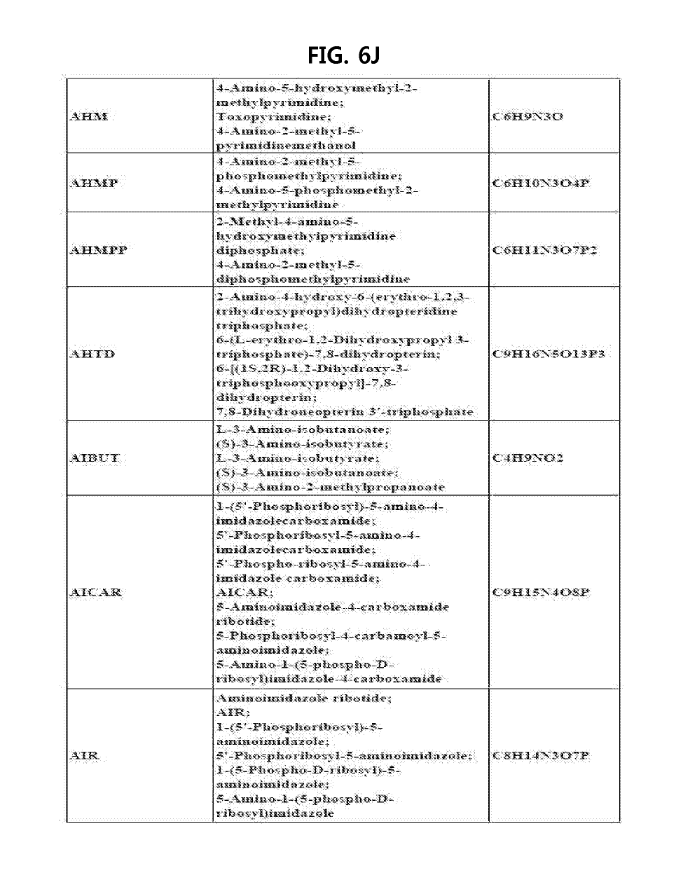

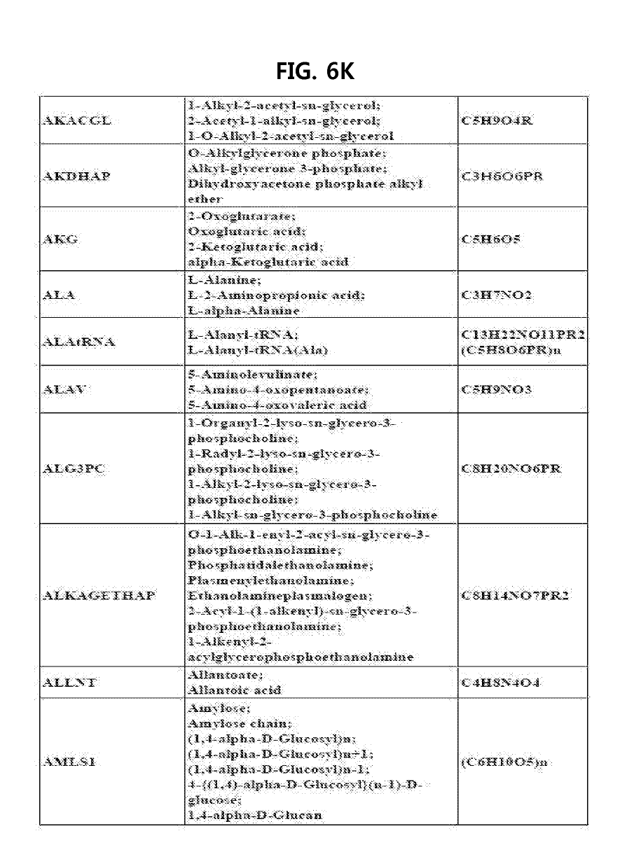

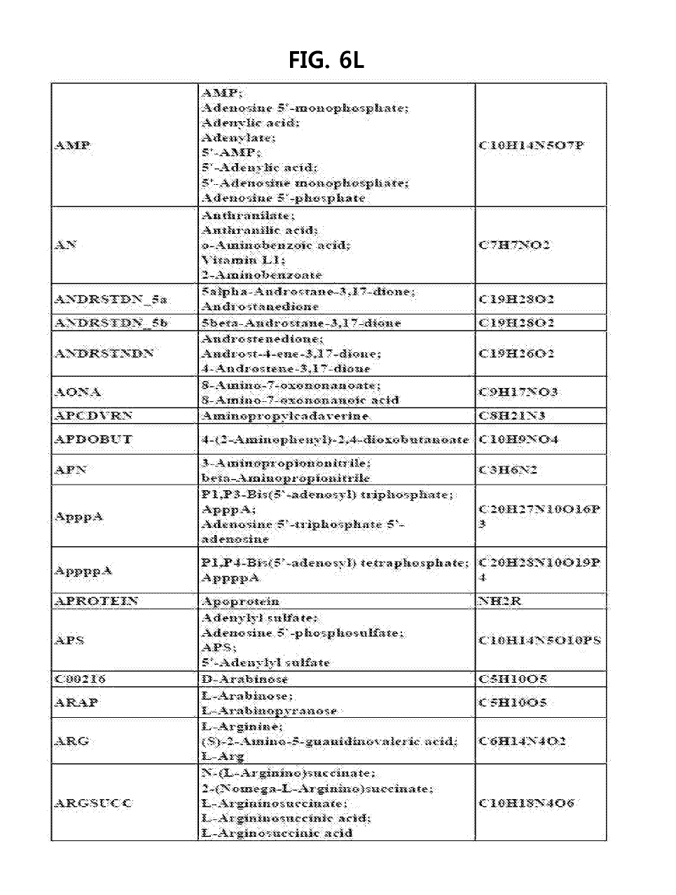

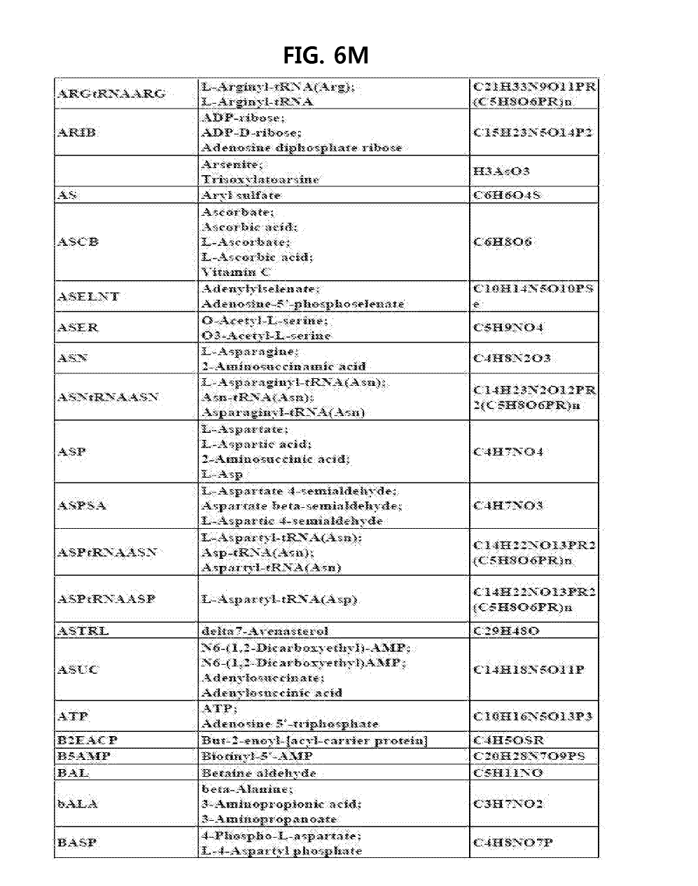

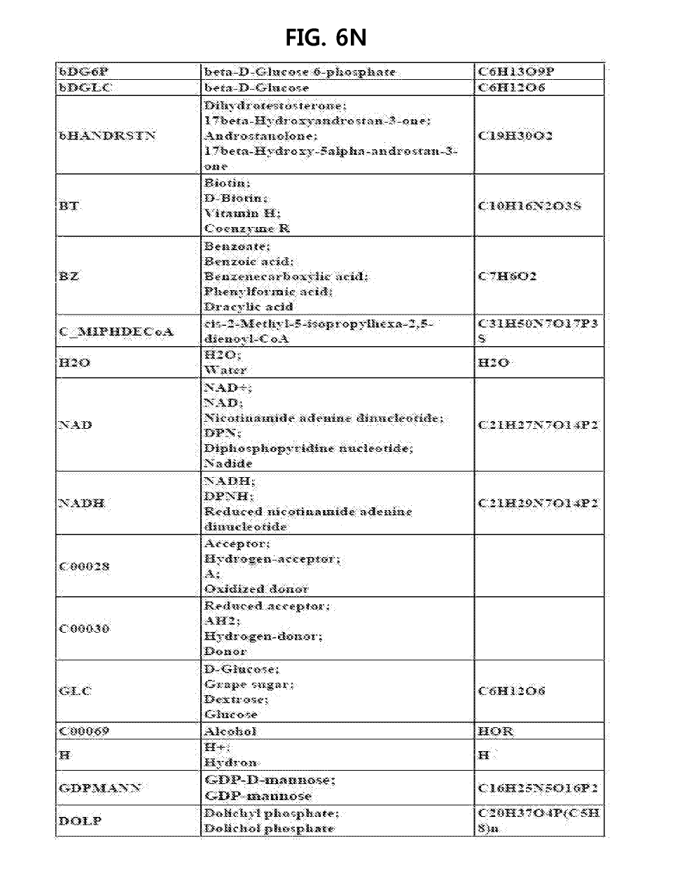

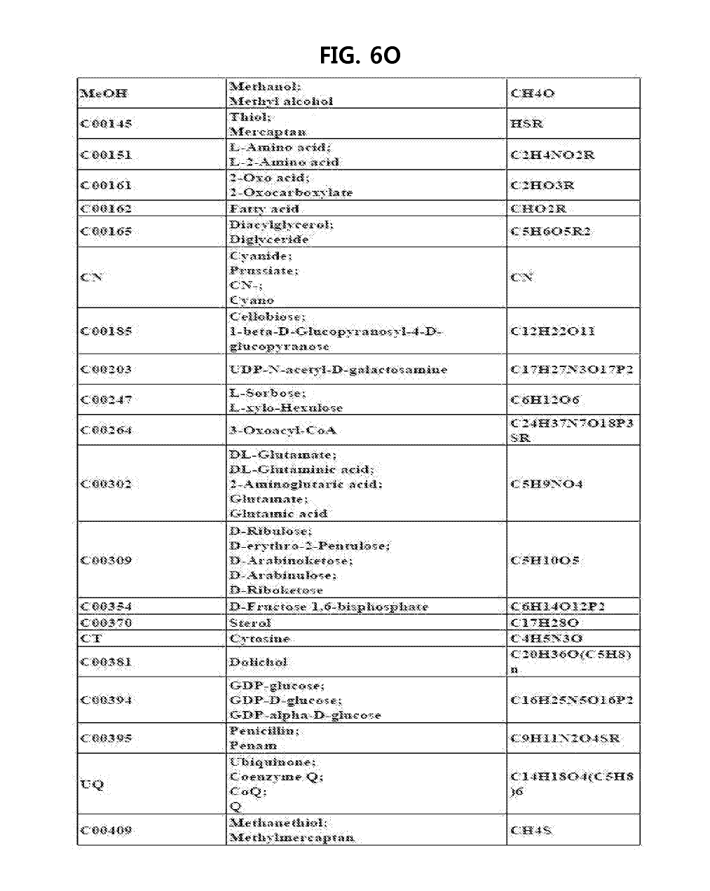

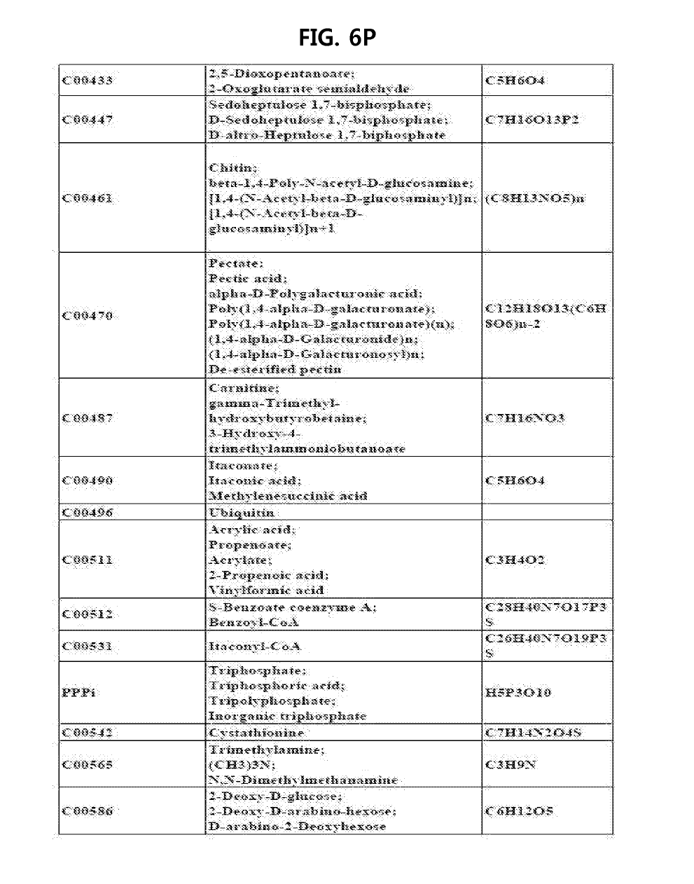

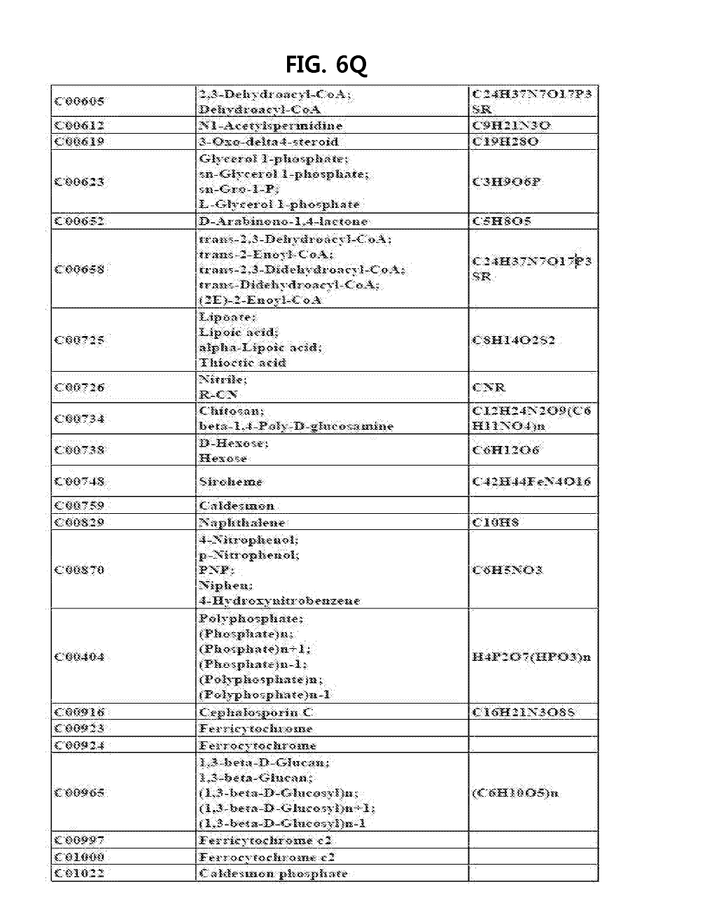

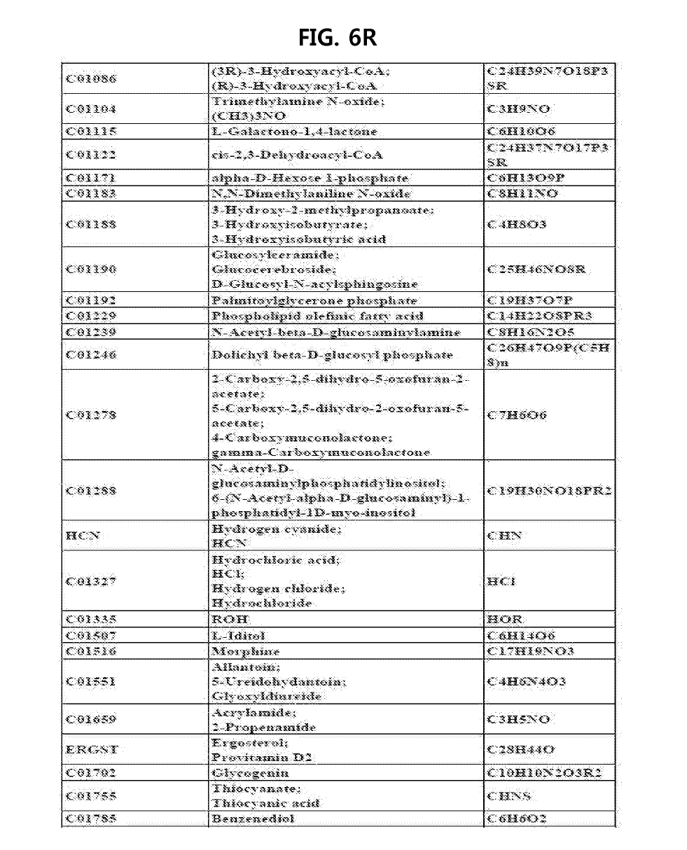

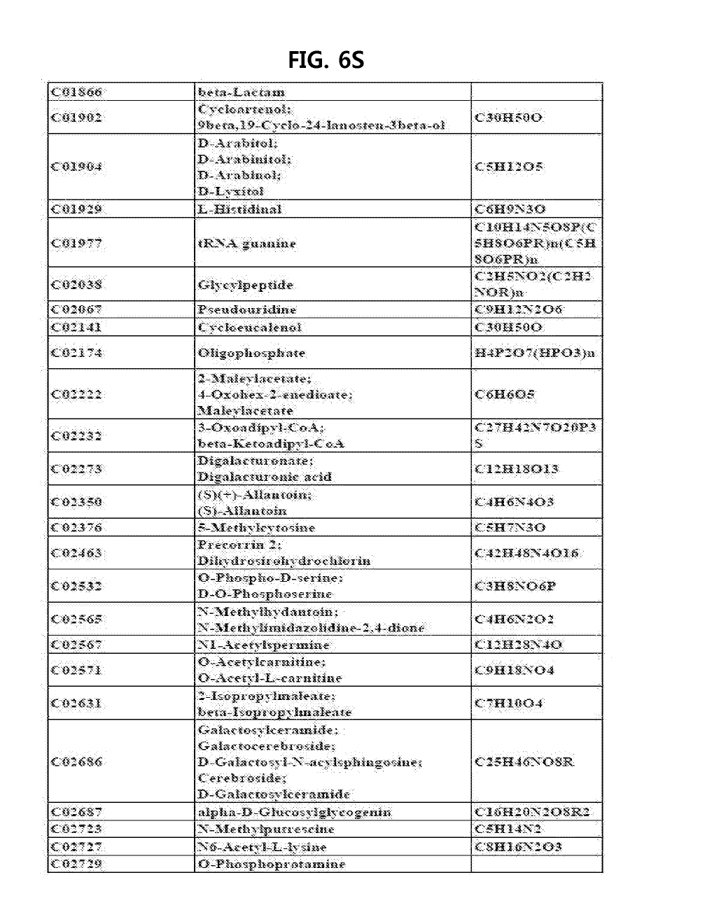

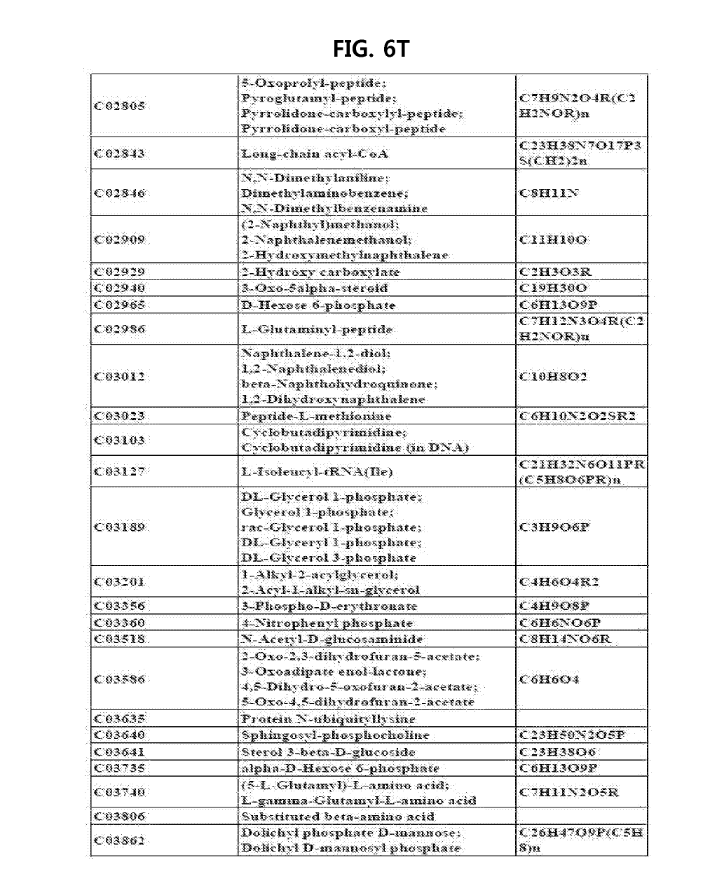

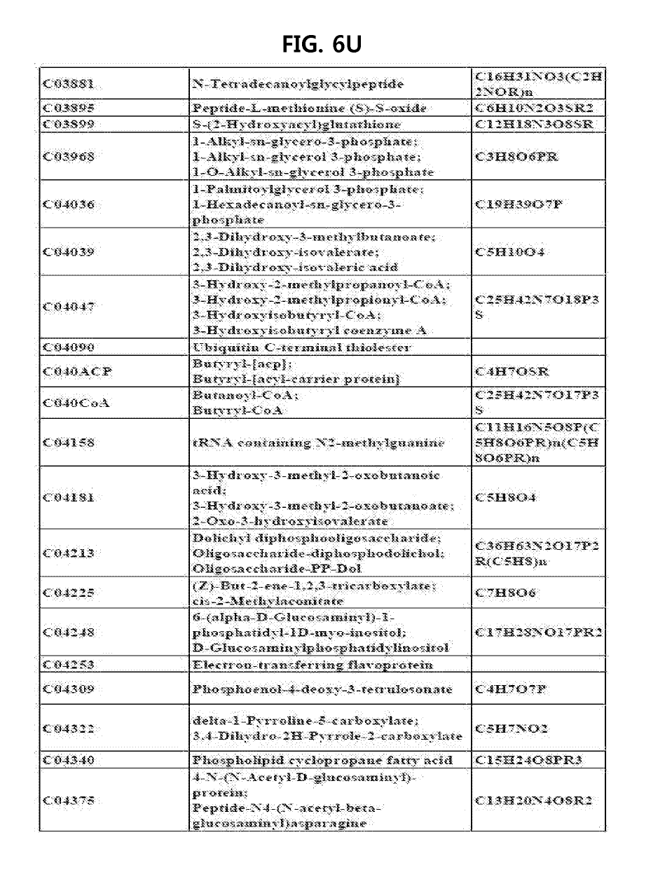

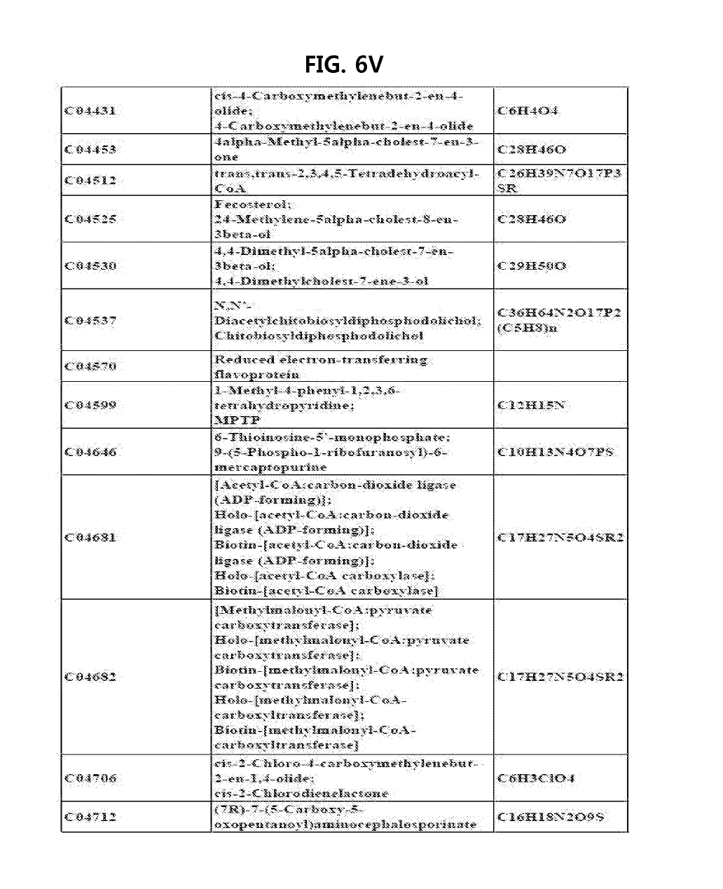

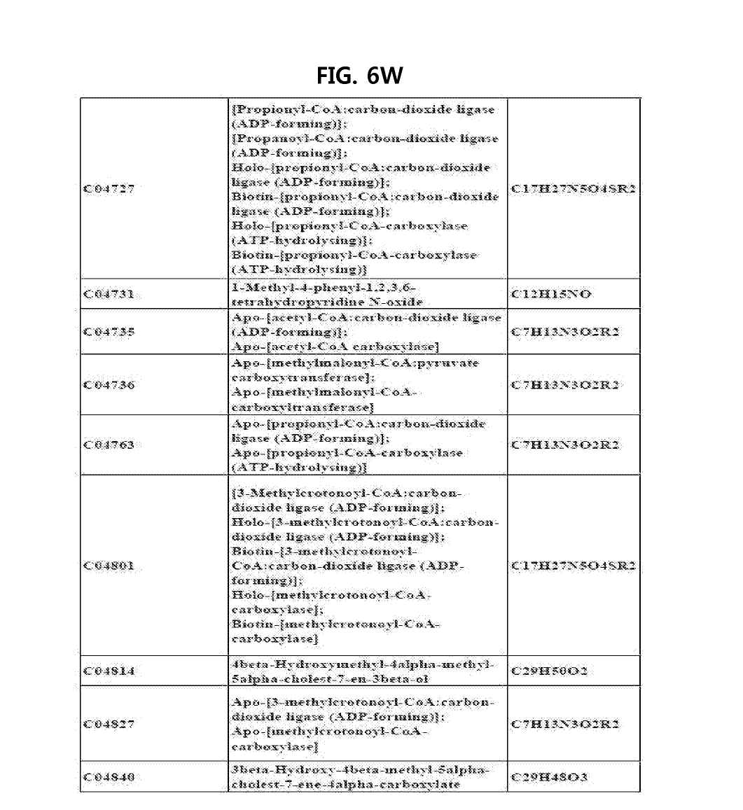

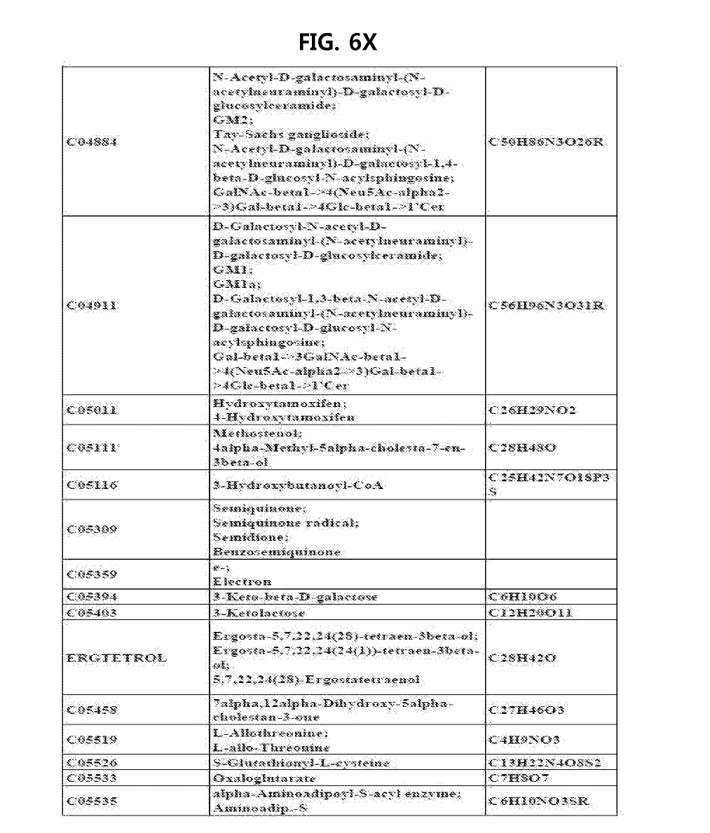

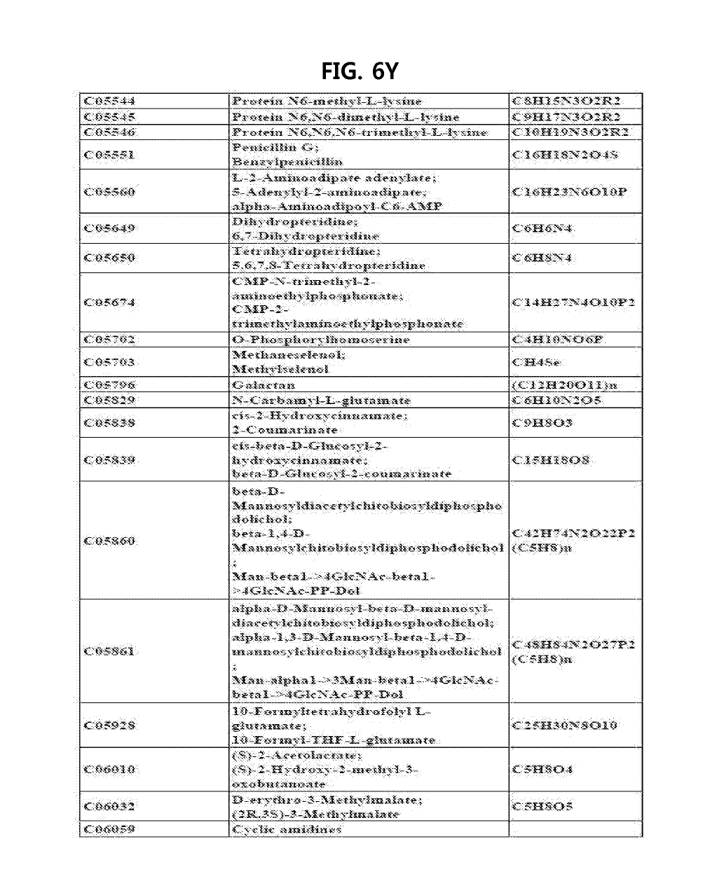

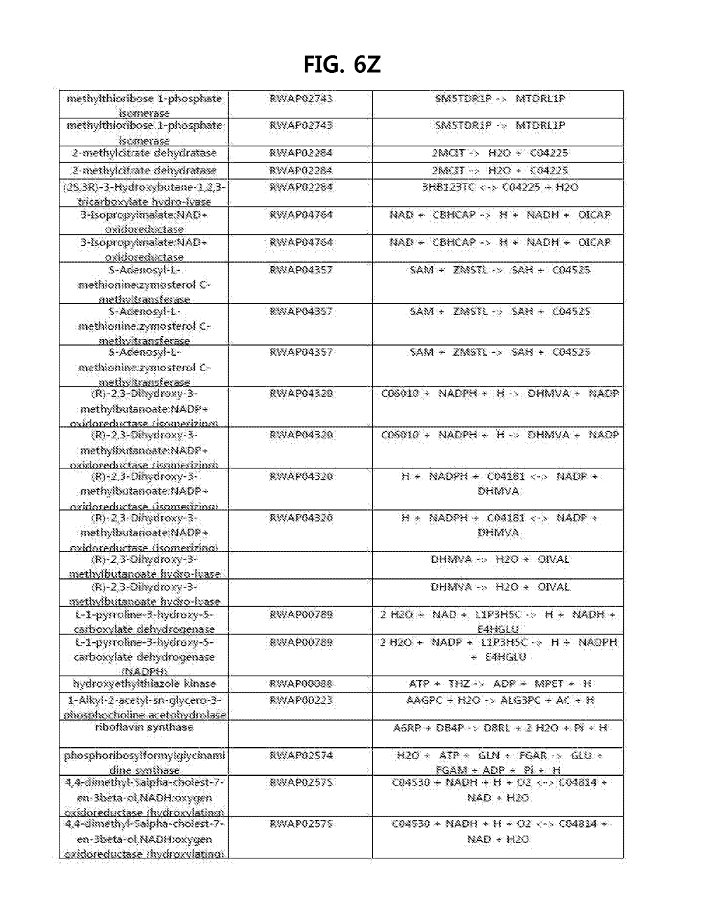

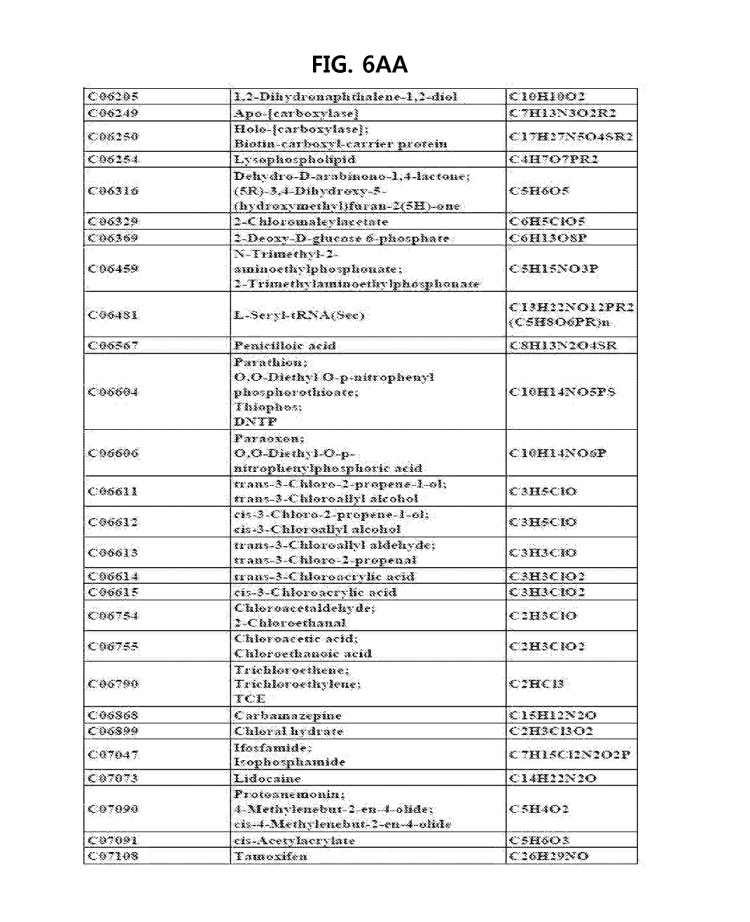

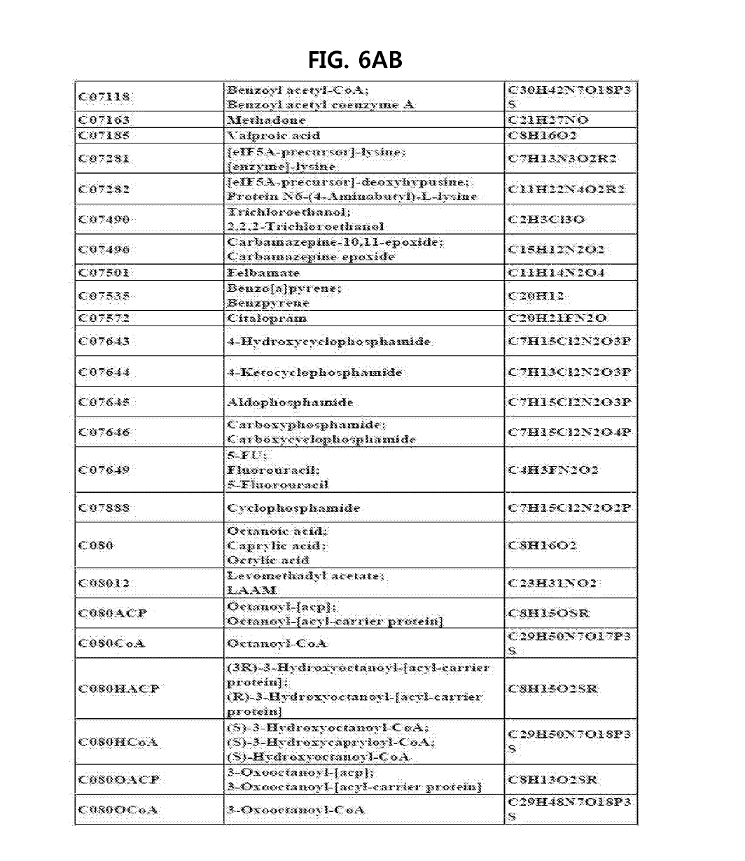

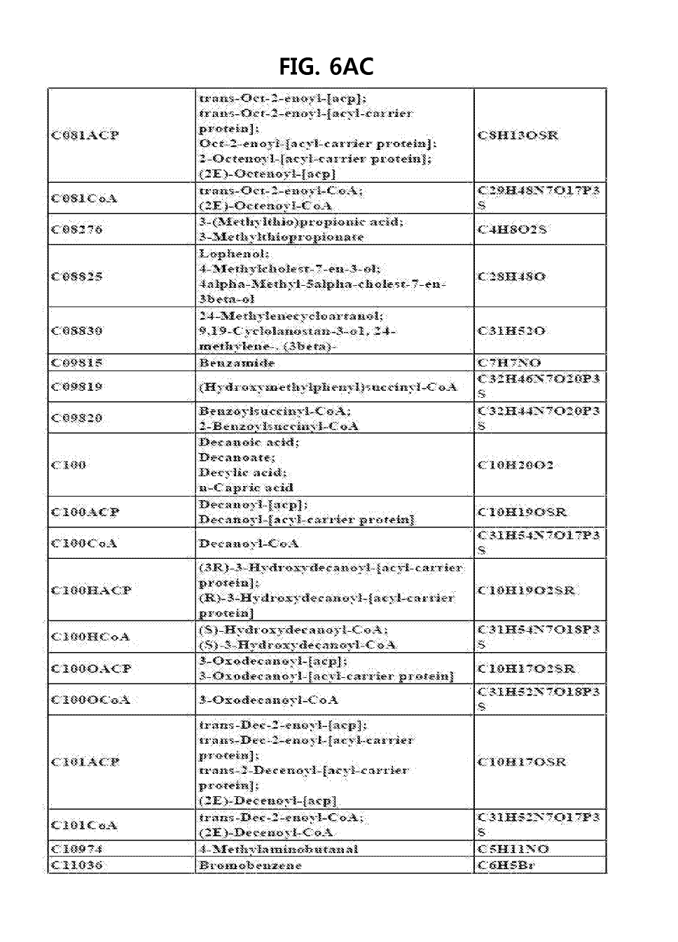

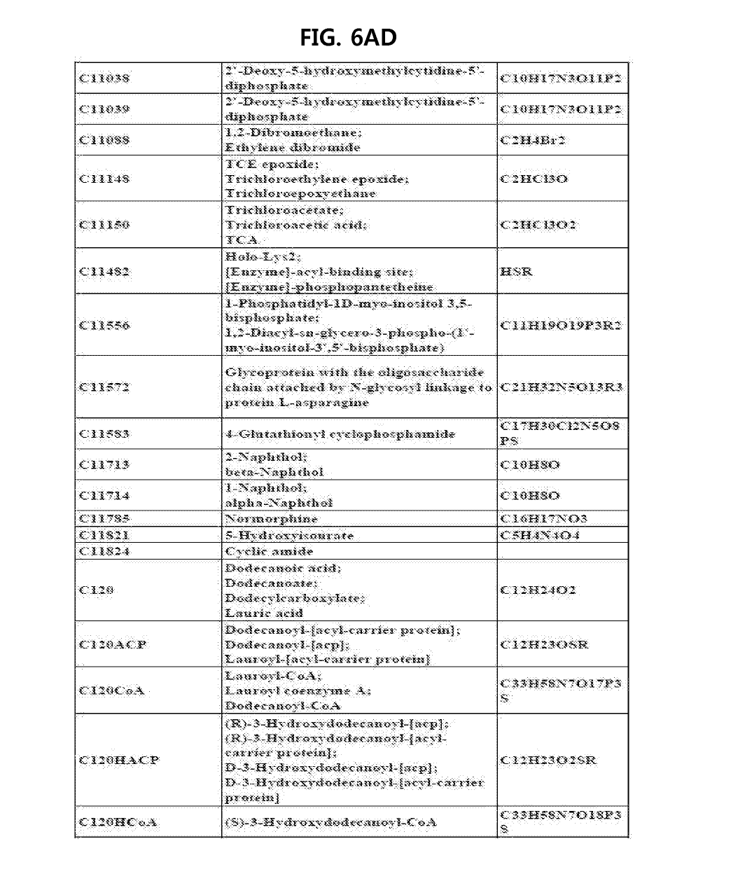

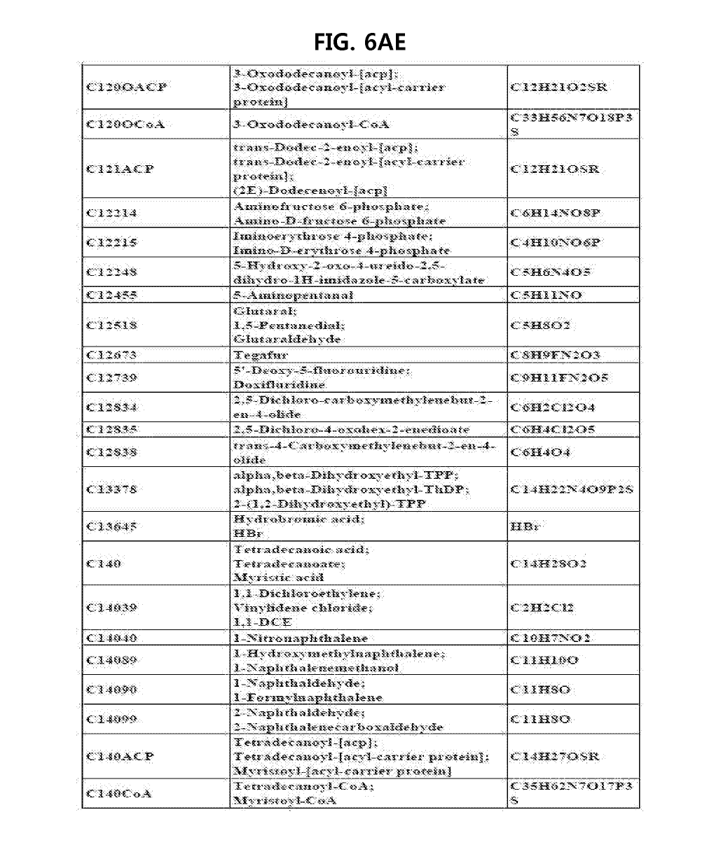

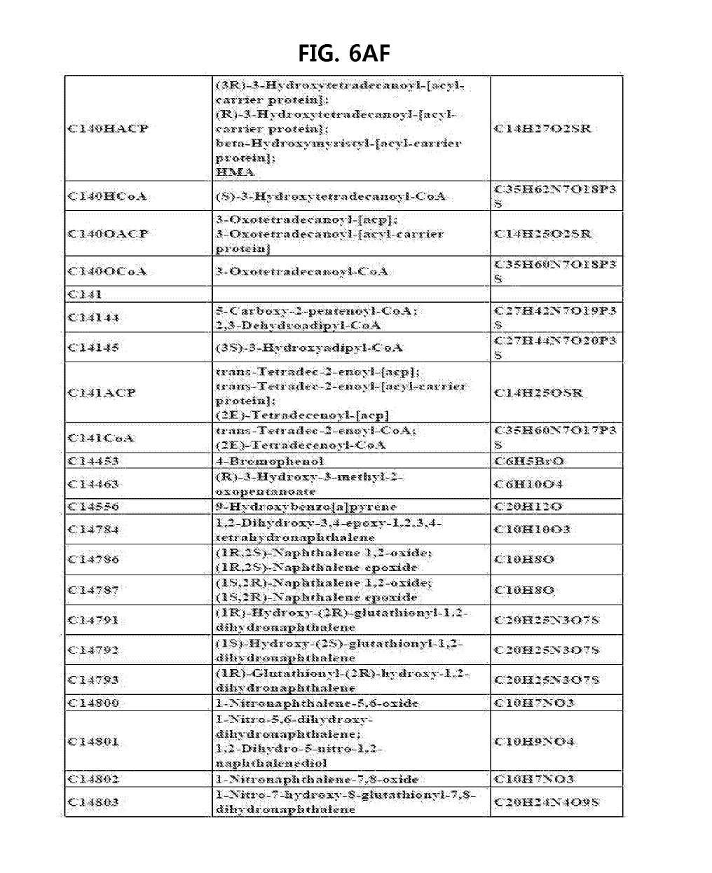

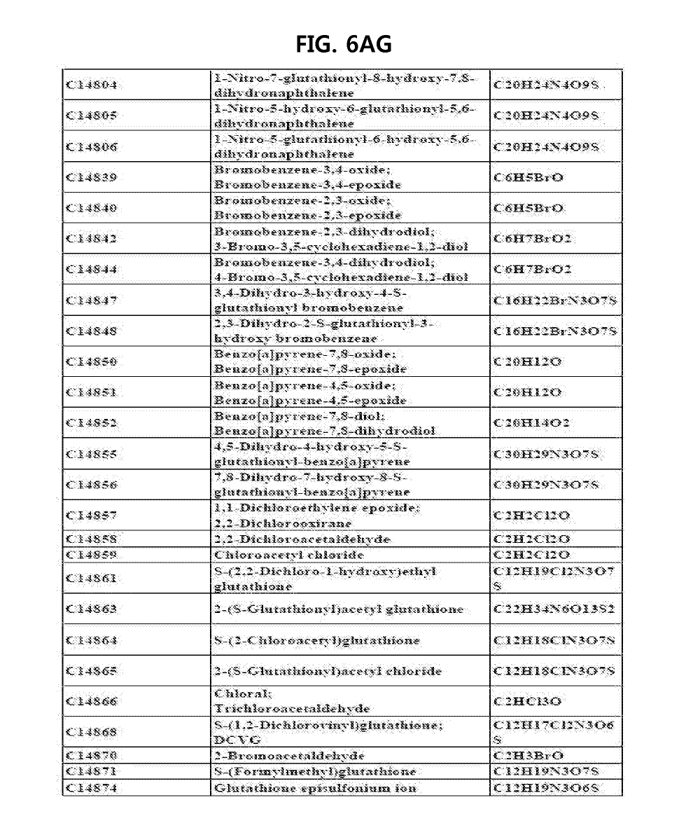

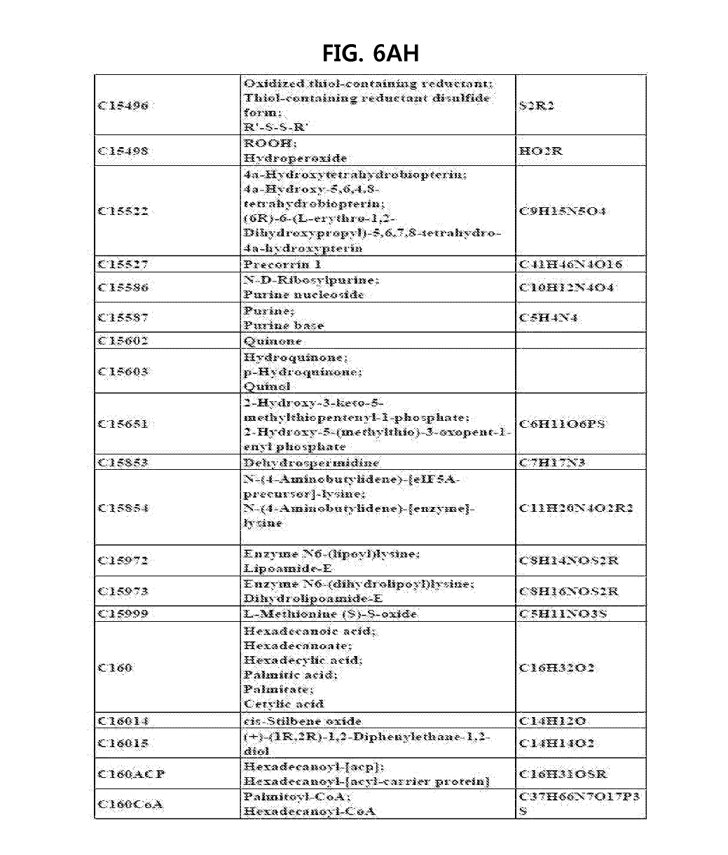

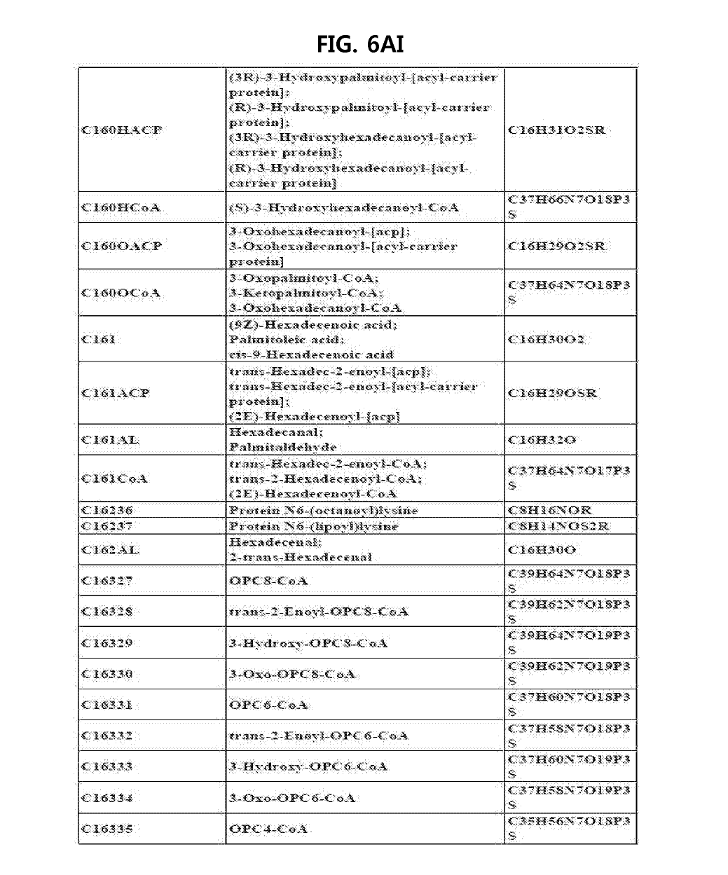

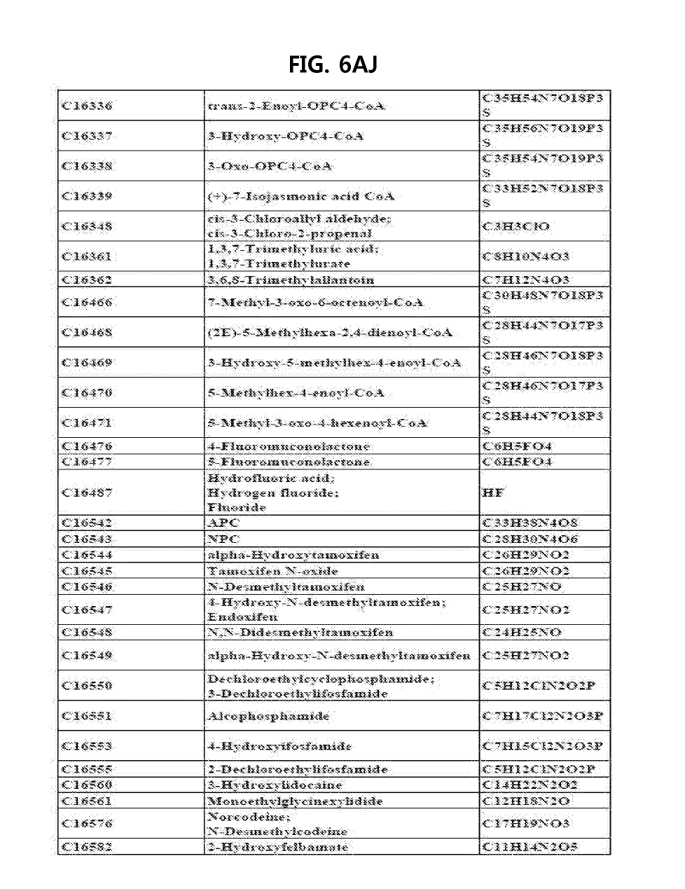

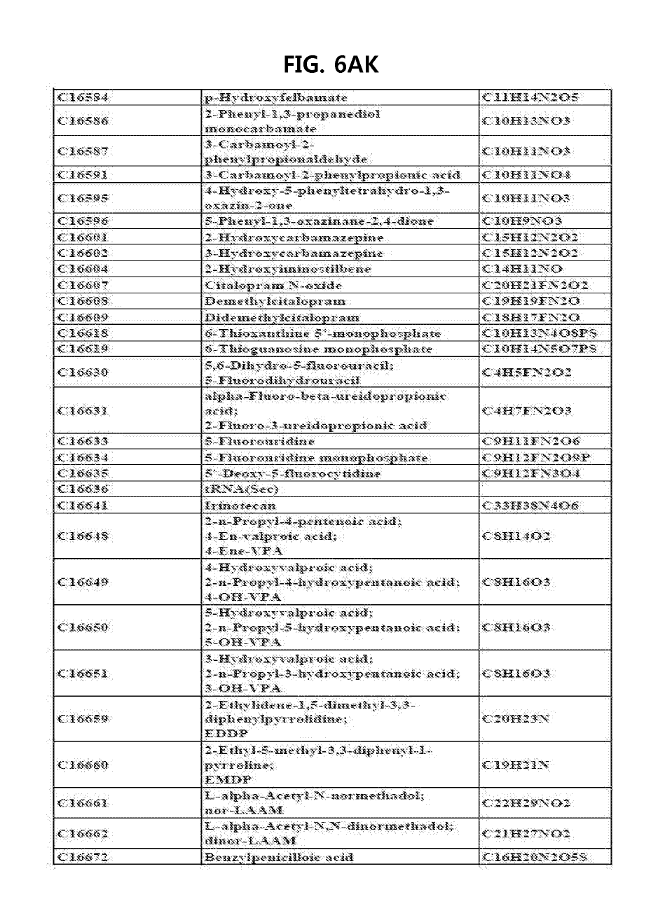

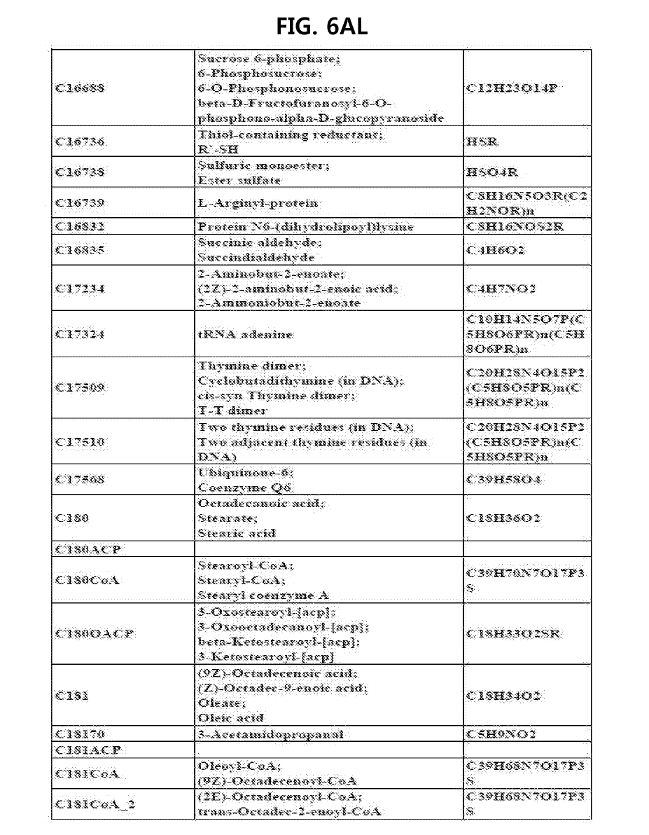

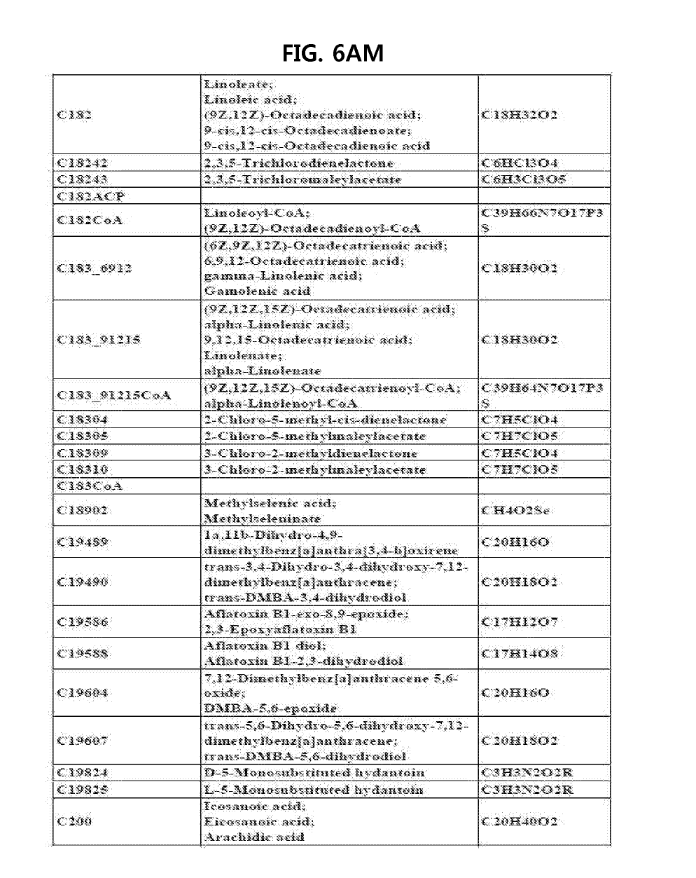

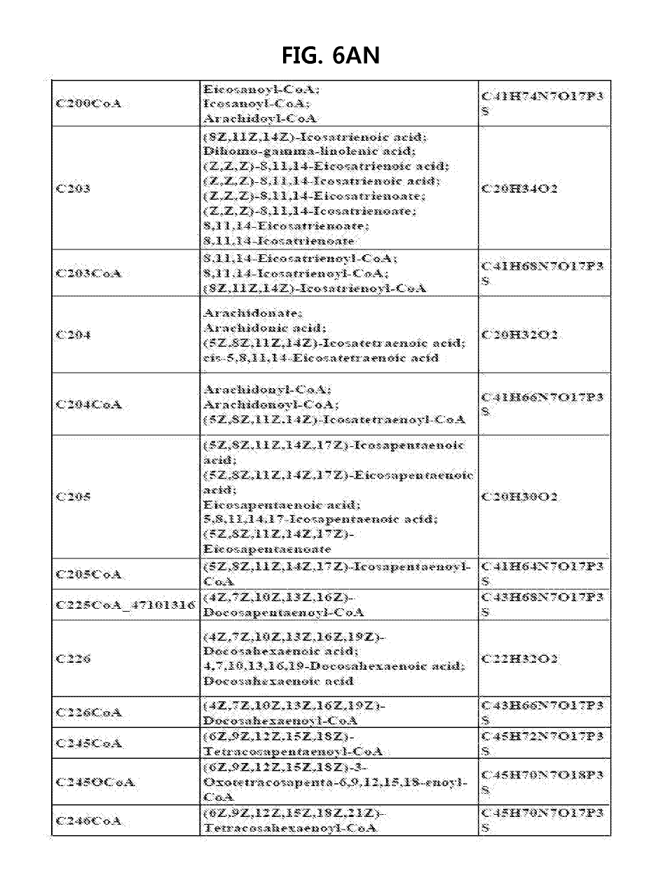

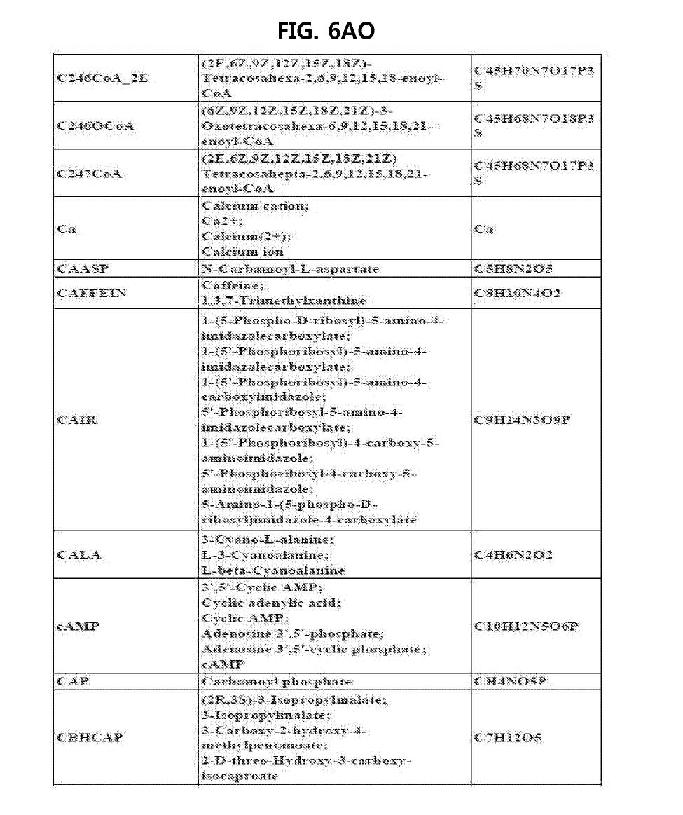

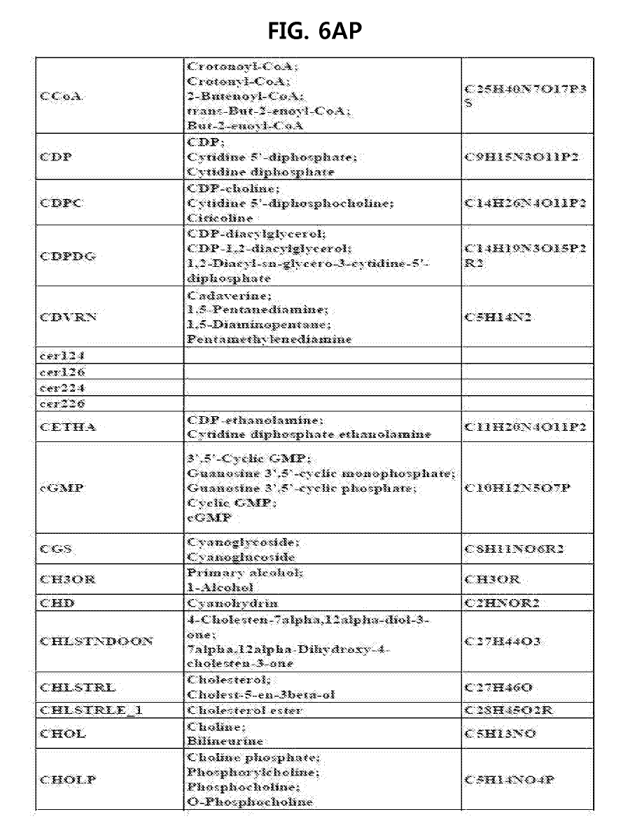

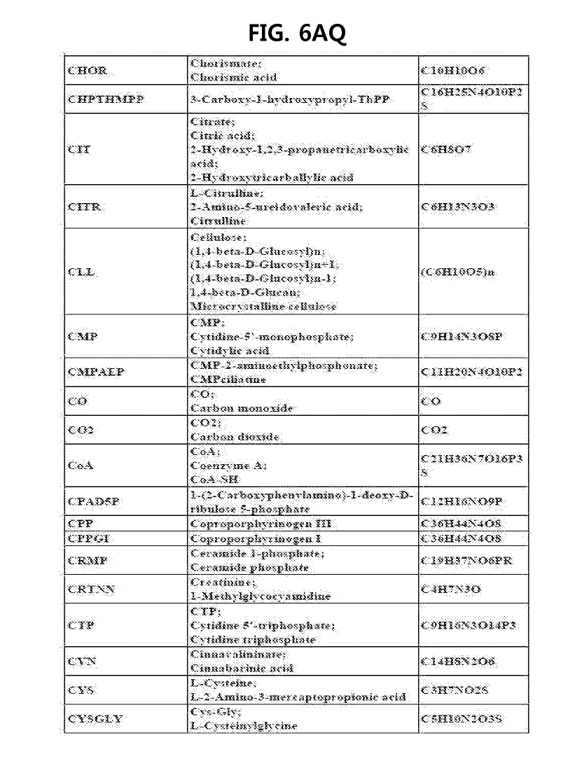

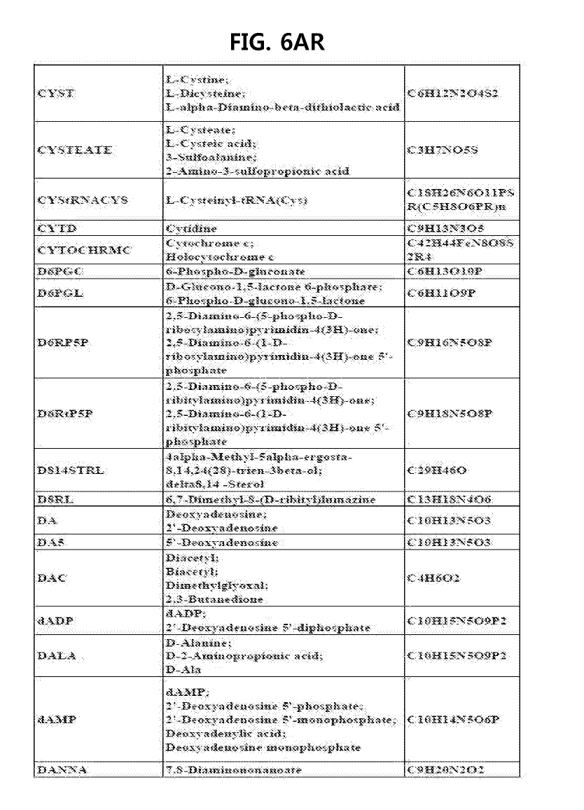

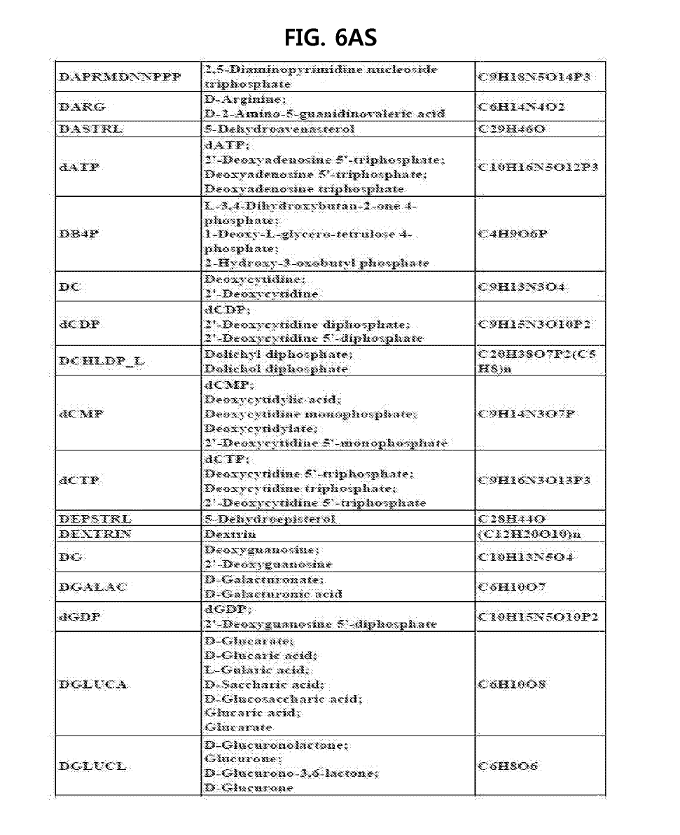

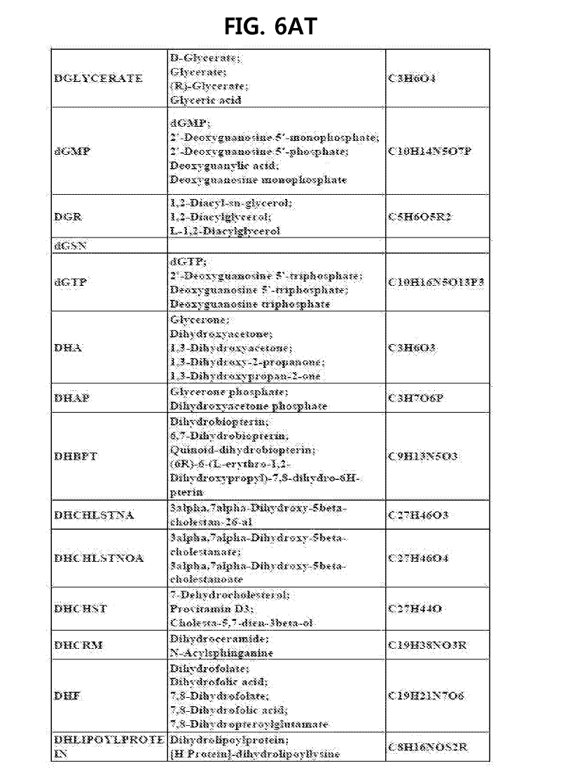

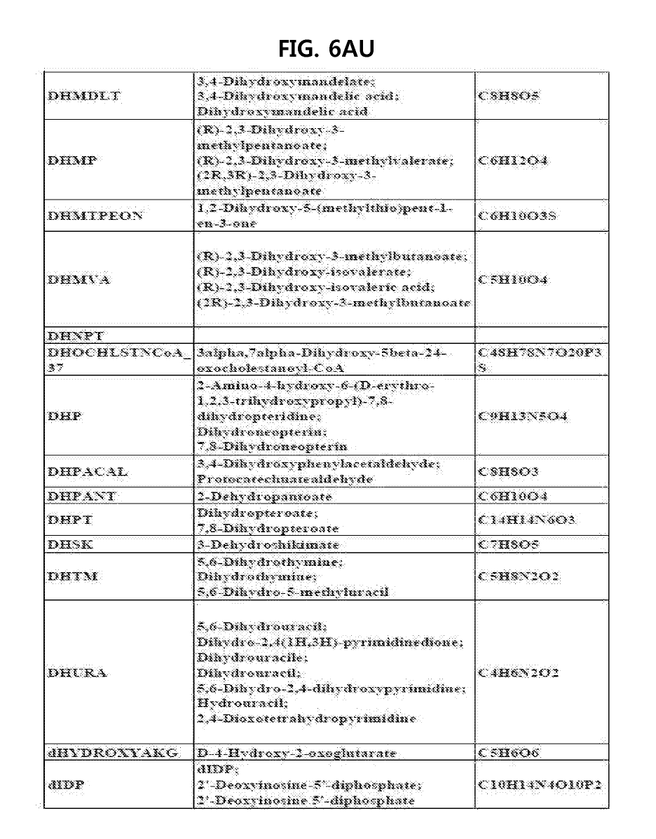

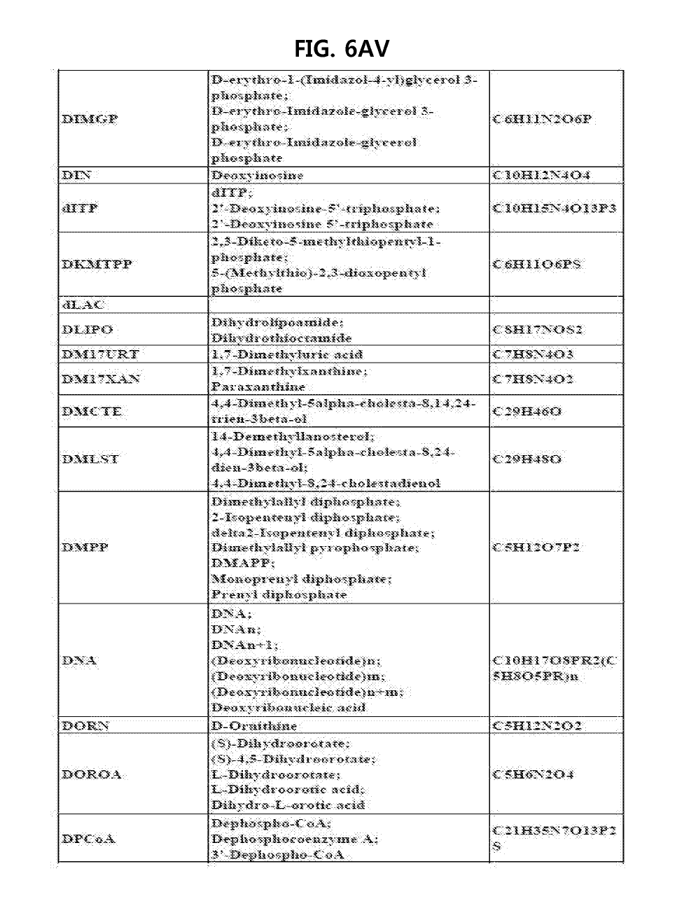

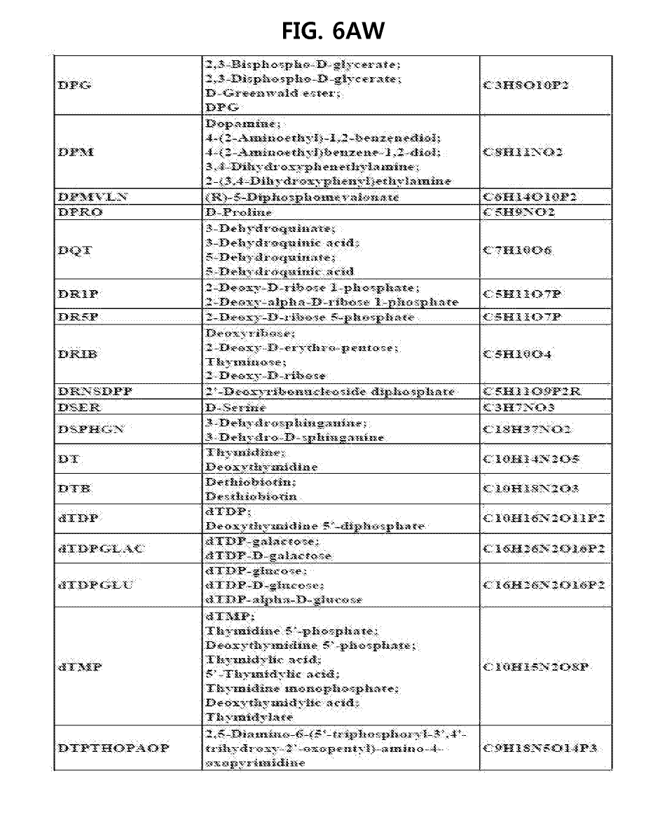

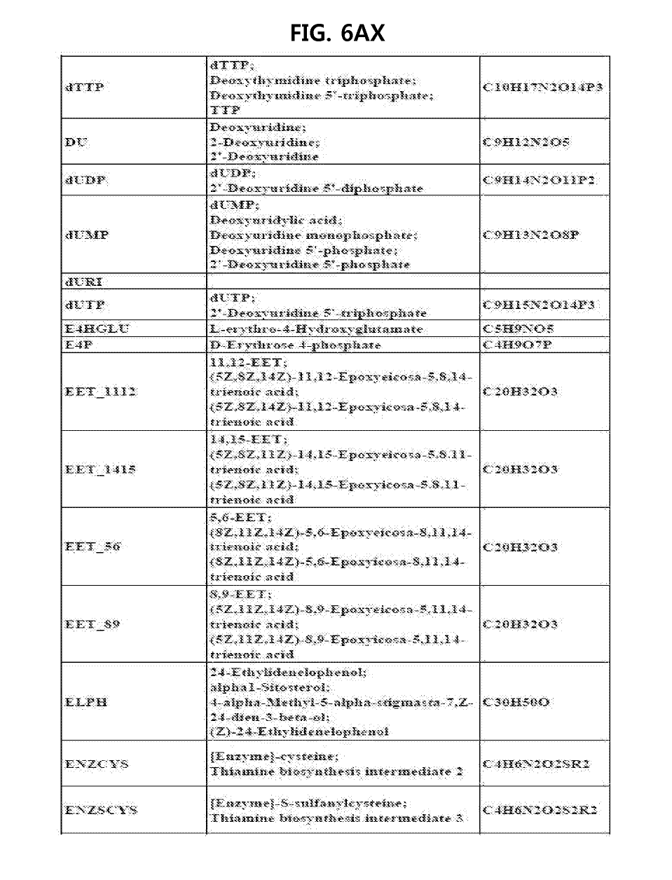

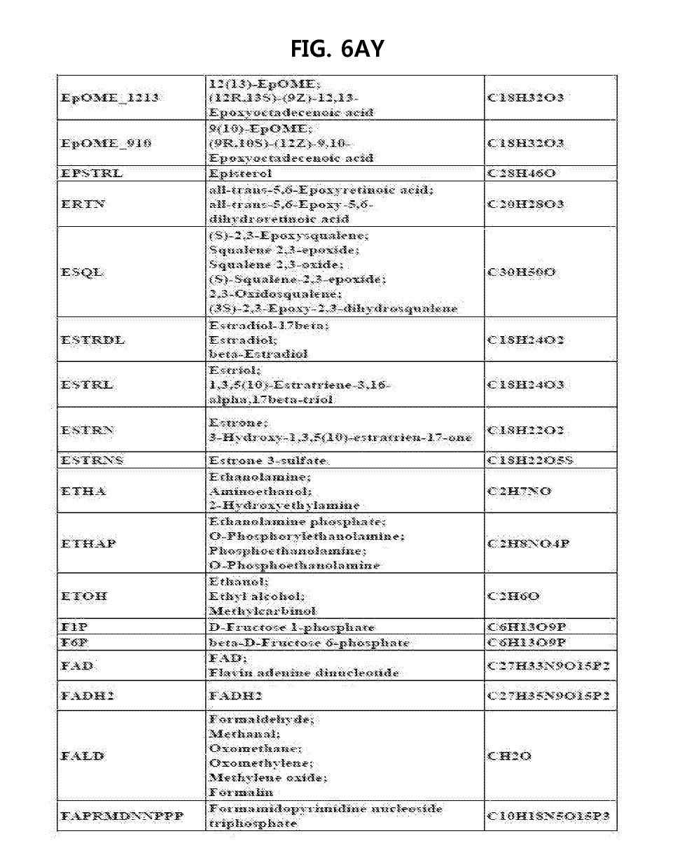

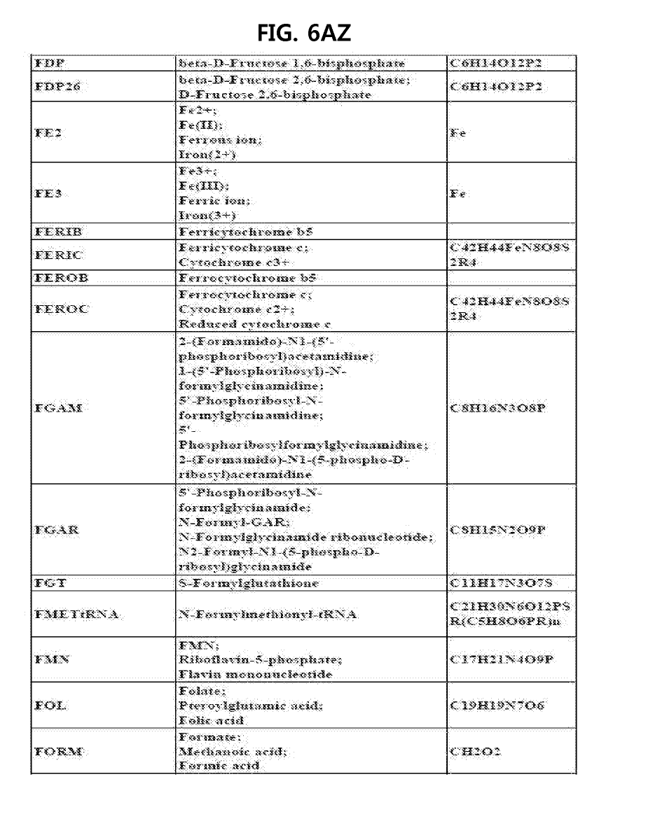

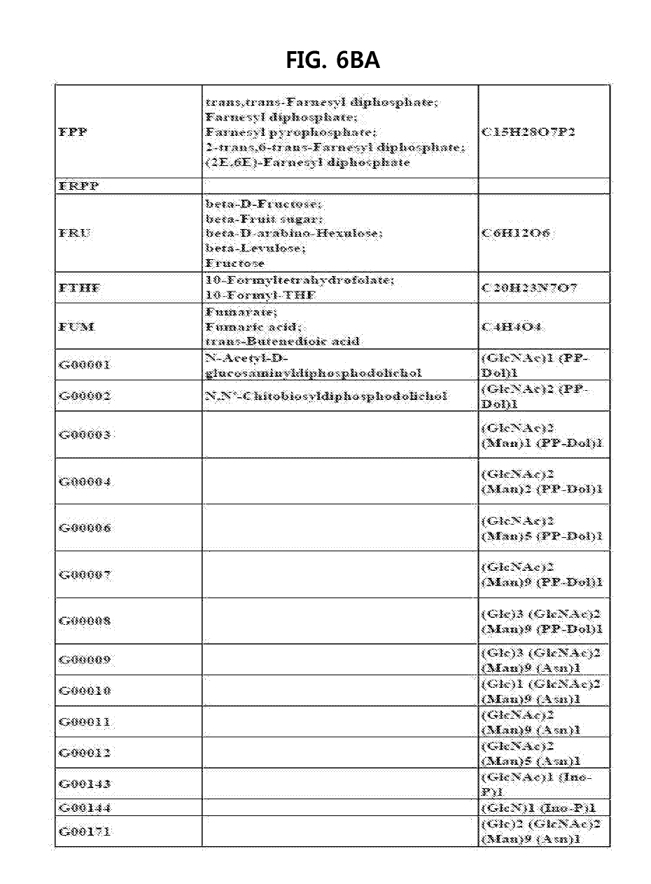

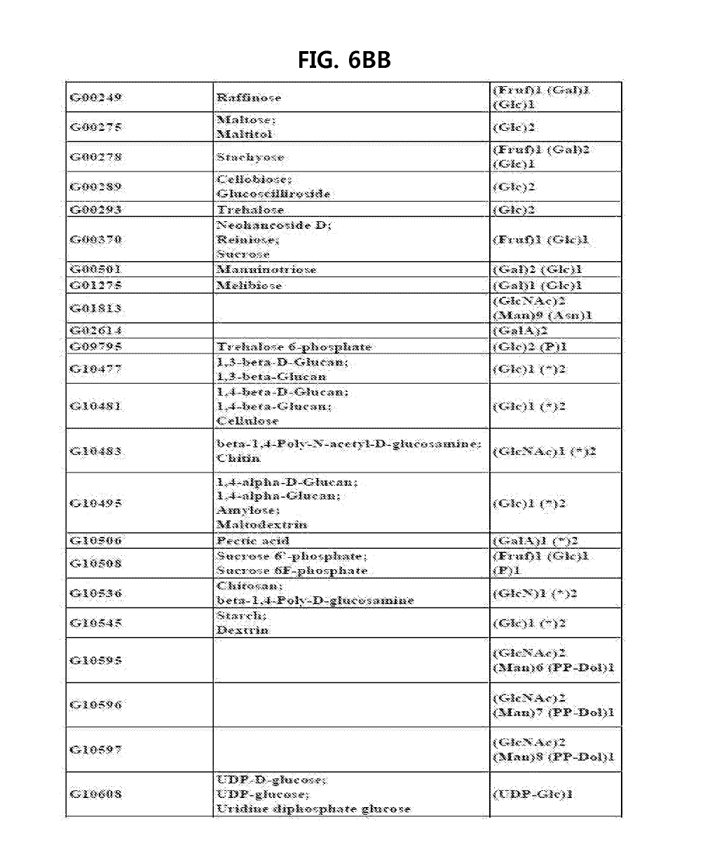

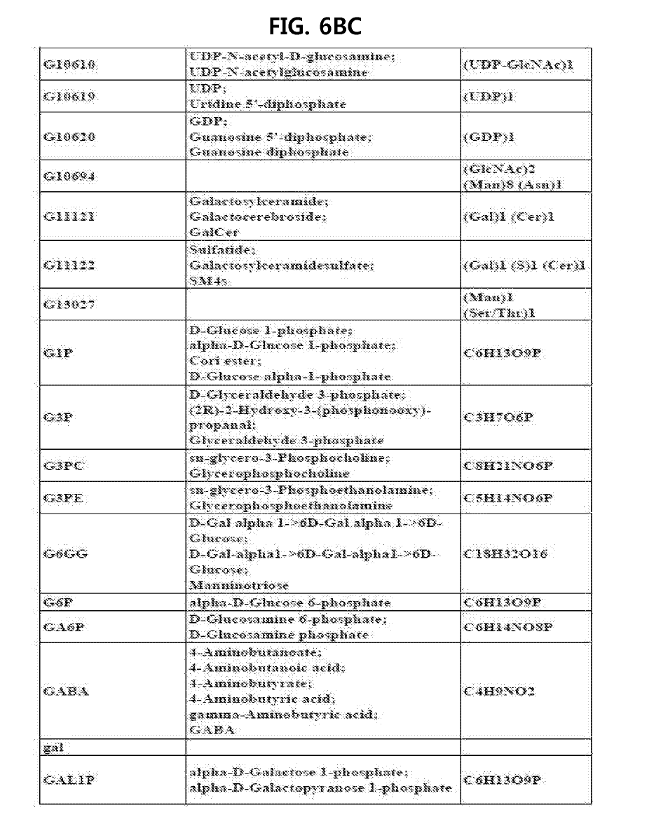

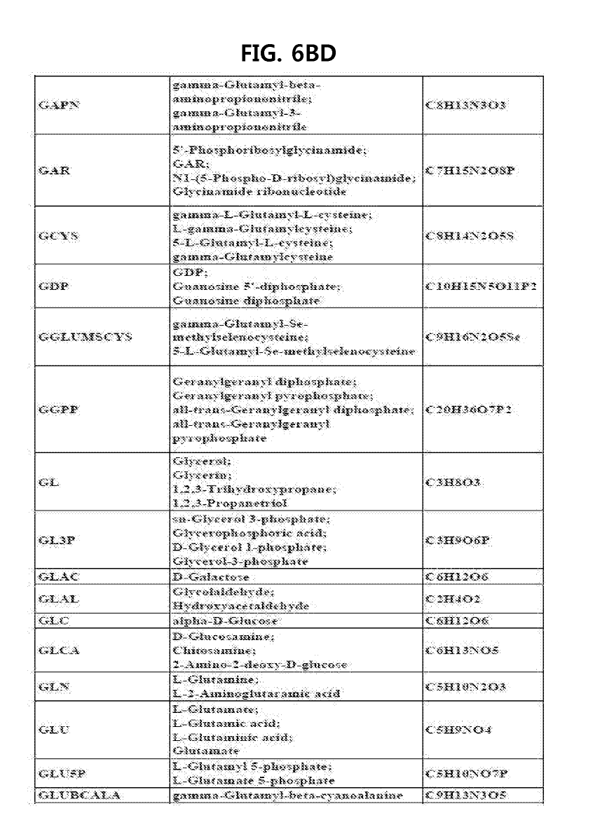

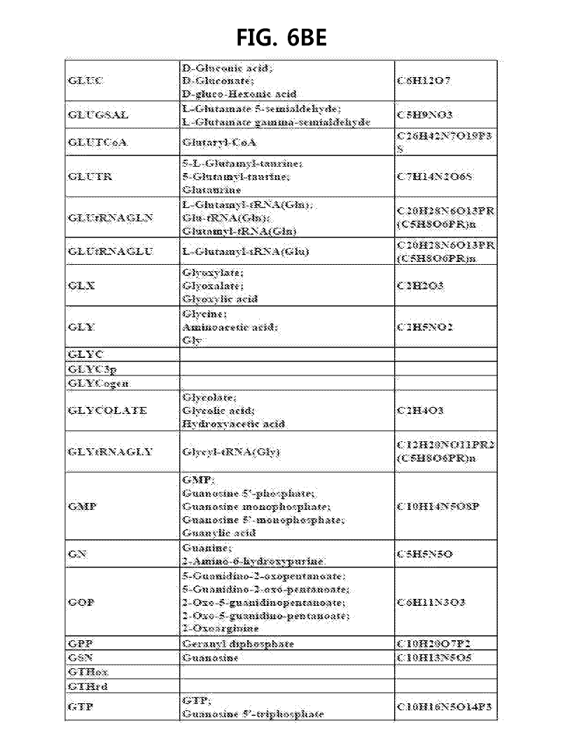

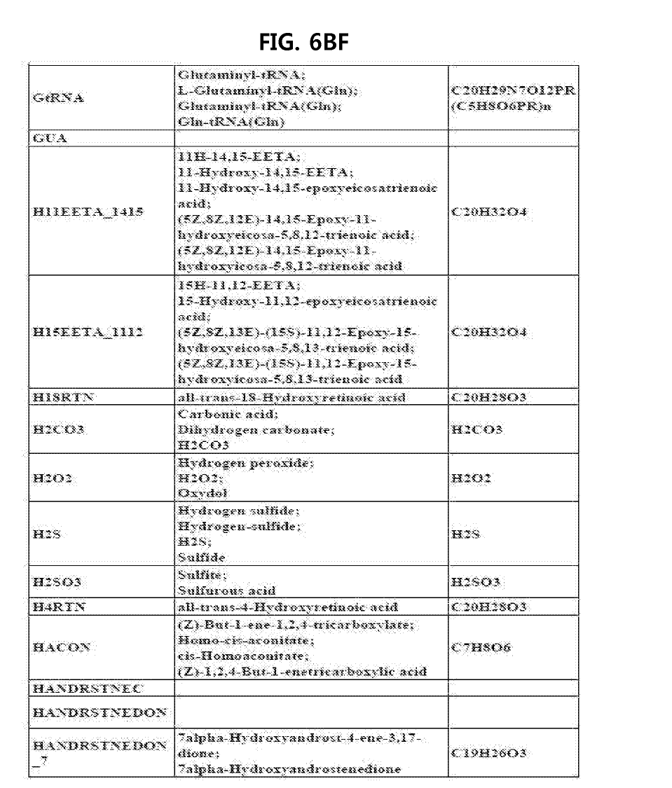

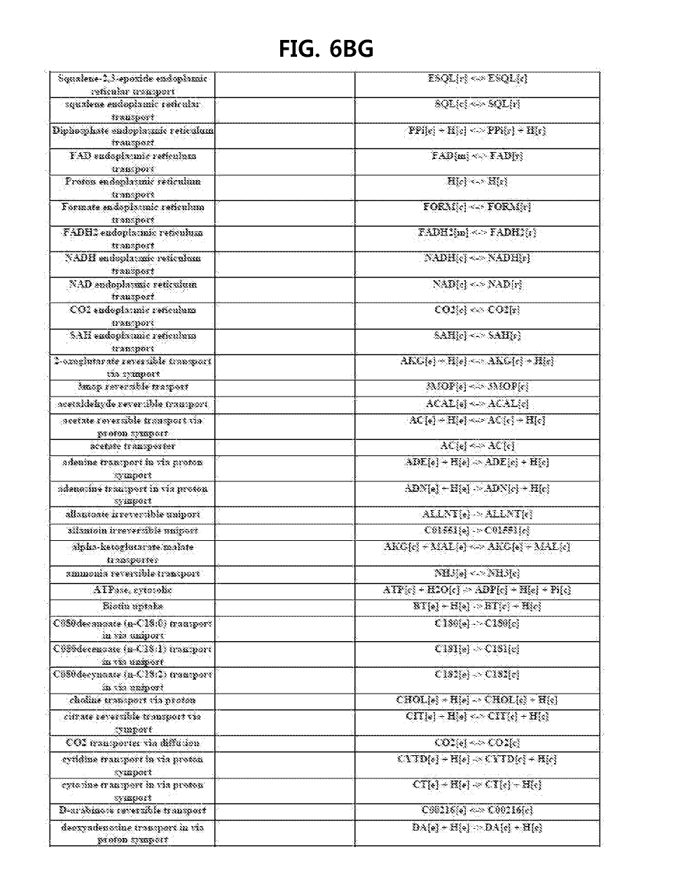

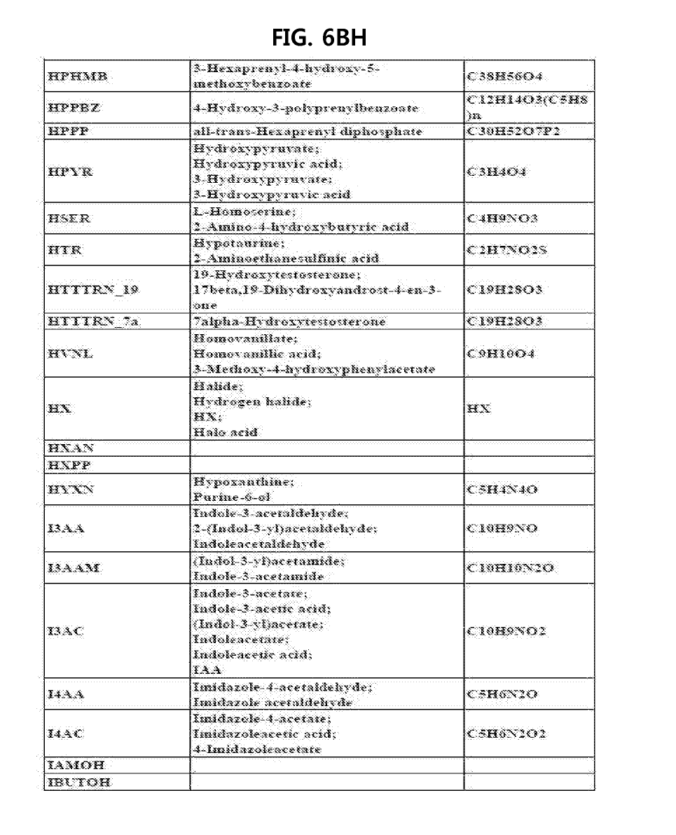

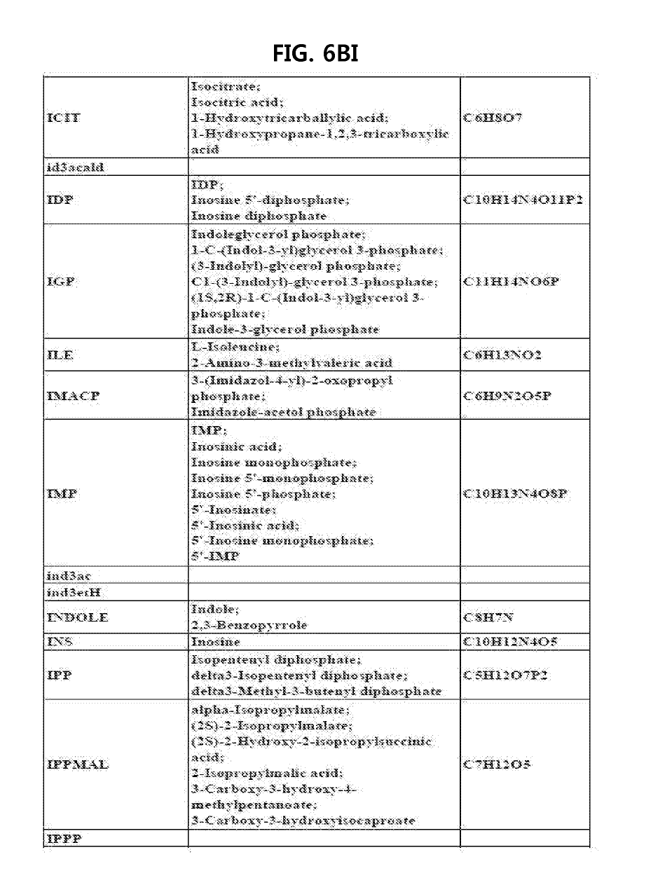

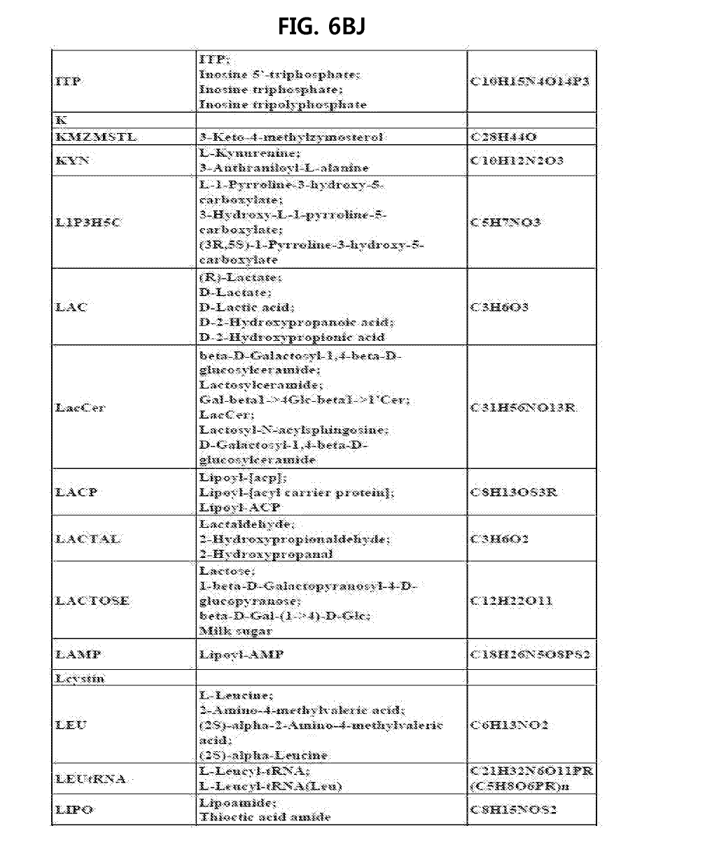

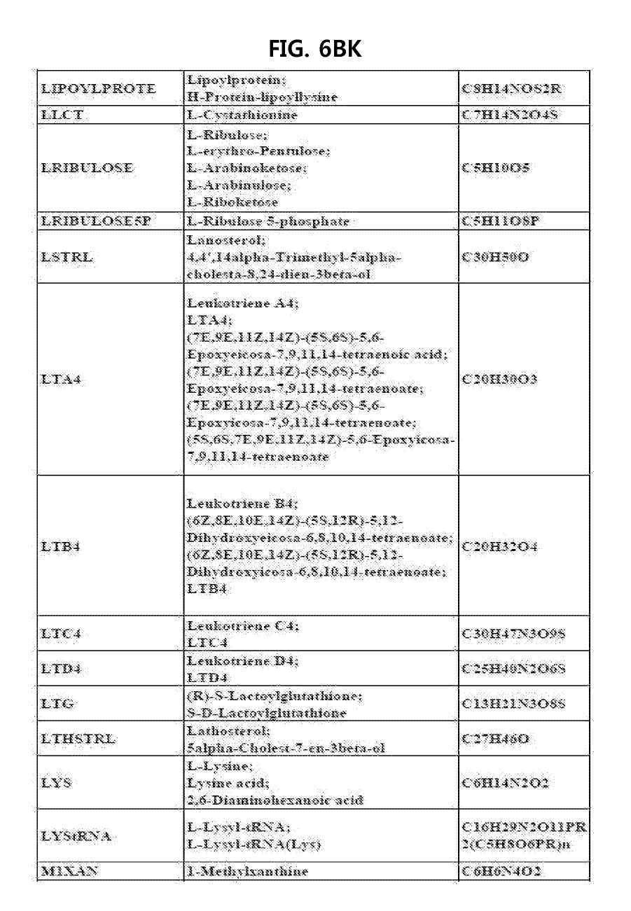

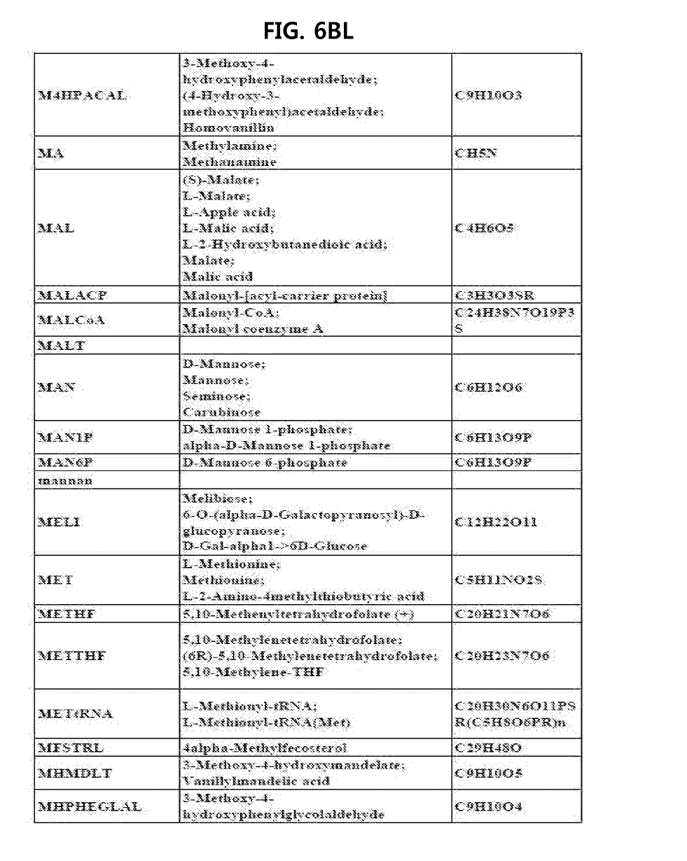

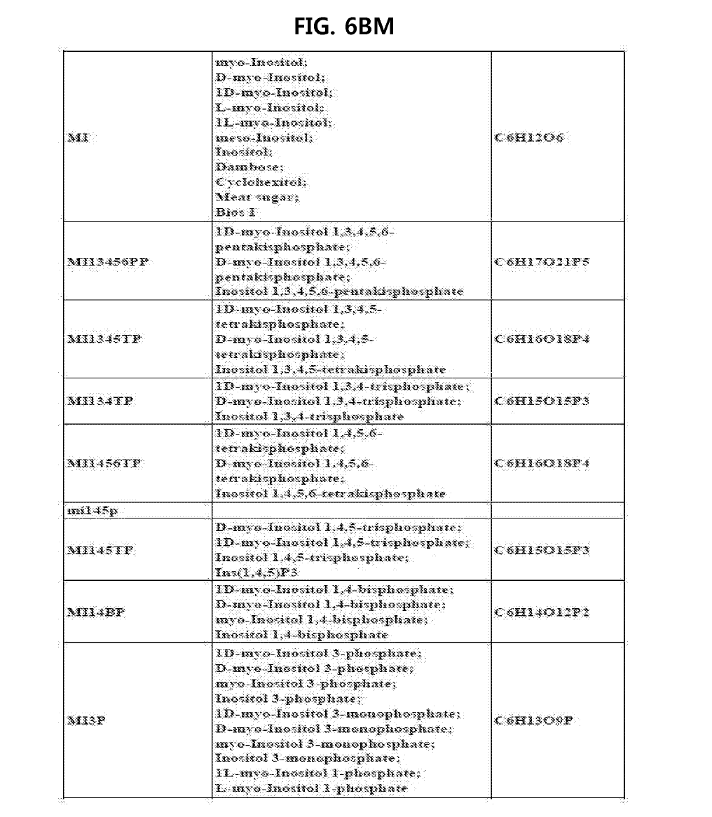

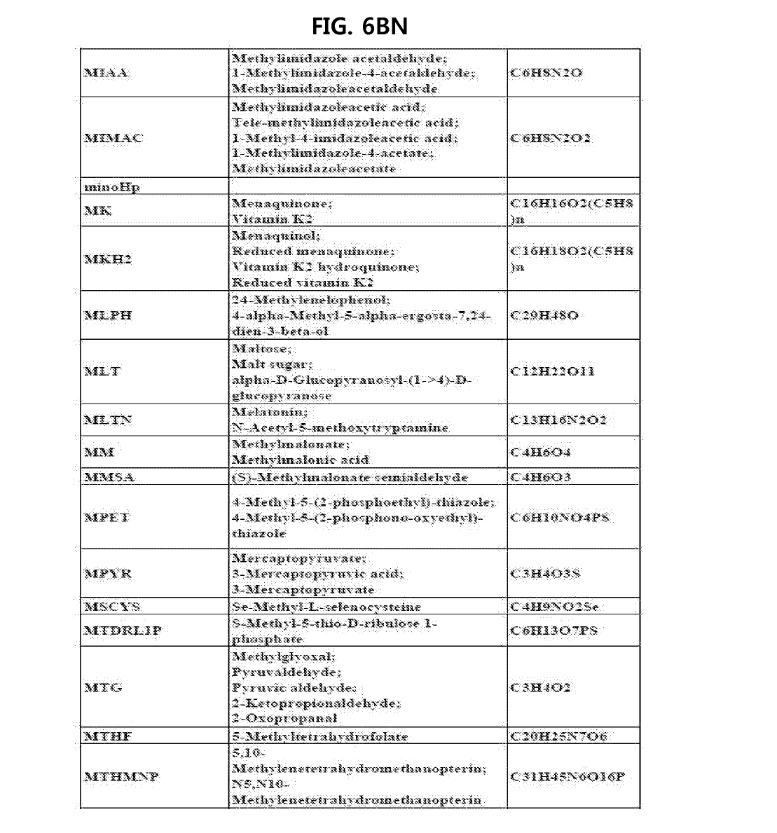

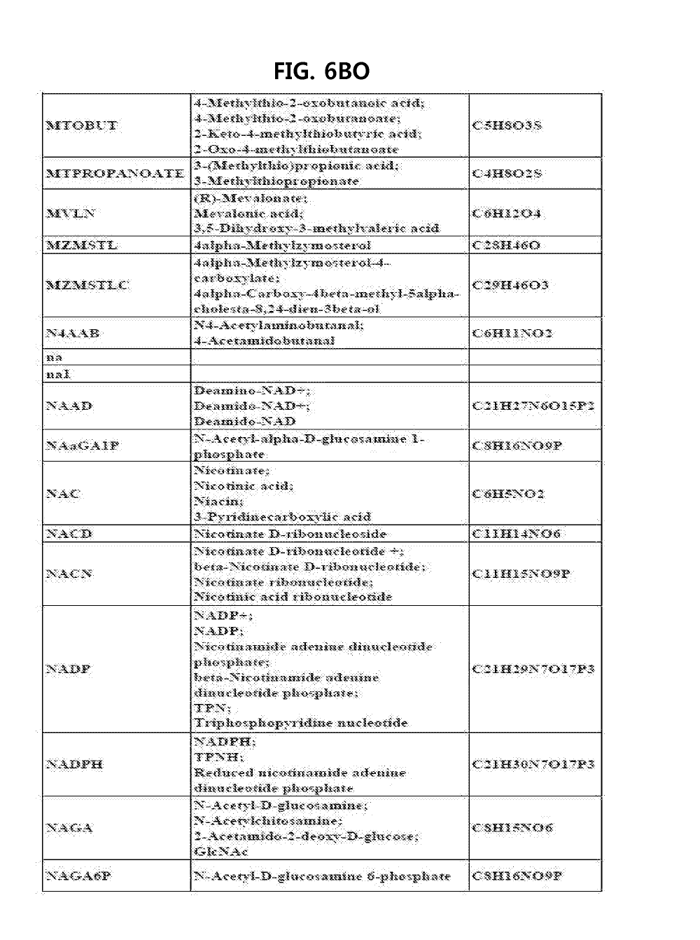

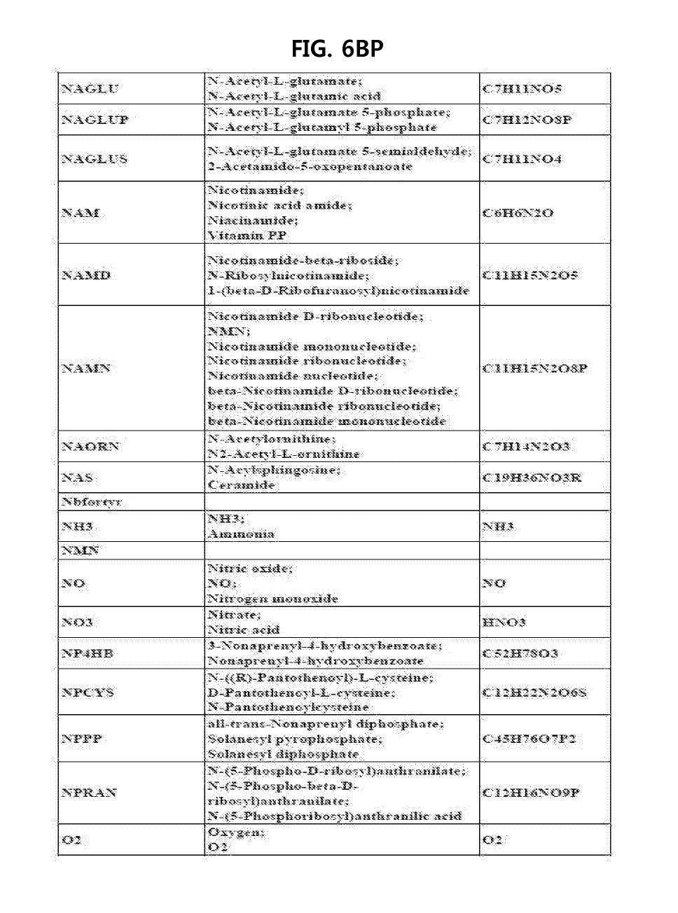

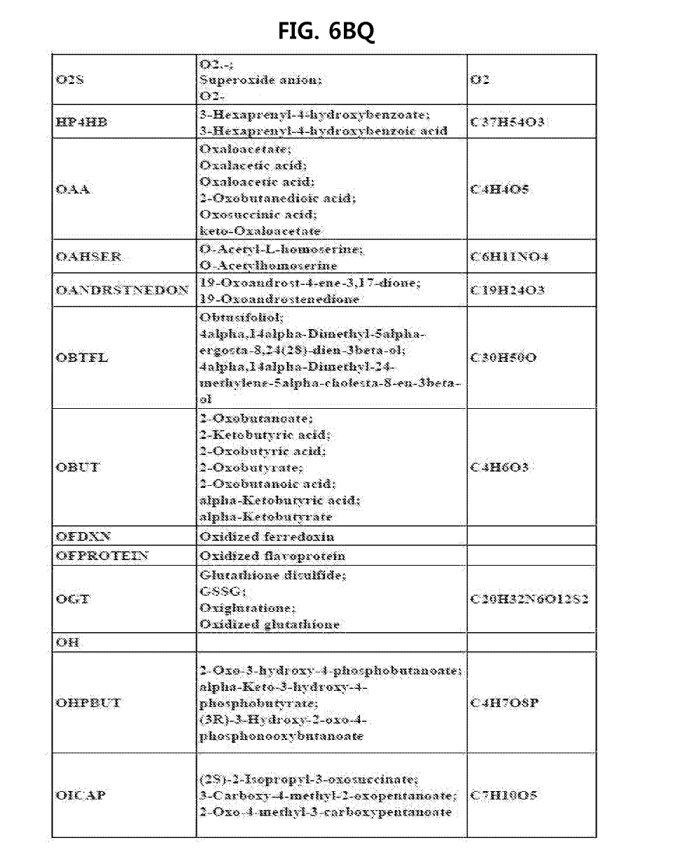

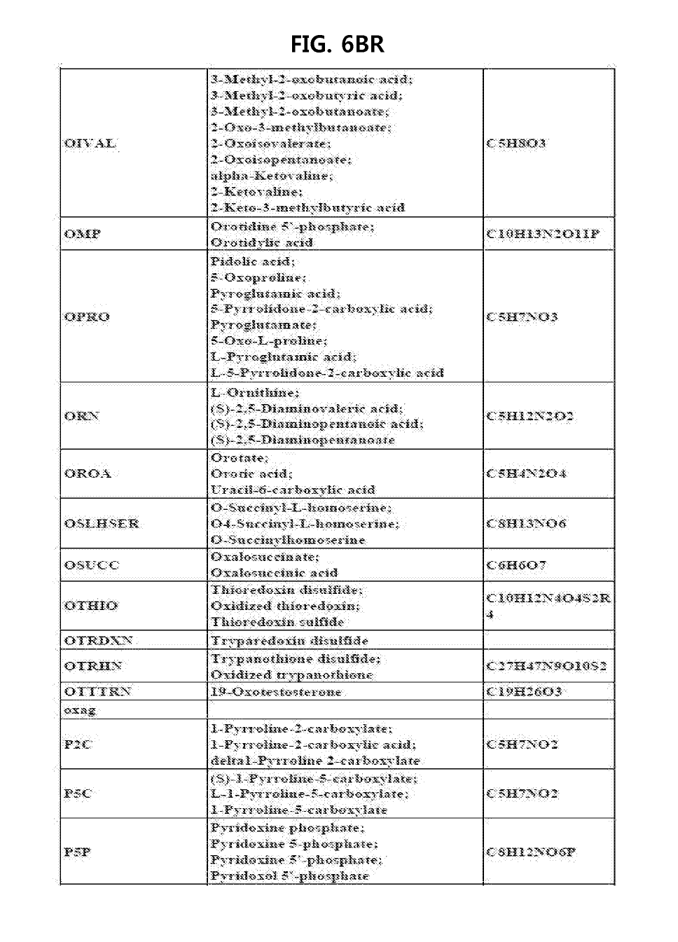

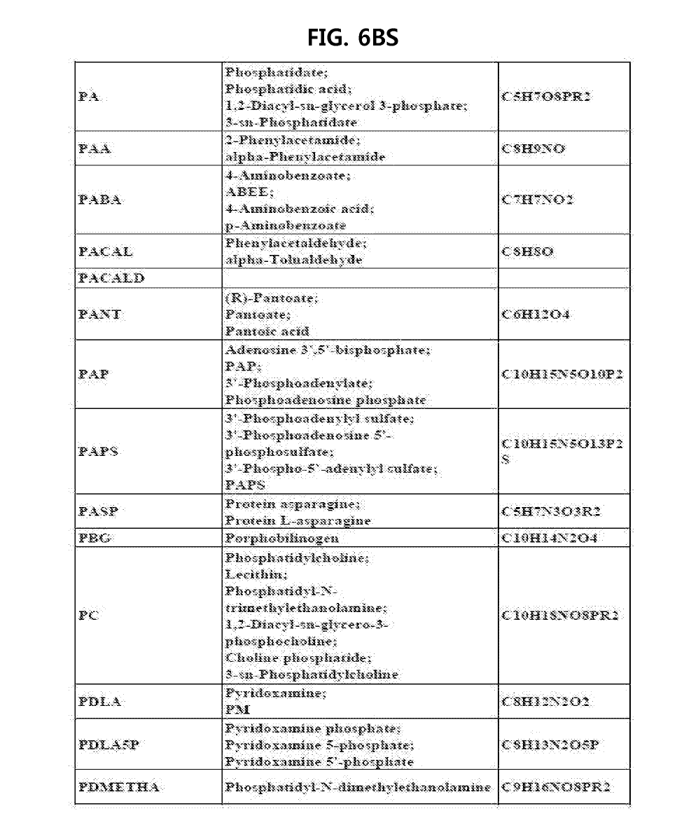

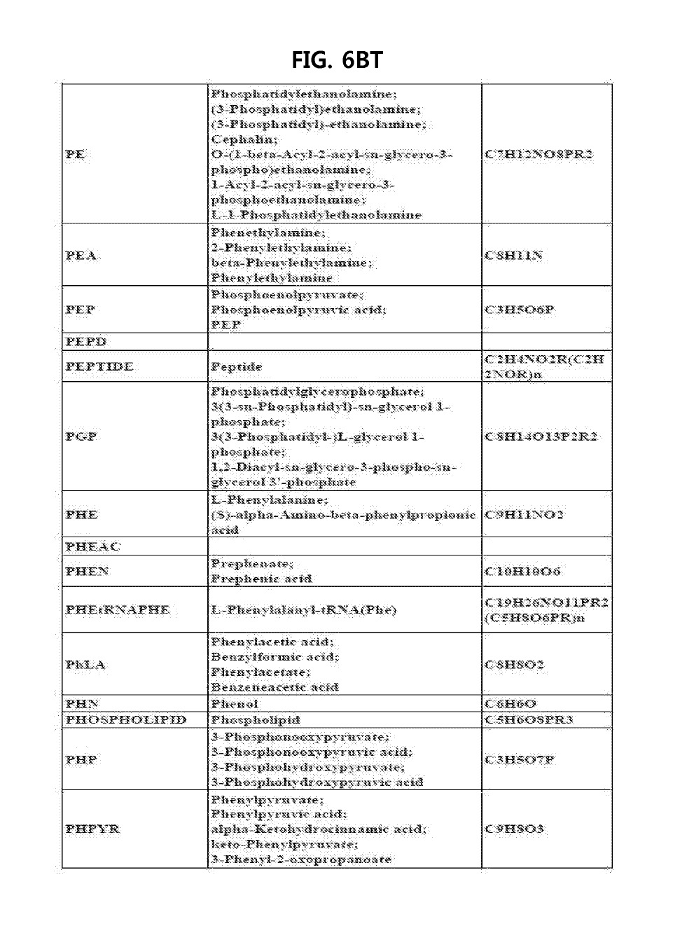

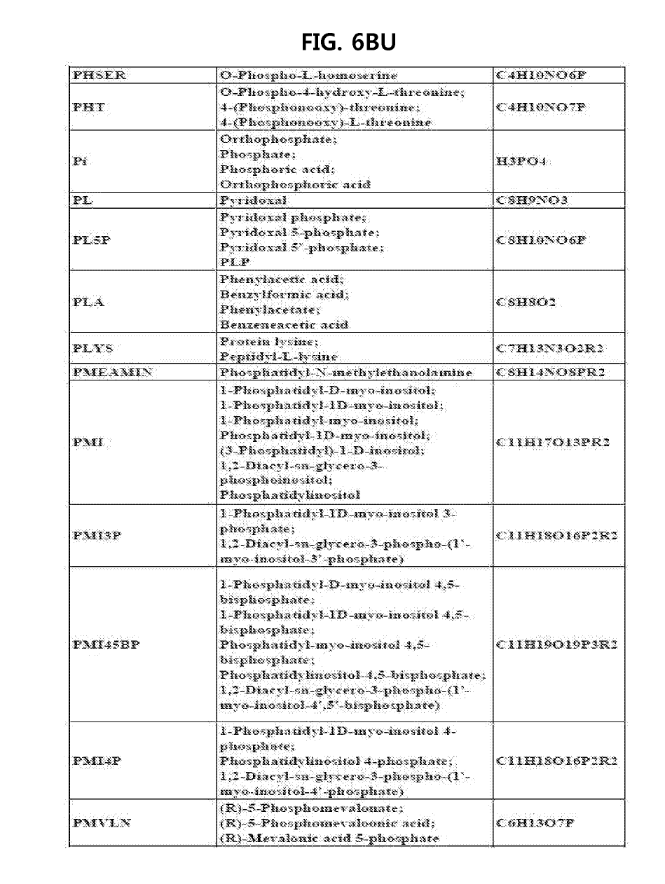

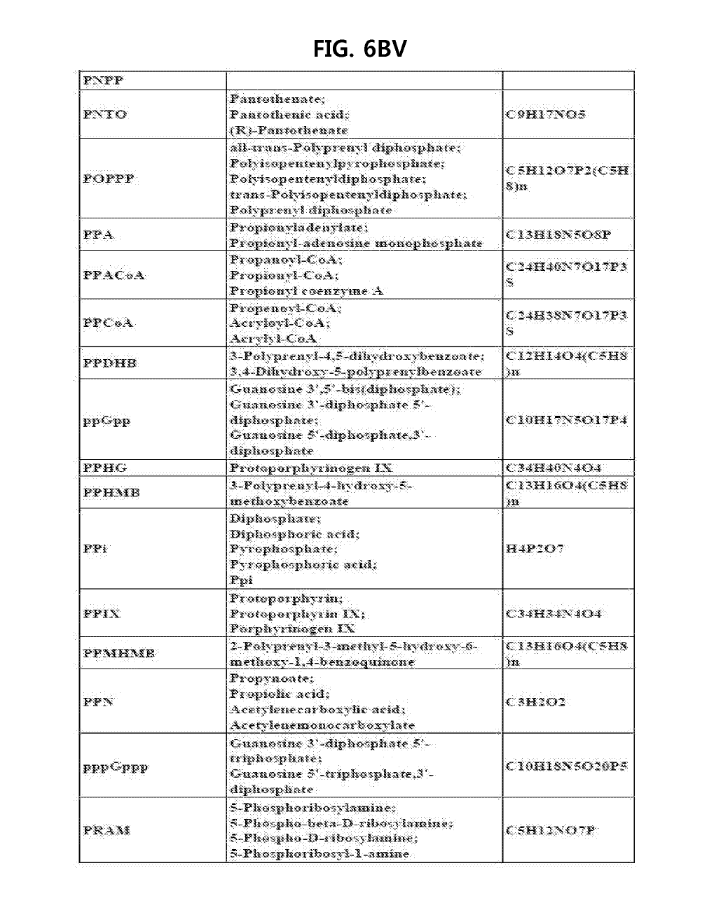

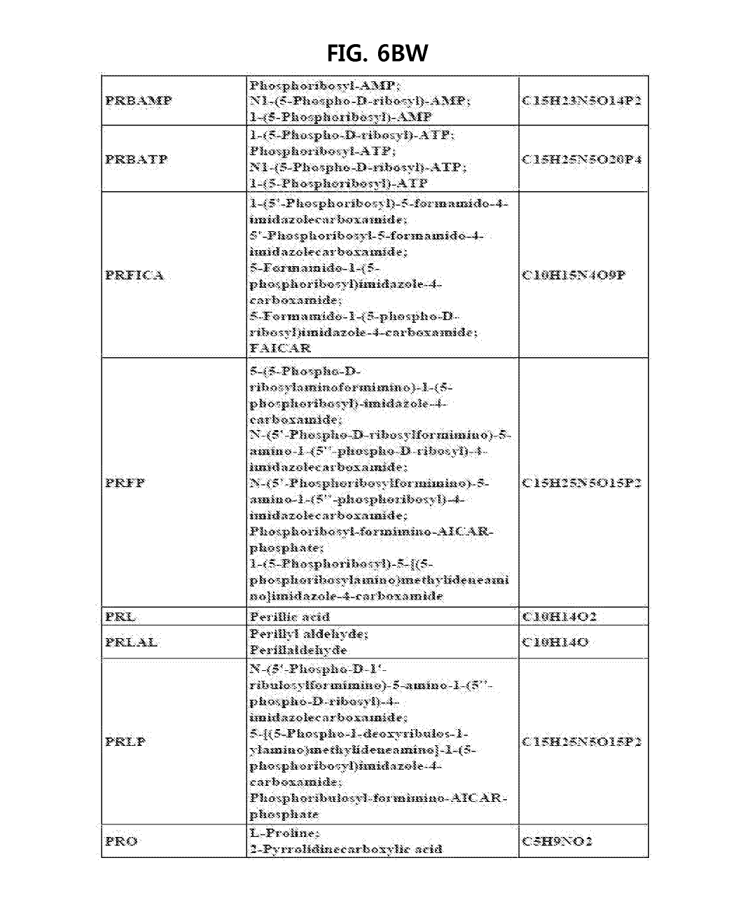

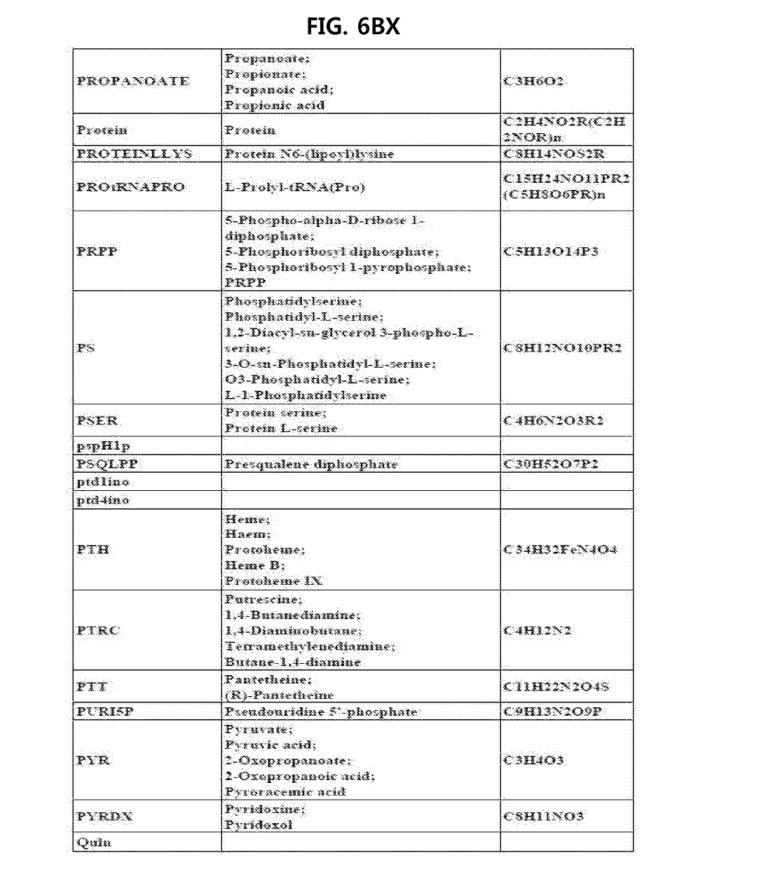

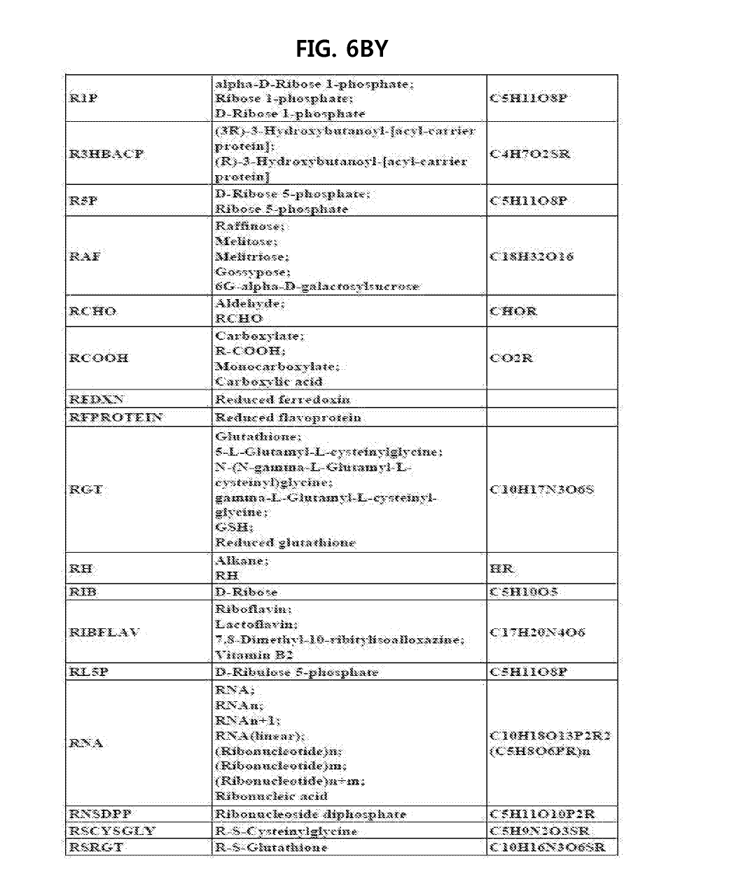

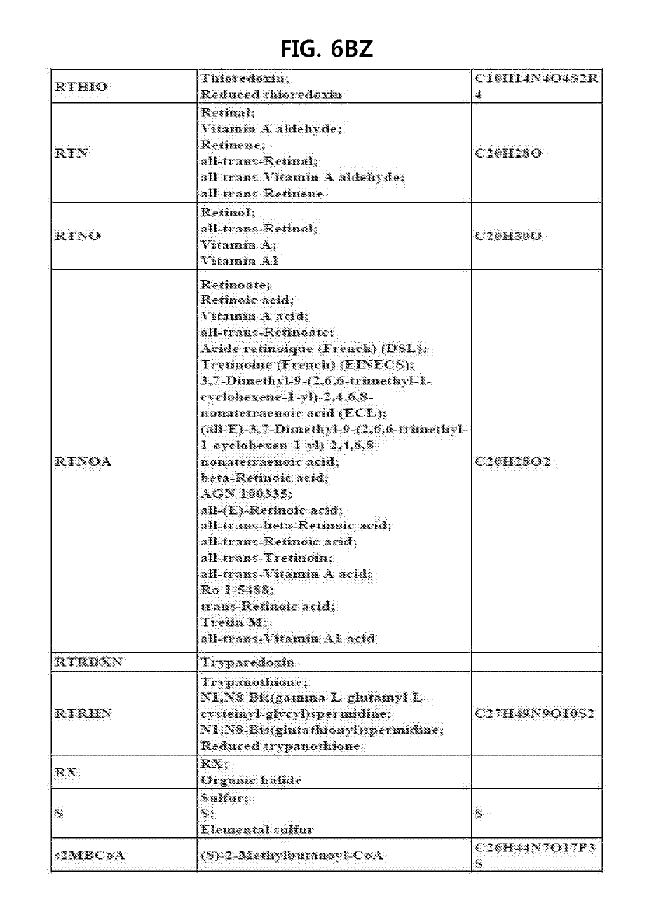

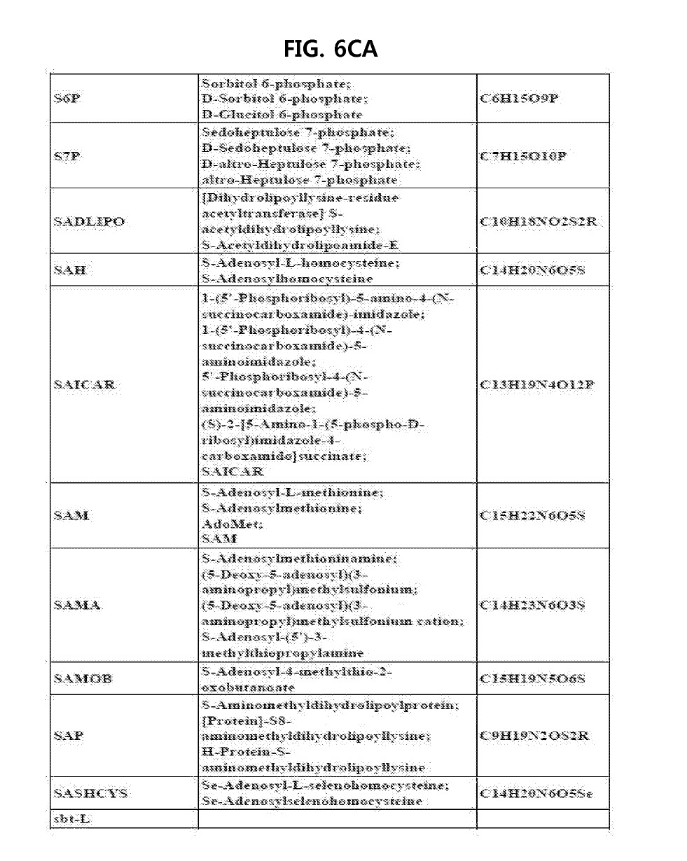

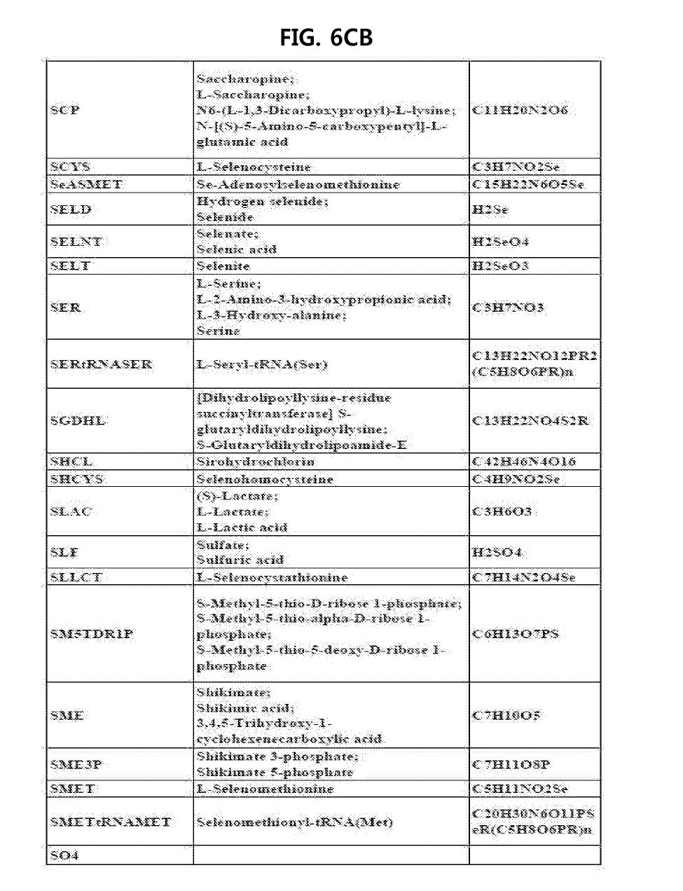

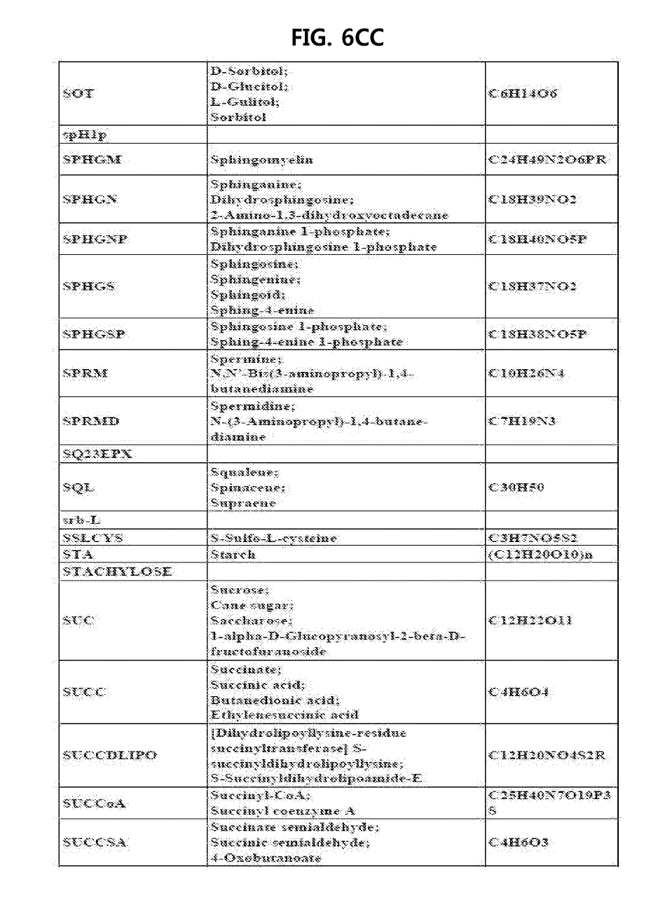

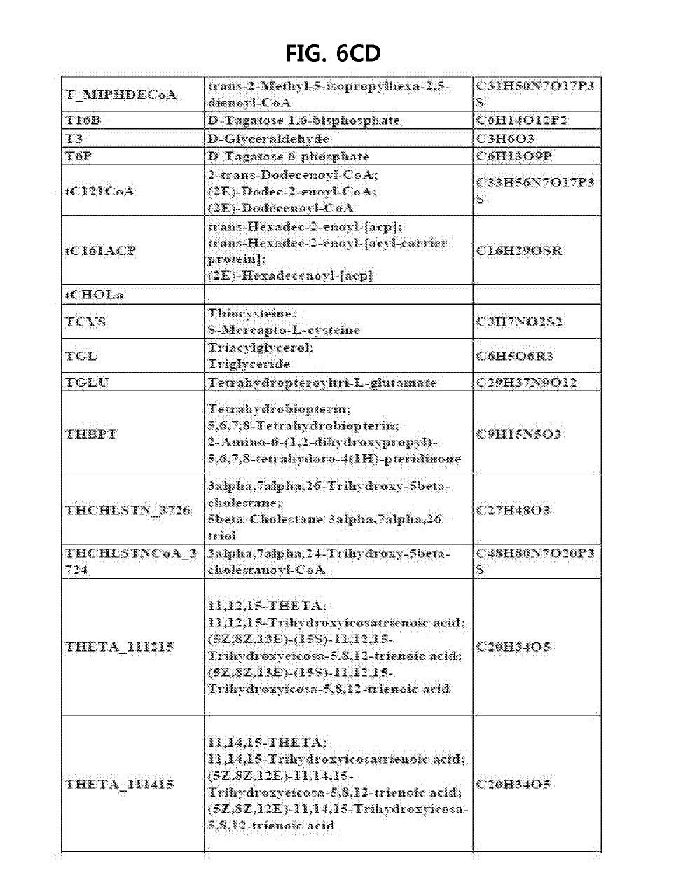

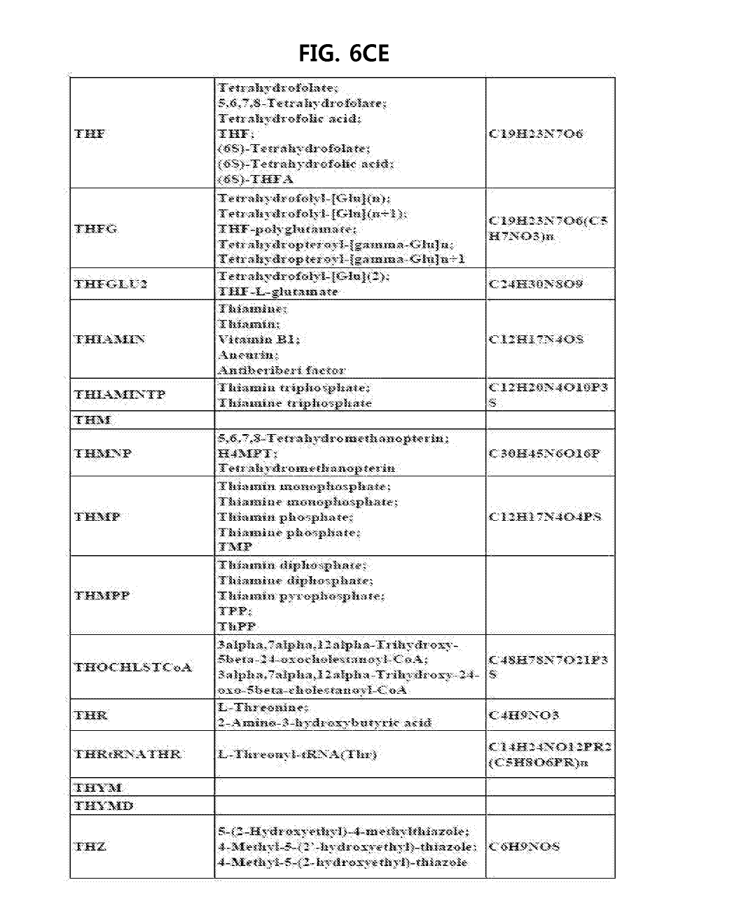

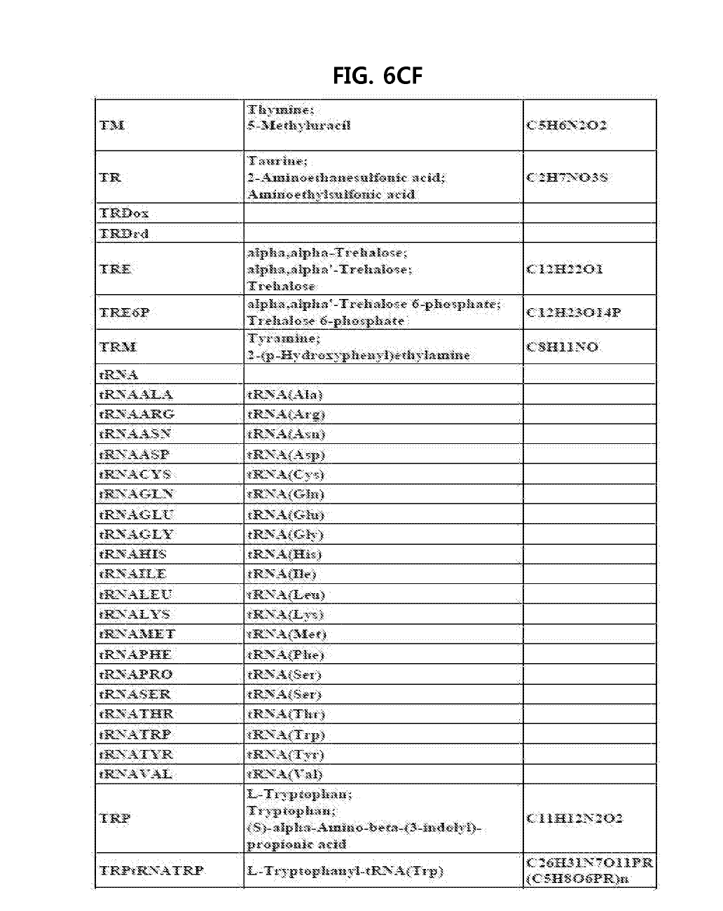

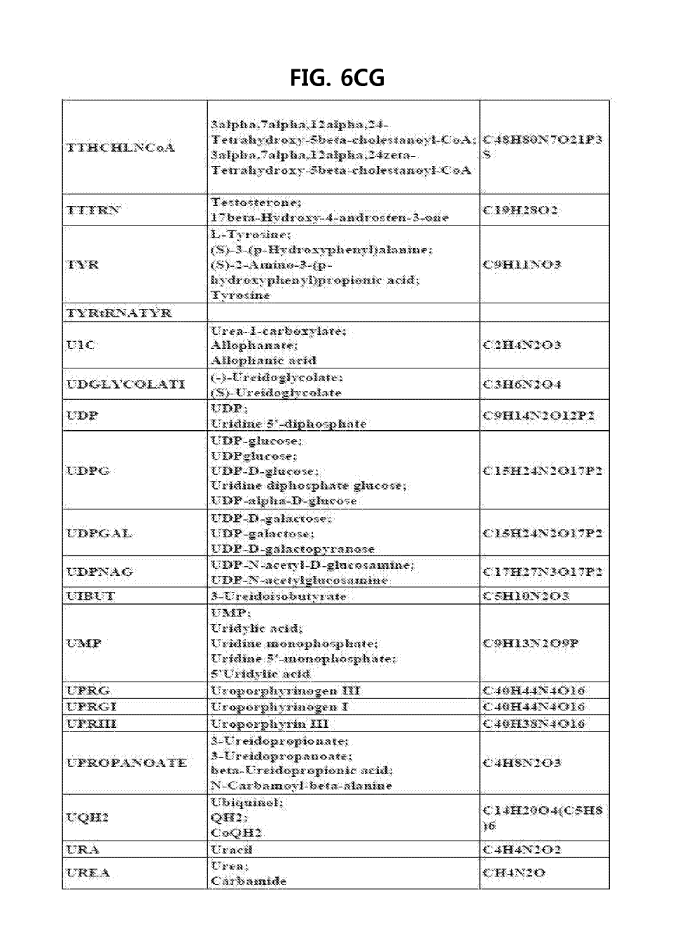

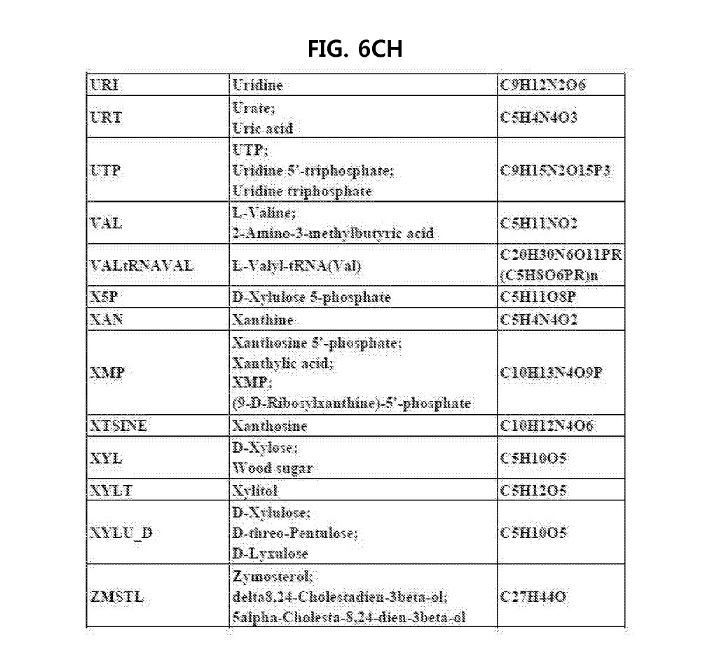

TABLE 1
GPR relationships used for the constructed metabolic network

| | |
|---|---|
| Protein Reaction Network | See FIGS. 5A-5BX |
| Abbreviations | See FIGS. 6A-6CH |

TABLE 2
Macromolecular compositions of K. marxianus

| Component | g · g⁻¹ DW |
|---|---|
| Protoplast | |
| Protein | 0.546 |
| DNA | 0.007 |
| RNA | 0.107 |
| Lipids | 0.052 |
| Cell wall | |
| Carbohydrates | 0.265 |
| SUM | 1.000 |

TABLE 3
Amino Acid Compositions

| | % protein | MW, | mmol/g |
|---|---|---|---|
| Amino acid | (w/w) | g/mol | protein |
| Alanine | 0.068 | 71.09 | 0.959 |
| Arginine | 0.046 | 156.20 | 0.296 |
| Asparagine | 0.033 | 114.12 | 0.286 |
| Aspartate | 0.033 | 115.10 | 0.286 |
| Cysteine | 0.004 | 103.16 | 0.034 |
| Glutamate | 0.047 | 128.15 | 0.366 |
| Glutamine | 0.047 | 129.15 | 0.366 |
| Glycine | 0.068 | 57.07 | 1.189 |
| Histidine | 0.034 | 137.16 | 0.251 |
| Isoleucine | 0.067 | 113.18 | 0.593 |
| Leucine | 0.086 | 113.18 | 0.760 |
| Lysine | 0.028 | 128.19 | 0.220 |
| Methionine | 0.019 | 131.21 | 0.148 |
| Phenylalanine | 0.070 | 147.19 | 0.477 |
| Proline | 0.116 | 97.13 | 1.198 |
| Serine | 0.056 | 87.09 | 0.648 |
| Threonine | 0.078 | 101.12 | 0.768 |
| Tryptophan | 0.001 | 186.23 | 0.004 |
| Tyrosine | 0.018 | 163.19 | 0.113 |
| Valine | 0.079 | 99.15 | 0.801 |

| | |
|---|---|
| Energy requirement for polymerisation(ATP): | 41.5 |

TABLE 4
DNA Compositions

| | mol/mol | MW | mmol/g |
|---|---|---|---|
| Nucleotide | DNA | g/mol | DNA |
| dAMP | 0.299 | 313.2 | 0.963 |
| dCMP | 0.201 | 289.2 | 0.647 |
| dTMP | 0.299 | 304.2 | 0.963 |
| dGMP | 0.201 | 329.2 | 0.647 |

| | |
|---|---|
| Energy requirement for polymerisation(ATP): | 4.40 |

TABLE 5
RNA Compositions

| | | mol/mol | | | | |
|---|---|---|---|---|---|---|
| | mRNA | RNA rRNA | tRNA | MW | mol/mol | mmol/g |
| Nucleotide | 5% | 75% | 20% | g/mol | RNA | RNA |
| AMP | 0.299 | 0.267 | | 329.2 | 0.215 | 0.835 |
| GMP | 0.201 | 0.258 | | 345.2 | 0.204 | 0.790 |
| CMP | 0.201 | 0.194 | | 305.2 | 0.156 | 0.604 |
| UMP | 0.299 | 0.281 | | 306.2 | 0.226 | 0.876 |

Energy requirement for polymerisation(ATP): 1.25

TABLE 6
Molecular mass of phospholipids composition

| | | MW, g/mol | |
|---|---|---|---|
| | | # of fatty acids | |
| Constituent | backbone | residues | total |
| Fecosterol | 398.664 | 0 | 398.66 |
| Phosphatidylinositol | 300.200 | 2 | 762.74 |
| Phosphatidylcholine | 223.207 | 2 | 685.75 |
| Phosphatidylserine | 223.121 | 2 | 685.66 |
| Phosphatidylethanolamine | 181.128 | 2 | 643.67 |
| Phosphatidylglycerol | 212.139 | 2 | 674.68 |
| Phosphatidic acid | 228.094 | 2 | 690.63 |
| Cardiolipin | 332.183 | 4 | 1257.26 |

TABLE 7
Composition of fatty acids in phospholipids

| | | | mmol/g | mol/mol |
|---|---|---|---|---|
| | g/g total | MW, | total fatty | total fatty |
| Fatty acid | fatty acids | g/mol | acids | acids |
| C08 | 0.1136484 | 144 | 0.79 | 0.183 |
| C10 | 0.047444 | 172 | 0.28 | 0.064 |
| C12 | 0.0432309 | 200 | 0.22 | 0.050 |
| C14 | 0.073 | 228 | 0.32 | 0.074 |
| C16 | 0.435 | 255 | 1.71 | 0.394 |
| C16:1 | | 253 | 0.00 | 0.000 |
| C18 | 0.288 | 283 | 1.02 | 0.235 |
| C18:1 | | 281 | 0.00 | 0.000 |

| | | | |
|---|---|---|---|
| Average molecular weight | 231 | SUM: | 0.70 |

TABLE 8
Micromolecule Compositions

| | | | mmol/g pool |
|---|---|---|---|
| | | g/g pool of small | of small |
| Molecular | MW, g/mol | molecules | molecules |
| NAD | 664.438 | 0.125 | 0.188 |
| NADP | 744.418 | 0.125 | 0.168 |
| COA | 767.534 | 0.125 | 0.163 |
| Q | 853.365 | 0.125 | 0.146 |
| THF | 445.434 | 0.125 | 0.281 |
| hemeA | 852.837 | 0.125 | 0.147 |
| FMN | 456.348 | 0.125 | 0.274 |
| FAD | 785.557 | 0.125 | 0.159 |

TABLE 9
Carbohydrate Compositions

| | | | mmol/g |
|---|---|---|---|
| Component | Molar ratio | MW, g/mol | carbohydrate |
| Chitin | 0.0 | 185 | 0.101 |
| a-Glucan | 0.3 | 162 | 3.026 |
| b-Glucan | 0.3 | 162 | 3.026 |

1. Protein Biosynthesis Equation (Mmol for Synthesizing 1 g of Protein):

0.959 ala_{—}c+0.266 arg_{—}c+0.286 asn_{—}c+0.286 asp_{—}c+0.034 cys_{—}c+0.366 gln_{—}c+0.366 glu_{—}c+1.189 gly_{—}c+0.251 his_{—}c+0.593 ile_{—}c+0.760 leu_{—}c+0.220 lys_{—}c+0.148 met_{—}c+0.477 phe_{—}c+1.198 pro_{—}c+0.648 ser_{—}c+0.768 thr_{—}c+0.004 trp_{—}c+0.113 tyr_{—}c+0.801 val_{—}c+41.5 atp_{—}c->41.5 adp_{—}c+41.5 pi_{—}c+PROTEIN

2. DNA Biosynthesis Equation (Mmol for Synthesizing 1 g of DNA):

0.963 datp_{—}n+0.647 dctp_{—}n+0.963 dttp_{—}n+0.647 dgtp_{—}n+26.0 atp_{—}n->DNA+26.0 adp_{—}n+26.0 pi_{—}n+3.223 ppi_{—}n

3. RNA Biosynthesis Equation (Mmol for Synthesizing 1 g of RNA):

0.835 atp_{—}n+0.790 gtp_{—}n+0.604 ctp_{—}n+0.8876 utp_{—}n+1.25 atp_{—}n->RNA+1.25 adp_{—}n+1.25 pi_{—}n+3.119 ppi_{—}n

4. Phospholipids Biosynthesis Equation (Mmol for Synthesizing 1 g of Phospholipids):

0.229 ptd1ino_{—}c+0.1 pc_{—}c+0.038 ps_{—}c+0.1 pe_{—}c+0.324 pa_{—}c+0.026 cl_{—}m+0.012 fecost_{—}r->PHOSPHOLIPID

5. Micromolecular Substance Biosynthesis Equation (Mmol for Synthesizing 1 g of Micromolecule):

0.188 NAD_{—}c+0.168 NADP_{—}c+0.163 CoA_{—}c+0.146 Q_{—}m+0.281 THF_{—}c+0.274 FMN_{—}c+0.159 FAD_{—}c+0.1 P5P_{—}c->COF

6. Carbohydrates Biosynthesis Equation (Mmol for Synthesizing 1 g of Carbohydrates):

3.026 Adglcn_{—}c+3.026 13BDglcn_{—}c+0.101 C00461+12.8 atp_{—}c->CARBOHYDRATE+12.8 adp_{—}c+12.8 pi_{—}c

Also, the biomass synthesis equation (cell growth equation) acquired from the compositions is as follows, and it was applied for a method of choosing an optimized metabolic pathway by maximizing the biomass synthesis equation in accordance with the present invention. The amounts of each of the categories of components listed in the biomass synthesis equation are based on the millimoles of the above percent compositions converted into grams.

7. Biomass synthesis equation (g for synthesizing 1 g of biomass)

0.56 PROTEIN+0.107 RNA+0.007 DNA+0.052 PHOSPHOLIPID+0.03 COF+0.110 CW+0.265 CARBOHYDRATE+70.37 ATP->BIOMASS+70.37 ADP+70.37 Pi

### Example 2

### Identification of Excellence of K. Marxianus as a 3HP Producing Strain and Estimation of 3HP Productivity Optimized Pathway Through Metabolic Network Construction and Metabolic Flux Analysis of K. marxianus

A draft metabolic network of K. marxianus was constructed using the strain's cell composition information and GPR relationships (gene-protein-reaction relationship) for enzyme reaction equations that were acquired based on genomic information of K. marxianus. Three enzyme reactions that may be introduced to K. marxianus were added for producing a metabolic product (3HP in this case) to construct a metabolic network of 3HP producing K. marxianus.

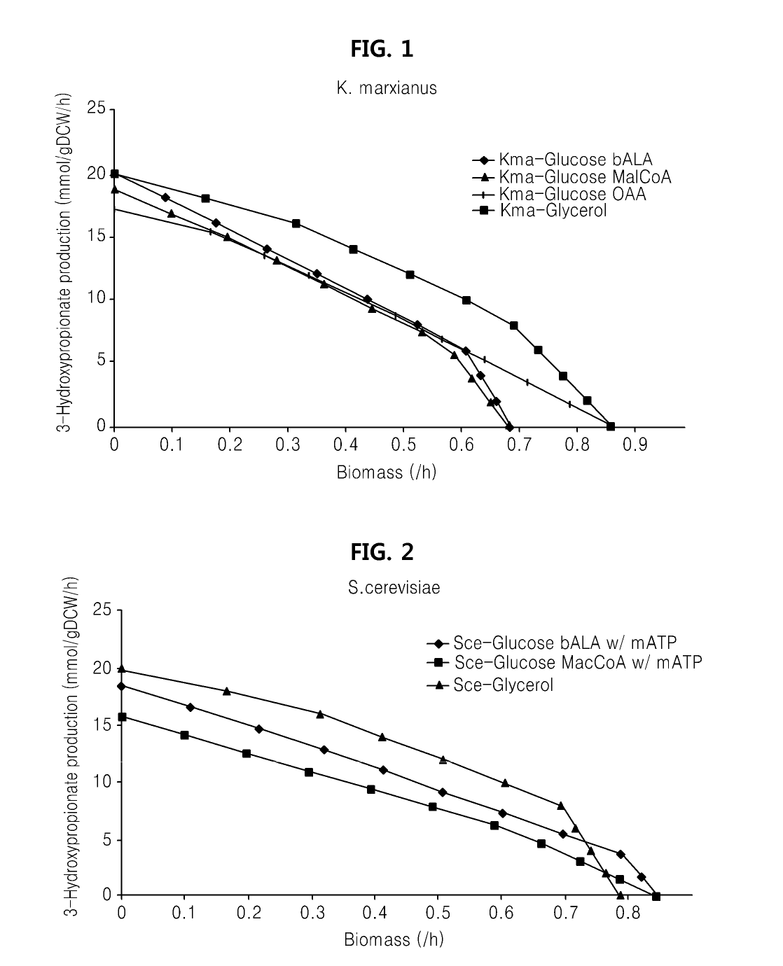

The constructed metabolic network was applied to select an external enzyme reaction that provides the most optimized metabolic pathway. At this time, a 3HP producing reaction rate and a cell growth rate were selected as object functions to identify whether the most appropriate optimized metabolic pathway is provided by identifying the trade-off relations between the two, and the trade-off graph is shown in FIG. 1.

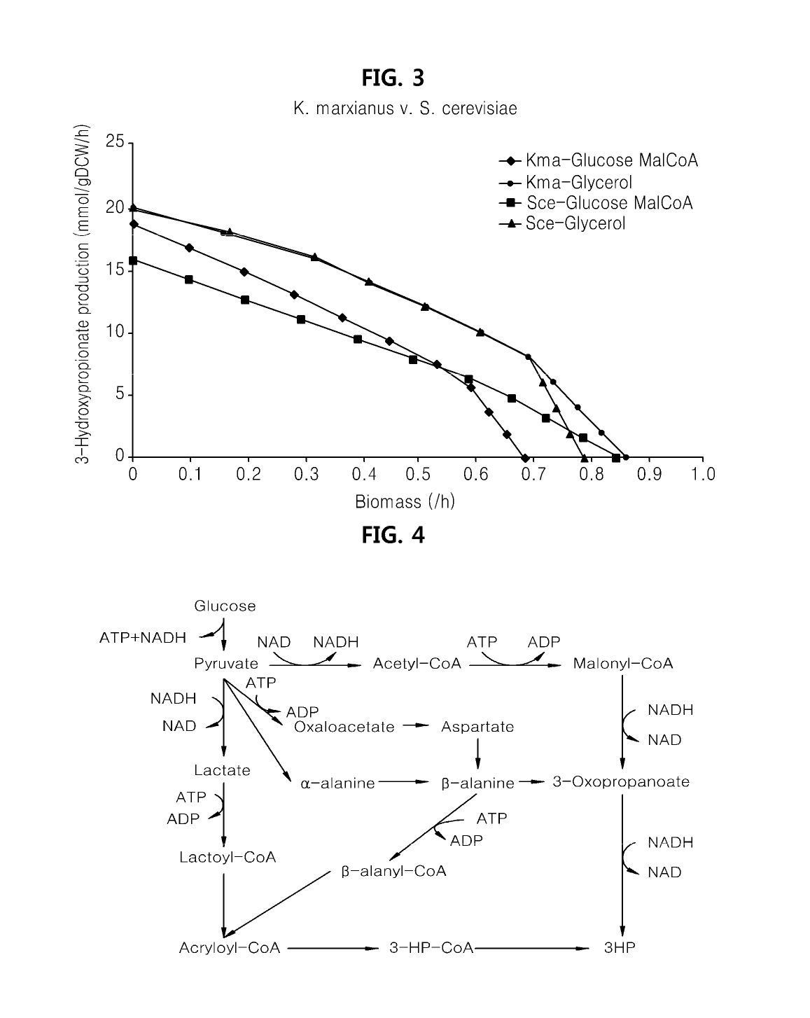

Also, a 3HP producing trade-off curve of S. cerevisiae, the most abundant yeast strain, was acquired and is shown in FIG. 2, and the graph that compared them is shown in FIG. 3. As a result, first, the 3HP productivity of the three external enzyme reactions introduced to K. marxianus were able to be compared through FIG. 1.

Next, it is identified from FIG. 3 that K. marxianus showed higher maximum production quantity of 3HP when Glucose Malonyl-CoA pathway was used for a 3HP production pathway for K. marxianus and S. cerevisiae, which shows that 3HP productivity for the newly developed 3HP producing K. marxianus strain is higher than that of the previously east strain.

Therefore, the suitability of K. marxianus strain developed through an external enzyme reaction introduction as an effective 3HP producing strain is demonstrated.

The abbreviations and official names of the metabolic products used for the present invention's metabolic network are organized in Table 1, FIGS. 5A-5BX and FIGS. 6A-6CH.

According to the present embodiment, the metabolic network model is useful in designing an optimized metabolic pathway to enhance the production of a desired metabolic product by analyzing metabolic flux and metabolic characteristics of 3HP producing microorganisms. Also, the method of designing an optimized metabolic pathway based on the metabolic network model of 3HP producing microorganisms according to the embodiment enables estimation to be done at a system level in a short time, unlike the productivity enhancing pathway search based on intuition and deduction of humans. Also, there are advantages of saving time and cost by introducing the external enzyme reactions designated by the method in 3HP producing strains and conveniently acquiring transformed organisms that may produce specific metabolic products such as 3HP with a high efficiency.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of identifying an optimized metabolic pathway for the production of a metabolic product, the method comprising:
(a) providing a metabolic network model in a microorganism and a biomass synthesis equation using at least one information of a culture condition of microorganisms, metabolic products produced by microorganisms, and cell composition of microorganisms, based on a database including information on biochemical reactions in which enzymes are involved in a metabolic network within microorganisms;
(b) providing a primary modified metabolic pathway by introducing a biochemical reaction pathway that does not exist in the acquired metabolic pathway;
(c) providing a secondary modified metabolic pathway by modifying at least one enzyme reaction in the primary modified metabolic pathway;
(d) acquiring information about a metabolic product and/or biomass produced by the secondary modified metabolic pathway;
(e) acquiring a metabolic product-biomass correlation equation based on the acquired metabolic product information and/or biomass information;
(f) repeating steps (c)-(e) for different enzyme reactions; and
(g) identifying the secondary modified metabolic pathway that provides the optimum metabolic pathway for production of the metabolic product based on the product-biomass correlation equation.

2. The method of claim 1, wherein the biochemical reaction pathway that does not exist in the acquired metabolic pathway is at least one pathway selected from the group consisting of a malonyl-CoA pathway, a β-alanine pathway, and a glycerol pathway.

3. The method of claim 2, wherein introducing the malonyl-CoA pathway comprises introducing 3-oxopropanoate:NADP+oxidoreductase (EC 1.2.1.18) and 3-hydroxypropionate dehydrogenase (EC 1.1.1.59).

4. The method of claim 2, wherein introducing the β-alanine pathway comprises introducing 3-hydroxypropionate dehydrogenase (EC 1.1.1.59).

5. The method of claim 2, wherein introducing the glycerol pathway comprises introducing glycerol dehydratase (EC 4.2.1.30) and aldehyde dehydrogenase (EC 1.2.1.3).

6. The method of claim 1, wherein modifying at least one enzyme reaction in step (c) comprises removing one or more enzymes from the metabolic pathway.

7. The method of claim 1, wherein the microorganism is Kluyveromyces marxianus.

8. The method of claim 1, wherein the culture condition of the microorganism is any one selected from the group consisting of a carbon source, a nitrogen source, and an oxygen condition used by the microorganisms.

9. The method of claim 8, wherein the carbon source is any one selected from the group consisting of glucose, fructose, mannose, and galactose.

10. The method of claim 8, wherein the nitrogen source is any one selected from the group consisting of an amino acid, amide, amine, a nitrate salt, and an ammonium salt.

11. The method of claim 8, wherein the oxygen condition is an aerobic condition, a low-oxygen condition, or an anaerobic condition.

12. The method of claim 1, wherein the metabolic product is 3-hydroxypropionate.

13. The method of claim 7, wherein the microorganism is Kluyveromyces marxianus and has a biomass synthesis equation of Reaction Equation I:
0.56 PROTEIN+0.107 RNA+0.007 DNA+0.052 PHOSPHOLIPID+0.03 COF+0.110 CW+0.265 CARBOHYDRATE+70.37 ATP->BIOMASS+70.37 ADP+70.37 Pi.

14. A microorganism comprising an optimized secondary modified metabolic pathway identified by the method of claim 1.

15. The microorganism of claim 14, wherein the microorganism is Kluyveromyces marxianus.

16. The microorganism of claim 14, wherein the microorganism produces 3-hydroxypropionate.

17. A method of producing a specific metabolic product using a microorganism, the method comprising:
culturing the microorganism of claim 14; and
collecting a specific metabolic product from the culture.

18. The method of claim 17, wherein the specific metabolic product is 3-hydroxypropionate.

19. A method of identifying an optimized metabolic pathway for the production of a metabolic product, the method comprising:
(a) providing a metabolic network model of a microorganism;
(b) identifying a metabolic pathway in the network model;
(c) providing a primary modified metabolic pathway in the network model by introducing a simulated biochemical reaction pathway that did not previously exist in the network model;
(d) modifying at least one enzyme reaction in the primary modified metabolic pathway to provide a secondary modified metabolic pathway;
(e) determining the effect of the secondary modified metabolic pathway on the production of a metabolic product, growth of the microorganism, or both;
(f) calculating a metabolic product-biomass correlation equation based on the information;
(g) repeating steps (d)-(f) for different enzyme reactions in the primary modified metabolic pathway; and
(h) identifying the secondary modified metabolic pathway that provides the optimized metabolic pathway for production of the metabolic product based on the product-biomass correlation equation.
